(19) United States
(12) Patent Application Publication
(10) Pub. No.: US 2024/0360522 A1
(43) Pub. Date: Oct. 31, 2024

(54) IDENTIFYING MICROBIAL SIGNATURES AND GENE EXPRESSION SIGNATURES

(71) Applicant: Rutgers, The State University of New Jersey, New Brunswick, NJ (US)

(72) Inventors: Bassel Ghaddar, Highland Park, NJ (US); Subhajyoti De, Princeton Junction, NJ (US)

(73) Assignee: Rutgers, The State University of New Jersey, New Brunswick, NJ (US)

(21) Appl. No.: 18/287,776

(22) PCT Filed: Apr. 21, 2022
(86) PCT No.: PCT/US2022/025829

§ 371 Date: Oct. 20, 2023

Related U.S. Application Data
(60) Provisional application No. 63/177,696, filed on Apr. 21, 2021.

(51) Int. Cl. C12Q 1/689; C12Q 1/6886; C12Q 1/6895

(52) U.S. Cl. CPC C12Q 1/689; C12Q 1/6886; C12Q 1/6895; C12Q 2600/118; C12Q 2600/158

(57) ABSTRACT

Disclosed herein are systems and methods for identifying biomarkers. Biomarker identification can be achieved while increasing efficiency and decreasing data and computation complexity but maintaining accuracy. Such biomarker identification can be achieved via analysis of differential gene expression, such as determined using single cell RNA-sequencing data sets.

### CROSS-REFERENCE TO RELATED APPLICATIONS

This application claims priority to U.S. Provisional Application No. 63/177,696, filed Apr. 21, 2021, which is herein incorporated by reference in its entirety.

### ACKNOWLEDGMENT OF GOVERNMENT SUPPORT

This invention was made with government support under Contract number R21 CA248122 awarded by the National Institutes of Health. The government has certain rights in the invention.

### FIELD

The field relates to methods of identifying and using microbial signatures and gene expression signatures for diagnosing cancer and predicting cancer patient outcomes, and for identifying an infection in a subject, such as by query and reference inputs.

### OVERVIEW

The microbiome contributes to numerous aspects of human health and disease, including oncogenesis. While it is uncertain whether the healthy pancreas harbors its own microbiome, emerging evidence indicates that bacteria and fungi can translocate to the pancreas and induce local and systemic changes that promote the development of pancreatic ductal adenocarcinoma (PDA) (Vitiello et al. Trends in Cancer 5: 670-676, 2019; Wei et al. Mol. Cancer 18: 1-15, 2019). Microbiota products alter gene regulation (Yoshimoto et al. Nature 499: 97-101, 2013) and lead to DNA damage (Ogrendik, Gastrointest. tumors 3: 125-127, 2017), stimulate pattern recognition receptors that potentiate mutant KRAS signaling (Ochi et al. J. Exp. Med. 209: 1671-1687, 2012; Zambirinis et al. Cell Cycle, 12: 1153-1154, 2013), and can induce both inflammation and immunosuppression (Pushalkar et al. Cancer Discov. 8: 403-416, 2018; Zambirinis et al. J. Exp. Med. 212: 2077-2094, 2015; Aykut et al. Nature, 574: 264-267, 2019; Seifert et al. Nature, 532: 245-249, 2016). Microbiota within PDA also may confer resistance to therapies, including deactivating gemcitabine via microbial cytidine deaminase (Geller et al. Science, 357(6356):1156-1160, 2017), while antibiotic-induced reduction of the gut microbiome may increase sensitivity to immune checkpoint inhibitors (Pushalkar et al. Cancer Discov. 8: 403-416, 2018; Sethi et al. Gastroenterology 155: 33-37.e6, 2018; Thomas et al. Carcinogenesis 39: 1068-1078, 2018).

Several barriers limit the systematic investigation of the microbiome in PDA patients (Sethi et al. Gastroenterology 156: 2097-2115.e2, 2019). First, many intestinal microbes are difficult to culture in vivo (Suau et al. Appl. Environ. Microbiol. 65(11):4799-807, 1999). Second, microbiome composition can differ vastly (Ericsson et al. PLoS One, 10: e0116704, 2015; De Filippo et al. Proc. Natl. Acad. Sci. 107(33):14691-6, 2010; Nguyen et al. Dis. Model. Mech. 8(1): 1-16, 2015), and there are few model systems that sufficiently recapitulate tumor-microbiome interactions in humans (Mallapaty, Lab Anim. 46: 373-377, 2017; Saluja et al. Gastroenterology 144: 1194-1198, 2013). Third, the possibility of sample contamination post-surgery complicates data interpretation (de Goffau et al. Nat. Microbiol. 3: 851-853, 2018; Zinter et al. Microbiome 7: 1-5, 2019). Recently, using The Cancer Genome Atlas (TCGA), (Poore et al. Nature 579: 567-574, 2020) discovered cancer-type specific microbial signatures, and (Nejman et al. Science, 368(6494):973-980, 2020) identified tumor-specific intracellular bacteria through 16S rRNA profiling of hundreds of tumors. However, these studies analyzed genomic data from bulk tissue samples, which do not capture microbial-somatic cell enrichments, associations with cell-type specific activities, or microbial contributions to inter-cellular communication networks. In particular, PDA is characterized by a fibrotic stroma comprising the majority of tumor volume, which makes disentangling cellular relationships difficult by bulk profiling (Moffitt et al. Nat. Genet. 47: 1168-1178, 2015). As a result, the inventors develop SAHMI (Single-cell Analysis of Host-Microbiome Interactions) to examine patterns of human-microbiome interactions in the pancreatic tumor microenvironment at single cell resolution using genomic approaches.

### SUMMARY

The Summary is provided to introduce a selection of concepts in a simplified form that are further described below in the Detailed Description. The Summary is not intended to identify key features or essential features of the claimed subject matter, nor is it intended to be used to limit the scope of the claimed subject matter.

In one embodiment, a computer-implemented method of identifying biomarkers for diagnosing cancer in a subject comprises receiving single cell RNA sequencing datasets for at least two cohorts, wherein at least one cohort comprises one or more cancer subjects and at least one cohort comprises one or more non-cancer subjects; identifying microbial genera using the datasets, wherein the identifying generates at least one microbial genera signature for the one or more cancer subjects and at least one microbial genera signature for the one or more non-cancer subjects; and selecting microbial genera differentially present in the at least one microbial genera signature for the one or more cancer subjects compared to the at least one microbial genera signature for the one or more non-cancer subjects, wherein the selecting generates a differentiating microbial genera signature that distinguishes a cancer subject from a non-cancer subject. Such an embodiment may further comprise receiving a single cell RNA sequencing dataset for a subject at risk of having a cancer; identifying a set of microbial genera in the dataset for the subject at risk of having the cancer; and comparing the differentiating microbial genera signature to the set of microbial genera identified in the dataset from the subject at risk of having the cancer; thereby determining whether the subject at risk of having the cancer has the cancer.

In another embodiment, a computer-implemented method of identifying biomarkers for predicting a survival outcome in a cancer subject, comprises receiving single cell RNA sequencing datasets for at least two cohorts, wherein at least one cohort comprises one or more poor survival outcome cancer subjects and at least one cohort comprises one or more good survival outcome cancer subjects; identifying microbial genera in the datasets, wherein the identifying generates at least one microbial genera signature for the one or more good survival outcome cancer subjects and at least one microbial genera signature for the one or more poor survival outcome cancer subjects; and selecting microbial genera differentially present in the at least one microbial genera signature for the one or more good survival outcome cancer subjects compared to the at least one microbial genera signature for the one or more poor survival outcome cancer subjects, wherein the selecting generates a differentiating microbial genera signature that distinguishes a good survival outcome subject from a poor survival outcome subject. Such an embodiment can further comprise receiving a single cell RNA sequencing dataset for the cancer subject; identifying a set of microbial genera in the dataset for the cancer subject; and comparing the differentiating microbial genera signature to the set of microbial genera identified in the dataset from the cancer subject; thereby predicting whether the cancer subject will have a good survival outcome or a poor survival outcome.

In yet another embodiment, a computer-implemented method of determining T-cell microenvironment reaction in a cancer subject, comprises receiving a single cell RNA sequencing dataset for T-cells from the subject; determining the expression level of one or more of the genes of Table 2 in the T-cells; and comparing the expression level of the one or more genes of Table 2 in the T-cells to a control using a random forest model, thereby classifying the individual T-cells as infection microenvironment reactive or tumor microenvironment reactive.

In another embodiment, a cancer diagnosing biomarker identification system comprises one or more processors; and memory coupled to the one or more processors, wherein the memory comprises computer-executable instructions causing the one or more processors to perform a process comprising receiving single cell RNA sequencing datasets for at least two cohorts, wherein at least one cohort comprises one or more cancer subjects and at least one cohort comprises one or more non-cancer subjects; identifying microbial genera in the datasets, wherein the identifying generates at least one microbial genera signature for the one or more cancer subjects and at least one microbial genera signature for the one or more non-cancer subjects; selecting microbial genera differentially present in the at least one microbial genera signature for the one or more cancer subjects compared to the at least one microbial genera signature for the one or more non-cancer subjects, wherein the selecting generates a differentiating microbial genera signature that distinguishes a cancer subject from a non-cancer subject; receiving a single cell RNA sequencing dataset for a subject at risk of having a cancer; identifying a set of microbial genera in the dataset for the subject at risk of having the cancer; and comparing the differentiating microbial genera signature to the set of microbial genera identified in the dataset from the subject at risk of having the cancer; thereby determining whether the subject at risk of having the cancer has the pancreatic cancer.

In a further embodiment, one or more computer-readable media have encoded thereon computer-executable instructions that, when executed, cause a computing system to perform a cancer diagnosing biomarker identification method comprising receiving single cell RNA sequencing datasets for at least two cohorts, wherein at least one cohort comprises one or more cancer subjects and at least one cohort comprises one or more non-cancer subjects; identifying microbial genera in the datasets, wherein the identifying generates at least one microbial genera signature for the one or more cancer subjects and at least one microbial genera signature for the one or more non-cancer subjects; selecting microbial genera differentially present in the at least one microbial genera signature for the one or more cancer subjects compared to the at least one microbial genera signature for the one or more non-cancer subjects, wherein the selecting generates a differentiating microbial genera signature that distinguishes a cancer subject from a non-cancer subject; receiving a single cell RNA sequencing dataset for a subject at risk of having a cancer; identifying a set of microbial genera in the dataset for the subject at risk of having the cancer; and comparing the differentiating microbial genera signature to the set of microbial genera identified in the dataset from the subject at risk of having the cancer; thereby determining whether the subject at risk of having the cancer has the cancer.

In another embodiment, a cancer survival outcome biomarker identification system comprises one or more processors; and memory coupled to the one or more processors, wherein the memory comprises computer-executable instructions causing the one or more processors to perform a process comprising receiving single cell RNA sequencing datasets for at least two cohorts, wherein at least one cohort comprises one or more poor survival outcome cancer subjects and at least one cohort comprises one or more good survival outcome cancer subjects; identifying microbial genera in the datasets, wherein the identifying generates at least one microbial genera signature for the one or more good survival outcome cancer subjects and at least one microbial genera signature for the one or more poor survival outcome cancer subjects; and selecting microbial genera differentially present in the at least one microbial genera signature for the one or more good survival outcome cancer subjects compared to the at least one microbial genera signature for the one or more poor survival outcome cancer subjects, wherein the selecting generates a differentiating microbial genera signature that distinguishes a good survival outcome subject from a poor survival outcome subject; receiving a single cell RNA sequencing dataset for the cancer subject; identifying a set of microbial genera in the dataset for the cancer subject; and comparing the differentiating microbial genera signature to the set of microbial genera identified in the dataset from the cancer subject; thereby predicting whether the cancer subject will have a good survival outcome or a poor survival outcome.

In a further embodiment, one or more computer-readable media have encoded thereon computer-executable instructions that, when executed, cause a computing system to perform a perform a cancer survival outcome biomarker identification method comprising receiving single cell RNA sequencing datasets for at least two cohorts, wherein at least one cohort comprises one or more poor survival outcome cancer subjects and at least one cohort comprises one or more good survival outcome cancer subjects; identifying microbial genera in the datasets, wherein the identifying generates at least one microbial genera signature for the one or more good survival outcome cancer subjects and at least one microbial genera signature for the one or more poor survival outcome cancer subjects; selecting microbial genera differentially present in the at least one microbial genera signature for the one or more good survival outcome cancer subjects compared to the at least one microbial genera signature for the one or more poor survival outcome cancer subjects, wherein the selecting generates a differentiating microbial genera signature that distinguishes a good survival outcome subject from a poor survival outcome subject; receiving a single cell RNA sequencing dataset for the cancer subject; identifying a set of microbial genera in the dataset for the cancer subject; and comparing the differentiating microbial genera signature to the set of microbial genera identified in the dataset from the cancer subject; thereby predicting whether the cancer subject will have a good survival outcome or a poor survival outcome.

In another embodiment, a computer-implemented method of identifying a microbe or virus in a sample comprises receiving a single cell RNA sequencing dataset for the sample, detecting microbial or viral nucleic acids in the dataset, and identifying the microbe or virus in the sample when a microbial or viral nucleic acid indicative of the presence of the microbe or virus is detected in the dataset. In yet another embodiment, a computer-implemented method of diagnosing a subject with an infectious disease caused by a microbe or a virus comprises receiving a single cell RNA sequencing dataset for a sample from the subject, detecting microbial or viral nucleic acids in the dataset, and identifying the microbe or virus in the sample when a microbial or viral nucleic acid indicative of the presence of the microbe or virus is detected in the dataset, thereby diagnosing the subject with the infectious disease.

In another embodiment, a microbe or virus identification system comprises one or more processors; and memory coupled to the one or more processors, wherein the memory comprises computer-executable instructions causing the one or more processors to perform a process comprising receiving a single cell RNA sequencing dataset for a sample, detecting microbial or viral nucleic acids in the dataset, and identifying the microbe or virus in the sample when a microbial or viral nucleic acid indicative of the presence of the microbe or virus is detected in the dataset. In a further embodiment, one or more computer-readable media have encoded thereon computer-executable instructions that, when executed, cause a computing system to perform a perform a microbe or virus identification method comprising receiving a single cell RNA sequencing dataset for a sample, detecting microbial or viral nucleic acids in the dataset, and identifying the microbe or virus in the sample when a microbial or viral nucleic acid indicative of the presence of the microbe or virus is detected in the dataset.

In yet another embodiment, an infectious disease diagnosis system comprises one or more processors; and memory coupled to the one or more processors, wherein the memory comprises computer-executable instructions causing the one or more processors to perform a process comprising receiving a single cell RNA sequencing dataset for the subject, detecting microbes and/or viruses in the dataset, and identifying the microbe or virus when the presence of the microbe or the virus is detected in the dataset. In a further embodiment, one or more computer-readable media have encoded thereon computer-executable instructions that, when executed, cause a computing system to perform a perform an infectious disease diagnosis method comprising receiving a single cell RNA sequencing dataset for the subject, detecting microbes and/or viruses in the dataset, and identifying the microbe or virus when the presence of the microbe or the virus is detected in the dataset.

In some embodiments, the identifying microbial genera in the datasets or the detecting a microbe or a virus in the dataset further comprises (i) mapping reads from the single cell RNA sequencing dataset (such as a dataset for a sample from a subject) to microbial and/or viral genomes using a metagenomics classifier, thereby assigning a genus and/or species identity to each read in the dataset; (ii) for each genus and/or species identified in (i): (a) comparing the number of reads assigned and the number of minimizers assigned; (b) comparing the number of minimizers assigned and the number of unique minimizers assigned; and (c) comparing the number of reads assigned and the number of unique minimizers assigned; and (iii) classifying the genus and/or species as a true positive result when a correlation value for each comparison in (ii)(a)-(ii)(c) is positive, and when a number of reads detected for the species is greater in the single cell RNA sequencing dataset as compared to a control.

As described herein, a variety of other features and advantages can be incorporated into the technologies as desired.

### BRIEF DESCRIPTION OF THE DRAWINGS

FIG. 1 is a block diagram of an example system determining whether a subject at risk of having a cancer (such as a pancreatic cancer) has the cancer, predicting whether a cancer subject (such as a pancreatic subject) will have a good survival outcome or a poor survival outcome, and/or determining T-cell microenvironment reaction (reactivity) in a subject.

FIG. 2 is a flowchart of an example method determining whether a subject at risk of having a cancer (such as a pancreatic cancer) has the cancer, predicting whether a cancer subject (such as a pancreatic subject) will have a good survival outcome or a poor survival outcome, and/or determining T-cell microenvironment reaction (reactivity) in a subject.

FIG. 3 is a block diagram of an example system identifying differential microbial genera signatures.

FIG. 4 is a flowchart of an example method identifying differential microbial genera signatures.

FIG. 5 is a block diagram of an example system determining whether a subject at risk of having a cancer (such as a pancreatic cancer) has the cancer.

FIG. 6 is a flowchart of an example method determining whether a subject at risk of having a cancer (such as a pancreatic cancer) has the cancer.

FIG. 7 is a block diagram of an example system identifying microbial diversity gene signatures.

FIG. 8 is a flowchart of an example method identifying microbial diversity gene signatures.

FIG. 9 is a block diagram of an example system determining whether a cancer subject (such as a pancreatic subject) will have a good survival outcome or a poor survival outcome.

FIG. 10 is a flowchart of an example method determining whether a cancer subject (such as a pancreatic subject) will have a good survival outcome or a poor survival outcome.

FIG. 11 is a block diagram of an example system identifying differential T-cell microenvironment reactivity signatures.

FIG. 12 is a flowchart of an example method identifying differential T-cell microenvironment reactivity signatures.

FIG. 13 is a block diagram of an example system determining T-cell microenvironment reactivity.

FIG. 14 is a flowchart of an example method determining T-cell microenvironment reactivity.

FIGS. 15A-15G show detection and validation of a distinct and diverse PDA microbiome. (FIG. 15A) Study design. See also Table 1. PDA, pancreatic ductal adenocarcinoma. (FIG. 15B) Differential abundances of microbial changes in pancreatic disease and in previously reported putative laboratory contaminants; boxplots show median (line), 25^{th} and 75th percentiles (box) and 1.5×IQR (whiskers). Points represent outliers. N=nonmalignant tissues (n=11), T=tumors (n=24) (Wilcoxon test, ns=p>0.05, *p<0.05,**p<0.01, ***p<0.001, ****p<0.0001). (FIG. 15C) Comparisons of bacterial abundance in pancreatic tissues across multiple studies using differing technologies. Lower triangle=Spearman correlation of study-level abundances, upper triangle=overlap coefficient of present/absent genera. Columns indicate the number of samples and rows indicate the number of genera passing quality filters. scRNAseq=single-cell RNA sequencing, TCGA=The Cancer Genome Atlas. (FIG. 15D) Bar plots of relative abundances of genera in the Peng cohort. (FIG. 15E) Differentially present bacterial and fungal genera in nonmalignant vs. tumor samples computed from a linear model with tissue status, total metagenomic counts, and sample composition as covariates. Data shown for genera with abundance>10⁻³ or those listed in FIG. 15B. DE Coef, differential expression coefficient, Q, adjusted-p value. (FIG. 15F) Uniform manifold approximation and projection (UMAP) of barcodes tagging bacterial (left, n=23,4466 barcodes) and fungal (right, n=4,312 barcodes) DNA, colored by tissue status (N, nonmalignant, T, tumor). (FIG. 15G) Alpha-diversity of nonmalignant (N) and tumor (T) microbiomes, based in Shannon and Simpson scores. Box plots are as above, with Wilcoxon testing.

FIGS. 16A-16G show that microbes are associated with particular host cells and correlate with immune infiltration and diversity. (FIG. 16A) UMAP of barcodes tagging bacterial (left, n=23,4466 barcodes) and fungal (right, n=4,312 barcodes) DNA, colored by associated somatic-cell type. (FIG. 16B) Circos-plot of significant microbe-somatic cell enrichments identified at the single-barcode level by Wilcoxon testing. The ribbon width correlates with enrichment strength. (FIG. 16C) Statistically significant microbe-somatic cell enrichments in subsampled vs. cell-type label-shuffled (random) data in two data sets of scRNAseq, and the number of enrichments shared between the two studies. Two distributions were compared by applying Wilcoxon test. Bars, mean number of enrichments, Error-bars, bootstrapped 95% confidence intervals. (FIG. 16D) ROCs for random forest predictions of barcode cell-types using microbiome profiles alone. Curves colored by cell type. AUC, area under the curve. (FIG. 16E) Somatic cellular composition prediction using 34 sample-level microbiome abundances. Each point represents a normalized cell-type level in sample, colored as in FIG. 16D. (FIG. 16F) Self-assembling manifold (SAM) principal component analysis for individual somatic-cell types based on transcriptome. Cells colored by their data-driven cluster assignment, with immune types annotated: GC, germinal center, DC, dendritic cell, MP, macrophage, Th17, T-helper 17, TCM, T-central memory, TEM, T-effector memory, Treg, T-regulatory, Tfh, T-follicular helper, NK, natural killer. (FIG. 16G) Spearman correlations of microbial (Shannon) diversity and somatic cellular fraction (top) or somatic cellular diversity (bottom) in the same sample. Somatic cell diversity was calculated using cluster assignments from FIG. 16F. TME, tumor microenvironment.

FIGS. 17A-17H show that specific microbe abundances correlate with co-localized cell-type specific gene expression. (FIG. 17A) Unsupervised dot-plots represent significant correlations between normal and tumor-specific microbes and receptor gene expression in their co-localized cell-types: Rows, differentially expressed microbe genera from FIG. 15E; columns, receptor gene expression levels; triangles, positive, circle, negative correlation. Colors represent the cell-type for the correlation. Boxes added to highlight significant clusters, with significant KEGG-pathway enrichments indicated. (FIG. 17B) Volcano plots for correlations between individual microbe abundances and gene expression (top, individual cells) or pathway scores (bottom, averaged cell-type scores), colored by point density. (FIG. 17C) Heatmap of Spearman correlations between sample-level microbial abundances and inflammation-related gene expression. (FIG. 17D) Network of microbe-cell-specific pathway and pathway-pathway associations. Nodes represent either microbe or cell-specific pathway score, with edges linking nodes with significant correlations (Irl>0.5, p<0.05). Nodes are colored by cell-type and shaped by their pathway category: Blue edges, negative correlation. See also FIG. 9. (FIG. 17E) Edge centrality computed from FIG. 17D. Colors based on node linkages connecting a microbe (orange) or only connecting somatic pathways (grey). (FIG. 17F) Linkage of bacterial abundances and gene expression in Peng and TCGA samples. Bacteroides and LYZ gene expression and (FIG. 17G) Campylobacter and Hippo signaling. (FIG. 17H) Number of statistically significant, shared microbe-gene or pathway associations between the Peng cohort (Peng et al. Cell Res. 29(9):725-738, 2019) and TCGA (Poore et al. Nature 579: 567-574, 2020) in subsampled vs. sample-label shuffled data. Bars, mean number of enrichments, Error-bars, bootstrapped 95% confidence intervals (n=500, Wilcoxon-test).

FIGS. 18A-18C show microbe abundances that correlate with cell-type specific pathway activity scores. Unsupervised dot-plots representing biologically and statistically significant Spearman correlations (Irl>0.5, p<0.05, t-test) between normal and tumor-specific microbes and pathways in their co-localized cell-types. Key: Rows, differentially expressed microbe genera (FIG. 15E); Columns, KEGG pathways; Triangles, positive, Circle, negative correlation; Colors, cell-type (FIG. 16F) in which the correlation existed. (FIG. 18A, FIG. 18B) Non-metabolic pathways; (FIG. 18C) metabolic pathways.

FIGS. 19A-19H show T-cell characteristics, microenvironment features, and microbiome-clinical associations. (FIG. 19A) Training and test datasets used to create a random forest model to distinguish between T-cells infection vs. tumor microenvironment reaction based on their gene expression profiles. (FIG. 19B) ROC curve indicating exceptional model performance on test datasets; AUC, area under the curve. Inset: Confusion matrix of model assignments; rows, predicted, columns, true values. (FIG. 19C) Bar-plot of predicted T-cell microenvironment reaction in the Peng cohort. (FIG. 19D) Pseudotime analysis of samples based on microbiome profiles and cell-specific pathway scores identifies distinct states: NS, normal state, TS, tumor state representing data-driven PDA subtypes with distinct molecular, microbiome, and clinical characteristics. Arrows indicate microbiome and clinical differences amongst TS1-3, based on t-tests and Fisher's test. (FIG. 19E) Circular heatmap of microbiome/pathway differences for the four states. Rows represent microbe or cell-specific pathway; Columns represent the four states, with NS outermost, followed by TS1, 2, 3. Average microbe expression or pathway score: Red, high; Blue, low. (FIG. 19F) Example pathway and microbiome changes in the four states as samples progress along pseudotime. Points represent individual samples colored by their state. (FIG. 19G) Confusion matrix showing the utility of a 6-gene signature in classifying Peng (Peng et al. Cell Res. 29(9):725-738, 2019) samples as high or low microbiome diversity. (FIG. 19H) Kaplan-Meier plots of TCGA (left) and ICGC PDA (center) cohorts stratified by predicted microbial diversity, and (right) survival curves for TCGA PDA cohorts stratified by microbiome diversity directly measured from the same samples by Poore et al. (Poore et al. Nature 579: 567-574, 2020) (TCGA observed).

FIGS. 20A-20G show quality measures and metagenomic read statistics. (FIG. 20A) Uniform manifold approximation and projection (UMAP) of somatic cells clustered by transcriptomes profiles and colored by sample type (left panel, N=nonmalignant, T=tumor), patient sample (middle panel), and cell-type (right panel). (FIG. 20B) Percent of bacterial reads resolved to the genus level that were discarded due to being PCR duplicates, having low genera abundance, or not passing the multi-study filter. The remaining reads were retained for downstream analysis. (FIG. 20C) Processed metagenomic vs. somatic gene counts; N=nonmalignant, T=tumor. (FIG. 20D) Boxplots of metagenomic read counts in nonmalignant (N) and tumor (T) samples showing median (line), 25th and 75th percentiles (box) and 1.5×IQR (whiskers). (FIG. 20E) Boxplots showing metagenomic counts per cell type in nonmalignant (N) and tumor (T) samples. Inset: Percentage of metagenomes that are somatic cell-associated in nonmalignant (N) and tumor (T) samples. Boxplots show median (line), 25th and 75th percentiles (box) and 1.5×IQR (whiskers). (FIG. 20F) UMAP plot of metagenomic barcodes from three pancreas single-cell RNA sequencing datasets colored by study of origin. Peng N=nonmalignant Peng samples, Peng T=tumor Peng samples. (FIG. 20G) UMAP plot of bacterial and fungal metagenomic barcodes from the Peng cohort. Red=barcodes from tumors, blue=barcodes from nonmalignant samples, circles=bacteria-only barcodes, squares=fungi-only barcodes, triangles=bacteria and fungi barcodes.

FIGS. 21A-21B shows cell-type and sample cellular composition predictions with null models. (FIG. 21A) Sensitivity vs. specificity curves for random forest predictions of label-shuffled barcode cell-types using barcode metagenomic profiles. Curves are colored by cell type. AUC, area under the curve. (FIG. 21B) Distribution of R-squared values from 100 null models using 34 sample-level abundances to predict sample somatic cellular composition. Null models were created by shuffling sample labels.

FIGS. 22A-22E show microbiome associations with numerous somatic cellular activities. (FIG. 22A) Ranked pathway enrichments from biologically and statistically significant (Irl>0.5, p<0.05) microbe-gene pathway correlations in individual cells. (FIG. 22B) Heatmap showing Spearman correlation coefficients between microbes and total antimicrobial gene expression. (FIG. 22C) Volcano plot of microbe-pathway correlations between all average cell-type specific microbe levels and cell-type specific pathways. (FIG. 22D) Heatmap showing Spearman correlation coefficients for significant correlations from FIG. 22C with IrI>0.5 and p<0.05 for pathways involving malignant ductal 2 cells. (FIG. 22E) Heatmap showing correlations from FIG. 22C with IrI>0.5 and p<0.05 for all pathways and cell-types.

FIG. 23 shows a network of correlations between microbes and cell-type specific cancer-related pathway scores. Nodes represent either a microbe or cell-type specific pathway. Edges represent a significant correlation between nodes, defined as IrI>0.5 and p<0.05 for microbe-pathway correlations, and IrI>0.75 and p<0.05 for pathway-pathway correlations. A higher cutoff was used for pathway-pathway correlations to account for overlapping gene sets in some pathways. Nodes are colored by their somatic or microbial cell-type, shaped by their pathway category (or otherwise microbe), and sized proportionally to their number of edges. Grey edges represent positive correlations, and blue edges represent negative correlations.

FIG. 24 shows a pseudotime analysis of tumor microenvironments using pathway scores alone. Average cell-type specific pathway scores for cancer-related pathways were used to order entire tumor microenvironments along a progressive process. The same branching pattern with distinct clusters emerges as when microbiome profiles are included (see FIG. 19D).

FIG. 25 shows detection of known infections using scRNA-seq data from a variety of tissue types and pathogens. Box plots show read counts per million assigned microbiome reads for infected versus uninfected samples in multiple benchmark datasets with either a known pathogen (either introduced or clinically identified). Boxplots show the median (horizontal line), 25th and 75th percentiles (box), and 1.5× the interquartile range (IQR) (whiskers) for each experiment. Points represent outliers. Statistical significance was determined using Wilcoxon testing (p<0.001).

FIGS. 26A-26D show criteria for detecting and de-noising microbiome signals. (FIG. 26A) Sequencing reads from true species have positive relationships between (1) the number of reads assigned and number of minimizers assigned, (2) number of minimizers assigned and number of unique minimizers assigned, and (3) number of reads assigned and number of unique minimizers assigned. Data are shown for the benchmark datasets tested. (FIG. 26B) Table detailing benchmark dataset metadata and Spearman correlation coefficients from FIG. 26A. (FIG. 26C) Scatter plot showing the relationship between the three correlations from FIG. 26A for all species detected in the benchmark datasets. Each point represents a species. Extension of the cloud of points into low correlation values indicates the presence of abundant false positive results. Concentration of points at high values suggest the presence of other species, including contaminants. (FIG. 26D) Scatter plot showing the relationship between the three correlations in FIG. 26A for microbiomes detected in cell line experiments taken as benchmark negative controls. Any species shown in this scatter plot are contaminants or false positives. In test samples, species not detected above the thresholds found in negative controls were assumed to be false positive or contaminant species.

FIG. 27 is a block diagram of an example computing system in which described embodiments can be implemented.

FIG. 28 is a block diagram of an example cloud computing environment that can be used in conjunction with the technologies described herein.

### DETAILED DESCRIPTION

### Example 1—Overview

Microorganisms are detected in multiple tissue types, such as cancer tissues, including in tumors of the pancreas and other putatively sterile organs. However, it remains unclear whether bacteria and fungi preferentially associate with specific tissue contexts and whether they influence oncogenesis or anti-tumor responses in humans. SAHMI was developed herein as a novel framework to analyze host-microbiome interactions in the tumor microenvironment using single-cell sequencing data. Interrogating human pancreatic ductal adenocarcinomas (PDA) and nonmalignant pancreatic tissues identified an altered and diverse tumor microbiome, capturing both novel and known PDA-associated microbes detected with other technologies. Certain microbes showed preferential association with specific somatic cell-types, and their abundances correlated with select receptor gene expression and cancer hallmark activities in host cells. Nearly all tumor-infiltrating lymphocytes had infection-reactive transcriptional profiles, which may contribute to the lack of efficacy of immune checkpoint inhibitors. Pseudotime analysis suggested tumor-microbial co-evolution and identified three tumor modalities with distinct microbial, molecular, and clinical characteristics. Finally, using multiple independent datasets, a signature of increased intra-tumoral microbial diversity predicted patients at risk of poor survival. Collectively, tumor-microbiome cross-talk appears to modulate pancreatic cancer disease course with implications for clinical management.

### Example 2—Example Biomarkers

In any of the examples herein, the described biomarkers can take the form of one or more microbial genera, one or more genes, and/or one or more pathways. In practice, a pathway can comprise a set of a plurality of gene identifiers that identify real-world genes as described herein. Such genes are grouped together in the pathway by their involvement in the same biological pathway, or by proximal location on a chromosome. The technologies herein can comprise identifying (e.g., discovering) candidate biomarkers, where the identifying comprises selecting (e.g., filtering) a set of biomarkers, for example based on identification and/or expression of one or more of the biomarkers between cohorts having characteristics of interest as described herein.

In any of the examples herein, phenotypes of interest can include a variety of phenotypes, such as the presence or absence of a cancer in a subject, a poor or good survival outcome in a subject having cancer, and/or T-cell reactivity. In practice, phenotypes can depend on a variety of factors, including gene expression information. Therefore, gene expression data can be used in the examples herein to identify phenotypes.

In one example, analysis of nucleic acid sequences at the individual cell level, such as using scRNA-seq as described herein, allows for identification of subjects that have a cancer, such as pancreatic cancer, and/or determination of a survival outcome (e.g., poor or good) in a subject that has cancer, based on the presence of particular microbes associated with individual cells analyzed from tumor tissue, wherein microbe abundances are increased or decreased relative to a control (such as normal tissue of the same cell type). In one example, the presence of particular microbes in higher amounts in the tumor cells (e.g., pancreatic cancer cells), such as an increase in Prevotella, Megamonas, Spiroplasma, Bacteroides, Polaribacter, Arcobacter, Acinetobacter, Clostridium, Chryseobacterium, Lactobacillus, Paenibacillus, Flavobacterium, Vibrio, Mycoplasma, Campylobacter, Streptococcus, Fusobacterium, Buchnera, Streptomyces, Bacillus, Kluyveromyces, Sphingobacterium, Saccharomyces, Thermothielavioides, Colletotrichum, and/or Aspergillus nucleic acid molecules relative to a control (such as normal tissue of the same cell type, such as normal pancreas tissue), can indicate the presence of cancer and/or a poor survival outcome. In another example, the presence of particular microbes in lower amounts in the tumor cells (e.g., pancreatic cancer cells), such as a decrease in Staphylococcus, Paraccocus, Burkholderia, Klebsiella, Pasteurella, and Ralstonia nucleic acid molecules relative to a control (such as normal tissue of the same cell type, such as normal prostate cancer), indicates the presence of cancer.

In the examples herein, a poor survival outcome corresponds to a median survival of 603 days and increased microbial diversity in a sample from the subject. In other examples herein, a good survival outcome corresponds to a median survival of 1502 days and reduced microbial diversity in a sample from the subject.

In some embodiments, expression levels of a set of six genes (the six-gene signature) is used to classify the subject as having a poor or good survival outcome. The six-gene signature can be used to classify the sample as having low or high microbial diversity. In specific embodiments, the genes of the six-gene signature are nth like DNA glycosylase 1 (NTHL1; e.g., GENBANK® Accession No. U81285.1), ly6/PLAUR domain-containing protein 2 (LYPD2; e.g., GENBANK® Accession No. AY358432.1), mucin-16 (MUC16; e.g., GENBANK® Accession No. AF414442.2), C2 calcium-dependent domain-containing protein 4B (C2CD4B; e.g., GENBANK® Accession No. BM023530.1), flavin containing dimethylaniline monooxygenase 3 (FMO3; e.g., GENBANK® Accession No. BC032016.1), and interleukin-1 receptor-like 1 (IL1RL1; e.g., GENBANK® Accession No. AB012701.3). In other specific embodiments, increased expression of one or more of IL1RL1, C2CD4B, FMO3, or NTHL1 compared to a control, and/or decreased expression of one or more of LYPD2 or MUC16 compared to the control indicates high microbial diversity in the subject and classifies the subject as having a poor survival outcome. In yet another specific embodiment, decreased expression of one or more of IL1RL1, C2CD4B, FMO3, or NTHL1 compared to a control, and/or increased expression of one or more of LYPD2 or MUC16 compared to the control indicates low microbial diversity in the subject and classifies the subject as having a good survival outcome. In some embodiments, classifying the subject as having a poor or good survival outcome comprises calculating the Shannon diversity index for the sample based on expression levels of the set of six genes in the sample compared to a control, thereby determining the microbial diversity of the sample. The control can be any control sample as disclosed herein. In one example the control is individual non-cancerous/normal cells of the same tissue type, or values (or a range of values) that represents expression for each of NTHL1, LYPD2, MUC16, C2CD4B, FMO3, and ILIRL1 in such cells.

In another example, T-cells, which can be identified using biological markers known to one of ordinary skill in the art, can be classified as described herein as microbe-responsive or tumor-responsive. In some embodiments, the T-cells are tumor-infiltrating T-cells. T-cells that are classified as tumor-responsive can indicate that the subject may be responsive to a therapy that targets a particular type of T-cell.

In yet another example, analysis of nucleic acid sequences at the individual cell level, such as using scRNA-seq as described herein, allows for identification of infectious agents, such as microbes (such as bacteria or fungi) or viruses, in a subject suspected of having an infectious disease caused by the infectious agent. In one example, the presence of nucleic acid molecules for a particular microbe or virus in higher amounts in the sample from the subject (e.g., cells from a subject suspected of having an infectious disease), such as an increase in Candida albicans, lentivirus (such as human immunodeficiency virus (HIV)), Helicobacter pylori, alphaherpesvirus, Mycobacterium leprae, Mycobacterium tuberculosis, Salmonella enterica, or coronavirus (such as MERS or SARS, such as SARS-CoV or SARS-CoV-2) relative to a control can indicate the presence of the infectious agent. In particular examples, analysis of nucleic acid sequences at the individual cell level allows for identification of such infectious agents without a need for a control.

### Example 3—Examples System Implementing Identifying Biomarkers

Example systems for implementing identifying biomarkers of phenotypes (such as a patient having cancer or a cancer patient having a poor or a good survival outcome) via analysis of microbial and gene expression information from a sample using single-cell sequencing data are disclosed herein. Example systems can include a processor coupled to memory, such as memory with computer-executable instructions for identifying treatment-response biomarkers.

Example systems can include training and use of expression data via analysis of single cell RNA sequencing data to generate biomarkers, such as a microbial signature and/or a gene signature, for identification of phenotypes (such as the presence or absence of cancer, such as pancreatic cancer). In practice, biomarker identification can be trained and used independently or in tandem. For example, a system can be trained and then deployed to be used independent of any training activity, or the system can continue to be used after deployment. In practice, the system can receive expression data, which can be used to generate a microbial and/or gene expression signature for one or more phenotypes (such as the presence or absence of cancer, such as pancreatic cancer, or good versus poor survival in a pancreatic cancer patient). The system can then receive additional expression data, for which a microbial and/or gene expression signature can be used via comparison to one or more previously identified biomarkers to determine one or more phenotypes (such as the presence or absence of cancer, such as pancreatic cancer, or good versus poor survival in a pancreatic cancer patient).

In practice, a system receives expression data for at least one subject or group of subjects. The subject or group can have a known or an unknown phenotype (such as the presence or absence of cancer, such as pancreatic cancer, or a good versus poor survival outcome in a pancreatic cancer patient), such as for system training or use.

In examples, a system can use expression data to identify differential microbial and/or gene expression datapoints. Differential microbial and/or gene expression signatures can also be generated. Various types of signatures are possible with various indicia of differentiation.

In practice, the systems disclosed herein can vary in complexity with additional functionality, more complex components, and the like. The described systems can also be networked via wired or wireless network connections to a global computer network (e.g., the Internet). Alternatively, systems can be connected through an intranet connection (e.g., in a corporate environment, government environment, educational environment, research environment, or the like).

The systems disclosed herein can be implemented in conjunction with any of the hardware components described herein, such as computing systems described below (e.g., processing units, memory, and the like). In any of the examples herein, the inputs, outputs, signatures (such as differential microbial and/or gene expression signatures, or pathway signatures), trained identifiers (such as microbial genera and/or gene identifiers), information about signatures (such as expression data or information about differential microbial and/or gene expression signatures, and pathway signatures), and the like can be stored in one or more computer-readable storage media or computer-readable storage devices. The technologies described herein can be generic to the specifics of operating systems or hardware and can be applied in any variety of environments to take advantage of the described features.

### Example 4—Example Method Implementing Identifying Biomarkers

Example methods implementing identifying biomarkers of phenotypes (such as the presence or absence of cancer, such as pancreatic cancer, or good versus poor survival in a pancreatic cancer patient) are disclosed herein.

Example methods include both training and use of expression data via analysis of differential expression to generate biomarkers, such as microbial genera signatures, gene expression signatures (such as microbial diversity gene signatures), T-cell microenvironment reactivity signatures, and/or pathway signatures, for phenotype identification (such as the presence or absence of cancer, such as pancreatic cancer, or good versus poor survival in a cancer patient, such as a pancreatic cancer patient; or such as the presence or absence of an infectious agent in a sample, such as in a sample from a subject suspected of having an infection caused by the infectious agent). However, in practice, either phase of the technology can be used independently (e.g., a system can be trained and then deployed to be used independently of any training activity) or in tandem (e.g., training continues after deployment).

In examples, expression data are received. Gene expression data can take the form described herein.

Further, expression data can be received with or without additional processing. For examples, the method can include normalizing, transforming, or reducing redundancy in the data. Other processing steps are possible.

In examples, the methods can include generating differential microbial genera and/or gene expression signatures using expression data (such as by identifying, for example using a differential identifier). In practice, expression data are input into a differential identifier, and differential microbial, gene expression, and/or pathway signatures are output.

In examples, the methods can include generating microbial, gene expression, and/or pathway signatures using differential gene expression data, such as by determining (for example, using a differential identifier). In practice, differential microbial, gene expression, and/or pathway signatures can be input into a differential identifier, and differential microbial, gene expression, and/or pathway signatures can be output.

In examples, the methods can include generating a pathway signature, such as by determining (for example, using a pathway enrichment identifier). In practice, pathway signatures can be input into a comprehensive pathway enrichment identifier, and a comprehensive pathway signature can be output.

### Example 5—Example Expression Data

In any of the examples herein, expression data can take a variety of forms. For example, expression data can include level of expression associated with a gene, such as a list of one or more genes or set of genes, in which each gene is associated with a level of expression. In practice, digital expression data or a digital representation of expression data can be used as input to the technologies. In practice, expression data can take the form of a digital or electronic item such as a file, binary object, digital resource, or the like.

Example expression data can include gene or gene expression data, such as a direct or an indirect measure of genes or gene expression. For example, transcriptomic data can be used as a measure of gene expression. In specific, non-limiting examples, genomic data can include nucleic acid-based data, such as mRNA or miRNA data.

Data obtained using various techniques can be used in the methods herein. For example, gene expression can be detected and quantitated using RNA sequencing (RNA-seq), such as single cell RNA-seq (scRNA-seq) (see Stark, et al., Nat Rev Genet. 2019;20, 631-656; Haque, et al., Genome Med. 2017;9(75)). RNA-seq is most frequently used for analyzing differential gene expression between samples. In traditional RNA-seq analyses, the process of analyzing differential gene expression via RNA-seq begins with RNA extraction (such as from a tumor sample, such as a pancreatic cancer sample), followed by mRNA enrichment or ribosomal RNA depletion. cDNA is then synthesized, and an adaptor-ligated sequencing library is prepared. The library is sequenced to a read depth of, for example, 10-30 million reads per sample on a high-throughput platform (such as an Illumina platform). The sequencing reads (most often in the form of FASTQ files) are computationally aligned and/or assembled to a transcriptome. The reads are most often mapped to a known transcriptome or annotated genome, matching each read to one or more genomic coordinates. This process is often accomplished using alignment tools such as STAR, TopHat, or HISAT, which each rely on a reference genome. If no genome annotation containing known exon boundaries is available (such as if a reference genome annotation is missing or is incomplete), or if reads are to be associated with transcripts rather than genes, aligned reads can be used in a transcriptome assembly step using tools such as StringTie or SOAPdenovo-Trans. Tools such as Sailfish, Kallisto, and Salmon can associate sequencing reads directly with transcripts, without the need for a separate quantification step. Next, reads that have been mapped to transcriptomic or genomic locations are quantified using tools such as RSEM, CuffLinks, MMSeq, or HTSeq, or the alignment-free direct quantification tools Sailfish, Kallisto, or Salmon. Quantification results are often combined into an expression matrix, with one row for each expression feature (gene or transcript) and one column for each sample, with values being read counts or estimated abundances. Samples are then filtered and normalized to account for differences in expression patterns, read depth, and/or technical biases. Significant changes in expression of individual genes and/or transcripts between sample groups are then statistically modeled using one or more of various tools and computational methods.

scRNA-seq enables the systematic identification of cell populations in a tissue. Short sequences or barcodes may be added during library preparation or by direct RNA ligation, before amplification, to mark a sequence read as coming from a specific starting molecule or cell, such as in scRNA-seq experiments. In a scRNA-seq analysis, a tissue sample (such as a pancreatic tissue sample, such as a pancreatic cancer tissue sample) is dissociated, single cells are separated, and RNA from each individual cell is converted to cDNA (and can be labelled during reverse transcription) and then amplified (typically using PCR) for sequencing. The synthesized cDNA is used as the input for library preparation. Amplified nucleic acids can also be labelled with barcodes (such as using single-cell combinatorial indexing RNA sequencing or split-pool ligation-based transcriptome sequencing). Tissue dissociation may be accomplished using methods known in the art, such as mechanical disaggregation and/or enzymatic dissociation, such as enzymatic dissociation using collagenase and/or DNase. Similarly, single cells can be separated using known methods, such as flow-cytometry, wherein cells can be flow-sorted directly into micro-plates containing lysis buffer. Individual cells can also be captured in microfluidic chips or loaded into nano-well devices (e.g., by Poisson distribution), isolated, and merged into droplets (containing reagents) via droplet-microfluidic isolation (such as Drop-Seq or InDrop). Isolated single cells are then lysed such that RNA can be released for cDNA synthesis.

Expression data can further include gene or gene expression data from a variety of sources, such as private or publicly accessible databases. For example, databases can include general or specialized databases, such as databases specific for species, taxa, or subject, for example, cancer subjects (such as the Cancer Genome Atlas or the Genomics Data Commons database, portal.gdc.cancer.gov).

Further, in any of the examples herein, expression data can be used with or without additional processing. For example, the methods can include normalization or variance-stabilizing transformation. Other processing is possible, such as centering, standardization, log transformation, rank transformation, and the like.

In any of the examples herein, expression data or its representation can be stored in a database (such as a genomic data database). The database can include expression data with or without additional processing. In particular examples, expression data are stored as a raw or processed RNA-seq data (such as RNA-seq counts, for example, normalized or transformed RNA-seq counts). Precompiled expression data databases may also be used. For example, an application that already has access to a database of pre-computed expression data can take advantage of the technologies without having to compile such a database. Such a database can be available locally, at a server, in the cloud, or the like. In practice, a different storage mechanism than a database can be used (such as a sequence table, index, or the like).

### Example 6—Example Subjects

In any of the examples herein, expression data can include data for a variety of subjects or groups of subjects. In practice, subjects can be single subjects or a part of a group (such as a group with a common feature or characteristic, or a cohort).

In examples, data for subjects or groups can be used for training. For example, subjects or groups can include known features or phenotypes, such as for training and validation thereof (for example, training or validation subjects, groups, or cohorts). In specific, non-limiting examples, subjects or groups have a disease, such as cancer or a specific type of cancer (or a malignant tumor characterized by abnormal or uncontrolled cell growth, such as a pancreatic cancer).

In examples, data for subjects or groups can be used to identify subjects with a feature or phenotype. In practice, subjects or groups can include unknown features or phenotypes, which can then be identified using a trained system (for example, query subjects, groups or cohorts). For example, subjects or groups can have a disease, such as cancer or a specific type of cancer (or a malignant tumor characterized by abnormal or uncontrolled cell growth, such as a pancreatic cancer), and a trained system can be used to identify subjects or groups with a phenotype of interest (such as a good or poor survival outcome, such as a good or poor survival outcome in a subjecting with pancreatic cancer).

### Example 7—Example Samples

The disclosed methods can include obtaining a biological sample from the subject. In examples, “sample” can refer to part of a tissue that is either the entire tissue, or a diseased or healthy portion of the tissue. The sample can include cells (such as mammalian and microbial cells) and associated includes nucleic acid molecules. Such samples include, but are not limited to, tissue from biopsies (including formalin-fixed paraffin-embedded tissue), autopsies, and pathology specimens; sections of tissues (such as frozen sections or paraffin-embedded sections taken for histological purposes); body fluids, such as blood, sputum, serum, ejaculate, or urine, or fractions of any of these; and so forth. In one example, the sample is a fine needle aspirate.

In one particular example, the sample from the subject is a tissue biopsy sample. In another specific example, the sample from the subject is a pancreatic tissue sample. In some examples, the sample includes T cells from the subject, such as a subject with cancer.

In several embodiments, the biological sample is from a subject suspected of having a cancer, such as pancreatic, stomach cancer, colon cancer, breast cancer, uterine cancer, bladder, head and neck, kidney, liver, ovarian, pancreas, prostate, kidney, or rectum cancer. In some embodiments, the biological sample is a tumor sample or a suspected tumor sample. For example, the sample can be a biopsy sample from at or near or just beyond the perceived leading edge of a tumor in a subject. Testing of the sample using the methods provided herein can be used to confirm the location of the leading edge of the tumor in the subject. This information can be used, for example, to determine if further surgical removal of tumor tissue is appropriate, and/or if certain treatments or treatment methods are appropriate for use in the subject.

In other embodiments, the biological sample is from a subject suspected of having an infection, such as a Candida albicans, human immunodeficiency virus (HIV), Helicobacter pylori, alphaherpesvirus, Mycobacterium leprae, Mycobacterium tuberculosis, Salmonella enterica, or a coronavirus (such as MERS or SARS, such as SARS-CoV or SARS-CoV-2) infection.

As described herein, samples obtained from a subject (such as pancreatic tissue samples, such as pancreatic cancer samples) can be compared to a control. In some embodiments, the control is a cancer sample (such as a pancreatic cancer sample) obtained from a subject or group of subjects known to have had good survival outcomes (or poor survival outcomes). In some embodiments, the control is an infectious disease sample obtained from a subject or group of subjects known to have the infectious disease. In other embodiments, the control is a standard or reference value based on an average of historical values. In some examples, the reference values are an average expression (such as RNA expression) value for each of a microbe- and/or cancer-related molecule (such as molecules useful for detecting microbes of one or more genera, such as genera Prevotella, Megamonas, Spiroplasma, Bacteroides, Polaribacter, Arcobacter, Acinetobacter, Clostridium, Chryseobacterium, Lactobacillus, Paenibacillus, Flavobacterium, Vibrio, Mycoplasma, Campylobacter, Streptococcus, Fusobacterium, Buchnera, Streptomyces, Bacillus, Kluyveromyces, Sphingobacterium, Saccharomyces, Thermothielavioides, Colletotrichum, Aspergillus, Staphylococcus, Paraccocus, Burkholderia, Klebsiella, Pasteurella, and/or Ralstonia) and/or housekeeping genes, in a cancer sample (such as a pancreatic cancer sample) obtained from a subject or group of subjects known to have or to have had cancer. In other embodiments, the reference values are an average expression (such as RNA expression) value for each of an infectious disease-related molecule (such as molecules useful for detecting microbes of one or more genera, such as genera Candida, Helicobacter, Mycobacterium, or Salmonella, or molecules useful for detecting one or more viruses, such as a lentivirus, alphaherpesvirus, or coronavirus).

In some examples, the reference values are an average expression (such as RNA expression) value for each of NTHL1, LYPD2, MUC16, C2CD4B, FMO3, and IL1RL1 in a cancer sample (such as a pancreatic cancer sample) obtained from a subject or group of subjects known to have or to have had cancer, or a corresponding non-cancer sample of the same tissue type.

In some examples, the reference values are an average expression (such as RNA expression) value for each of the genes listed in Table 2 in T cells obtained from a subject or group of subjects known to have or to have had cancer (such as T cells from or near the tumor), or T cells from a subject known not to have cancer.

In some embodiments, the control is a non-cancer sample (such as a non-cancer sample of the same tissue type as the cancer) obtained from a subject or group of subjects known to not have cancer. In other embodiments, the control is a non-infectious disease sample obtained from a subject or group of subjects known to not have the infectious disease.

Samples can be obtained from a subject, for example, from infectious disease patients or from cancer patients (such as pancreatic cancer patients) who have undergone tumor resection as a form of treatment. In some embodiments, cancer samples (such as pancreatic cancer samples) are obtained by biopsy. Biopsy samples can be fresh, frozen or fixed, such as formalin-fixed and paraffin embedded. Samples can be removed from a patient surgically, by extraction (for example by hypodermic or other types of needles), by microdissection, by laser capture, or by other means.

In some examples, the sample is used to generate a suspension of individual cells, such that nucleic acid molecules can be sequenced for individual cells. In some examples, individual cells are bar coded.

In some examples, proteins and/or nucleic acid molecules (e.g., DNA, RNA, miRNA, mRNA) are isolated or purified from the cancer sample (such as a pancreatic cancer sample) and non-cancer sample. In some examples, the cancer sample (such as a pancreatic cancer sample) is used directly, or is concentrated, filtered, or diluted. In other examples, proteins and/or nucleic acid molecules (e.g., DNA, RNA, miRNA, mRNA) are isolated or purified from the sample from the subject suspected of having the infectious disease and a control sample. In some examples, the sample from the subject suspected of having the infectious disease is used directly, or is concentrated, filtered, or diluted.

### Example 8—Example System

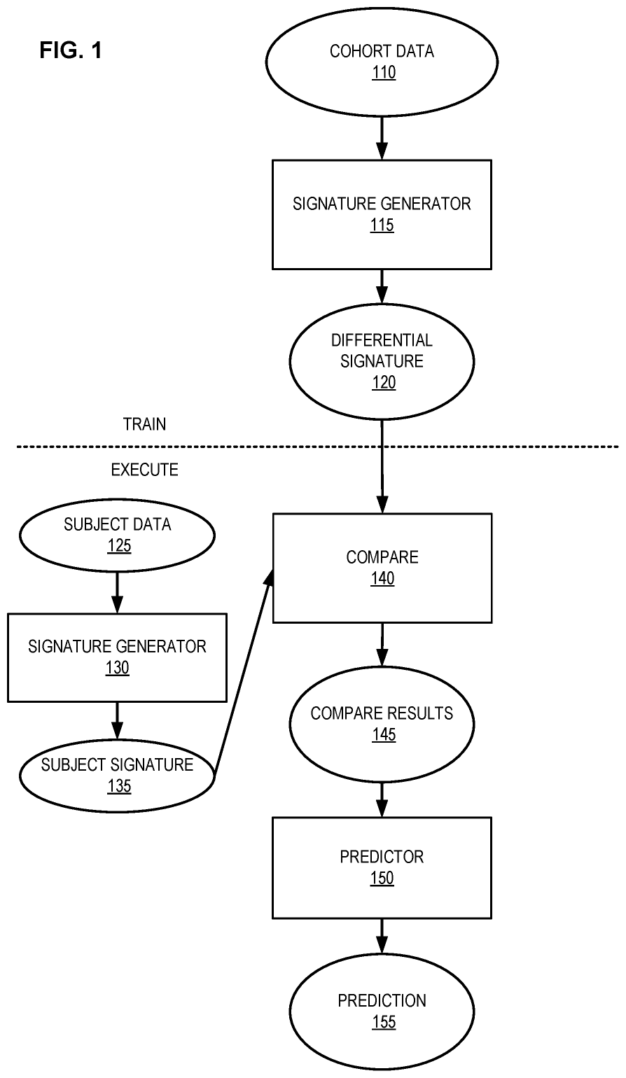

FIG. 1 is a block diagram showing a basic system 100 that can be used to implement determining whether a subject at risk of having a cancer (such as a pancreatic cancer) has the cancer, predicting whether a cancer subject (such as a pancreatic subject) will have a good survival outcome or a poor survival outcome, and/or determining T-cell microenvironment reaction (reactivity) in a subject as described herein. The system 100 can be implemented in a computing system as described herein.

In the training phase of the example, a signature generator 115 receives cohort data 110, such as scRNA-seq reads, for example scRNA-seq reads in the form of FASTQ files, and generates a differential signature 120, such as a differential gene expression signature that can distinguish amongst subjects of the cohort having a phenotype or phenotypes of interest (such as subjects having a pancreatic cancer and subjects that do not have a pancreatic cancer). In the execution phase of the example, a signature generator 130 receives subject data 125 and generates a subject-specific signature. In some embodiments, the signature generator 115 of the training phase is the same as or different than the signature generator 130 of the execution phase. The subject signature is compared 140 to the differential signature, and a predictor 150 receives the results of the comparison 145. The predictor 150 then generates a prediction based on the comparison.

As described herein, in some embodiments, a differential signature (such as a microbial genera signature) can be compared to a subject signature to determine whether a subject that has a cancer (such as pancreatic cancer) or does not have a cancer. In other embodiments, a differential signature (such as a microbial diversity gene signature) can be compared to a subject signature to predict whether the subject (such as a subject that has pancreatic cancer) has a poor survival outcome or a good outcome. In yet another embodiment, a differential signature (such as a T-cell microenvironment reactivity signature) can be compared to a subject signature to determine T-cell microenvironment reaction in a sample from the subject.

In practice, cohorts are compared that comprise subjects having a phenotype of phenotypes of interest. For example, cohort 1 can comprise subjects having a cancer (such as a pancreatic cancer) and cohort 2 can comprise subjects that do not have the cancer. In another example, cohort 1 can comprise subjects that have a good survival outcome (for example, pancreatic cancer subjects that have a known good survival outcome) and cohort 2 can comprise subjects that have a poor outcome (for example, pancreatic cancer subjects that have a known poor survival outcome).

As described herein, the system 100 has been successful in identifying differential microbial genera signatures and in determining if a subject has a cancer, such as a pancreatic cancer; in identifying differential microbial diversity gene signatures and in predicting a survival outcome (such as a good or poor survival outcome) in a subject; and in identifying T-cell microenvironment reactivity signatures and in predicting T-cell microenvironment reaction in a sample from a subject.

In practice, the systems shown herein, such as system 100, can vary in complexity, with additional functionality, more complex components, and the like. For example, there can be additional functionality within the signal generator 115 and/or 130, the comparison function 140, and the predictor function 150. Additional components can be included to implement security, redundancy, load balancing, report design, and the like.

The described computing systems can be networked via wired or wireless network connections, including the Internet. Alternatively, systems can be connected through an intranet connection (e.g., in a corporate environment, government environment, or the like).

The system 100 and any of the other systems described herein can be implemented in conjunction with any of the hardware components described herein, such as the computing systems described below (e.g., processing units, memory, and the like). In any of the examples herein, the data sets, signatures, pathways, and the like can be stored in one or more computer-readable storage media or computer-readable storage devices. The technologies described herein can be generic to the specifics of operating systems or hardware and can be applied in any variety of environments to take advantage of the described features.

### Example 9—Example Method

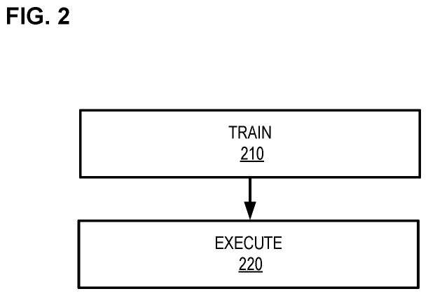

FIG. 2 is a flowchart of an example method 200 determining whether a subject at risk of having a cancer (such as a pancreatic cancer) has the cancer, predicting whether a cancer subject (such as a pancreatic subject) will have a good survival outcome or a poor survival outcome, and/or determining T-cell microenvironment reaction (reactivity) in a subject, and can be implemented, for example, in the system of that shown in FIG. 1.

In the example, at 210, a system is trained. For example, a model can be trained based on old input data to predict future outcomes based on new input data. In practice, the model can include one or more signatures as described herein.

At 220, the system executes. For example, new input data can be input to a trained model that provides an output prediction as described herein.

Further training can be implemented after execution in the form of supervised or unsupervised learning (e.g., actual results can be used instead of predicted results to further train the model).

In practice, the training and executing acts can be implemented by the same or different parties. For example, one party may perform training and then provide the trained model to be executed by another party. As such, the technologies can be described from a training perspective, an execution perspective, or both. For example, a model can be trained as described herein. Such a model can then be applied to generate predictions. Alternatively, a trained model (e.g., generated earlier) can be received and applied to generate predictions.

The method 200 can incorporate any of the methods or acts by systems described herein to achieve the described technologies.

The method 200 and any of the other methods described herein can be performed by computer-executable instructions (e.g., causing a computing system to perform the method) stored in one or more computer-readable media (e.g., storage or other tangible media) or stored in one or more computer-readable storage devices. Such methods can be performed in software, firmware, hardware, or combinations thereof. Such methods can be performed at least in part by a computing system (e.g., one or more computing devices).

The illustrated actions can be described from alternative perspectives while still implementing the technologies.

### Example 10—Example System Identifying Differential Microbial Genera Signatures

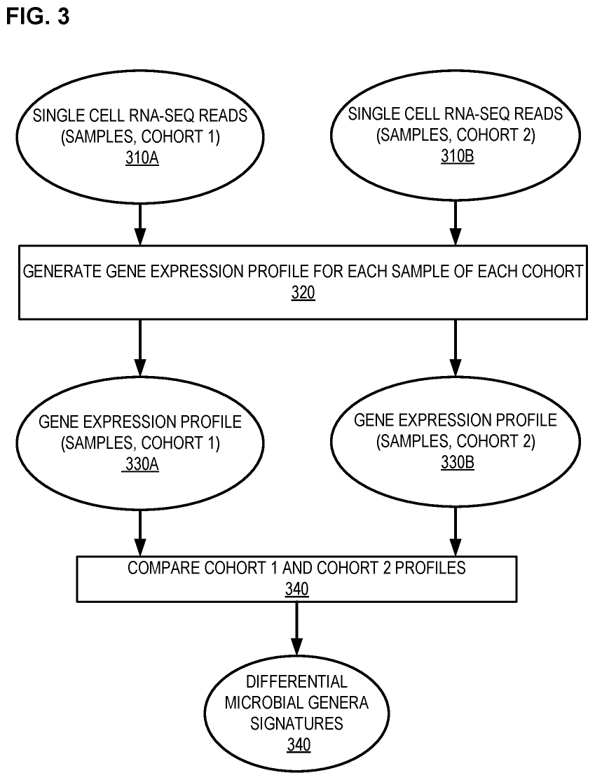

FIG. 3 is a block diagram showing a basic system 300 that can be used to implement identification of microbial genera signatures as described herein. The system 300 can be implemented in a computing system as described herein.

In the example, scRNA-seq reads, for example scRNA-seq reads in the form of FASTQ files, of a first cohort 310A and scRNA-seq reads of a second cohort 310B are used to generate gene expression profiles for each sample in each cohort 320. The gene expression profiles for cohort 1 330A and cohort 2 330B are compared 340, and a differential microbial genera signature 340 is generated. Such signatures can be used, for example, to distinguish subjects of cohort 1 from subjects of cohort 2, such as based on a subject's phenotype or phenotypes of interest.

Such signatures can comprise ranked values for multiple microbial genera or genes. Microbial genera (as represented by gene expression information) present in subjects with cancer and in subjects without cancer can be compared so that scores, ranks, or both of the microbial genera can reflect a given microbial genus' differential abundance between the subject groups.

The example shows scRNA-seq reads for a first 310A and second 310B cohort. In practice, cohorts are compared that comprise subjects having a phenotype of phenotypes of interest. For example, cohort 1 can comprise subjects having a cancer (such as a pancreatic cancer) and cohort 2 can comprise subjects that do not have the cancer. As described herein, the system 300 has been successful in identifying differential microbial genera signatures that can distinguish between a subject having a cancer (such as pancreatic cancer) and a subject that does not have a cancer.

In practice, the systems shown herein, such as system 300, can vary in complexity, with additional functionality, more complex components, and the like. For example, there can be additional functionality within generating gene expression profiles for each sample of each cohort 320 and in comparing cohort 1 and cohort 2 profiles 340. Additional components can be included to implement security, redundancy, load balancing, report design, and the like.

The described computing systems can be networked via wired or wireless network connections, including the Internet. Alternatively, systems can be connected through an intranet connection (e.g., in a corporate environment, government environment, or the like).

The system 300 and any of the other systems described herein can be implemented in conjunction with any of the hardware components described herein, such as the computing systems described below (e.g., processing units, memory, and the like). In any of the examples herein, the data sets, signatures, pathways, and the like can be stored in one or more computer-readable storage media or computer-readable storage devices. The technologies described herein can be generic to the specifics of operating systems or hardware and can be applied in any variety of environments to take advantage of the described features.

### Example 11—Example Method Identifying Microbial Signatures

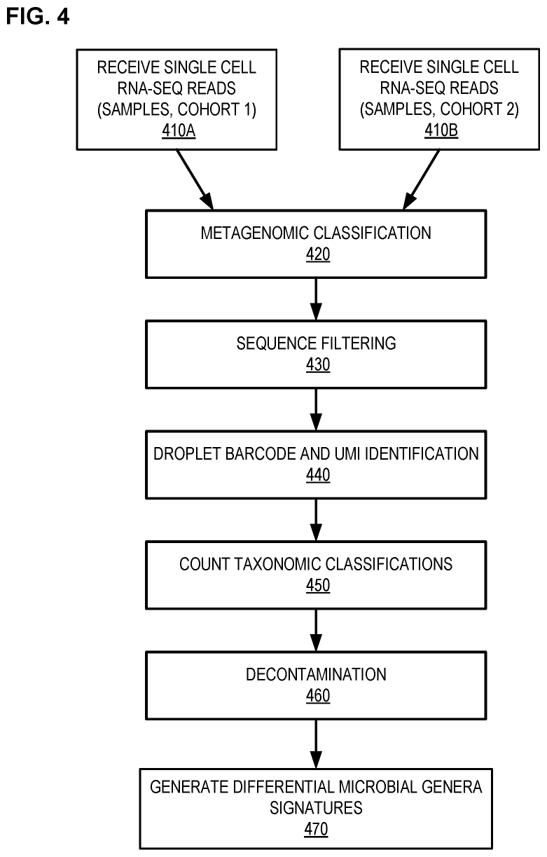

FIG. 4 is a flowchart of an example method 400 identifying microbial genera signatures and can be implemented, for example, in the system of that shown in FIG. 1.

In the example, a metagenomic classification 420 receives scRNA-seq reads, for example scRNA-seq reads in the form of FASTQ files, of a first cohort 410A and scRNA-seq reads of a second cohort 410B. The reads (sequences) are filtered 430, and droplet barcodes and unique molecular identifiers (UMI) are identified 440. Taxonomic classifications are counted 450 and decontaminated 460. In some embodiments, decontamination is done by comparing genera identified in one sample to those identified in, for example, other scRNAseq data of the same organ type, or to those identified by Poore et al. (2020) in TCGA or by Nejman et al. (2020) from 16s-rRNA sequencing of the same organ type. Genera found exclusively in the sample being analyzed are identified as possible contaminants and are removed from further analyses.

Differential microbial genera signatures are output that can distinguish subjects of cohort 1 from subjects of cohort 2, such as based on a subject's phenotype or phenotypes of interest (such as a subject that has a cancer, such as a pancreatic cancer, and a subject that does not have the cancer). Such signatures can comprise ranked values for multiple microbial genera. Microbial genera (as represented by gene expression information) present in subjects with cancer and in subjects without cancer can be compared so that scores, ranks, or both of the microbial genera can reflect a given microbial genus' differential abundance between the subject groups. Outputs can be used as described herein to distinguish between a subject that has a cancer (such as pancreatic cancer) and a subject that does not have a cancer.

In generating differential microbial genera signatures, a microbial genera signature may be generated for each sample in each data set received. For example, reads from scRNA-seq experiments are mapped to the subject (e.g., human) genome and the resulting transcriptomic signatures can be clustered (for example, using the Seurat (Stuart et al. Cell, 177: 1888-1902.e21, 2019) R package with default parameters) and somatic cell types annotated and quantitated.

In generating differential microbial genera signatures, microbial genera signatures from each sample in each data set (such as from each sample in each cohort) are compared as described herein, to identify differentially expressed metagenomes, such as between tumor and non-tumor (and/or non-malignant) samples. For example, cell counts can be log 1p normalized and scaled. In some examples, microbes can be included in a differential microbial genera signature if they are found to be differentially present in either tumors or control samples and if their abundance is >10′ or if they are custom selected. Microbiome abundances per sample can be normalized, centered and unit-scaled. Normalized and scaled cell counts, pathway scores, and microbiome abundances for all samples can be combined into a matrix and used as input to, for example, Monocle's pseudotime functions (Trapnell et al. Nat. Biotechnol. 32: 381-386, 2014), using expressionFamily=uninormal( ) and norm_method=“none”. Numerical microbiome and clinical parameters can be compared across the resulting states using a t-test, and categorical parameters using Fisher's test.

Subsequently, microbial signatures are generated that can distinguish tumor from non-tumor (or non-malignant) samples. As described herein the method 400 has been successful in identifying useful microbial signatures.

The method 400 can incorporate any of the methods or acts by systems described herein to achieve the described technologies.

The method 400 and any of the other methods described herein can be performed by computer-executable instructions (e.g., causing a computing system to perform the method) stored in one or more computer-readable media (e.g., storage or other tangible media) or stored in one or more computer-readable storage devices. Such methods can be performed in software, firmware, hardware, or combinations thereof. Such methods can be performed at least in part by a computing system (e.g., one or more computing devices).

The illustrated actions can be described from alternative perspectives while still implementing the technologies.

### Example 12—Example System Determining If a Subject Has a Cancer

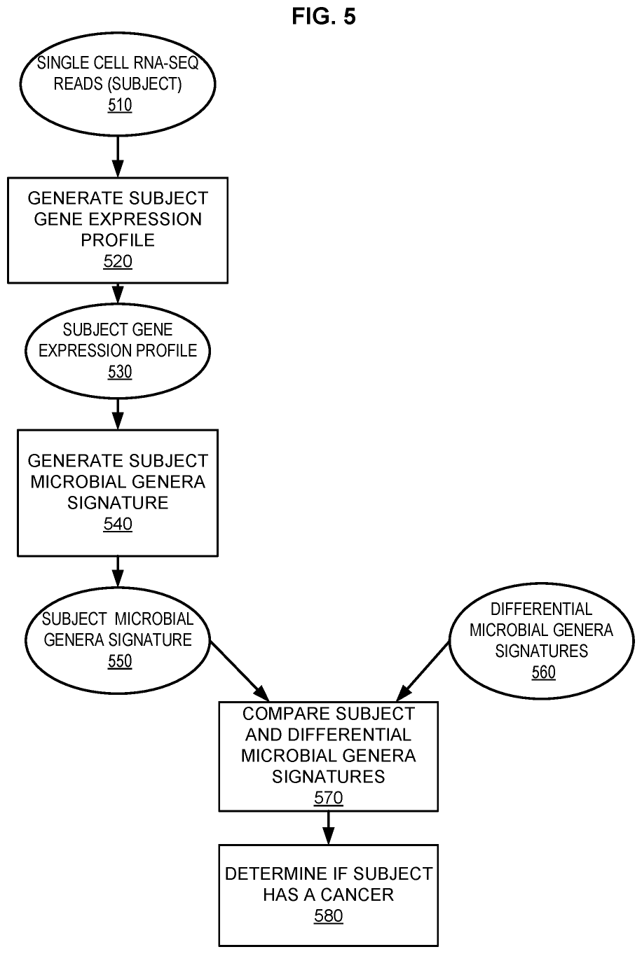

FIG. 5 is a block diagram showing a basic system 500 that can be used to implement determining whether a subject at risk of having a cancer (such as a pancreatic cancer) has the cancer as described herein. The system 500 can be implemented in a computing system as described herein. In the example, scRNA-seq reads from a subject 510 are used to generate gene expression profiles 520 for each sample from the subject. The gene expression profile or profiles 530 are used to generate a microbial genera signature 540 for each sample from the subject and/or for the samples from subject combined. The subject's microbial genera signature or signatures are compared 570 to a differential microbial genera signature 560 (such as a signature generated using the system of FIG. 1 or FIG. 3). The subject is determined to have the cancer or to not have the cancer 580 based on the similarity or dissimilarity of the subject (and/or sample) microbial genera signature and the differential microbial genera signature.

As described herein, the system 500 has been successful determining if a subject has a cancer, such as a pancreatic cancer.

In practice, the systems shown herein, such as system 500, can vary in complexity, with additional functionality, more complex components, and the like. For example, there can be additional functionality within generating gene expression profiles for each sample from the subject 520, in comparing subject and differential microbial genera signatures 570, and in determining if the subject has a cancer 580. Additional components can be included to implement security, redundancy, load balancing, report design, and the like.

The described computing systems can be networked via wired or wireless network connections, including the Internet. Alternatively, systems can be connected through an intranet connection (e.g., in a corporate environment, government environment, or the like).

The system 500 and any of the other systems described herein can be implemented in conjunction with any of the hardware components described herein, such as the computing systems described below (e.g., processing units, memory, and the like). In any of the examples herein, the data sets, signatures, pathways, and the like can be stored in one or more computer-readable storage media or computer-readable storage devices. The technologies described herein can be generic to the specifics of operating systems or hardware and can be applied in any variety of environments to take advantage of the described features.

### Example 13—Example Method of Determining if a Subject Has a Cancer

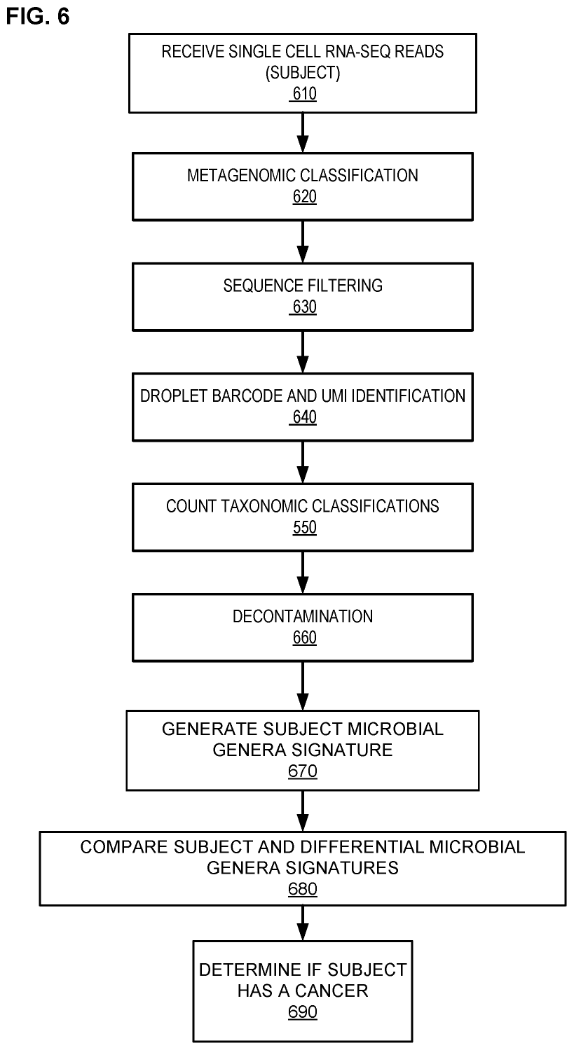

FIG. 6 is a flowchart of an example method 600 for determining if a subject at risk of having a cancer has the cancer (such as a pancreatic cancer), and can be implemented, for example, in the system of that shown in FIG. 1 or FIG. 5.

In the example, a metagenomic classification 620 receives scRNA-seq reads, for example scRNA-seq reads in the form of FASTQ files, of a subject 610. The reads (sequences) are filtered 630, and droplet barcodes and unique molecular identifiers (UMI) are identified 640. Taxonomic classifications are counted 650 and decontaminated 660. In some embodiments, decontamination is done by comparing genera identified in one sample to those identified in, for example, other scRNAseq data of the same organ type, or to those identified by Poore et al. (2020) in TCGA or by Nejman et al. (2020) from 16s-rRNA sequencing of the same organ type. Genera found exclusively in the sample being analyzed are identified as possible contaminants and are removed from further analyses.

A subject microbial genera signature is then generated 670. Such signatures can comprise ranked values for multiple microbial genera. The subject's microbial genera signature or signatures are compared 680 to a differential microbial genera signature (such as a signature generated using the system of FIG. 1 or FIG. 3). The subject is determined to have the cancer or to not have the cancer 690 based on the similarity or dissimilarity of the subject (and/or sample) microbial genera signature and the differential microbial genera signature.

In generating a microbial genera signature for the subject and/or for each sample received from the subject individually, reads from scRNA-seq experiments are mapped to the subject (e.g., human) genome and the resulting transcriptomic signatures can be clustered (for example, using the Seurat (Stuart et al. Cell, 177: 1888-1902.e21, 2019) R package with default parameters) and somatic cell types annotated and quantitated. Microbiome abundances per sample can be normalized, centered and unit-scaled. Normalized and scaled cell counts, pathway scores, and microbiome abundances for all samples can be combined into a matrix and used as input to, for example, Monocle's pseudotime functions (Trapnell et al. Nat. Biotechnol. 32: 381-386, 2014), using expressionFamily=uninormal( ) and norm_method=“none”.

As described herein the method 600 has been successful in determining if a subject has a cancer (such as pancreatic cancer) or does not have a cancer.

The method 600 can incorporate any of the methods or acts by systems described herein to achieve the described technologies.

The method 600 and any of the other methods described herein can be performed by computer-executable instructions (e.g., causing a computing system to perform the method) stored in one or more computer-readable media (e.g., storage or other tangible media) or stored in one or more computer-readable storage devices. Such methods can be performed in software, firmware, hardware, or combinations thereof.

Such methods can be performed at least in part by a computing system (e.g., one or more computing devices).

The illustrated actions can be described from alternative perspectives while still implementing the technologies.

### Example 14—Example System Identifying Microbial Diversity Gene Signatures

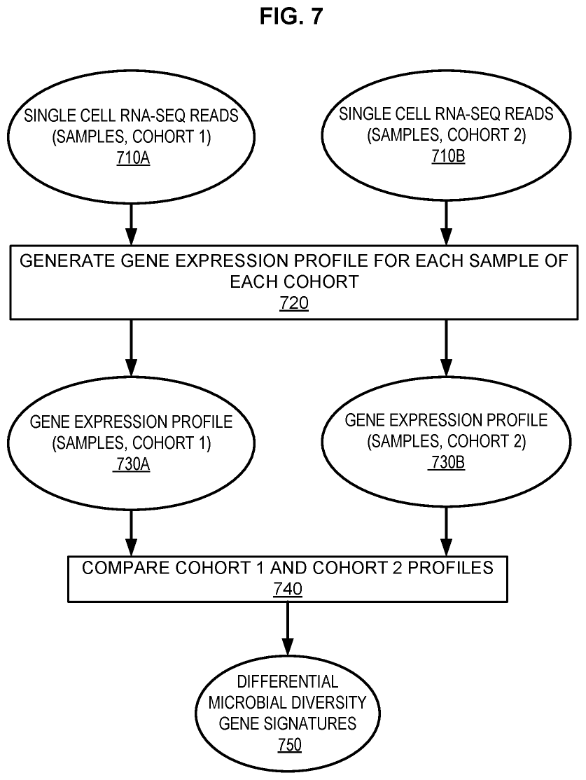

FIG. 7 is a block diagram showing a basic system 700 that can be used to implement identification of microbial diversity gene signatures as described herein. The system 700 can be implemented in a computing system as described herein.

In the example, scRNA-seq reads, for example scRNA-seq reads in the form of FASTQ files, of a first cohort 710A and scRNA-seq reads of a second cohort 710B are used to generate gene expression profiles for each sample in each cohort 720. The gene expression profiles for cohort 1 730A and cohort 2 730B are compared 740, and a differential microbial diversity gene signature 740 is generated. Such signatures can be used, for example, to distinguish subjects of cohort 1 from subjects of cohort 2, such as based on a subject's phenotype or phenotypes of interest.

Such signatures can comprise ranked values for multiple microbial genera or genes. Microbial genera (as represented by gene expression information) present in subjects with cancer and in subjects without cancer can be compared so that scores, ranks, or both of the microbial genera can reflect a given microbial genus' differential abundance between the subject groups.

In practice, cohorts are compared that comprise subjects having a phenotype of phenotypes of interest. For example, cohort 1 can comprise cancer subjects (such as pancreatic cancer subjects) with a known poor outcome and cohort 2 can comprise cancer subjects (such as pancreatic cancer subjects) with a known good outcome. As described herein, the system 700 has been successful in identifying differential microbial genera signatures that can distinguish between a cancer subject (such as pancreatic cancer subject) with a poor outcome and a cancer subject (such as pancreatic cancer subject) with a good outcome.

In practice, the systems shown herein, such as system 700, can vary in complexity, with additional functionality, more complex components, and the like. For example, there can be additional functionality within generating gene expression profiles for each sample of each cohort 720 and in comparing cohort 1 and cohort 2 profiles 740. Additional components can be included to implement security, redundancy, load balancing, report design, and the like.

The described computing systems can be networked via wired or wireless network connections, including the Internet. Alternatively, systems can be connected through an intranet connection (e.g., in a corporate environment, government environment, or the like).

The system 700 and any of the other systems described herein can be implemented in conjunction with any of the hardware components described herein, such as the computing systems described below (e.g., processing units, memory, and the like). In any of the examples herein, the data sets, signatures, pathways, and the like can be stored in one or more computer-readable storage media or computer-readable storage devices. The technologies described herein can be generic to the specifics of operating systems or hardware and can be applied in any variety of environments to take advantage of the described features.

### Example 15—Example Method Identifying Microbial Diversity Gene Signatures

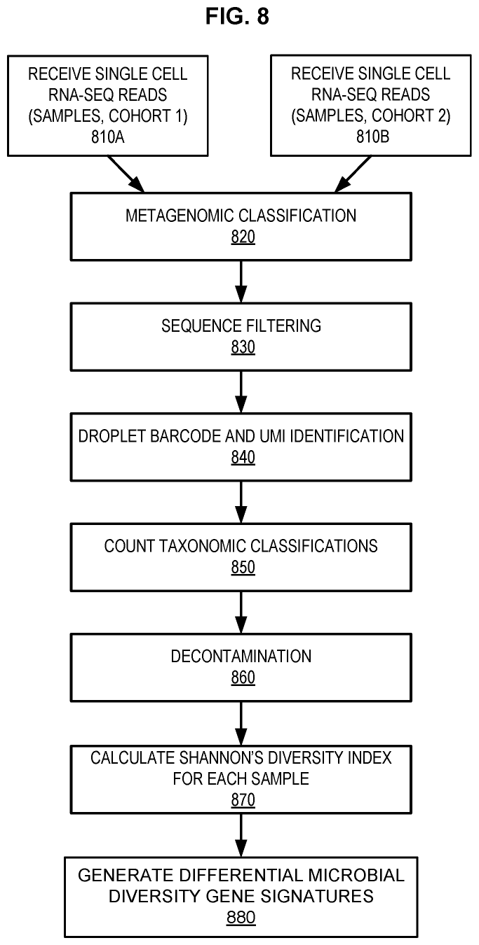

FIG. 8 is a flowchart of an example method 800 identifying microbial diversity gene signatures and can be implemented, for example, in the system of that shown in FIG. 1 or FIG. 7.

In the example, a metagenomic classification 820 receives scRNA-seq reads, for example scRNA-seq reads in the form of FASTQ files, of a first cohort 810A and scRNA-seq reads of a second cohort 810B. The reads (sequences) are filtered 830, and droplet barcodes and unique molecular identifiers (UMI) are identified 840. Taxonomic classifications are counted 850 and decontaminated 860. Such signatures can comprise ranked values for multiple microbial genera.

At 870, Shannon's diversity index is calculated for each sample. The Shannon diversity index (H) is a mathematical measure that is used to characterize species diversity in a community, and accounts for both species richness (the number of species present) and evenness (relative abundances of different species) present in the community. Most often, the proportion of species i relative to the total number of species (pᵢ) is calculated and multiplied by the natural logarithm of the proportion (lnpᵢ). The result is then summed across species and multiplied by −1:

$$H = - {\sum }_{i = 1}^{k} {p}_{i} log ( {p}_{i} )$$

In some embodiments, Shannon's equitability (E_{H}) can be determined by dividing H by the maximum diversity (log(k)). This normalizes the Shannon diversity index to a value between 0 and 1, with 1 being complete evenness of species in the community. In other words, an index value of 1 means that all species groups have the same frequency.

$${E}_{H} = \frac{H}{log ( k )}$$

At 880, microbial diversity gene signatures are generated. In generating such signatures, genes are identified that are differentially expressed between samples that are classified as having a high or low microbial diversity based on Shannon's diversity index as calculated for each sample.

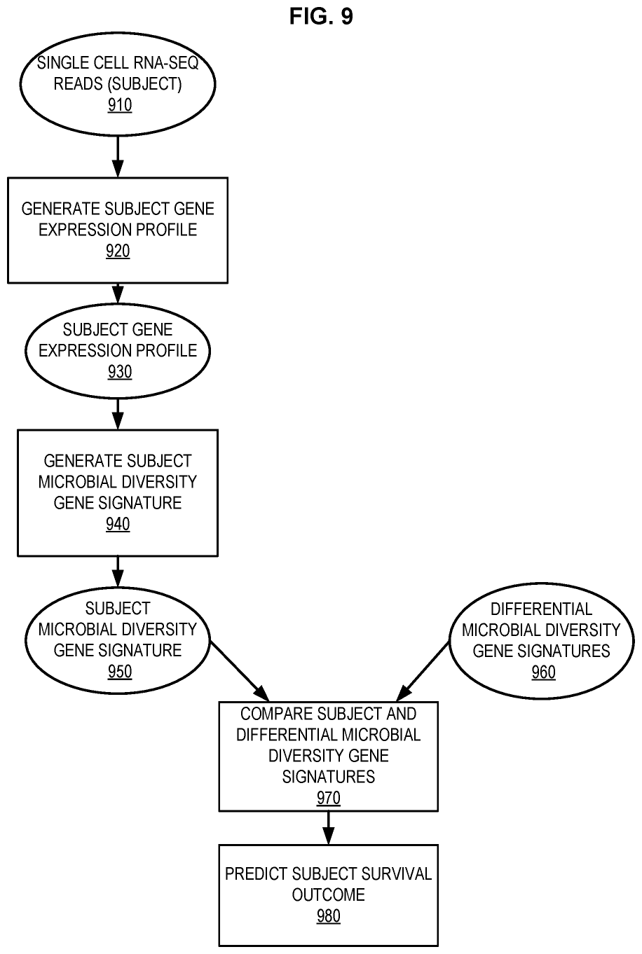

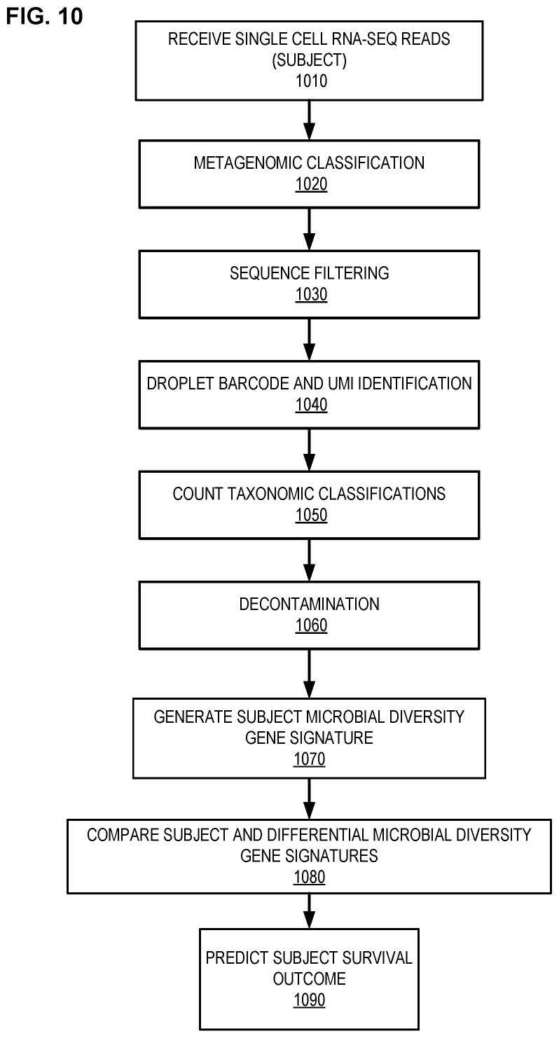

As described herein the method 800 has been successful in identifying differential microbial diversity gene signatures that can be used to predict survival outcomes in subjects whose survival outcome is not yet known, such as using the system of FIG. 9 or the method of FIG. 10.

The method 800 can incorporate any of the methods or acts by systems described herein to achieve the described technologies.

The method 800 and any of the other methods described herein can be performed by computer-executable instructions (e.g., causing a computing system to perform the method) stored in one or more computer-readable media (e.g., storage or other tangible media) or stored in one or more computer-readable storage devices. Such methods can be performed in software, firmware, hardware, or combinations thereof.

Such methods can be performed at least in part by a computing system (e.g., one or more computing devices).

The illustrated actions can be described from alternative perspectives while still implementing the technologies.

### Example 16—Example System Predicting a Survival Outcome in a Subject

FIG. 9 is a block diagram showing a basic system 900 that can be used to implement determining whether a cancer subject (such as a pancreatic subject) will have a good survival outcome or a poor survival outcome as described herein. The system 900 can be implemented in a computing system as described herein. In the example, scRNA-seq reads from a subject 910 are used to generate gene expression profiles 920 for each sample from the subject. The gene expression profile or profiles 930 are used to generate a microbial diversity gene signature 940 for each sample from the subject and/or for the samples from subject combined. The subject's microbial diversity gene signature or signatures are compared 970 to a differential microbial diversity gene signature 960 (such as a signature generated using the system of FIG. 1 or FIG. 7). The subject is determined to have a good survival outcome or a poor survival outcome 980 based on the similarity or dissimilarity of the subject (and/or sample) microbial genera signature and the differential microbial genera signature.

As described herein, the system 900 has been successful determining if a subject has a cancer, such as a pancreatic cancer.

In practice, the systems shown herein, such as system 900, can vary in complexity, with additional functionality, more complex components, and the like. For example, there can be additional functionality within generating gene expression profiles for each sample from the subject 920, in comparing subject and differential microbial genera signatures 970, and in predicting the survival outcome of the subject 980. Additional components can be included to implement security, redundancy, load balancing, report design, and the like.

The described computing systems can be networked via wired or wireless network connections, including the Internet. Alternatively, systems can be connected through an intranet connection (e.g., in a corporate environment, government environment, or the like).

The system 900 and any of the other systems described herein can be implemented in conjunction with any of the hardware components described herein, such as the computing systems described below (e.g., processing units, memory, and the like). In any of the examples herein, the data sets, signatures, pathways, and the like can be stored in one or more computer-readable storage media or computer-readable storage devices. The technologies described herein can be generic to the specifics of operating systems or hardware and can be applied in any variety of environments to take advantage of the described features.

### Example 17—Example Method of Predicting a Survival Outcome in a Subject

FIG. 10 is a flowchart of an example method 1000 identifying microbial biomarkers and can be implemented, for example, in the system of that shown in FIG. 1 or FIG. 8.

In the example, a metagenomic classification 1020 receives scRNA-seq reads, for example scRNA-seq reads in the form of FASTQ files, of a subject 1010. The reads (sequences) are filtered 1030, and droplet barcodes and unique molecular identifiers (UMI) are identified 1040. Taxonomic classifications are counted 1050 and decontaminated 1060, and a subject microbial diversity gene signature is generated 1070 as described herein (such as in Examples 15 and 16. The subject's microbial diversity gene signature or signatures are compared 1080 to a differential microbial diversity gene signature (such as a signature generated using the system of FIG. 1 or FIG. 8). The subject is predicted to have a good survival outcome or a poor survival outcome 1090 based on the similarity or dissimilarity of the subject (and/or sample) microbial diversity gene signature and the differential microbial diversity gene signature.

In other embodiments, Shannon's diversity score as calculated for the subject or for each sample from the subject can be used to predict a survival outcome in the subject. In such examples, a Shannon's diversity score indicating high microbial diversity in the sample (such as compared to a control, such as a sample from a subject with a good or poor survival outcome) can indicate a poor survival outcome in the subject, and a Shannon's diversity score indicating low microbial diversity in the sample (such as compared to a control, such as a sample from a subject with a good or poor survival outcome) can indicate a good survival outcome in the subject

As described herein the method 1000 has been successful in predicting if a cancer subject has a poor or good survival outcome.

The method 1000 can incorporate any of the methods or acts by systems described herein to achieve the described technologies.

The method 1000 and any of the other methods described herein can be performed by computer-executable instructions (e.g., causing a computing system to perform the method) stored in one or more computer-readable media (e.g., storage or other tangible media) or stored in one or more computer-readable storage devices. Such methods can be performed in software, firmware, hardware, or combinations thereof.

Such methods can be performed at least in part by a computing system (e.g., one or more computing devices).

The illustrated actions can be described from alternative perspectives while still implementing the technologies.

### Example 18—Example System Identifying Differential T-cell Microenvironment Reactivity Signatures

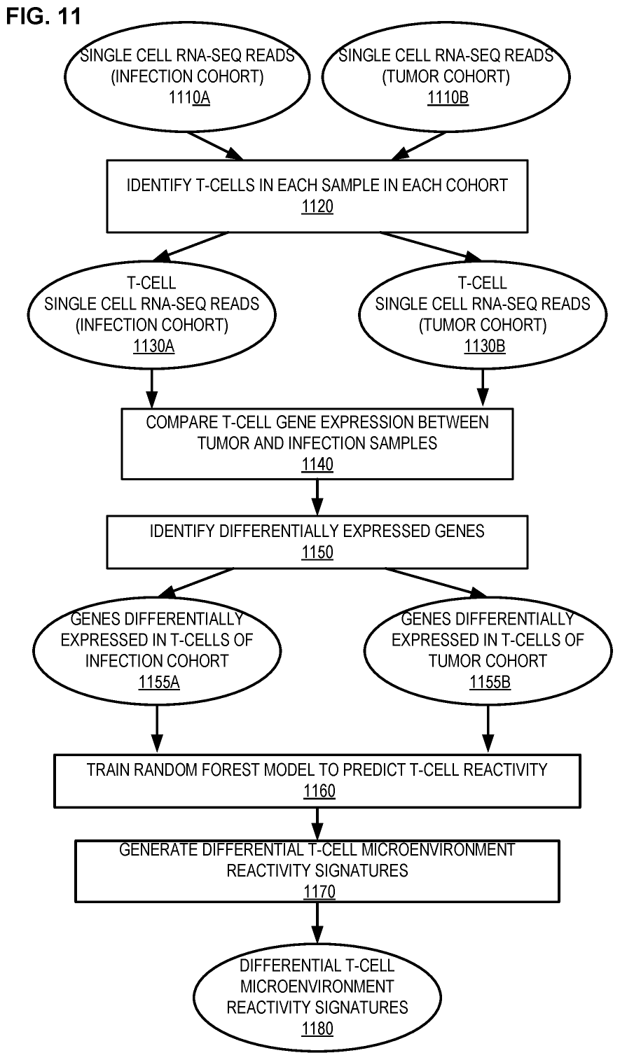

FIG. 11 is a block diagram showing a basic system 1100 that can be used to implement identification of differential T-cell microenvironment reactivity signatures as described herein. The system 1100 can be implemented in a computing system as described herein.

In the example, scRNA-seq reads, for example scRNA-seq reads in the form of FASTQ files, of a first cohort 1110A (wherein subjects in the cohort have an infection) and scRNA-seq reads of a second cohort 1110B (wherein subjects in the cohort have a tumor) are used to identify T-cell reads for each sample in each cohort 1120. The T-cell scRNA-seq reads from the infection cohort 1130A and the tumor cohort 1130B are compared 1140 and genes differentially expressed between the cohorts are identified 1150. Genes differentially expressed in the infection cohort 1155A and genes differentially expressed in the tumor cohort 1155B are used to train a random forest model to predict T-cell reactivity 1160 as described herein, and a differential T-cell microenvironment reactivity signature is generated that can distinguish between infection microenvironment reactive T-cells and tumor microenvironment reactive T-cells. Such signatures can comprise ranked values for multiple genes.

As described herein, the system 1100 has been successful in identifying differential T-cell microenvironment reactivity signatures that can distinguish between infection microenvironment reactive T-cells and tumor microenvironment reactive T-cells.

In practice, the systems shown herein, such as system 1100, can vary in complexity, with additional functionality, more complex components, and the like. For example, there can be additional functionality within identifying T-cells in each sample in each cohort 1120, training a random forest model to predict T-cell reactivity 1160, and generating differential T-cell microenvironment reactivity signatures. Additional components can be included to implement security, redundancy, load balancing, report design, and the like.

The described computing systems can be networked via wired or wireless network connections, including the Internet. Alternatively, systems can be connected through an intranet connection (e.g., in a corporate environment, government environment, or the like).

The system 1100 and any of the other systems described herein can be implemented in conjunction with any of the hardware components described herein, such as the computing systems described below (e.g., processing units, memory, and the like). In any of the examples herein, the data sets, signatures, pathways, and the like can be stored in one or more computer-readable storage media or computer-readable storage devices. The technologies described herein can be generic to the specifics of operating systems or hardware and can be applied in any variety of environments to take advantage of the described features.

### Example 19—Example Method Identifying Differential T-cell Microenvironment Reactivity Signatures

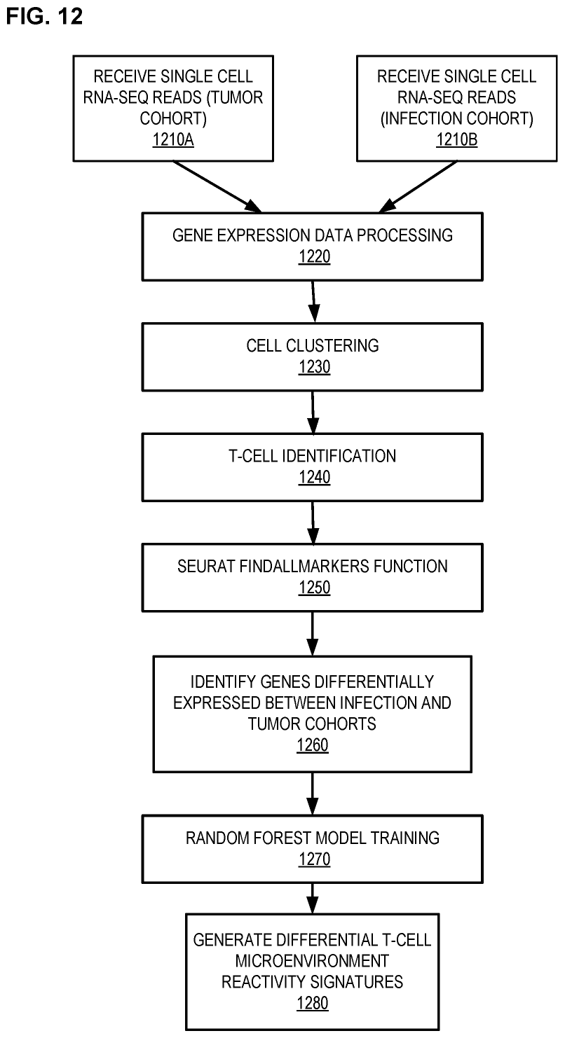

FIG. 12 is a flowchart of an example method 1200 that can be used to implement identification of differential T-cell microenvironment reactivity signatures, for example, in the system of that shown in FIG. 1 or FIG. 11.

In the example, a gene expression data processing step 1220 receives both scRNA-seq reads from subjects having an infection 1210A and scRNA-seq reads from subjects having a tumor 1210B, for example as FASTQ files. Data are processed using the standard Seurat pipeline; gene expression counts for each cell are log normalized for total sequencing counts using the NormalizeData function, 2000 highly variable genes are selected using the FindVariableGenes function, and cells are clustered 1230 based on transcriptomic profiles by sequentially using the RunPCA, RunUMAP, FindNeighbors, and FindClusters functions. T-cells are identified 1240 using known markers (Nirmal et al. Cancer Immunol. Res. 6(11):1388-1400, 2018). The FindAllMarkers function from Seurat 1250 is used to identify genes differentially expressed 1260 in T-cells between tumor and infection samples. Genes differentially expressed in T-cells of the infection cohort and the tumor cohort are used to train a random forest model to predict T-cell reactivity 1270 as described herein, and a differential T-cell microenvironment reactivity signature is generated 1280 that can distinguish between infection microenvironment reactive T-cells and tumor microenvironment reactive T-cells. Such signatures can comprise ranked values for multiple genes.

As described herein the method 1200 has been successful in predicting if a cancer subject has a poor or good survival outcome.

The method 1200 can incorporate any of the methods or acts by systems described herein to achieve the described technologies.

The method 1200 and any of the other methods described herein can be performed by computer-executable instructions (e.g., causing a computing system to perform the method) stored in one or more computer-readable media (e.g., storage or other tangible media) or stored in one or more computer-readable storage devices. Such methods can be performed in software, firmware, hardware, or combinations thereof. Such methods can be performed at least in part by a computing system (e.g., one or more computing devices).

The illustrated actions can be described from alternative perspectives while still implementing the technologies.

### Example 20—Example System Determining T-cell Microenvironment Reactivity

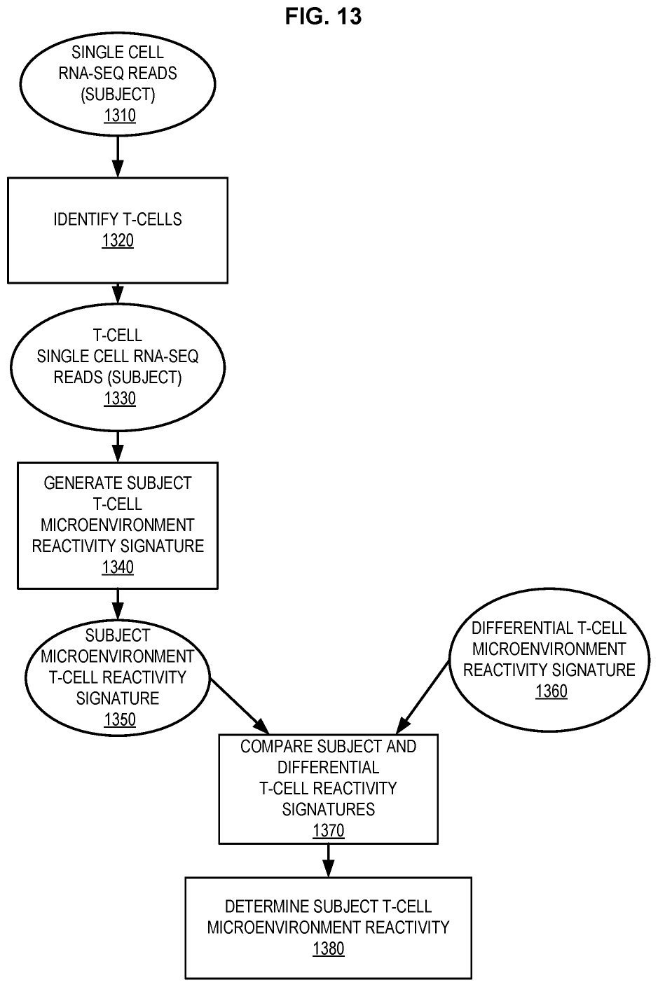

FIG. 13 is a block diagram showing a basic system 1300 that can be used to implement determination of T-cell microenvironment reactivity (also referred to herein as T-cell reactivity) as described herein. The system 1300 can be implemented in a computing system as described herein.

In the example, a T-cell identification step 1320 receives scRNA-seq reads from a subject 1310, for example as FASTQ files. The T-cell scRNA-seq reads 1330 from the subject are used to generate a T-cell microenvironment reactivity signature 1340 for each T-cell from the subject, for each sample from the subject, and/or for the subject as a whole. Such signatures can comprise ranked values for multiple genes.

The T-cell microenvironment reactivity signature or signatures are compared 1370 to a differential T-cell microenvironment reactivity signature 1360 (such as a signature generated using the system of FIG. 1 or FIG. 8). The T-cells of the subject or of the sample from the subject are individually determined to be infection microenvironment reactive T-cells and tumor microenvironment reactive T-cells based on the similarity or dissimilarity of the T-cell microenvironment reactivity signature and the differential T-cell microenvironment reactivity signature.

As described herein, the system 1300 has been successful in determining whether T-cells from a subject are infection microenvironment reactive T-cells and tumor microenvironment reactive T-cells.

In practice, the systems shown herein, such as system 1300, can vary in complexity, with additional functionality, more complex components, and the like. For example, there can be additional functionality within identification of T-cells 1320, or within generating one or more T-cell microenvironment reactivity signatures for the subject or the individual T-cells of the subject. Additional components can be included to implement security, redundancy, load balancing, report design, and the like.

The described computing systems can be networked via wired or wireless network connections, including the Internet. Alternatively, systems can be connected through an intranet connection (e.g., in a corporate environment, government environment, or the like).

The system 1300 and any of the other systems described herein can be implemented in conjunction with any of the hardware components described herein, such as the computing systems described below (e.g., processing units, memory, and the like). In any of the examples herein, the data sets, signatures, pathways, and the like can be stored in one or more computer-readable storage media or computer-readable storage devices. The technologies described herein can be generic to the specifics of operating systems or hardware and can be applied in any variety of environments to take advantage of the described features.

### Example 21—Example Method Determining T-cell Microenvironment Reactivity

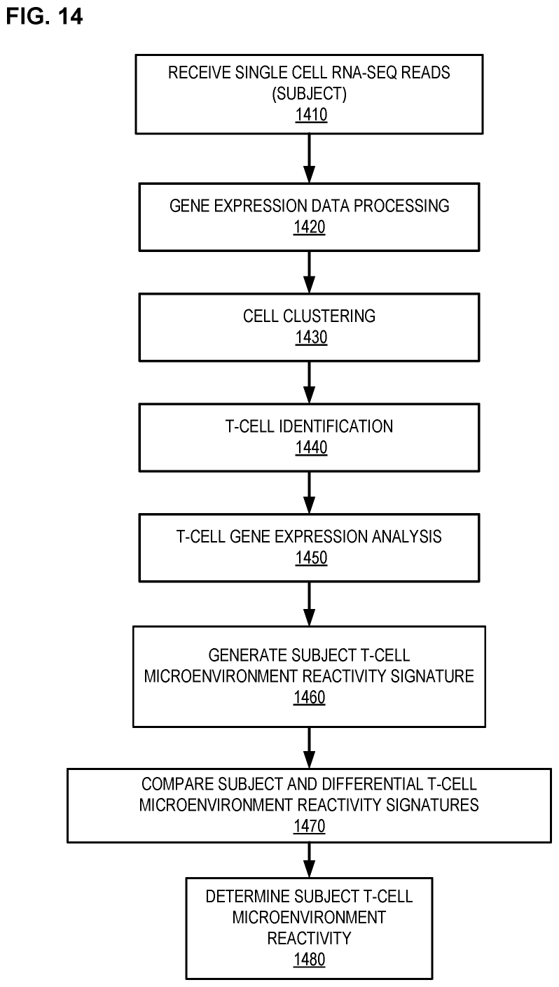

FIG. 14 is a flowchart of an example method 1400 for determining T-cell microenvironment reactivity and can be implemented, for example, in the system of that shown in FIG. 1 or FIG. 13.

In the example, a gene expression data processing step 1420 receives both scRNA-seq reads from a subject 1410, for example as FASTQ files. Data are processed using the standard Seurat pipeline; gene expression counts for each cell are log normalized for total sequencing counts using the NormalizeData function, 2000 highly variable genes are selected using the FindVariableGenes function, and cells are clustered 1230 based on transcriptomic profiles by sequentially using the RunPCA, RunUMAP, FindNeighbors, and FindClusters functions. T-cells are identified 1240 using known markers (Nirmal et al. Cancer Immunol. Res. 6(11):1388-1400, 2018). The T-cell microenvironment reactivity signature is generated 1460 by using a pretrained random forest classifier. The subject's T-cell microenvironment reactivity signature or signatures are compared 1470 to a differential T-cell microenvironment reactivity signature (such as a signature generated using the system of FIG. 1 or FIG. 13). The T-cells of the subject or of the sample from the subject are determined (individually and/or as a whole) to be infection microenvironment reactive T-cells and tumor microenvironment reactive T-cells based on the similarity or dissimilarity of the T-cell microenvironment reactivity signature and the differential T-cell microenvironment reactivity signature.

As described herein the method 1400 has been successful in predicting if a cancer subject has a poor or good survival outcome.

The method 1400 can incorporate any of the methods or acts by systems described herein to achieve the described technologies.

The method 1400 and any of the other methods described herein can be performed by computer-executable instructions (e.g., causing a computing system to perform the method) stored in one or more computer-readable media (e.g., storage or other tangible media) or stored in one or more computer-readable storage devices. Such methods can be performed in software, firmware, hardware, or combinations thereof. Such methods can be performed at least in part by a computing system (e.g., one or more computing devices).

The illustrated actions can be described from alternative perspectives while still implementing the technologies.

### Example 22—Example Implementation of Receiving Expression Data

Any of the examples herein can include receiving a variety of genomic data, such as expression data, such as gene expression data (for example, one or more datasets that include one or more datapoints). In practice, expression data can include data on genes or sets of genes. For example, a targeted set of genes or a genome-wide set of genes can be included.

In practice, receiving expression data can include expression data for at least one subject (such as a subject with a known survival outcome, or a training subject, or a subject with an unknown survival outcome, or a query subject) or at least one group of subjects (such a group of subjects with a common feature or characteristic, or a cohort). In specific, non-limiting examples, receiving expression data can include genomic data, such as sequencing data, for at least 2 cohorts, such as cohorts with a different disease status or with different phenotypes (for example, 2 cohorts with the same disease but different survival outcome phenotypes). For example, FIG. 3 shows receiving 310A an scRNA-seq reads data set for a first cohort (such as a cohort of cancer subjects, such as pancreatic cancer subjects) and receiving 310B an scRNA-seq data set for a second cohort (such as a cohort of subjects that do not have cancer). In examples, receiving expression data can include expression data for a subject or subjects with a common feature or characteristic, such as a disease (for example, cancer, or a malignant tumor characterized by abnormal or uncontrolled cell growth, such as pancreatic cancer or lung cancer) and/or a survival outcome phenotype (for example, a cancer patient or cohort of patients having pancreatic cancer and good survival outcomes, or a cancer patient or cohort of patients having pancreatic cancer and poor survival outcomes).

In specific, non-limiting examples, receiving expression data can include expression data for single subjects or a group of subjects with a common disease (such as cancer, for example, a malignant tumor characterized by abnormal or uncontrolled cell growth, such as pancreatic cancer or lung cancer).

In practice, receiving expression data can include a variety of processing steps. In examples, processing steps can include normalization, transformation (such as stabilized variance, P value or M value transformation, log transformation, z-score, or rank transformation), redundancy reduction (for example, based on statistical factor, such as a highest coefficient of variation), centering, standardization, logit transformation, bias correction, background correction, and the like.

### Example 23—Example Implementation of Identifying Differential Expression Datapoints

Any of the examples herein can include identifying differential expression data (for example, differential gene expression datapoints in a dataset), such as by a differential identifier. In practice, one or more differential expression signatures can be generated. For example, FIG. 4 shows generating differential microbial genera signatures 470 that can distinguish between a subject that has a cancer (such as a pancreatic cancer) and a subject that does not have the cancer.

In examples, differential expression data or datapoints can include differential expression of genes or sets of genes. For example, genes in which an amount of one or more of its expression products (for example, transcripts, such as mRNA) is higher or lower in one sample (such as a test sample, such as a pancreatic cancer sample) as compared to another sample (such as a control sample or a reference standard, for example, a healthy subject or subjects or a subject or subjects with a disease and/or survival outcome phenotype, such as a subject or subjects with good survival outcomes, or a subject or subjects with poor survival outcomes, or a historical control, or standard reference value or range of values). In practice, differential expression can include an increase or a decrease in expression of a gene or genes. Differential expression can include a quantitative increase or a decrease in expression, for example, a statistically significant increase or decrease.

In examples, various methods can be used to identify differential genes for differential expression signatures. For example, scRNA-seq data (such as described herein) for a gene or a set of genes can be compared.

In practice, a variety of processing steps can also be applied. For example, processing can include a quantitative comparison. For example, a statistical comparison can be used, such as a t-statistic (for example, using a two-tailed t-test, such as a Student's or Welch's t-test, for example, a two-tailed Welch's t-test) or other statistical comparison, such as a Wilcoxon-Mann-Whitney test. Thus, genes or a set of genes associated with level of gene expression as described herein can be input into a differential identifier, and a list of genes or set of genes, in which each gene is associated with a level of differential expression can be output, such as a differential gene expression signature.

In practice, differential expression signatures can be output with a variety of forms. For example, a ranked list (such as based on level of differentiation), a list of genes with significance assigned, or a list of genes that meet an applied cut-off threshold (such as based on level of differentiation). Other forms are possible. For example, where gene differentiation is quantified (for example, producing positive values for overexpression and producing negative values for underexpression), differential expression signatures can include absolute valued differential expression signatures or signed differential expression signatures.

In any of the examples herein, a variety of differential expression signatures can be generated for genes or a set of genes. In practice, one or more than one differential expression signature can be generated for genes or a set of genes. In examples, more than one differential expression signature can be generated for more than one list of genes or a set of genes, such as during training. In examples, a single sample expression signature can be generated for a single list of genes or a set of genes, such as during use or validation.

In practice, differential expression signatures can include various genes or sets of genes. For example, a targeted set of genes (such as for use or validation, for example, genes associated with a survival outcome phenotype, T-cell reactivity, and/or pathways in a pathway signature) or a genome-wide set of genes can be included (such as for training, for example, using gene or gene sets associated with microbial organisms, gene or gene sets associated with T-cells, or gene or genes sets of biological pathways, such as included in general or specific biological pathways databases, for example, EcoCyc, KEGG, RegulonDB, MetaCyc STRINGDB, PANTHER, Gene Ontology, REACTOME, MSigDb, Ingenuity Knowledge Base, NCI PID, WikiPathways, Small Molecule Pathway DB, ConsensusPathDB, Pathway Commons, or the like, such as described in Garcia-Campos et al., Front. Physiol, 6(383), 2015, incorporated herein by reference in its entirety).

### Example 24—Example Implementation of Determining Biological Pathways Enriched Differential Genomic Signatures

Any of the examples herein can include determining biological pathways enriched in a differential expression signature, such as by a pathway enrichment identifier. In practice, one or more genomic or epigenomic signatures can be generated. For example, Example 25 describes pathway enrichment associated with microbial gene expression.

In practice, biological pathways enriched in a differential expression signature can be determined in a variety of ways. For example, genes or a set of genes in a differential expression signature can be compared with genes in biological pathways, such as included in general or specific biological pathways databases, for example, EcoCyc, KEGG, RegulonDB, MetaCyc STRINGDB, PANTHER, Gene Ontology, REACTOME, MSigDb, Ingenuity Knowledge Base, NCI PID, WikiPathways, Small Molecule Pathway DB, ConsensusPathDB, Pathway Commons, or the like (for example, as described in Garcia-Campos et al., Front. Physiol, 6(383), 2015, incorporated herein by reference in its entirety).

In practice, a variety of processing steps can also be applied. For example, processing can include a quantitative comparison. In examples, a statistical comparison can be used, such as the Kolmogorov-Smirnov statistic, Mann-Whitney test, t-tests (for example, Welch's or Student's t-test), chi-square, Fisher's exact test, binomial, probability, hypergeometric distribution, z-score, permutation analysis, kappa statistics and the like. Other enrichment analysis tools or algorithms can be used, such as singular, gene set, or modular enrichment analysis. In specific, non-limiting examples, gene set enrichment analysis can be used (such as with differential expression signatures that include genes or gene sets that are ranked based on level of differential expression), for example, gene set enrichment analysis (GSEA), ErmineJ, FatiScan, MEGO, PAGE, MetaGF, Go-Mapper, ADGO, or the like (such as described in Huang et al., Nucleic Acids Res. 37(1): 1-13, 2009, incorporated herein by reference in its entirety).

In practice, output pathway signatures can take a variety of forms. For example, pathway signatures can include a list of pathways enriched in differential expression signatures. In practice, the list of pathways can include a variety of possible pathways. In examples, possible pathways can include the pathways listed in one or more general or specific pathway databases (for example, EcoCyc, KEGG, RegulonDB, MetaCyc STRINGDB, PANTHER, Gene Ontology, REACTOME, MSigDb, Ingenuity Knowledge Base, NCI PID, WikiPathways, Small Molecule Pathway DB, ConsensusPathDB, Pathway Commons, or the like, such as described in Garcia-Campos et al., Front. Physiol, 6(383), 2015, incorporated herein by reference in its entirety), such as during training. In examples, possible pathways can include pathways listed in a pathway signature (such as pathway signatures disclosed herein), such as during use or validation, for example, in single sample pathway signatures or in pathway signatures associated with a disease, such as pancreatic cancer.

In examples, enriched pathways can be quantified based on the level of enrichment in differential expression signatures. For example, an enrichment score (such as a normalized enrichment score) or a p value can be associated with the enriched pathways in the pathway signature output. Other forms are possible, for example, quantified gene expression of the genes in the enriched pathways can be the output.

In examples, output pathway signatures can be generated based on absolute valued differential expression signatures or signed differential expression signatures. Thus, pathway signature output can also include absolute valued pathway signatures or signed pathway signatures. Single sample pathway signature output can also be signed or absolute valued.

### Example 25—Example Implementation

SAHMI framework for detection of microbial entities from scRNAseq data: SAHMI (Single-cell Analysis of Host-Microbiome Interactions) was developed to estimate microbial diversity and to analyze patterns of human-microbiome interactions in tumor microenvironments at single cell resolution. SAHMI has four modules: (i) quantitation and annotation of microbial entities at multiple taxonomic levels from scRNAseq data with accompanying quality control filters; (ii) annotation of somatic cells and detection of preferential associations between microbial entities and host somatic cells; (iii) detection of significant associations between microbial profiles and the activities of signaling genes and cellular processes in host cells and at the tissue level; and (iv) analysis of associations between the sample microbiome and clinical attributes.

Annotation of somatic cells from scRNAseq data: SAHMI mapped the reads from single cell sequencing experiments to the host (e.g., human) genome and used the resulting transcriptomic signatures to cluster and annotate somatic cell types. Somatic cell clustering was done using the Seurat (Stuart et al. Cell, 177: 1888-1902.e21, 2019) R package with default parameters.

Quantitation and annotation of microbial entities: Metagenomic classification of paired-end reads from single-cell RNA sequencing fastq files was done using Kraken 2 (Wood et al. Genome Biol. 20: 257, 2019) with the default bacterial and fungal databases (Appendix I). The algorithm found exact matches of candidate 31-mer genomic substrings to the lowest common ancestor of genomes in a reference metagenomic database. Mapped metagenomic reads then underwent a series of filters. ShortRead (Morgan et al. Bioinformatics 25: 2607-2608, 2009) was used to remove low complexity reads (<20 non-sequentially repeated nucleotides), low quality reads (PHRED score<20), and PCR duplicates tagged with the same unique molecular identifier and cellular barcode. Non-sparse cellular barcodes were then selected by using an elbow-plot of barcode rank vs. total reads, smoothed with a moving average of 5, and with a cutoff at a change in slope<10⁻³, in a manner analogous to how cellular barcodes are typically selected in single-cell sequencing data (CellRanger (10× Genomics), Drop-seq Core Computational Protocol v2.0.0 (McCarroll laboratory)). Lastly, taxizedb (Chamberlain et al. Tools for Working with ‘Taxonomic’ Databases, 2020) was used to obtain full taxonomic classifications for all resulting reads, and the number of reads assigned to each clade was counted.

Normalization and identification of differentially expressed metagenomes: Sample-level normalized metagenomic levels were calculated as log 2 (counts/total_counts*10,000+1). For analyses that compared cell-level metagenome and somatic gene expression, the default Seurat normalization was used. To identify bacterial and fungal genera that were differentially present in case samples compared to controls, a linear model was constructed to predict sample-level normalized genera levels as a function of tissue status, somatic cellular composition (to account for potential tropisms), and total metagenomic reads. Cellular counts and total metagenomic counts were log-normalized prior to model fitting.

Microbe-gene/pathway association: Correlations were done on three levels: (1) between microbe and gene or pathway levels within individual cells grouped by cell-type, (2) between the average microbe and gene or pathway level in a given cell-type, and (3) between total sample microbe levels and gene expression. Under the default SAHMI settings, at the individual cell-level, correlations were only done between microbes and somatic genes that were co-expressed in at least 50 of the same cell-type. Kyoto Encyclopedia of Genes and Genomes (KEGG) (Kanehisa et al. Nucleic Acids Res. 45: D353-D361, 2017) pathway enrichments from cell-level gene correlations were calculated for significant correlations with Irl>0.5 and adjusted p-value<0.05 using clusterProfiler (Yu et al. Omi. A J. Integr. Biol. 16: 284-287, 2012). Correlations between microbe levels and KEGG pathway scores were also examined at the individual cell and averaged-cell type levels. Pathway scores were calculated as the mean of root-mean scaled normalized gene expression to avoid a single-gene dominating a pathway score. Pathway scores in a cell-type were only calculated for pathways in which at least half the genes were detected.

Microbiome-host cell composite pathways networks: Microbiome and pathway association data were used to construct an interaction network using igraph (Csardi et al. InterJournal Complex Syst. 1695: 1696, 2006) in which nodes were either averaged cell-type specific microbe levels or KEGG pathway scores, and edges represented significant correlations.

Pseudotime inferences: SAHMI uses a minimum spanning tree-based approach (Trapnell et al. Nat. Biotechnol. 32: 381-386, 2014) to order entire tissue microenvironments based on their cellular counts, KEGG pathway activities, and microbiome abundances. Cell counts were logip normalized and scaled. Microbes were included if they were found to be differentially present in either tumors or control samples and if their abundance was >₁0′ or if they were custom selected. Microbiome abundances per sample were normalized as stated above, centered, and unit-scaled. Normalized and scaled cell counts, pathway scores, and microbiome abundances for all samples were combined into a single matrix and used as input to Monocle's pseudotime function (Trapnell et al. Nat. Biotechnol. 32: 381-386, 2014), using expressionFamily=uninormal( ) and norm_method=“none”. Numerical microbiome and clinical parameters were compared across the resulting states using a t-test, and categorical parameters using Fisher's test.

Survival and clinical covariate analyses: The microbiome Shannon diversity index was calculated for each sample, and the samples were divided according to whether the microbiome Shannon index was greater than the mean index for the cohort (classified as “high” diversity) or less than (classified as “low” diversity). Patients were stratified by their predicted microbial diversity, and the survminer package (github.conVkassambara/survminer/) was used to test the relationship with survival.

Cohort selection and metagenomic inferences: Single-cell RNA sequencing data were obtained for 24 human pancreatic ductal adenocarcinomas (PDA) and 11 control pancreas tissues (non-PDA lesions) from Peng et al. (Peng et al. Cell Res. 29(9):725-738, 2019). In that cohort, pancreatic tumor or tissue samples were collected during pancreatectomies or pancreatoduodenectomies (Table 1, patient characteristics). The samples were checked for batch effects at the levels of sample and somatic cell type clusters. The cohort had 100-500 million reads per sample, of which a substantial proportion did not map to the human genome, and these reads were used for metagenomic analyses. scRNAseq data from two additional studies that focused on the normal pancreas (Baron et al. Cell Syst. 3: 346-360.e4, 2016; Muraro et al. Cell Syst. 3: 385-394.e3, 2016) were obtained and processed similarly. Data were also obtained on microbial genera classified from bulk-RNA sequencing of pancreatic adenocarcinoma (PAAD) from TCGA (Poore et al. Nature 579: 567-574, 2020) (selecting counts and normalized expression values of TCGA genera passing all decontamination steps), and genera classified from 16S rRNA sequencing of pancreatic cancer in a recent large-scale study (Nejman et al. Science, 368(6494):973-980, 2020) (normalized expression of genera passing all filters except the multi-study filter). Decontamination was done by comparing genera identified in one sample to those identified in other scRNAseq data of the same organ type, or to those identified by Poore et al. (2020) in TCGA or by Nejman et al. (2020) from 16s-rRNA sequencing of the same organ type. Genera found exclusively in the sample being analyzed were identified as possible contaminants and were removed from further analyses.

TABLE 1
Clinical characteristics of PDA patients and control samples
profiled by scRNA-seq. (Peng et al. ell Res. 29(9): 725-738, 2019)

| | | | | CA19- | | | | Max | TNM | | | | |
|---|---|---|---|---|---|---|---|---|---|---|---|---|---|
| | Pathologic | | Age | 9 | | Pro- | | Diameter | Classifi- | Stag- | P | | P |
| Sample | Diagnosis | Sex | (years) | (U/ml) | DM | cedure | Location | (mm) | cation | ing | Inv | VI | Inf |
| T1 | moderately-poorly | M | 64 | 86 | N | LDP | body | 26 | T4N2M0 | III | Y | Y | Y |
| | differentiated PDAC | | | | | | | | | | | | |
| T2 | well differentiated | M | 52 | 46.3 | N | PD | head | 20 | T1cN1M0 | IIB | Y | N | Y |
| | PDAC | | | | | | | | | | | | |
| T3 | moderately-poorly | F | 58 | 49.2 | Y | PD | uncinate | 22 | T2N0M0 | IB | N | N | Y |
| | differentiated PDAC | | | | | | process | | | | | | |
| T4 | moderately | F | 72 | 40.4 | Y | LDP | body | 14 | T1cN1M0 | IIB | N | N | Y |
| | differentiated PDAC | | | | | | | | | | | | |
| T5 | well-moderately | F | 65 | 37 | Y | PD | uncinate | 29 | T2N0M0 | IB | N | N | Y |
| | differentiated PDAC | | | | | | process | | | | | | |
| T6 | moderately-poorly | M | 64 | 155.1 | N | ODP | tail | 91 | T3N0M0 | IIA | N | N | Y |
| | differentiated PDAC | | | | | | | | | | | | |
| T7 | moderately | M | 70 | <0.6 | Y | ODP | body | 80 | T3N1M0 | IIB | N | N | Y |
| | differentiated PDAC | | | | | | | | | | | | |
| T8 | moderately-poorly | F | 66 | 82.5 | N | PD | uncinate | 17 | T1cN2M0 | III | N | N | N |
| | differentiated PDAC | | | | | | process | | | | | | |
| T9 | moderately-poorly | M | 36 | 11.2 | N | PD | head | 26 | T2N0M0 | IIA | Y | Y | Y |
| | differentiated PDAC | | | | | | | | | | | | |
| T10 | poorly differentiated | M | 61 | 972.8 | Y | PD | uncinate | 40 | T2N1M0 | IB | Y | Y | Y |
| | PDAC | | | | | | process | | | | | | |
| T11 | moderately-poorly | M | 51 | 211.1 | N | ODP | body and | 76 | T3N1M0 | IIB | Y | Y | Y |
| | differentiated PDAC | | | | | | tail | | | | | | |
| T12 | poorly differentiated | M | 54 | 146.1 | N | PD | uncinate | 50 | T3N2M0 | III | Y | Y | Y |
| | PDAC | | | | | | process | | | | | | |
| T13 | moderately-poorly | F | 58 | 21.9 | Y | PD | head | 30 | T2N1M0 | IIB | Y | N | Y |
| | differentiated PDAC | | | | | | | | | | | | |
| T14 | well differentiated | F | 67 | 77 | Y | PD | head | 33 | T2N1M0 | IIB | Y | Y | Y |
| | PDAC | | | | | | | | | | | | |
| T15 | well differentiated | F | 54 | 18.4 | N | LPD | head | 23 | T2N1M0 | IIB | Y | N | Y |
| | PDAC | | | | | | | | | | | | |
| T16 | poorly differentiated | F | 56 | 42.9 | N | LDP | body | 30 | T2N1M0 | IIB | Y | Y | Y |
| | PDAC | | | | | | | | | | | | |
| T17 | moderately | F | 71 | 209.3 | N | LDP | body and | 30 | T2N0M0 | IB | Y | N | N |
| | differentiated PDAC | | | | | | tail | | | | | | |
| T18 | moderately-poorly | F | 68 | 112.3 | Y | ODP | body | 28 | T2N0M0 | IB | Y | Y | Y |
| | differentiated PDAC | | | | | | | | | | | | |
| T19 | well-moderately | F | 59 | 93.9 | N | LPD | head | 35 | T2N0M0 | IB | Y | Y | Y |
| | differentiated PDAC | | | | | | | | | | | | |
| T20 | moderately | M | 59 | 2.2 | N | PD | head | 43 | T3N1M0 | IIB | Y | Y | Y |
| | differentiated PDAC | | | | | | | | | | | | |
| T21 | moderately-poorly | M | 59 | 528.6 | Y | LPD | head | 35 | T2N0M0 | IB | Y | Y | Y |
| | differentiated PDAC | | | | | | | | | | | | |
| T22 | moderately | F | 67 | 234.5 | N | ODP | body | 27 | T2N0M0 | IB | Y | N | Y |
| | differentiated PDAC | | | | | | | | | | | | |
| T23 | moderately-poorly | M | 54 | 312.2 | Y | PD | head | 27 | T2N1M0 | IIB | Y | Y | Y |
| | differentiated PDAC | | | | | | | | | | | | |
| T24 | moderately | F | 44 | 14.4 | N | PD | head | 20 | T1cN0M0 | IB | Y | N | Y |
| | differentiated PDAC | | | | | | | | | | | | |
| N1 | normal | F | 64 | 7.5 | N | ODP | tail | 50 | NA | NA | N | N | N |
| | pancreas/mucinous | | | | | | | | | | | | |
| | cystic neoplasia | | | | | | | | | | | | |
| N2 | normal | M | 55 | 171.2 | N | PPPD | descending | 11 | NA | NA | N | Y | N |
| | pancreas/small | | | | | | duodenum | | | | | | |
| | intestine papillary | | | | | | | | | | | | |
| | adenocarcinoma | | | | | | | | | | | | |
| N3 | normal | M | 50 | 6.4 | N | PD | descending | 20 | NA | NA | N | N | N |
| | pancreas/duodenal | | | | | | duodenum | | | | | | |
| | intraepithelial | | | | | | | | | | | | |
| | neoplasia | | | | | | | | | | | | |
| N4 | normal | M | 53 | 4.5 | N | LDP | body and | 40 | NA | NA | N | N | N |
| | pancreas/pancreatic | | | | | | tail | | | | | | |
| | neuroendocrine | | | | | | | | | | | | |
| | tumor | | | | | | | | | | | | |
| N5 | normal | F | 52 | 9 | N | LDP | body and | 24 | NA | NA | N | N | N |
| | pancreas/serous | | | | | | tail | | | | | | |
| | cystic neoplasia | | | | | | | | | | | | |
| N6 | normal | F | 31 | 29.5 | N | ODP | body | 22 | NA | NA | N | N | N |
| | pancreas/solid | | | | | | | | | | | | |
| | pseudopapillary | | | | | | | | | | | | |
| | tumor | | | | | | | | | | | | |
| N7 | normal | F | 42 | 12.7 | N | LDP | tail | 94 | NA | NA | N | N | N |
| | pancreas/mucinous | | | | | | | | | | | | |
| | cystic neoplasia | | | | | | | | | | | | |
| N8 | normal | M | 41 | 6 | N | LDP | body and | 76 | NA | NA | N | N | N |
| | pancreas/solid | | | | | | tail | | | | | | |
| | pseudopapillary | | | | | | | | | | | | |
| | tumor | | | | | | | | | | | | |
| N9 | normal | M | 34 | 23.8 | N | LDP | tail | 22 | NA | NA | N | N | N |
| | pancreas/pancreatic | | | | | | | | | | | | |
| | neuroendocrine | | | | | | | | | | | | |
| | tumor | | | | | | | | | | | | |
| N10 | normal | F | 65 | 193.3 | N | PD | common | NA | T3N0M0 | IIA | N | N | N |
| | pancreas/choledocha | | | | | | bile duct | | | | | | |
| | 1 neuroendocrine | | | | | | | | | | | | |
| | tumors | | | | | | | | | | | | |
| N11 | normal | F | 30 | NA | N | LDP | body | 33 | NA | NA | N | N | N |
| | pancreas/solid | | | | | | | | | | | | |
| | pseudopapillary | | | | | | | | | | | | |
| | tumor | | | | | | | | | | | | |

DM: Diabetes Mellitus;
LDP: Laparoscopic distal pancreatectomy;
ODP: Open distal pancreatectomy;
PD: Pancreatoduodenectomy;
LPD: Laparoscopic pancreatoduodenectomy;
PPPD: Pylorus preserved pancreatoduodenectomy;
P Inv: Perineural Invasion;
VI: Vascular Invasion;
P Inf: Peripancreatic Infiltration.

Quality control analysis, comparative analyses, and benchmarking: To mitigate the influence of classification errors, contamination, noise, and batch effects, total genus abundances were examined, and genera sequenced with different technologies across multiple studies were compared. Specifically, metagenomes from the Peng et al. (Peng et al. Cell Res. 29(9):725-738, 2019) cohort were compared to those from (i) two other single-cell studies of the normal pancreas (Baron et al. Cell Syst. 3: 346-360.e4, 2016; Muraro et al. Cell Syst. 3: 385-394.e3, 2016). classified using our pipeline, (ii) genera classified from bulk-RNA sequencing of the TCGA pancreatic cancer (TCGA-PAAD) (Poore et al. Nature 579: 567-574, 2020), and (iii) genera classified from 16S rRNA sequencing of pancreatic cancer (Nejman et al. Science, 368(6494):973-980, 2020), as described above. Genera in the single-cell datasets were only retained if they were present at a frequency greater than 10⁻⁴ and if they were detected in two or more independent studies. Pancreas-specific taxa were retained regardless of country of origin or other possible batch effects, although this approach risks filtering out individual specific or low-prevalence taxa.

To compare filtered microbial profiles across studies, the overlap coefficient of any two sets was calculated as overlap(X, Y)=intersect(X, Y)/min(IXI, IYI). Study-level microbial abundances were compared with Spearman correlations and microbial detection was compared with the overlap coefficient. Harmonic mean p-values for combining dependent Spearman correlation associated p-values were calculated using the harmonicmeanp package (Wilson, Proc. Natl. Acad. Sci. 116(4):1195-1200, 2019). Literature reported microbial changes in pancreatic disease were obtained from Table 1 in Thomas et al. (Thomas et al. Nat. Rev. Gastroenterol. Hepatol. 17: 53-64, 2020) A list of putative laboratory contaminants was obtained from Poore et al. (Poore et al. Nature, 579: 567-574, 2020), who performed extensive statistical analysis and literature research to identify common contaminants.

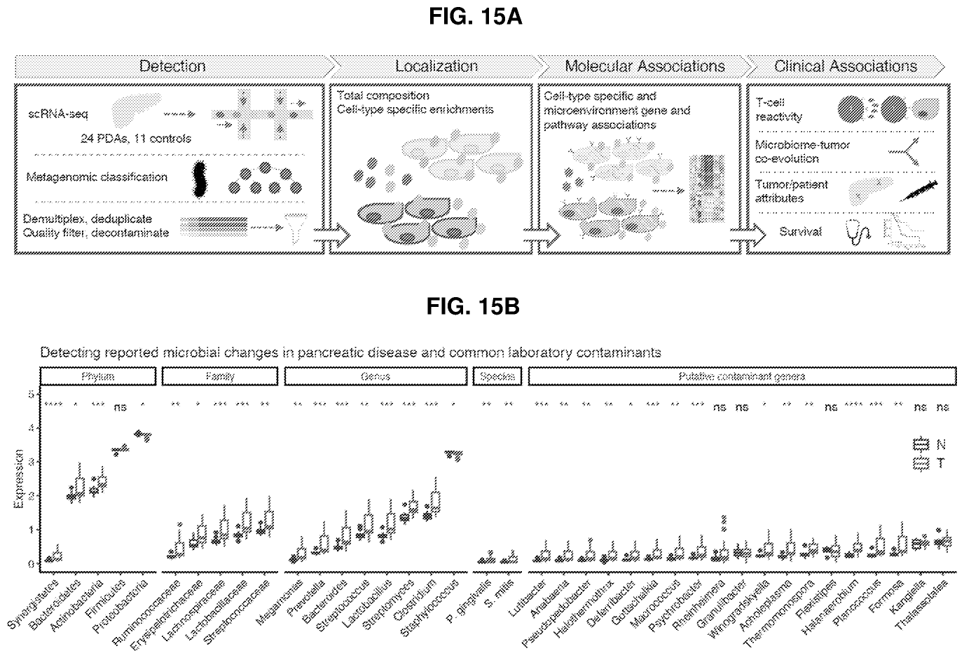

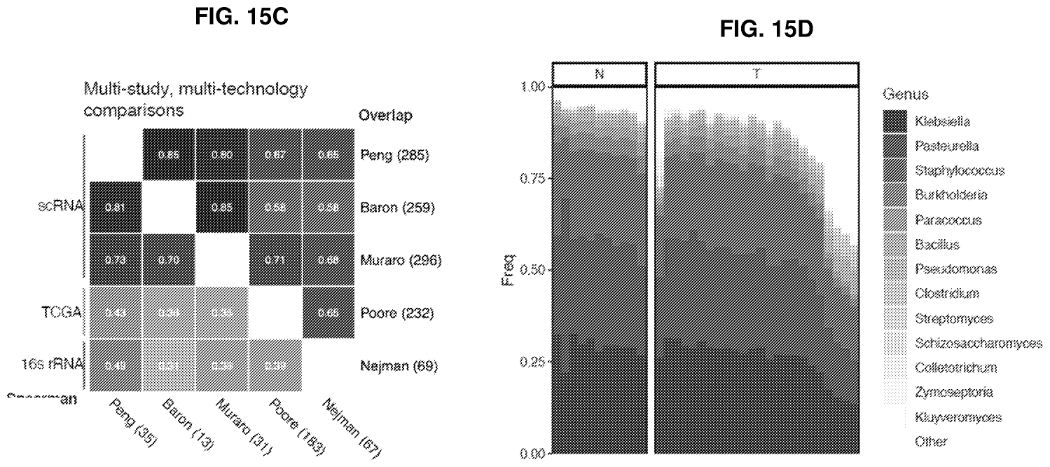

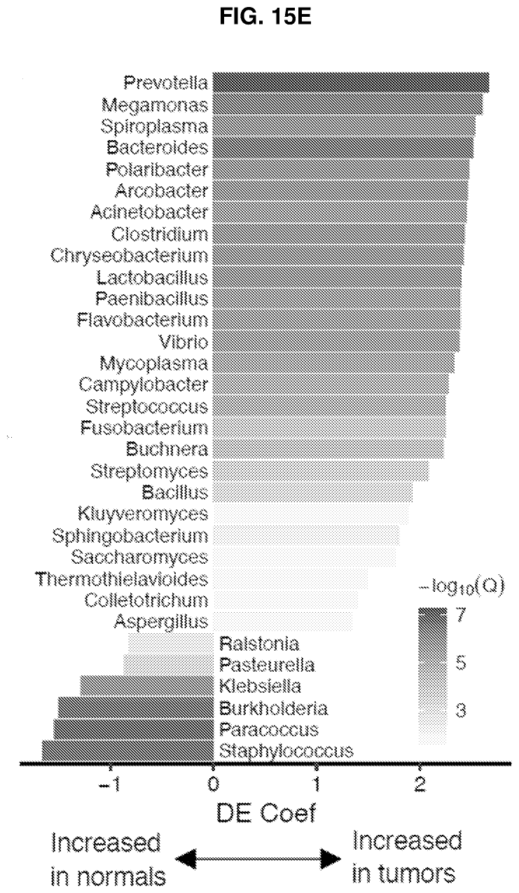

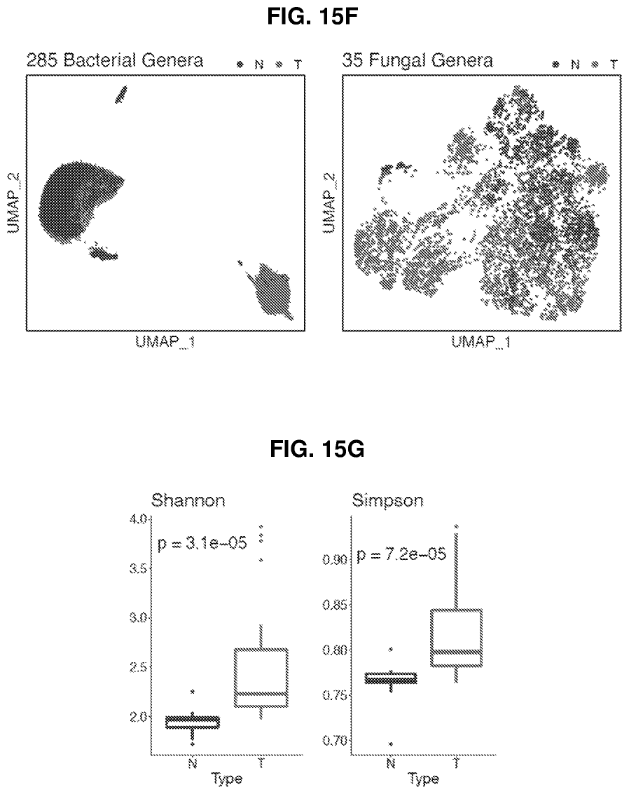

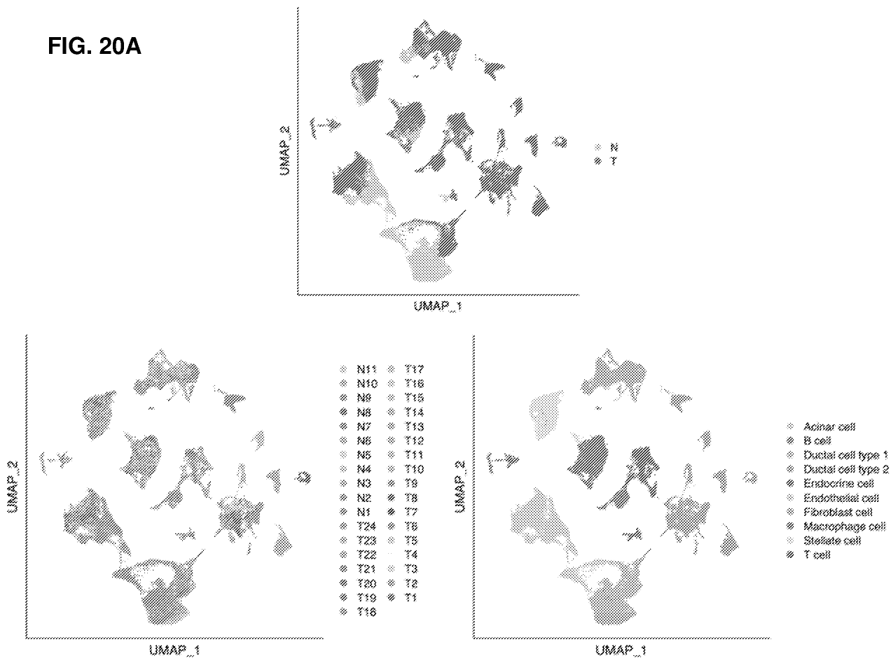

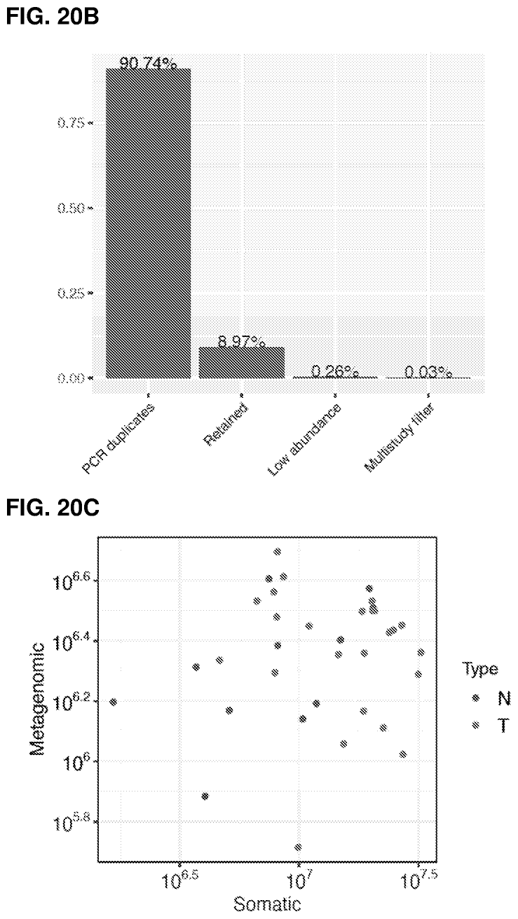

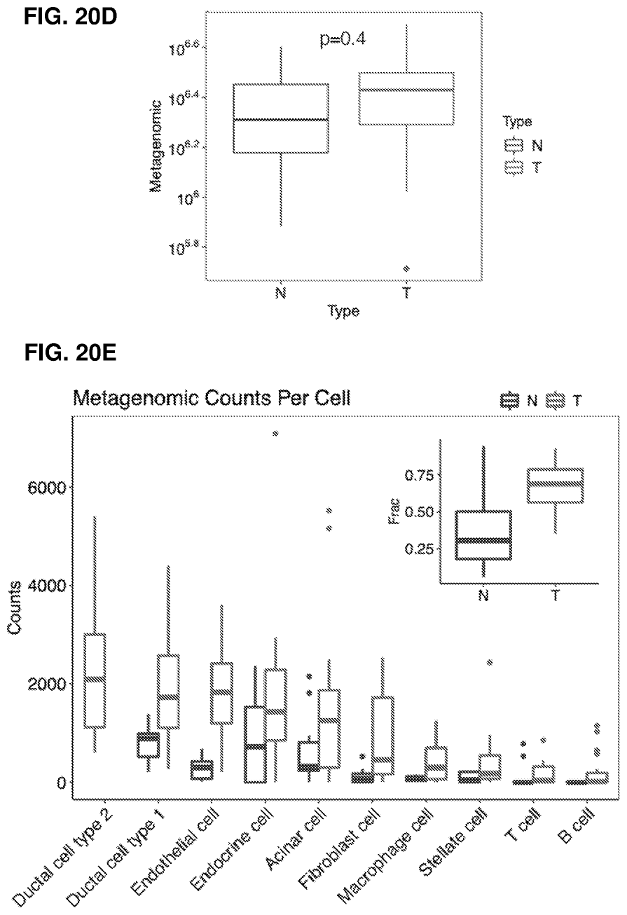

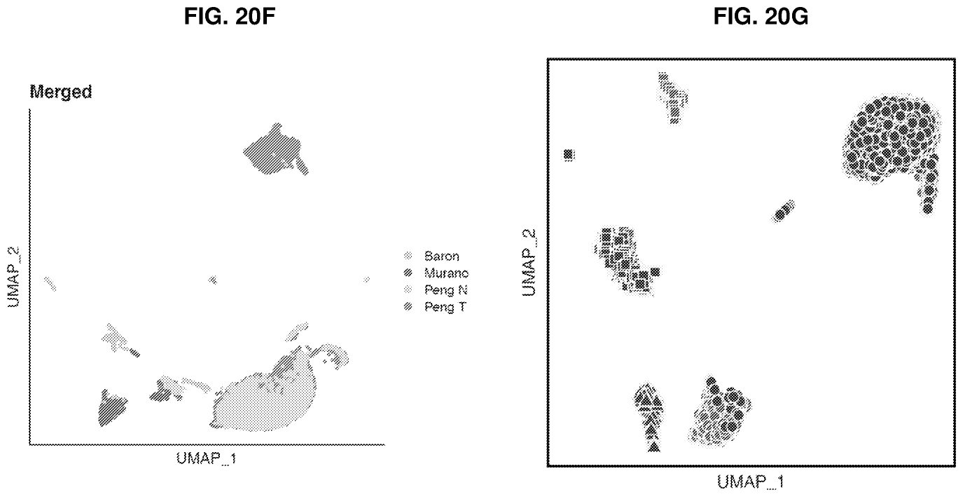

Metagenomic differences between tumor and non-tumor samples: As described above, SAHMI was used for normalization and identification of differentially expressed metagenomes between pancreatic tumors and non-malignant samples. Cellular counts and total metagenomic counts were log-normalized prior to model fitting. Tissue status was modeled as three groups: normal, tumor group 1 (tumors whose microbiome appeared broadly similar to that of nonmalignant samples), and tumor group 2 (tumors with markedly different microbiomes). These three groups were defined based on barcode clustering in the bacterial (FIG. 15F) and combined bacterial and fungal UMAP plots (FIG. 20G). Differentially present genera were identified as those with nonzero tissue-status coefficients (adjusted p<0.05). Figures in which differentially expressed genera are highlighted include statistically significant genera with either abundances>10′ or literature-reported microbial associations to pancreatic cancer summarized in a recent review (Thomas et al. Nat. Rev. Gastroenterol. Hepatol. 17: 53-64, 2020).

Somatic cell-type and sample cellular composition predictions: Somatic cell clustering was done by SAHMI as described above. The somatic gene expression count matrix and cell type annotations were taken from the original study (Peng et al. Cell Res. 29(9):725-738, 2019). To ensure that gene count data were consistent regardless of the preprocessing pipeline, for five samples, gene counts were derived from raw fastq files using the Drop-seq Core Computational Protocol v2.0.0 from the McCarroll laboratory with default parameters. Briefly, barcodes with low quality bases were filtered out, the resulting transcripts were aligned to GRCH37 using the splice-aware STAR aligner (Dobin et al. Bioinformatics 29: 15-21, 2013), and gene-level counts and cell-containing barcodes were estimated. Somatic cell clusters were then obtained using Seurat and were compared to those from the Peng et al. (Peng et al. Cell Res. 29(9):725-738, 2019) processed data and showed no major differences.

Identifying somatic cellular sub-clusters was done using the self-assembling manifolds (SAM) (Tarashansky et al. Elife, 8: 1-29, 2019) package in Python, which reduces the dimensionality of a dataset using an iterative approach that emphasizes features that discriminate across clusters. Each somatic cell-type was processed independently, whereby SAM reduced the data dimensionality and Seurat was used to find clusters in the resulting principal component reduction, using resolution=0.4 to capture only the major sub-clusters that were made of multiple samples. SAM was chosen because of its demonstrated good performance and because it produced interpretable sub-clusters, which were annotated using known markers.

Barcode cell-type predictions were done for the subset of cell-associated barcodes (13,848/23,546 total). Barcodes were identified as cell-associated if the same microbiome-tagging barcode also tagged somatic cellular RNA and was retained during analysis of the host cells and assigned a cell-type label based on its somatic gene expression signatures. A random forest model was then trained to classify each barcode's associated somatic cell type based on its microbiome profile. To account for the large cell-type class imbalance in microbiome-tagging barcodes during model training (the majority of microbiome reads co-localized with epithelial and endothelial cells and few with immune cells), 150 barcodes from each cell-type were selected for training, and then the resulting model was used to predict the remaining 11,984 barcodes. Receiver-operator curves were calculated using the pROC (Robin et al. BMC Bioinformatics, 12: 77, 2011) R package. Multiple run of this procedure produced nearly identical receiver-operator curves.

Tumor microenvironment somatic cellular composition was predicted using least absolute shrinkage and selection operator (LASSO) linear regression from the glmnet (Simon et al. J. Stat. Software, 39(5):1-13, 2011) R package. The model underwent 10-fold cross-validation using the ‘cv.glmnet’ function over a range of lambdas from exp(−0.5, −3) and alpha=1. LASSO regression with the same optimization parameters was also attempted 500 times to predict sample-label shuffled data.

Validation of cell-type enrichments across datasets: Metagenomic enrichments in somatic cell-types were determined using the FindAllMarkers function in Seurat, which calculates log-fold changes of normalized bacterial or fungal levels in each cell-type relative to all others and associated enrichment p-values using Wilcoxon rank-sum tests. To assess the significance and reproducibility of these enrichments, for two pancreatic single-cell datasets (Peng et al. Cell Res. 29(9):725-738, 2019; Baron et al. Cell Syst. 3: 346-360.e4, 2016), 80% of the cells were subsampled, the total number of statistically significant microbiome-cell-type enrichments were found, and then the cell-type labels and similarly calculated enrichments were randomized. This was repeated 500 times, and the distributions of the total number of enrichments found in each dataset from actual vs. shuffled data were compared, as well as the number of shared enrichments, using the Wilcoxon test.

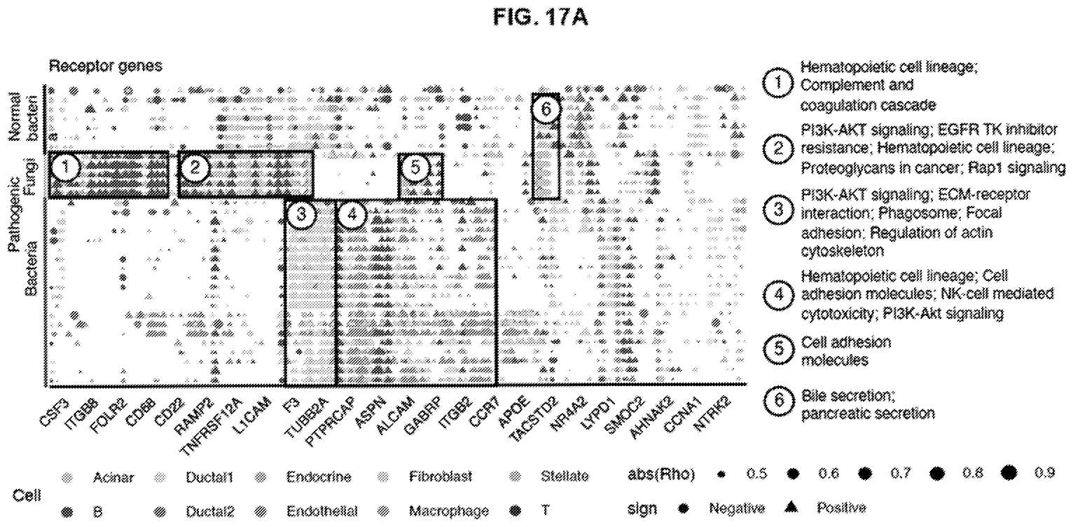

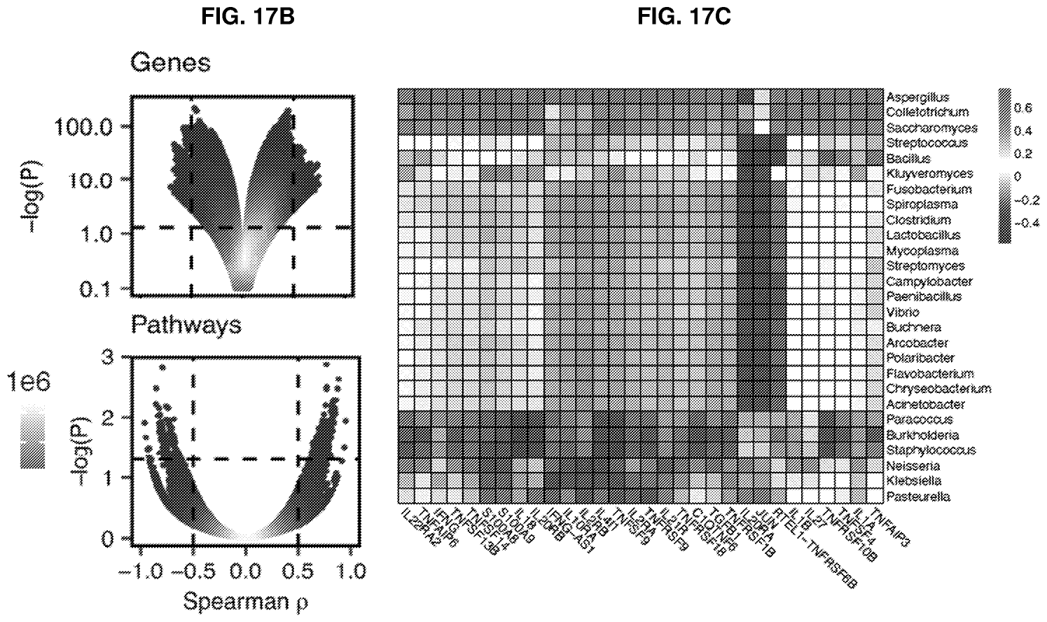

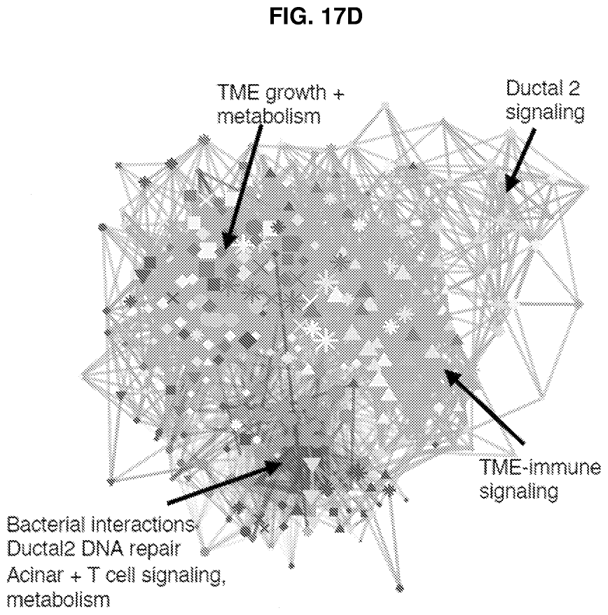

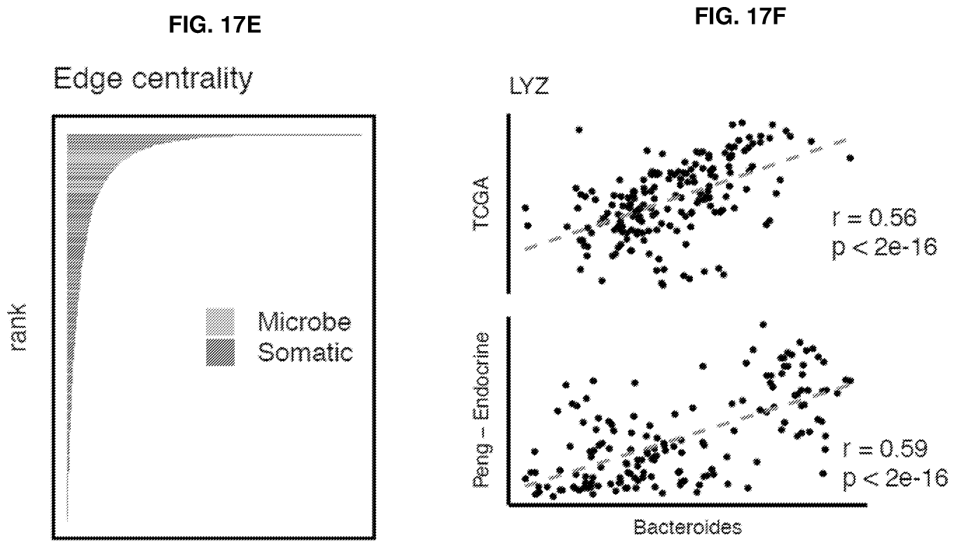

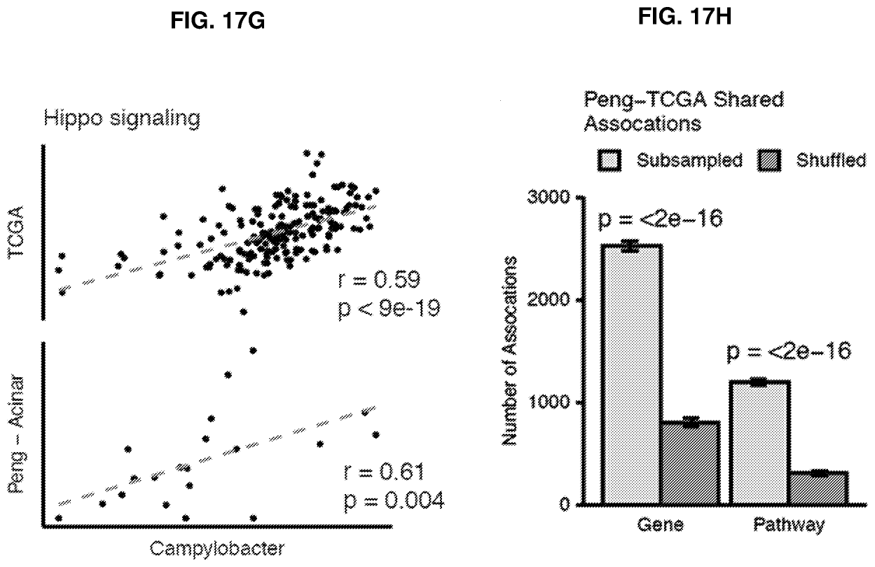

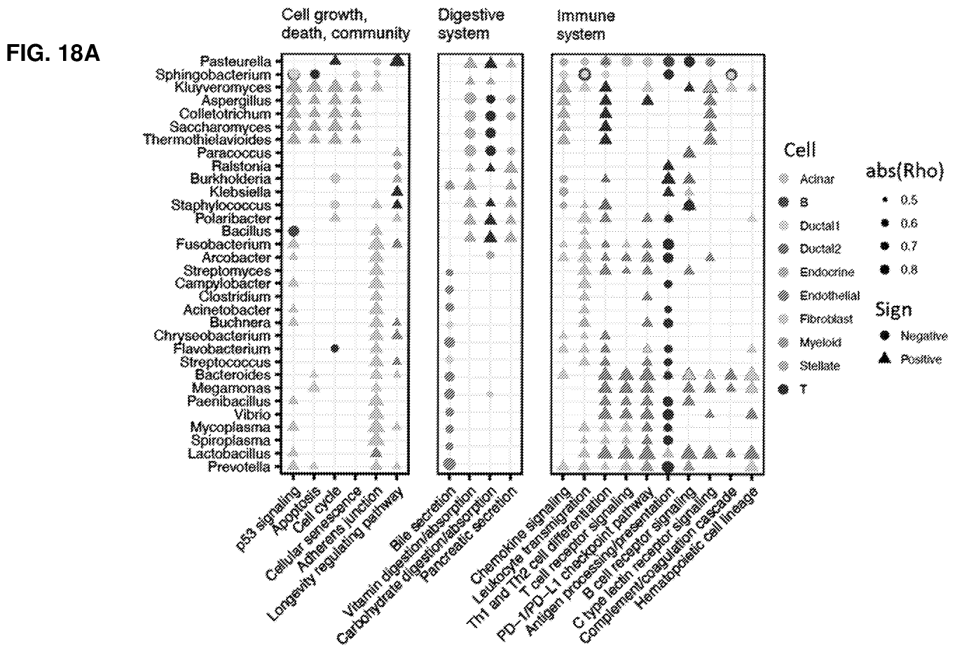

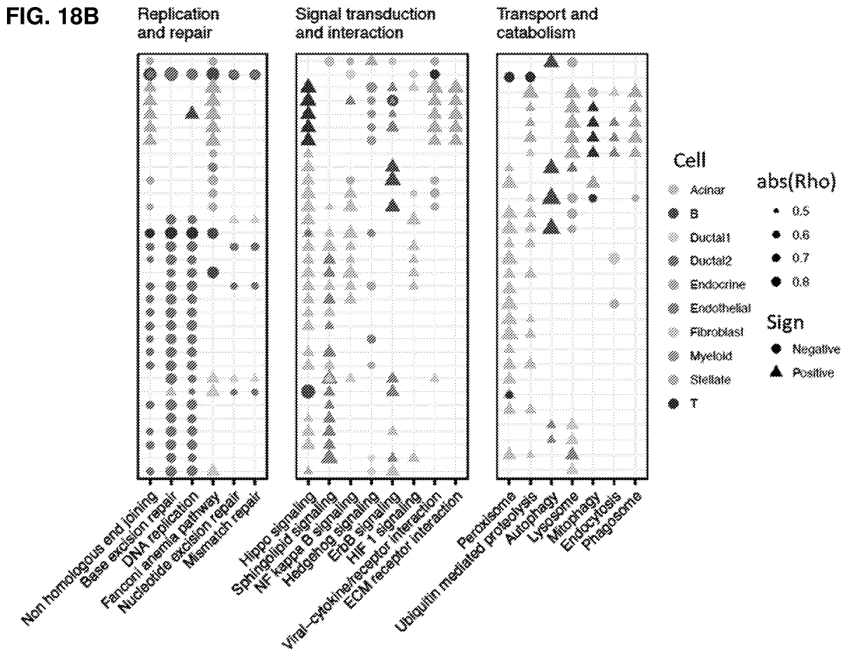

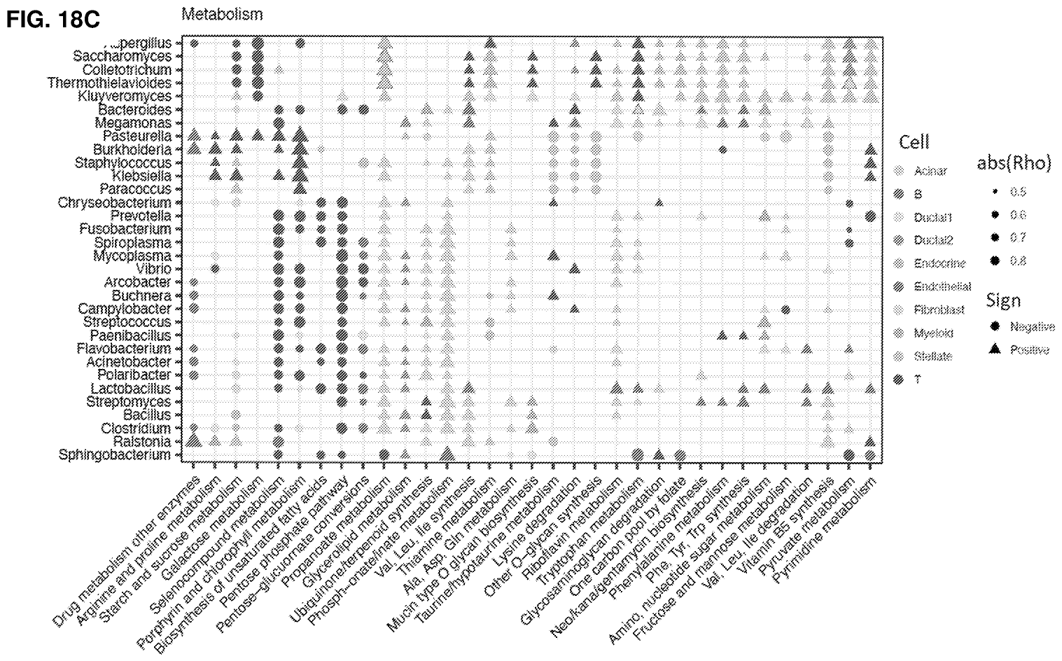

Association between microbes and cellular processes: Associations between microbial entities and cellular processes were analyzed in pancreatic tumors and non-malignant samples as stated above. Microenvironment-level correlations were examined between total microbes and inflammatory or antimicrobial genes. Inflammatory genes were obtained from Smillie et al. (Smillie et al. Cell, 178: 714-730.e22, 2019) and receptor and antimicrobial genes were obtained from GeneCards (Stelzer et al. Curr. Protoc. Bioinforma. 54: 1.30.1-1.30.33, 2016). Pathway score correlations in FIGS. 18A-18C were grouped by KEGG groupings, and data were collected for pathways relevant to pancreatic function and cancer hallmarks; these pathways were: cell growth, death, community, digestive system, immune system, replication and repair, signal transduction and interaction, transport and catabolism, and metabolism. Only pancreas or cancer-related pathways shown in FIGS. 18A-18C were included in the FIG. 17D network. Microbe-cell-specific pathway edges were included if the correlation had a Spearman coefficient Irl>0.5 and adjusted p-value<0.05. Because some KEGG pathways can be inter-related or include overlapping gene sets, pathway-pathway edges were included between pathways correlated with Spearman Irl>0.75 and adjusted p-value<0.05. Edge centrality was calculated using igraph (Csardi et al. InterJournal Complex Syst. 1695: 1696, 2006).

Validation of microbe-gene and pathway associations: The significant correlations between microbes and genes and pathways found in the Peng et al. (Peng et al. Cell Res. 29(9):725-738, 2019) cohort were compared to correlations between gene expression or pathways scores from the pancreatic cancer samples in the TCGA and the affiliated microbiome levels estimated by Poore et al. (Poore et al. Nature, 579: 567-574, 2020). Normalized gene expression data for TCGA pancreatic cancer (PAAD) samples were obtained via RTCGAToolbox (Samur, PLoS One, 9: e106397, 2014). A small number of common microbe-gene/pathway correlations were identified with Spearman Irl>0.5 and adjusted p-value<0.05 at both the individual cell level and the averaged cell-type level in Peng et al (Peng et al. Cell Res. 29(9):725-738, 2019) compared to TCGA. The number of common statistically significant (t-test, p<0.05) microbe-gene/pathway correlations in Peng vs. TCGA were compared, regardless of correlation strength. In 500 iterations, 80% of both datasets were subsampled, averaged cell-type microbe and gene or pathway levels in Peng et al (Peng et al. Cell Res. 29(9):725-738, 2019) and microbe and bulk gene or pathway levels in TCGA were correlated, and the number of statistically significant correlations shared by both datasets was calculated. This process was repeated with shuffled sample labels and the distributions of common correlations were compared using Wilcoxon testing in subsampled vs. shuffled data.

T-cell reactivity analysis: A random forest model was trained and validated to classify tumor-reactive vs. microbe-reactive T-cells based on their gene expression profiles. The model was trained using single-cell RNA sequencing data of T-cells isolated from peripheral blood mononuclear cells from patients with bacterial sepsis (singlecell.broadinstitute.org/single_cell; SCP548) or from primary lung adenocarcinomas (E-MTAB-6149), which were previously shown to have low microbiome burden (Poore et al. Nature, 579: 567-574, 2020; Nejman et al. Science, 368(6494):973-980, 2020). Processed gene expression data were analyzed using Seurat (Stuart et al. Cell, 177: 1888-1902.e21, 2019); cells were clustered based on transcriptomic profiles, and T-cells were identified using known markers (Nirmal et al. Cancer Immunol. Res. 6(11):1388-1400, 2018). The FindAllMarkers function from Seurat was used to identify˜500 genes differentially expressed in T-cells from lung cancer and sepsis patients. 1000 T-cells from each study were subsampled and the rank order of the ˜500 differentially expressed genes (Table 2) was used to train a random forest model to classify tumor-reactive or microbe-reactive T-cells. The model was then validated using the remaining T-cells from the lung cancer and sepsis studies, as well as 6 other datasets with either known microbial stimulation or cancer with low-microbiome burden: bladder cancer (GSE149652), melanoma (GSE120575), glioblastoma (GSE131928), pilocytic astrocytoma (SCP271), Salmonella stimulation (GSM3855868), and Candida stimulation (eqtlgen.org/candida.html). Given the model's exceptional accuracy in classifying over 100,000 T-cells from new datasets, it was then used to predict T-cell reactivity from the Peng et al. cohort.

TABLE 2
Exemplary genes (T-cell microenvironment reaction signature, used to
classify T-cells isolated from a subject as tumor-reactive or microbe-
reactive. “Mean decrease accuracy” for a gene indicates the change in
model classification accuracy when the value of the gene is randomly
permuted.

| | Gene | Mean Decrease Accuracy |
|---|---|---|
| 1 | S100A8 | 0.092773561 |
| 2 | RPL41 | 0.078648903 |
| 3 | RPL39 | 0.039672861 |
| 4 | S100A9 | 0.028971284 |
| 5 | RPS27 | 0.009858452 |
| 6 | RPS29 | 0.00877185 |
| 7 | NKG7 | 0.008558657 |
| 8 | TYROBP | 0.007349671 |
| 9 | RPS28 | 0.006257825 |
| 10 | LYZ | 0.005155002 |
| 11 | RPS26 | 0.004307184 |
| 12 | S100A12 | 0.003465595 |
| 13 | LST1 | 0.002760927 |
| 14 | GNLY | 0.002602244 |
| 15 | TMSB10 | 0.002425835 |
| 16 | RPL13A | 0.002377481 |
| 17 | EEF1A1 | 0.002302028 |
| 18 | FCN1 | 0.002029348 |
| 19 | MYL6 | 0.001801459 |
| 20 | PLEKHJ1 | 0.00170777 |
| 21 | CLTB | 0.001534479 |
| 22 | RPL24 | 0.001495799 |
| 23 | ST13 | 0.001426953 |
| 24 | RGS19 | 0.001284512 |
| 25 | RPL36A | 0.001254065 |
| 26 | RPS7 | 0.001246853 |
| 27 | DNAJC7 | 0.001213409 |
| 28 | GRN | 0.001194567 |
| 29 | ATP5G3 | 0.001172354 |
| 30 | CANX | 0.001162368 |
| 31 | C1orf56 | 0.001147811 |
| 32 | H3F3A | 0.001121218 |
| 33 | KLRD1 | 0.001087927 |
| 34 | RPL13 | 0.001066884 |
| 35 | PAK2 | 0.001064609 |
| 36 | FRG1 | 0.001055478 |
| 37 | TMEM256 | 0.001021827 |
| 38 | RPS9 | 0.000996953 |
| 39 | LPAR6 | 0.000961476 |
| 40 | BCLAF1 | 0.000931859 |
| 41 | RPS16 | 0.000921339 |
| 42 | MIEN1 | 0.000908645 |
| 43 | TMEM179B | 0.000891395 |
| 44 | SNHG9 | 0.000876477 |
| 45 | STAT1 | 0.000855168 |
| 46 | ATP5G2 | 0.000842925 |
| 47 | RPS4X | 0.000839862 |
| 48 | S100A11 | 0.000834713 |
| 49 | RPL15 | 0.000830827 |
| 50 | AHNAK | 0.000826019 |
| 51 | SMS | 0.000824325 |
| 52 | COX4I1 | 0.000822374 |
| 53 | HMHA1 | 0.000816084 |
| 54 | HSBP1 | 0.000812709 |
| 55 | YIPF4 | 0.000803081 |
| 56 | RPL29 | 0.000801736 |
| 57 | LCP1 | 0.000801253 |
| 58 | SNRPE | 0.000774927 |
| 59 | SVIP | 0.000771892 |
| 60 | RPL19 | 0.000764541 |
| 61 | FCER1G | 0.000744006 |
| 62 | CAPZA2 | 0.0007394 |
| 63 | CFL1 | 0.000732052 |
| 64 | EDF1 | 0.000720073 |
| 65 | VCAN | 0.000719759 |
| 66 | SDF2L1 | 0.000718715 |
| 67 | KRTCAP2 | 0.000713555 |
| 68 | CBX3 | 0.000713553 |
| 69 | NUCKS1 | 0.000702455 |
| 70 | RPL14 | 0.000702164 |
| 71 | DNAJC19 | 0.000695716 |
| 72 | RPLP1 | 0.000694564 |
| 73 | PGAM1 | 0.000689222 |
| 74 | C5orf56 | 0.00068649 |
| 75 | SPCS3 | 0.000685822 |
| 76 | MBP | 0.000676305 |
| 77 | HNRNPH1 | 0.000671656 |
| 78 | POLR2K | 0.00066548 |
| 79 | GNAI2 | 0.000656285 |
| 80 | SRRM2 | 0.00065613 |
| 81 | ZNHIT1 | 0.000654315 |
| 82 | SUB1 | 0.000644202 |
| 83 | LITAF | 0.000625774 |
| 84 | RPL36AL | 0.000625117 |
| 85 | CRIP1 | 0.000621146 |
| 86 | NDUFB11 | 0.000617543 |
| 87 | MOB1A | 0.000607107 |
| 88 | NDUFB4 | 0.000601115 |
| 89 | CST3 | 0.000595673 |
| 90 | SUMO2 | 0.000594374 |
| 91 | SRSF5 | 0.000593552 |
| 92 | NHP2 | 0.000584724 |
| 93 | HINT1 | 0.000583941 |
| 94 | LTB | 0.000574929 |
| 95 | CALM2 | 0.000564717 |
| 96 | EIF4B | 0.000564267 |
| 97 | COX20 | 0.000564044 |
| 98 | ARL5A | 0.000558315 |
| 99 | SYTL1 | 0.000553772 |
| 100 | PGLS | 0.000552433 |
| 101 | AIF1 | 0.000536204 |
| 102 | FGFBP2 | 0.000518878 |
| 103 | PRDM1 | 0.000513088 |
| 104 | UXT | 0.000511949 |
| 105 | C9orf16 | 0.000510293 |
| 106 | SNRPF | 0.00050393 |
| 107 | GZMH | 0.000501027 |
| 108 | POLR2F | 0.000498148 |
| 109 | NBEAL1 | 0.000494553 |
| 110 | SPN | 0.000492723 |
| 111 | TOMM7 | 0.000492541 |
| 112 | GABARAP | 0.000491839 |
| 113 | C17orf89 | 0.000488652 |
| 114 | GNB2 | 0.00048578 |
| 115 | CTSS | 0.000483926 |
| 116 | IFITM2 | 0.000483421 |
| 117 | CHCHD10 | 0.00047783 |
| 118 | VPS29 | 0.00047611 |
| 119 | JTB | 0.000471909 |
| 120 | APRT | 0.00046291 |
| 121 | RPL23A | 0.000460485 |
| 122 | CUTA | 0.000455038 |
| 123 | PTPN4 | 0.000454714 |
| 124 | OXLD1 | 0.000454202 |
| 125 | UBE2D1 | 0.000450914 |
| 126 | CYBB | 0.000447317 |
| 127 | RPS17 | 0.000442033 |
| 128 | PTMA | 0.000435696 |
| 129 | CD164 | 0.00043541 |
| 130 | C19orf70 | 0.000434591 |
| 131 | TSC22D4 | 0.000434491 |
| 132 | PSIP1 | 0.00042833 |
| 133 | PAN3 | 0.000423481 |
| 134 | TRMT112 | 0.000422168 |
| 135 | RPS3A | 0.00042108 |
| 136 | SLC9A3R1 | 0.000420697 |
| 137 | TCEA1 | 0.000420685 |
| 138 | FGR | 0.000418293 |
| 139 | HNRNPU | 0.000417556 |
| 140 | NDUFB3 | 0.000415965 |
| 141 | GPX4 | 0.000415181 |
| 142 | CHCHD5 | 0.000411257 |
| 143 | TES | 0.000410229 |
| 144 | ANAPC16 | 0.00040612 |
| 145 | DDX18 | 0.000405842 |
| 146 | FAU | 0.000401403 |
| 147 | ZC3HAV1 | 0.000384626 |
| 148 | HLA.DRA | 0.000383825 |
| 149 | BIN2 | 0.000382106 |
| 150 | DDX17 | 0.000375848 |
| 151 | HP1BP3 | 0.000373013 |
| 152 | PTPRC | 0.000367906 |
| 153 | RPL17 | 0.000365804 |
| 154 | PPIA | 0.000364396 |
| 155 | CCL5 | 0.000357919 |
| 156 | COX6A1 | 0.00035501 |
| 157 | LSM7 | 0.000352817 |
| 158 | RPL23 | 0.000341939 |
| 159 | STT3B | 0.000340606 |
| 160 | ZNF428 | 0.000339031 |
| 161 | VAMP8 | 0.000338092 |
| 162 | RPL6 | 0.000337001 |
| 163 | CD8A | 0.000334106 |
| 164 | POLR2I | 0.000333499 |
| 165 | ARHGAP30 | 0.000332356 |
| 166 | TTC14 | 0.000332236 |
| 167 | RPS18 | 0.000331036 |
| 168 | LSM6 | 0.000328714 |
| 169 | SSR4 | 0.00032843 |
| 170 | CLEC2B | 0.000324736 |
| 171 | GPSM3 | 0.000324493 |
| 172 | SRSF9 | 0.00032395 |
| 173 | PNRC1 | 0.000323715 |
| 174 | DUSP2 | 0.00032276 |
| 175 | LRRFIP1 | 0.000321934 |
| 176 | RNF213 | 0.000321411 |
| 177 | ERH | 0.000321181 |
| 178 | COX7A2 | 0.000321011 |
| 179 | NAA10 | 0.000317172 |
| 180 | PA2G4 | 0.000315746 |
| 181 | CDC42SE1 | 0.000313487 |
| 182 | NDUFB2 | 0.000311815 |
| 183 | FAM195B | 0.000311799 |
| 184 | NDUFB9 | 0.000311013 |
| 185 | RPL11 | 0.000304608 |
| 186 | JOSD2 | 0.000301649 |
| 187 | HMGN2 | 0.000298708 |
| 188 | SFPQ | 0.000294578 |
| 189 | BANF1 | 0.000292952 |
| 190 | ZNF207 | 0.000292714 |
| 191 | CHURC1 | 0.000292499 |
| 192 | SNX3 | 0.000289765 |
| 193 | NENF | 0.000287824 |
| 194 | C16orf13 | 0.000282382 |
| 195 | CKLF | 0.00028194 |
| 196 | CISD3 | 0.000281576 |
| 197 | RHOF | 0.000280805 |
| 198 | POLE4 | 0.000279025 |
| 199 | RPS5 | 0.00027819 |
| 200 | MYO1G | 0.00027809 |
| 201 | NDUFA1 | 0.000272964 |
| 202 | NOSIP | 0.00026912 |
| 203 | PDCD5 | 0.000266742 |
| 204 | EMP3 | 0.000266521 |
| 205 | SUN2 | 0.000263091 |
| 206 | AURKAIP1 | 0.000256714 |
| 207 | IKZF1 | 0.000255782 |
| 208 | UBXN11 | 0.000254844 |
| 209 | HMGN1 | 0.00025374 |
| 210 | MINOS1 | 0.000252667 |
| 211 | ABHD17A | 0.000251988 |
| 212 | RNASEH2C | 0.000251803 |
| 213 | C14orf2 | 0.000250531 |
| 214 | RASGRP2 | 0.000249522 |
| 215 | FMNL1 | 0.000247154 |
| 216 | CDKN2D | 0.000247119 |
| 217 | MTPN | 0.000246429 |
| 218 | TBCA | 0.00024378 |
| 219 | TTC19 | 0.000242335 |
| 220 | RPL36 | 0.000241997 |
| 221 | RPS13 | 0.000240079 |
| 222 | ATP5L | 0.000235236 |
| 223 | ANXA2R | 0.000233451 |
| 224 | ATOX1 | 0.000233108 |
| 225 | EIF4E | 0.000230816 |
| 226 | C7orf73 | 0.000229408 |
| 227 | TMC6 | 0.000228813 |
| 228 | TCF25 | 0.000225841 |
| 229 | DNAJB11 | 0.000225338 |
| 230 | TMEM219 | 0.000225184 |
| 231 | OAZ1 | 0.000220815 |
| 232 | RPS8 | 0.000220254 |
| 233 | CTSW | 0.000219513 |
| 234 | RPL38 | 0.000219489 |
| 235 | CBX6 | 0.000219195 |
| 236 | ATP5D | 0.000218966 |
| 237 | SPI1 | 0.000218858 |
| 238 | SEC61B | 0.000218251 |
| 239 | LINC00861 | 0.0002166 |
| 240 | CAPZA1 | 0.000216269 |
| 241 | MDM4 | 0.000215343 |
| 242 | ANKRD44 | 0.00021133 |
| 243 | LAMTOR4 | 0.000211294 |
| 244 | SRP9 | 0.000208176 |
| 245 | C19orf60 | 0.000207567 |
| 246 | OST4 | 0.000204408 |
| 247 | PTPN6 | 0.000202001 |
| 248 | LY6E | 0.000199901 |
| 249 | RPS21 | 0.000198975 |
| 250 | PSMB9 | 0.000198929 |
| 251 | NDUFB10 | 0.000198852 |
| 252 | ZEB2 | 0.000198632 |
| 253 | POLD4 | 0.000198133 |
| 254 | MIF | 0.000196685 |
| 255 | RTF1 | 0.000196359 |
| 256 | CLIC3 | 0.00019608 |
| 257 | RPS10 | 0.00019481 |
| 258 | PABPN1 | 0.000190371 |
| 259 | NOP10 | 0.000187697 |
| 260 | CNN2 | 0.000186634 |
| 261 | DSTN | 0.0001864 |
| 262 | SNF8 | 0.000184977 |
| 263 | LYAR | 0.000184208 |
| 264 | ZNF302 | 0.00018386 |
| 265 | COX6B1 | 0.000181034 |
| 266 | HNRNPC | 0.000179594 |
| 267 | WDR83OS | 0.000179507 |
| 268 | CMC1 | 0.000179313 |
| 269 | PIM1 | 0.000177959 |
| 270 | MBNL1 | 0.000177547 |
| 271 | RBL2 | 0.000177351 |
| 272 | GLIPR2 | 0.000177274 |
| 273 | PFN1 | 0.000176772 |
| 274 | POLR2J3 | 0.000175978 |
| 275 | TMEM167A | 0.000174243 |
| 276 | TGFB1 | 0.000173874 |
| 277 | IFITM1 | 0.000172206 |
| 278 | SNRPD2 | 0.000171796 |
| 279 | PRELID1 | 0.000171214 |
| 280 | RPL34 | 0.000170164 |
| 281 | PCNP | 0.000169875 |
| 282 | CDC42 | 0.000169503 |
| 283 | SSU72 | 0.000168608 |
| 284 | PTEN | 0.000166418 |
| 285 | ZFAS1 | 0.000165881 |
| 286 | UQCRH | 0.000164478 |
| 287 | C16orf54 | 0.000164119 |
| 288 | COX17 | 0.000160223 |
| 289 | ANAPC11 | 0.000156723 |
| 290 | CSK | 0.000156271 |
| 291 | FCGRT | 0.000155045 |
| 292 | RPL27 | 0.00015459 |
| 293 | LAMTOR2 | 0.000154483 |
| 294 | KRT10 | 0.000151949 |
| 295 | ARL6IP4 | 0.000151258 |
| 296 | IFI27L2 | 0.00014985 |
| 297 | ROMO1 | 0.000148865 |
| 298 | RPL28 | 0.000147802 |
| 299 | RNF167 | 0.000146421 |
| 300 | RPL30 | 0.000144795 |
| 301 | EIF5B | 0.000143641 |
| 302 | NCL | 0.000143211 |
| 303 | MMP24.AS1 | 0.000142412 |
| 304 | NDUFA13 | 0.000142261 |
| 305 | CFD | 0.000138063 |
| 306 | ATP5I | 0.000137571 |
| 307 | LINC00116 | 0.000136984 |
| 308 | TRAPPC1 | 0.000135245 |
| 309 | TSPO | 0.000133668 |
| 310 | DRAP1 | 0.000133384 |
| 311 | RPL27A | 0.000132097 |
| 312 | RAP1B | 0.000131245 |
| 313 | RPL12 | 0.000131086 |
| 314 | CAST | 0.000131013 |
| 315 | COMMD6 | 0.000128804 |
| 316 | CD14 | 0.000128137 |
| 317 | CNPY3 | 0.000126885 |
| 318 | RPS23 | 0.000126683 |
| 319 | COX7C | 0.000126265 |
| 320 | C11orf31 | 0.000126193 |
| 321 | TCEB2 | 0.000124652 |
| 322 | N4BP2L2 | 0.000124328 |
| 323 | TXNL4A | 0.000123254 |
| 324 | RPLP2 | 0.000122565 |
| 325 | FTL | 0.000122391 |
| 326 | HMGN3 | 0.00012163 |
| 327 | C19orf53 | 0.000119653 |
| 328 | TMA7 | 0.000119204 |
| 329 | PTP4A2 | 0.000118152 |
| 330 | ZRANB2 | 0.000117696 |
| 331 | COX7B | 0.000115701 |
| 332 | COX8A | 0.000115313 |
| 333 | VAMP2 | 0.000112998 |
| 334 | CST7 | 0.000112812 |
| 335 | MRPS21 | 0.00011245 |
| 336 | PPP3CA | 0.000111714 |
| 337 | DAZAP2 | 0.000110912 |
| 338 | LSM4 | 0.000110902 |
| 339 | DBI | 0.000110782 |
| 340 | TRA2B | 0.000109346 |
| 341 | NDUFA4 | 0.000109301 |
| 342 | TAOK3 | 0.000108586 |
| 343 | ATP5G1 | 0.000108582 |
| 344 | EFHD2 | 0.000106692 |
| 345 | FAM107B | 0.000106359 |
| 346 | FAM133B | 0.000104905 |
| 347 | ARPC5 | 0.000103902 |
| 348 | PYHIN1 | 0.000102734 |
| 349 | DOK2 | 0.00010235 |
| 350 | RPL22 | 0.000101582 |
| 351 | MRPL41 | 9.94E−05 |
| 352 | FLT3LG | 9.86E−05 |
| 353 | UBA52 | 9.81E−05 |
| 354 | PFDN5 | 9.78E−05 |
| 355 | TRAM1 | 9.76E−05 |
| 356 | POLR2J | 9.63E−05 |
| 357 | TOPORS.AS1 | 9.52E−05 |
| 358 | FIS1 | 9.50E−05 |
| 359 | PCBP1 | 9.50E−05 |
| 360 | TIMM13 | 9.11E−05 |
| 361 | SNRPG | 9.03E−05 |
| 362 | BRI3 | 9.00E−05 |
| 363 | ATP5J | 8.91E−05 |
| 364 | STK17B | 8.90E−05 |
| 365 | RPS15 | 8.87E−05 |
| 366 | BEST1 | 8.66E−05 |
| 367 | JAK1 | 8.66E−05 |
| 368 | RPS25 | 8.64E−05 |
| 369 | NDUFA2 | 8.38E−05 |
| 370 | CLEC2D | 8.18E−05 |
| 371 | FOXP1 | 8.16E−05 |
| 372 | STUB1 | 8.13E−05 |
| 373 | AAK1 | 7.98E−05 |
| 374 | SPON2 | 7.95E−05 |
| 375 | MRPL33 | 7.92E−05 |
| 376 | RPL21 | 7.92E−05 |
| 377 | SET | 7.89E−05 |
| 378 | POMP | 7.66E−05 |
| 379 | LSM5 | 7.51E−05 |
| 380 | KLF2 | 7.50E−05 |
| 381 | TMED2 | 7.40E−05 |
| 382 | TRAF3IP3 | 7.37E−05 |
| 383 | SRSF3 | 7.35E−05 |
| 384 | C19orf24 | 7.33E−05 |
| 385 | GPR65 | 7.32E−05 |
| 386 | PPDPF | 7.16E−05 |
| 387 | PRR13 | 7.15E−05 |
| 388 | COX5B | 7.13E−05 |
| 389 | ATP5E | 7.12E−05 |
| 390 | COTL1 | 7.09E−05 |
| 391 | RPS27A | 7.05E−05 |
| 392 | B3GAT2 | 6.84E−05 |
| 393 | ATP5EP2 | 6.80E−05 |
| 394 | CNOT7 | 6.79E−05 |
| 395 | SEPW1 | 6.62E−05 |
| 396 | H1FX | 6.59E−05 |
| 397 | PRPF4B | 6.56E−05 |
| 398 | GZMA | 6.53E−05 |
| 399 | SF1 | 6.44E−05 |
| 400 | COX6C | 6.29E−05 |
| 401 | PSAP | 6.28E−05 |
| 402 | ATP5J2 | 6.26E−05 |
| 403 | RPS19 | 6.26E−05 |
| 404 | CCDC85B | 6.24E−05 |
| 405 | GRK6 | 6.23E−05 |
| 406 | CD3G | 6.22E−05 |
| 407 | MYOIF | 6.21E−05 |
| 408 | GUK1 | 6.16E−05 |
| 409 | CD8B | 6.06E−05 |
| 410 | TRA2A | 6.05E−05 |
| 411 | SAMD3 | 6.03E−05 |
| 412 | IRF1 | 6.02E−05 |
| 413 | ATM | 5.99E−05 |
| 414 | LGALS1 | 5.98E−05 |
| 415 | PRF1 | 5.70E−05 |
| 416 | BCL11B | 5.69E−05 |
| 417 | RPL37A | 5.68E−05 |
| 418 | IL16 | 5.62E−05 |
| 419 | SUMO1 | 5.46E−05 |
| 420 | HCST | 5.45E−05 |
| 421 | TMSB4X | 5.43E−05 |
| 422 | YPEL3 | 5.20E−05 |
| 423 | PRDX5 | 5.20E−05 |
| 424 | RPS14 | 5.19E−05 |
| 425 | RPL35A | 5.10E−05 |
| 426 | CD47 | 4.89E−05 |
| 427 | NDUFA11 | 4.88E−05 |
| 428 | PNISR | 4.77E−05 |
| 429 | RPL32 | 4.65E−05 |
| 430 | SRM | 4.65E−05 |
| 431 | ETS1 | 4.62E−05 |
| 432 | CD52 | 4.61E−05 |
| 433 | SRRM1 | 4.57E−05 |
| 434 | NAA38 | 4.57E−05 |
| 435 | UQCR10 | 4.52E−05 |
| 436 | PCBP2 | 4.46E−05 |
| 437 | SH3BGRL3 | 4.40E−05 |
| 438 | MZT2B | 4.39E−05 |
| 439 | SSBP4 | 4.38E−05 |
| 440 | AGTRAP | 4.36E−05 |
| 441 | PYCARD | 4.30E−05 |
| 442 | PPP1CB | 4.27E−05 |
| 443 | S100A6 | 4.19E−05 |
| 444 | APOBEC3C | 4.14E−05 |
| 445 | NDUFS6 | 4.13E−05 |
| 446 | ARF6 | 4.10E−05 |
| 447 | ZYX | 4.09E−05 |
| 448 | SLIRP | 4.08E−05 |
| 449 | UBL5 | 4.06E−05 |
| 450 | RBX1 | 4.05E−05 |
| 451 | KLRG1 | 3.86E−05 |
| 452 | RPS15A | 3.85E−05 |
| 453 | AES | 3.84E−05 |
| 454 | CTNNB1 | 3.80E−05 |
| 455 | FUS | 3.76E−05 |
| 456 | BAX | 3.74E−05 |
| 457 | RSL24D1 | 3.58E−05 |
| 458 | RBBP4 | 3.54E−05 |
| 459 | CMPK1 | 3.52E−05 |
| 460 | TBC1D10C | 3.49E−05 |
| 461 | RPL31 | 3.47E−05 |
| 462 | PSME2 | 3.34E−05 |
| 463 | TNRC6B | 3.29E−05 |
| 464 | NEDD8 | 3.28E−05 |
| 465 | MYEOV2 | 3.28E−05 |
| 466 | RPL18A | 3.25E−05 |
| 467 | SCAF11 | 3.23E−05 |
| 468 | ITGB1 | 3.19E−05 |
| 469 | MT2A | 3.05E−05 |
| 470 | SEC62 | 2.99E−05 |
| 471 | RPS27L | 2.99E−05 |
| 472 | EIF5A | 2.98E−05 |
| 473 | RPL35 | 2.98E−05 |
| 474 | C6orf62 | 2.97E−05 |
| 475 | CDC42SE2 | 2.75E−05 |
| 476 | EPC1 | 2.69E−05 |
| 477 | GZMM | 2.69E−05 |
| 478 | GNG5 | 2.67E−05 |
| 479 | HOPX | 2.48E−05 |
| 480 | ATP6VOB | 2.48E−05 |
| 481 | FLNA | 2.46E−05 |
| 482 | CSNK1A1 | 2.46E−05 |
| 483 | NDUFC1 | 2.41E−05 |
| 484 | RPS24 | 2.35E−05 |
| 485 | SERPINA1 | 2.34E−05 |
| 486 | SRSF6 | 2.30E−05 |
| 487 | ANP32E | 2.16E−05 |
| 488 | C1orf162 | 2.15E−05 |
| 489 | CYBA | 2.13E−05 |
| 490 | KLRB1 | 2.13E−05 |
| 491 | ARGLU1 | 2.07E−05 |
| 492 | PET100 | 1.99E−05 |
| 493 | RPL37 | 1.92E−05 |
| 494 | RPS12 | 1.91E−05 |
| 495 | MIB2 | 1.91E−05 |
| 496 | EIF2S3 | 1.90E−05 |
| 497 | AP2S1 | 1.89E−05 |
| 498 | GZMB | 1.65E−05 |
| 499 | FAM49B | 1.65E−05 |
| 500 | UQCRQ | 1.64E−05 |
| 501 | FKBP2 | 1.64E−05 |
| 502 | NDUFB1 | 1.64E−05 |
| 503 | CEBPD | 1.63E−05 |
| 504 | PRMT2 | 1.63E−05 |
| 505 | VAMP5 | 1.62E−05 |
| 506 | PLAC8 | 1.61E−05 |
| 507 | CCL4 | 1.61E−05 |
| 508 | EIF1AX | 1.57E−05 |
| 509 | EIF3E | 1.55E−05 |
| 510 | ARRDC3 | 1.49E−05 |
| 511 | KTN1 | 1.38E−05 |
| 512 | XIST | 1.38E−05 |
| 513 | RAC1 | 1.37E−05 |
| 514 | ITGB2 | 1.37E−05 |
| 515 | BLOC1S1 | 1.36E−05 |
| 516 | PYURF | 1.35E−05 |
| 517 | ADD3 | 1.34E−05 |
| 518 | ATPIF1 | 1.30E−05 |
| 519 | SMDT1 | 1.11E−05 |
| 520 | CARD16 | 1.10E−05 |
| 521 | DDX6 | 1.05E−05 |
| 522 | NCF1 | 1.04E−05 |
| 523 | SLC25A37 | 8.44E−06 |
| 524 | MRPL52 | 8.40E−06 |
| 525 | NDUFA3 | 8.16E−06 |
| 526 | SEC61G | 8.05E−06 |
| 527 | MGEA5 | 7.99E−06 |
| 528 | STAG2 | 7.94E−06 |
| 529 | S100A4 | 7.78E−06 |
| 530 | C12orf75 | 5.46E−06 |
| 531 | AP1S2 | 5.39E−06 |
| 532 | IFITM3 | 5.31E−06 |
| 533 | TYMP | 5.25E−06 |
| 534 | MRPL23 | 5.24E−06 |
| 535 | YWHAZ | 3.56E−06 |
| 536 | ACTR2 | 3.13E−06 |
| 537 | RPL26 | 2.89E−06 |
| 538 | POLR2L | 2.77E−06 |
| 539 | LIMD2 | 2.73E−06 |
| 540 | SERF2 | 2.71E−06 |
| 541 | CEBPB | 2.38E−06 |
| 542 | PIP4K2A | 2.30E−06 |
| 543 | SARIA | 4.90E−07 |
| 544 | TMEM160 | 1.82E−07 |
| 545 | STXBP2 | 2.10E−08 |
| 546 | USMG5 | −3.23E−08 |
| 547 | ARPC4 | −7.70E−07 |
| 548 | NDUFB7 | −2.66E−06 |
| 549 | C4orf48 | −2.74E−06 |
| 550 | FAM65B | −4.73E−06 |
| 551 | GPX1 | −6.26E−06 |
| 552 | WTAP | −7.70E−06 |
| 553 | TMEM258 | −8.27E−06 |
| 554 | C9orf142 | −1.38E−05 |
| 555 | ZNF90 | −1.43E−05 |
| 556 | GSTP1 | −1.68E−05 |

Pseudotime analysis of entire tumor microenvironments: The samples were ordered in pseudotime using cell-type specific KEGG pathway scores for the cancer-related or pancreas-related pathways; these were pathways related to cell growth and death, cellular community, the digestive system, the immune system, replication and repair, signal transduction, and cellular transport and catabolism. Normalized and scaled cell counts, cancer- and pancreas-related pathway scores, and microbiome abundances for all 35 samples were combined into a single matrix and used as input for SAHMI's pseudotime functions. Normal and tumor states were clustered from the resulting branched dimensionality reduction representation, and the normal state (NS) and tumor state 1 (TS1) were manually split because they completely separated into ends of the same first branch of the pseudotime process. Numerical microbiome and clinical parameters were compared across the tumor states with t-tests, and categorical parameters were compared using Fisher's exact test.

Joint analysis of microbial diversity and survival: The microbiome Shannon diversity index was calculated for each sample in the Peng et al. cohort (Peng et al. Cell Res. 29(9):725-738, 2019). Patients were stratified by their predicted tumor microbial diversity and the survminer package (github.conVkassambara/survminer/) was used to test the relationship with survival and to plot Kaplan-Meier curves. The relationship between survival and microbial diversity was also tested in TCGA pancreatic cancers using microbial profiles directly estimated from TCGA data by Poore et al. (Poore et al. Nature 579: 567-574, 2020). The Shannon diversity index was calculated from TCGA microbiome count data for all genera that passed their quality filters.

Statistical analyses: All statistical analyses were performed using R version 3.6.1. All p-values were false-discovery rate (fdr)—corrected for multiple hypothesis using the p.adjust function with method=“fdr”, unless otherwise stated. The ggpubr package (github.com/kassambara/ggpubr) was used to compare group means with nonparametric tests and to perform multiple hypothesis correction for statistics that are noted in figures. P-values reported as <2.2×10⁻¹⁶ result from reaching the calculation limit for native R statistical test functions and indicate values below this number, not a range of values. Diversity calculations used the vegan package (github.com/vegandevs/vegan).

### Results and Discussion

This example describes a particular embodiment of the SAHMI (Single-cell Analysis of Host-Microbiome Interactions) method to examine patterns of human-microbiome interactions in the pancreatic tumor microenvironment at single cell resolution using genomic approaches.

Detection and validation of metagenomic reads in scRNAseq data: Single-cell Analysis of Host-Microbiome Interactions (SAHMI) was developed as a pipeline to reliably identify and annotate metagenomic reads in single-cell RNA sequencing experiments (scRNAseq) and to quantify microbial abundance in human tissue samples. SAHMI enables the systematic assessment of microbial diversity and patterns of microbe-host cell type interactions at single cell resolution in the tissue microenvironment (FIG. 15A, Example 1), with implications for tissue-level functions and pathological and clinical modalities.

First, SAHMI maps the reads from single cell sequencing experiments to the host genome and uses the resulting transcriptomic signatures to cluster and annotate somatic cell types (Dobin et al. Bioinformatics 29: 15-21, 2013; Stuart et al. Cell 177: 1888-1902.e21, 2019). Next, it compares the remaining unmapped reads to a reference microbiome database to detect exact matches, as implemented elsewhere (Wood et al. Genome Biol. 20: 257, 2019), and identifies microbial entities at the most precise taxonomic level possible, estimating their abundance. SAHMI implements a series of filters to remove low quality reads, potentially spurious entries, and laboratory contaminants, only reporting high confidence microbial taxa. The cellular barcodes allow for pairing of microbial entities with corresponding somatic cells at the resolution of single cells. Jointly analyzing the attributes of host cells and associated microbes, SAHMI enables analysis of microbiome and host interactions at multiple levels—from the resolution of individual cells to the level of inter-cellular interactions within the tissue sample microenvironment.

SAHMI was used herein to study tumor-microbiome interactions using scRNAseq data for 24 human pancreatic ductal adenocarcinomas (PDA) and 11 control pancreatic pathologies (non-PDA lesions) (Peng et al. Cell Res. 29(9):725-738, 2019); all samples were obtained during pancreatectomy or pancreatoduodenectomy (Table 1), and all were processed similarly. No batch affects were observed within or between tumor and non-tumor samples (FIG. 20A), mitigating concerns of differential contamination confounding microbiome inferences. These pancreatic tissues had 100-500 million total sequencing reads per sample; after applying multiple quality filters, SAHMI classified 3-10% as bacterial and <1% as fungal (FIG. 20B). SAHMI identified 285 bacterial and 35 fungal genera in PDA and pancreatic tissues, which were detected on 23,546 barcodes, of which 13,848 (58%) also detected RNA from host cells. There was no significant difference in filtered metagenomic read counts between tumor and control samples (FIGS. 20B-20D). However, 68% of microbiome reads from tumor samples were tagged with molecular barcodes which also tagged mRNAs in human somatic cell types, compared to 38% of reads from control samples (Wilcoxon, p=0.001, FIG. 20E). Malignant ductal cells were the cell-types with the highest concentration of metagenomic counts (FIG. 20E). These data indicate broad changes encompassing tissue-microbiome architectural, biochemical, or biophysical properties.

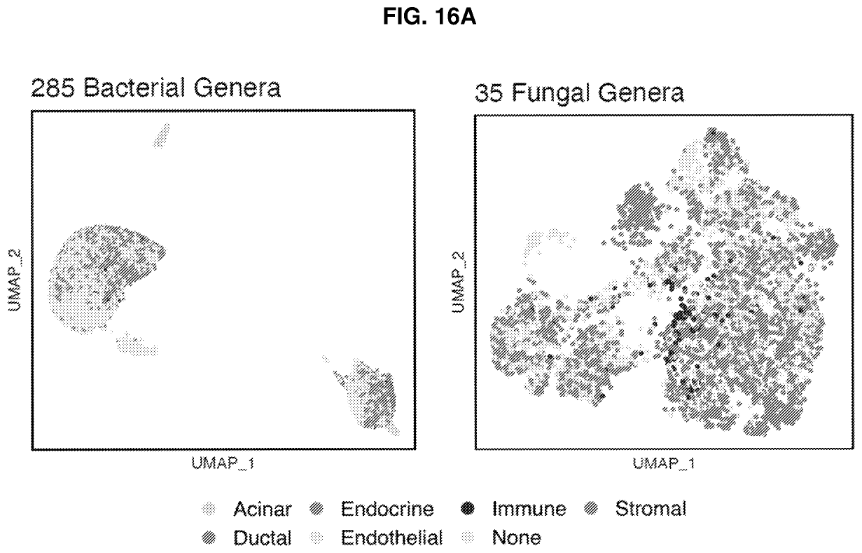

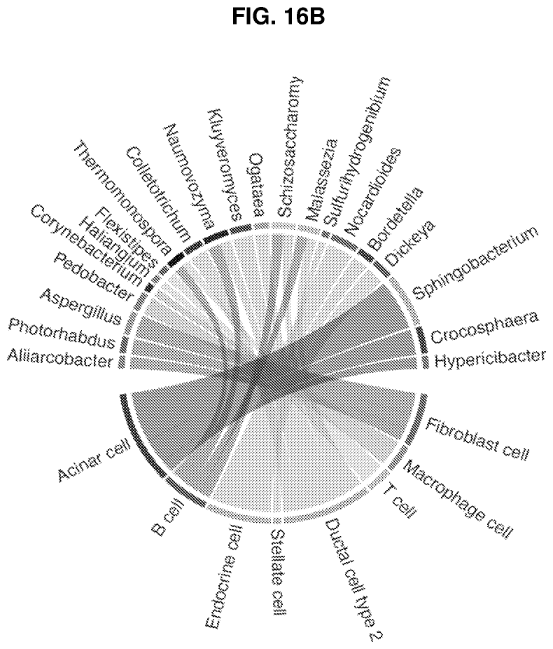

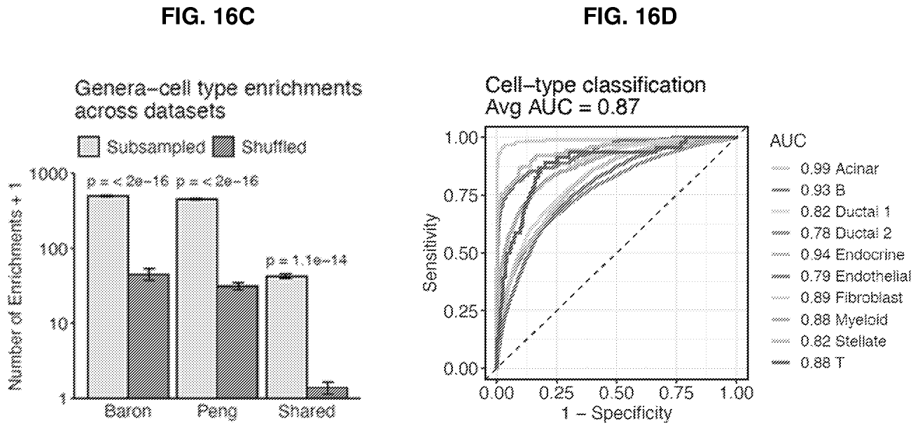

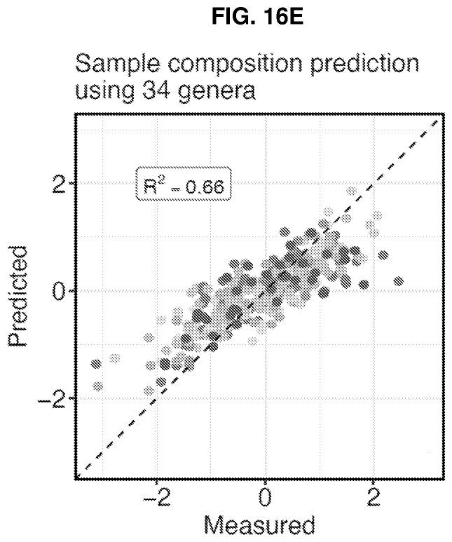

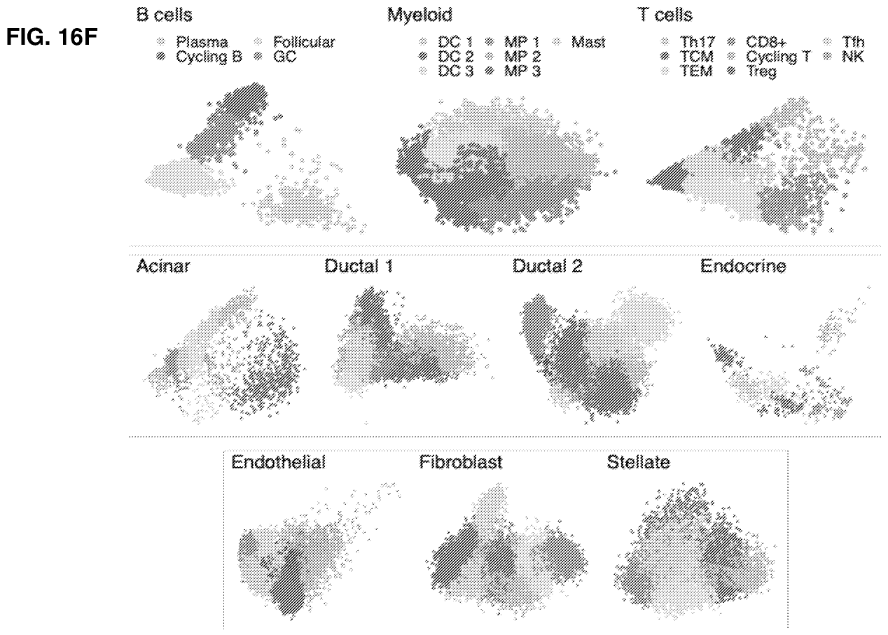

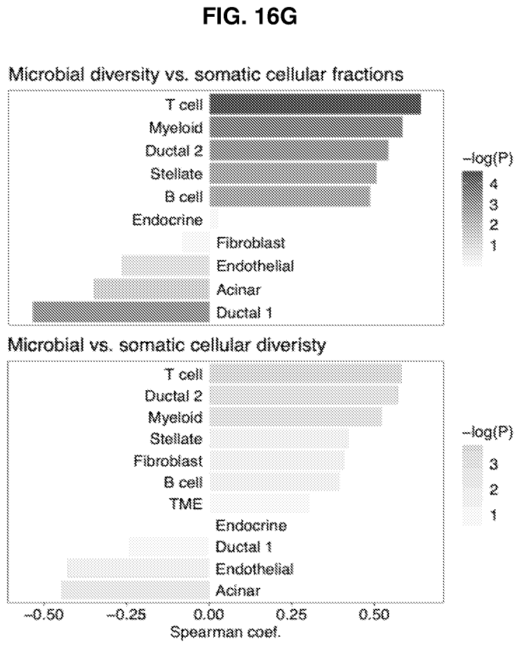

Multiple validation and benchmarking steps were used to ensure that observations were not due to sequencing artifacts or laboratory contamination. First, bacterial entities detected at the genus level from this cohort were compared to (i) entities estimated herein from two other studies that performed single cell sequencing of the normal pancreas (Baron et al. Cell Syst. 3: 346-360.e4, 2016; Muraro et al. Cell Syst. 3: 385-394.e3, 2016), (ii) entities determined from bulk-RNA sequencing data in The Cancer Genome Atlas (TCGA) (Poore et al. Nature, 579: 567-574, 2020), and (iii) entities determined from 16S-rRNA sequencing in a recent large-scale study (Nejman et al. Science, 368(6494):973-980, 2020)—for a total of 298 pancreatic samples sequenced with three different technologies. Excellent agreement was found, with bacterial compositions showing strong quantitative (mean spearman p=0.61, harmonic mean p-value=9×10⁻⁵², median p=1×10⁻⁵) and qualitative (mean overlap coefficient=0.70) concordance across all datasets (FIG. 15C), with greater consistency across the single-cell studies (p=0.75, harmonic p=4×10⁻⁵²). Next, 20 of 26 prior published differences in bacterial abundances in pancreatic disease samples were detected (Thomas et al. Nat. Rev. Gastroenterol. Hepatol. 17: 53-64, 2020) 19 of the 20 showed significant tumor-normal differences (FIG. 15B; Wilcoxon, p<0.05). The filtered reads were also examined for the putative common laboratory contaminants reported by Poore et al (Poore et al. Nature 579: 567-574, 2020). Only 19 (9.5%) of 201 detected putative contaminant genera passed the quality filters used herein. All were detected at low expression levels, and 14 of the 19 showed tumor-normal differences (Wilcoxon, p<0.05) (FIG. 15B). Finally, a substantial proportion of the identified microbes were preferentially associated with specific somatic cell types and their cellular activities. Microbiome profiles were also associated with tissue clinical attributes, consistent with collateral literature, as discussed below (FIGS. 16-19), and which cannot be explained by random sequencing artifacts or laboratory contamination. Taken together, these results indicate that SAHMI can reliably quantify microbial abundances from single-cell sequencing data of host tissues at a level comparable to other high-throughput methods, with the advantage of being able to simultaneously analyze somatic cellular gene expression and assess cell-type specific host-microbiome associations.

Pancreatic tumors and non-malignant tissues have distinct microbiomes: Metagenomic data were visualized using uniform manifold approximation and projection (UMAP), a nonlinear dimensionality reduction method that projects the barcode by genus data-table onto a 2-dimensional plane, clustering barcodes with similar metagenomic profiles. The individual bacterial and fungal UMAPs revealed global tumor-normal differences, as indicated by broad separation of tumor and nontumor-derived clusters, as well as multiple barcode clusters with distinct bacterial and fungal compositions (FIG. 15F). Notably, these clusters persisted when data for pancreatic samples from three independent cohorts were jointly analyzed (FIG. 20F), highlighting the consistent detection of a putative commensal microbiome in diverse pancreatic tissues that differs from that of PDAs. Alpha-diversity in the PDA microbiome was significantly increased compared to controls (FIG. 15G).

Specific microbial abundances were then compared between tumor and non-tumor samples using a linear model that includes disease status, total metagenomic counts, and somatic cell counts (to account for selective tropism) as covariates (FIG. 15E, see Methods). Three bacterial genera (Klebsiella spp., Pasteurella spp., Staphylococcus spp.) comprised >80% of the detected microbiome in all the samples from non-malignant illnesses and from most of the tumors (FIG. 15D). A subset of tumors had markedly different microbial compositions, characterized by a decrease in putative commensal genera and an expansion of several low-abundance taxa. These genera included several pathogens previously associated with human infection, with carcinogenesis, or with pancreatic cancer. For example, gut infections by Vibrio spp. (Baker-Austin et al. Nat. Rev. Dis. Prim. 4: 8, 2018) and Campylobacter spp. (Janssen et al. Clin. Microbiol. Rev. 21: 505-518, 2008) are known to cause local and systemic inflammation, Fusobacterium nucleatum is strongly associated with tumorigenesis in colorectal cancer (Sethi et al. Gastroenterology 156: 2097-2115.e2, 2019), Aspergillus spp. produces carcinogenic mycotoxins (Hedayati et al. Microbiology 153: 1677-1692, 2007), and other taxa, including Prevotella spp., Megamonas spp., Bacteroides spp., Streptococcus spp., Lactobacillus spp., Streptomyces spp., and Clostridium spp. have been associated with pancreatic disease in pre-clinical and epidemiological studies, via differential detection in the oral cavity, plasma, feces, or pancreas (Sethi et al. Gastroenterology, 156: 2097-2115.e2, 2019; Thomas et al. Nat. Rev. Gastroenterol. Hepatol. 17: 53-64, 2020). In total, these findings indicate that pancreatic tumors and non-malignant tissues differ in both microbiome community structure and composition.

Specific host cell-types are enriched with particular microbes: To examine whether bacteria and fungi in human pancreatic tissues are associated with specific host cell types, barcodes that tagged both metagenomic and somatic RNA were identified. It was observed that metagenomes whose barcodes originated from the same somatic cell-type clustered together in the prior UMAP plots (FIG. 16A), and that specific microbes were significantly enriched in particular cell-types (FIG. 16B). About 500 statistically significant microbiome-host cell-type enrichments (Table 3) were consistently found in two single-cell pancreas datasets (Peng et al. Cell Res. 29(9):725-738, 2019; Baron et al. Cell Syst. 3: 346-360.e4, 2016), of which ˜50 enrichments were shared across the datasets, which was significantly more than expected by chance when cell-type labels were shuffled (FIG. 16C, Peng: p<2×10⁻¹⁶, Baron: p<2×10⁻¹⁶, Shared: p=1.1×10⁻⁴, see Methods). These observations provided further support that the observed microbiome profiles were unlikely to be due to laboratory contaminations or sequencing artifacts, and they suggested the presence of select microbial tropisms with pancreatic cell types. The strongest examples were found between Sphingobacterium spp. and acinar cells (Wilcoxon, p=2e-52) and between Nocardioides spp. and endocrine cells (Wilcoxon, p=4e-26).

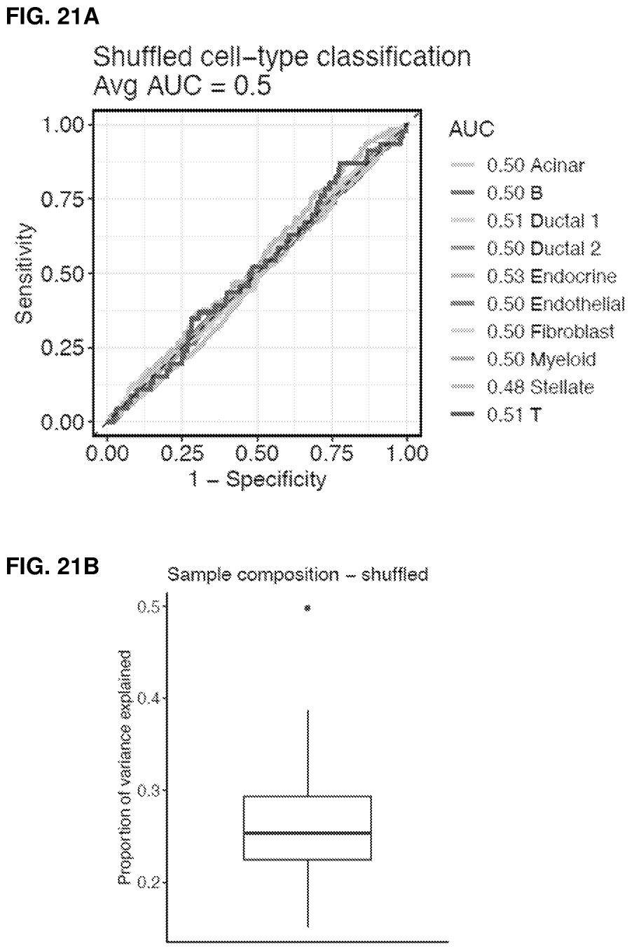

Strong cell type co-localization with particular microbes permitted prediction of barcode cell-types and sample cellular composition based solely on microbiome profiles. A random forest model to predict a barcode's somatic cell-type given its associated metagenomic composition achieved high accuracy in classifying all cell-types (AUC: 0.87; FIG. 16D), and regularized linear regression identified 34 genera whose sample-level abundances accurately predicted somatic cellular composition (r=0.81, FIG. 16E). In contrast, null models with shuffled sample labels performed poorly (FIGS. 21A-21B). These observations indicated tropisms between particular microbes and somatic cells in the pancreas, and provided further validation of microbiome detection from scRNAseq data using SAHMI.

TABLE 3
Cell-type microbiome enrichments.

| | | | Avg_ | | | P_val_ |
|---|---|---|---|---|---|---|
| Cluster | Genus | P_value | logFC | Pct. 1 | Pct. 2 | adj |
| None | Neisseria | 5.30E−21 | 0.483 | 0.935 | 0.935 | 1.89E−18 |
| None | Granulibacter | 3.93E−11 | 0.636 | 0.490 | 0.282 | 1.40E−08 |
| None | Thalassotalea | 3.81E−06 | 0.302 | 0.710 | 0.580 | 1.36E−03 |
| None | Iodobacter | 1.94E−05 | 0.329 | 0.305 | 0.181 | 6.91E−03 |
| None | Dermabacter | 2.01E−05 | 0.409 | 0.300 | 0.179 | 7.16E−03 |
| Fibroblast | Labilibaculum | 2.34E−21 | 0.753 | 0.680 | 0.421 | 8.32E−19 |
| Fibroblast | Edwardsiella | 1.20E−07 | 0.514 | 0.500 | 0.360 | 4.28E−05 |
| Fibroblast | Kangiella | 1.37E−07 | 0.387 | 0.740 | 0.624 | 4.88E−05 |
| Fibroblast | Solitalea | 2.12E−07 | 0.410 | 0.555 | 0.390 | 7.54E−05 |
| Fibroblast | Yarrowia | 4.47E−07 | 1.497 | 0.290 | 0.170 | 1.59E−04 |
| Fibroblast | Jiangella | 1.72E−06 | 0.343 | 0.410 | 0.270 | 6.11E−04 |
| Fibroblast | Pseudolysobacter | 2.68E−06 | 0.284 | 0.750 | 0.618 | 9.54E−04 |
| Fibroblast | Pochonia | 4.35E−05 | 1.704 | 0.290 | 0.201 | 1.55E−02 |
| Fibroblast | Saccharomyces | 4.59E−05 | 1.687 | 0.290 | 0.200 | 1.63E−02 |
| Fibroblast | Aspergillus | 7.40E−05 | 1.082 | 0.290 | 0.201 | 2.63E−02 |
| Fibroblast | Nakaseomyces | 1.15E−04 | 0.617 | 0.170 | 0.089 | 4.10E−02 |
| Macrophage | Pedobacter | 1.11E−31 | 1.332 | 0.895 | 0.662 | 3.95E−29 |
| Macrophage | Corynebacterium | 1.22E−09 | 0.522 | 0.795 | 0.700 | 4.34E−07 |
| Macrophage | Clostridium | 1.83E−08 | 0.276 | 0.985 | 0.968 | 6.51E−06 |
| Macrophage | Halomonas | 2.36E−08 | 0.480 | 0.885 | 0.854 | 8.39E−06 |
| Macrophage | Xanthomonas | 1.11E−07 | 0.286 | 0.975 | 0.957 | 3.95E−05 |
| Macrophage | Pseudolysobacter | 2.11E−07 | 0.397 | 0.720 | 0.621 | 7.51E−05 |
| Macrophage | Mycoplasma | 3.41E−07 | 0.335 | 0.935 | 0.894 | 1.21E−04 |
| Macrophage | Spiroplasma | 5.80E−07 | 0.260 | 0.900 | 0.809 | 2.06E−04 |
| Macrophage | Bacteroides | 8.84E−07 | 0.516 | 0.760 | 0.685 | 3.15E−04 |
| Macrophage | Campylobacter | 2.79E−06 | 0.263 | 0.950 | 0.905 | 9.93E−04 |
| Macrophage | Acinetobacter | 4.01E−06 | 0.265 | 0.930 | 0.888 | 1.43E−03 |
| Macrophage | Polaribacter | 1.68E−05 | 0.278 | 0.880 | 0.804 | 6.00E−03 |
| Macrophage | Proteus | 2.81E−05 | 0.272 | 0.695 | 0.586 | 1.00E−02 |
| Macrophage | Enterobacter | 4.94E−05 | 0.275 | 0.755 | 0.681 | 1.76E−02 |
| Macrophage | Helicobacter | 9.12E−05 | 0.286 | 0.765 | 0.700 | 3.25E−02 |
| Macrophage | Fusobacterium | 9.97E−05 | 0.296 | 0.925 | 0.906 | 3.55E−02 |
| Macrophage | Calothrix | 1.35E−04 | 0.315 | 0.655 | 0.600 | 4.79E−02 |
| Macrophage | Acetobacter | 1.83E−04 | 0.275 | 0.635 | 0.582 | 6.53E−02 |
| Endothelial | Ilyobacter | 6.51E−10 | 0.383 | 0.435 | 0.230 | 2.32E−07 |
| Endothelial | Rhodoferax | 2.76E−06 | 0.277 | 0.300 | 0.165 | 9.82E−04 |
| Endothelial | Desulfococcus | 5.43E−06 | 0.263 | 0.435 | 0.269 | 1.93E−03 |
| T_cell | Haliangium | 5.39E−18 | 0.556 | 0.842 | 0.714 | 1.92E−15 |
| T_cell | Flexistipes | 7.08E−12 | 0.604 | 0.597 | 0.437 | 2.52E−09 |
| T_cell | Xanthomonas | 9.12E−10 | 0.433 | 0.954 | 0.959 | 3.25E−07 |
| T_cell | Thermomonospora | 7.79E−07 | 0.525 | 0.531 | 0.440 | 2.77E−04 |
| Ductal_2 | Neisseria | 2.13E−17 | 0.411 | 0.970 | 0.932 | 7.59E−15 |
| Ductal_2 | Jiangella | 9.09E−16 | 0.625 | 0.520 | 0.259 | 3.24E−13 |
| Ductal_2 | Kineobactrum | 8.83E−13 | 0.458 | 0.465 | 0.237 | 3.15E−10 |
| Ductal_2 | Ustilago | 8.80E−09 | 0.633 | 0.325 | 0.169 | 3.13E−06 |
| Ductal_2 | Yarrowia | 6.10E−08 | 0.865 | 0.315 | 0.168 | 2.17E−05 |
| Ductal_2 | Pseudolysobacter | 2.13E−07 | 0.410 | 0.780 | 0.615 | 7.58E−05 |
| Ductal_2 | Iodobacter | 2.60E−07 | 0.265 | 0.340 | 0.178 | 9.25E−05 |
| Ductal_2 | Kluyveromyces | 7.89E−07 | 0.846 | 0.305 | 0.166 | 2.81E−04 |
| Ductal_2 | Saccharomyces | 1.30E−06 | 0.790 | 0.330 | 0.196 | 4.64E−04 |
| Ductal_2 | Pochonia | 1.55E−06 | 0.586 | 0.330 | 0.197 | 5.51E−04 |
| Ductal_2 | Pyricularia | 1.67E−06 | 0.362 | 0.325 | 0.184 | 5.96E−04 |
| Ductal_2 | Cryptococcus | 3.71E−06 | 0.326 | 0.330 | 0.196 | 1.32E−03 |
| Ductal_2 | Neurospora | 4.68E−06 | 0.259 | 0.330 | 0.196 | 1.66E−03 |
| Ductal_2 | Zymoseptoria | 5.37E−06 | 0.266 | 0.330 | 0.197 | 1.91E−03 |
| Ductal_2 | Encephalitozoon | 5.73E−06 | 0.650 | 0.330 | 0.194 | 2.04E−03 |
| Ductal_2 | Colletotrichum | 6.37E−06 | 0.503 | 0.330 | 0.197 | 2.27E−03 |
| Ductal_2 | Ogataea | 8.98E−06 | 0.568 | 0.325 | 0.195 | 3.20E−03 |
| Ductal_2 | Fusarium | 9.07E−06 | 0.319 | 0.330 | 0.195 | 3.23E−03 |
| Ductal_2 | Pararhodospirillum | 1.05E−05 | 0.314 | 0.695 | 0.561 | 3.73E−03 |
| Ductal_2 | Thermothielavioides | 1.11E−05 | 0.317 | 0.330 | 0.197 | 3.96E−03 |
| Ductal_2 | Lachancea | 2.08E−05 | 0.455 | 0.205 | 0.104 | 7.40E−03 |
| Ductal_2 | Thermothelomyces | 2.81E−05 | 0.401 | 0.305 | 0.185 | 9.99E−03 |
| Ductal_2 | Sporisorium | 2.91E−05 | 0.496 | 0.325 | 0.196 | 1.04E−02 |
| Ductal_2 | Sugiyamaella | 3.34E−05 | 0.468 | 0.320 | 0.191 | 1.19E−02 |
| Ductal_2 | Eremothecium | 1.11E−04 | 0.357 | 0.225 | 0.125 | 3.96E−02 |
| Stellate | Sulfurihydrogenibium | 3.96E−09 | 0.739 | 0.490 | 0.345 | 1.41E−06 |
| Stellate | Labilibaculum | 5.23E−08 | 0.449 | 0.585 | 0.431 | 1.86E−05 |
| Stellate | Nitrosomonas | 5.10E−07 | 0.431 | 0.380 | 0.249 | 1.82E−04 |
| Stellate | Kangiella | 8.26E−07 | 0.341 | 0.715 | 0.627 | 2.94E−04 |
| Stellate | Xenorhabdus | 6.53E−05 | 0.345 | 0.530 | 0.435 | 2.33E−02 |
| Stellate | Listeria | 7.29E−05 | 0.462 | 0.635 | 0.568 | 2.60E−02 |
| Endocrine | Nocardioides | 3.82E−49 | 1.993 | 0.845 | 0.444 | 1.36E−46 |
| Endocrine | Bordetella | 1.81E−48 | 1.161 | 0.810 | 0.393 | 6.45E−46 |
| Endocrine | Cupriavidus | 3.47E−37 | 0.972 | 0.895 | 0.529 | 1.23E−34 |
| Endocrine | Streptomyces | 1.28E−31 | 1.060 | 1.000 | 0.965 | 4.56E−29 |
| Endocrine | Muricauda | 3.33E−30 | 1.573 | 0.515 | 0.195 | 1.18E−27 |
| Endocrine | Dickeya | 2.20E−29 | 1.387 | 0.810 | 0.433 | 7.82E−27 |
| Endocrine | Hydrogenophaga | 4.51E−29 | 0.950 | 0.735 | 0.434 | 1.60E−26 |
| Endocrine | Pantoea | 8.14E−26 | 0.846 | 0.815 | 0.506 | 2.90E−23 |
| Endocrine | Actinoplanes | 1.36E−25 | 0.904 | 0.675 | 0.338 | 4.85E−23 |
| Endocrine | Hymenobacter | 1.67E−23 | 0.954 | 0.820 | 0.523 | 5.94E−21 |
| Endocrine | Achromobacter | 4.53E−23 | 0.967 | 0.630 | 0.316 | 1.61E−20 |
| Endocrine | Sorangium | 1.63E−18 | 0.899 | 0.635 | 0.349 | 5.79E−16 |
| Endocrine | Nonomuraea | 3.04E−18 | 0.768 | 0.530 | 0.274 | 1.08E−15 |
| Endocrine | Microbacterium | 5.45E−18 | 0.734 | 0.680 | 0.388 | 1.94E−15 |
| Endocrine | Raoultella | 3.56E−17 | 0.503 | 0.460 | 0.194 | 1.27E−14 |
| Endocrine | Chromobacterium | 6.67E−17 | 0.543 | 0.570 | 0.284 | 2.37E−14 |
| Endocrine | Amycolatopsis | 9.97E−17 | 0.734 | 0.590 | 0.313 | 3.55E−14 |
| Endocrine | Deinococcus | 3.07E−16 | 0.774 | 0.735 | 0.465 | 1.09E−13 |
| Endocrine | Micromonospora | 3.37E−16 | 0.927 | 0.835 | 0.611 | 1.20E−13 |
| Endocrine | Pseudolysobacter | 9.39E−16 | 0.449 | 0.870 | 0.606 | 3.34E−13 |
| Endocrine | Mycobacterium | 1.37E−14 | 0.603 | 0.910 | 0.684 | 4.89E−12 |
| Endocrine | Brachybacterium | 1.82E−14 | 0.671 | 0.455 | 0.225 | 6.47E−12 |
| Endocrine | Stenotrophomonas | 1.31E−13 | 0.598 | 0.705 | 0.467 | 4.67E−11 |
| Endocrine | Gordonia | 9.23E−13 | 0.574 | 0.455 | 0.233 | 3.29E−10 |
| Endocrine | Cellulomonas | 1.59E−12 | 0.585 | 0.575 | 0.336 | 5.64E−10 |
| Endocrine | Rathayibacter | 8.97E−12 | 0.750 | 0.455 | 0.253 | 3.19E−09 |
| Endocrine | Methylobacterium | 4.18E−11 | 0.456 | 0.845 | 0.686 | 1.49E−08 |
| Endocrine | Alistipes | 1.28E−10 | 0.644 | 0.335 | 0.166 | 4.56E−08 |
| Endocrine | Nocardia | 3.08E−10 | 0.664 | 0.670 | 0.465 | 1.09E−07 |
| Endocrine | Massilia | 5.28E−10 | 0.501 | 0.540 | 0.327 | 1.88E−07 |
| Endocrine | Rhodococcus | 6.60E−10 | 1.090 | 0.945 | 0.807 | 2.35E−07 |
| Endocrine | Solitalea | 8.45E−10 | 0.309 | 0.615 | 0.384 | 3.01E−07 |
| Endocrine | Frankia | 1.19E−09 | 0.760 | 0.490 | 0.303 | 4.24E−07 |
| Endocrine | Pseudonocardia | 6.48E−09 | 0.361 | 0.470 | 0.270 | 2.31E−06 |
| Endocrine | Actinomyces | 1.12E−08 | 0.617 | 0.635 | 0.447 | 4.00E−06 |
| Endocrine | Bradyrhizobium | 4.27E−08 | 0.722 | 0.630 | 0.461 | 1.52E−05 |
| Endocrine | Desulfovibrio | 7.84E−08 | 0.338 | 0.555 | 0.355 | 2.79E−05 |
| Endocrine | Mycolicibacterium | 1.01E−07 | 0.461 | 0.820 | 0.666 | 3.58E−05 |
| Endocrine | Paraburkholderia | 1.38E−07 | 0.501 | 0.555 | 0.378 | 4.91E−05 |
| Endocrine | Dermabacter | 2.02E−07 | 0.252 | 0.330 | 0.176 | 7.18E−05 |
| Endocrine | Blastochloris | 2.22E−07 | 0.304 | 0.270 | 0.133 | 7.91E−05 |
| Endocrine | Kitasatospora | 2.71E−07 | 0.611 | 0.435 | 0.293 | 9.64E−05 |
| Endocrine | Nocardiopsis | 3.67E−07 | 0.367 | 0.520 | 0.355 | 1.31E−04 |
| Endocrine | Bifidobacterium | 6.42E−07 | 0.391 | 0.825 | 0.651 | 2.29E−04 |
| Endocrine | Granulibacter | 1.10E−06 | 0.289 | 0.460 | 0.285 | 3.91E−04 |
| Endocrine | Myxococcus | 2.50E−06 | 0.469 | 0.460 | 0.315 | 8.88E−04 |
| Endocrine | Geobacillus | 2.56E−05 | 0.833 | 0.380 | 0.266 | 9.12E−03 |
| Endocrine | Bartonella | 8.02E−05 | 0.560 | 0.810 | 0.672 | 2.85E−02 |
| Endocrine | Dokdonia | 9.21E−05 | 0.342 | 0.435 | 0.301 | 3.28E−02 |
| B_cell | Magnetospirillum | 1.51E−25 | 0.741 | 0.795 | 0.568 | 5.37E−23 |
| B_cell | Rhodococcus | 3.76E−25 | 0.504 | 0.885 | 0.813 | 1.34E−22 |
| B_cell | Thermomonospora | 3.26E−23 | 0.667 | 0.715 | 0.422 | 1.16E−20 |
| B_cell | Virgibacillus | 1.35E−21 | 0.510 | 0.900 | 0.767 | 4.79E−19 |
| B_cell | Cercospora | 1.29E−15 | 1.154 | 0.340 | 0.144 | 4.59E−13 |
| B_cell | Ralstonia | 1.86E−14 | 0.269 | 0.960 | 0.941 | 6.62E−12 |
| B_cell | Malassezia | 3.70E−13 | 0.990 | 0.355 | 0.171 | 1.32E−10 |
| B_cell | Debaryomyces | 4.68E−13 | 0.383 | 0.210 | 0.063 | 1.67E−10 |
| B_cell | Naumovozyma | 6.53E−13 | 1.312 | 0.365 | 0.186 | 2.32E−10 |
| B_cell | Eremothecium | 4.93E−12 | 0.675 | 0.295 | 0.118 | 1.76E−09 |
| B_cell | Pyricularia | 4.98E−12 | 0.975 | 0.365 | 0.180 | 1.77E−09 |
| B_cell | Kluyveromyces | 8.13E−12 | 0.535 | 0.355 | 0.161 | 2.90E−09 |
| B_cell | Thermothielavioides | 1.00E−11 | 1.088 | 0.365 | 0.193 | 3.56E−09 |
| B_cell | Colletotrichum | 1.36E−11 | 1.036 | 0.365 | 0.194 | 4.85E−09 |
| B_cell | Schizosaccharomyces | 1.79E−11 | 1.111 | 0.365 | 0.194 | 6.39E−09 |
| B_cell | Sugiyamaella | 3.05E−11 | 0.813 | 0.365 | 0.187 | 1.09E−08 |
| B_cell | Sporisorium | 4.74E−11 | 0.688 | 0.365 | 0.192 | 1.69E−08 |
| B_cell | Torulaspora | 1.14E−10 | 0.273 | 0.175 | 0.055 | 4.07E−08 |
| B_cell | Zygosaccharomyces | 2.42E−10 | 0.452 | 0.210 | 0.076 | 8.60E−08 |
| B_cell | Thermothelomyces | 6.02E−10 | 0.548 | 0.360 | 0.180 | 2.14E−07 |
| B_cell | Fusarium | 6.62E−10 | 0.630 | 0.365 | 0.192 | 2.36E−07 |
| B_cell | Neurospora | 1.08E−09 | 0.770 | 0.365 | 0.192 | 3.84E−07 |
| B_cell | Zymoseptoria | 1.97E−09 | 0.717 | 0.365 | 0.194 | 7.01E−07 |
| B_cell | Cryptococcus | 8.46E−09 | 0.483 | 0.365 | 0.193 | 3.01E−06 |
| B_cell | Ogataea | 3.06E−08 | 0.564 | 0.365 | 0.191 | 1.09E−05 |
| B_cell | Encephalitozoon | 3.33E−08 | 0.597 | 0.360 | 0.191 | 1.19E−05 |
| B_cell | Haliangium | 6.72E−08 | 0.277 | 0.845 | 0.713 | 2.39E−05 |
| B_cell | Lachancea | 1.10E−07 | 0.422 | 0.225 | 0.102 | 3.92E−05 |
| B_cell | Ustilago | 4.83E−07 | 0.460 | 0.315 | 0.170 | 1.72E−04 |
| B_cell | Botrytis | 1.52E−06 | 0.534 | 0.295 | 0.153 | 5.41E−04 |
| B_cell | Thioalkalivibrio | 1.51E−05 | 0.284 | 0.740 | 0.656 | 5.38E−03 |
| Ductal_1 | Neisseria | 3.47E−20 | 0.384 | 0.990 | 0.930 | 1.23E−17 |
| Ductal_1 | Solitalea | 2.24E−09 | 0.407 | 0.595 | 0.386 | 7.98E−07 |
| Acinar | Sphingobacterium | 1.06E−118 | 3.943 | 0.985 | 0.574 | 3.78E−116 |
| Acinar | Pseudolabrys | 3.91E−58 | 0.907 | 0.405 | 0.062 | 1.39E−55 |
| Acinar | Pasteurella | 2.85E−38 | 0.849 | 0.985 | 0.973 | 1.01E−35 |
| Acinar | Crocosphaera | 9.18E−10 | 2.172 | 0.315 | 0.180 | 3.27E−07 |
| Acinar | Thalassotalea | 7.46E−09 | 0.673 | 0.700 | 0.581 | 2.65E−06 |
| Acinar | Nocardia | 1.81E−07 | 0.446 | 0.660 | 0.466 | 6.46E−05 |
| Acinar | Hypericibacter | 2.96E−06 | 0.925 | 0.445 | 0.305 | 1.06E−03 |
| Acinar | Chryseobacterium | 4.71E−06 | 0.276 | 0.830 | 0.927 | 1.68E−03 |

Cluster: cell type cluster;
P_val: enrichment p value;
Avg_logFC: average log fold change of the genus expression level in the cluster compared to all other clusters;
Pct. 1: % of cells in the cluster found with the genus;
Pct. 2: % of all other cells found with the genus;
P_val_adj: adjusted enrichment p value.

Microbiome diversity correlated with immune cell infiltration and diversity in the microenvironment: Next, the relationship between microbial diversity and tumor cellular composition was assessed. Within the tumor microenvironment (TME), both individual genera and total microbial diversity were significantly associated with abundances of particular somatic cell types, including immune cell infiltrations. Microbial diversity correlated with T-cell infiltration and also with the fraction of myeloid and malignant ductal 2 cells in the tumor. Microbial diversity was strongly negatively correlated with the presence of normal ductal 1 cells (FIG. 16F). Self-assembling manifolds (SAM) (Tarashansky et al. Elife, 8: 1-29, 2019) were then used to identify the major sub-populations within respective cell-types (FIG. 16G). These results indicated that microbial diversity strongly correlated with subpopulation diversity within T-cell, myeloid, and ductal type 2 cells and negatively correlated with diversity within other epithelial and endothelial cell-types (FIG. 16G). The positive correlations with immune and malignanT-cells suggested that a fraction of the TME immune response may in fact have been responding to local infection, and the negative associations with diversity within typical cells of the pancreas suggested possible phenotypic selection of ‘normal’-like cells within the TME. TME diversity in its totality was only weakly associated with microbial diversity, due to the opposing positive and negative associations (FIG. 16G).

Microbes were associated with specific biological processes in host cells: The microbial abundances that associated with host cell-type specific and sample-level gene expression and pathway activities were examined. The vast majority of microbes and genes or pathways showed no biologically or statistically significant correlations at either the level of the individual host cells or cell-types (FIG. 17B), but a subset showed strong correlations (Irl>0.5, adjusted p<0.05), indicating both known and novel microbiome-physiologic associations (Table 4). These results were analyzed at three levels.

TABLE 4
LASSO coefficients of sample-level microbiota abundances used to predict sample somatic cellular composition.

| | Acinar | B cells | Ductal1 | Ductal2 | Endocrine | Endothelial | Fibroblast | Myeloid | Stellate | T cells |
|---|---|---|---|---|---|---|---|---|---|---|
| Intercept | 0.0000 | 0.0000 | 0.0000 | 0.0000 | 0.0000 | 0.0000 | 0.0000 | 0.0000 | 0.0000 | 0.0000 |
| Aspergillus | −0.0146 | 0.2095 | −0.1373 | 0.2761 | 0.1620 | 0.0767 | 0.4063 | 0.3787 | 0.5688 | 0.4654 |
| Clostridium | −0.0347 | 0.0392 | −0.0443 | 0.0579 | 0.0499 | 0.0222 | 0.0395 | 0.0457 | 0.0818 | 0.0720 |
| Edwardsiella | 0.0032 | −0.0225 | 0.0177 | 0.0557 | −0.0060 | −0.0572 | 0.0161 | 0.0351 | −0.0017 | 0.0243 |
| Flexistipes | 0.0031 | 0.0034 | 0.0026 | −0.0019 | 0.0002 | 0.0034 | −0.0001 | 0.0023 | 0.0020 | 0.0024 |
| Granulibacter | 0.0336 | −0.0315 | 0.0363 | −0.0723 | −0.0075 | −0.0030 | −0.0798 | −0.0454 | −0.0549 | −0.0467 |
| Halanaerobium | −0.0286 | 0.0874 | −0.0309 | 0.1222 | 0.0661 | 0.0070 | 0.0471 | 0.1264 | 0.1360 | 0.1410 |
| Haliangium | −0.0165 | 0.0286 | −0.0498 | 0.0422 | 0.0076 | 0.0010 | −0.0040 | 0.0605 | 0.0154 | 0.0513 |
| Halomonas | 0.0097 | 0.1317 | −0.0361 | −0.0115 | −0.0650 | −0.0065 | −0.0496 | 0.0637 | −0.0190 | 0.0897 |
| Hypericibacter | 0.1030 | 0.0401 | 0.0641 | 0.0458 | 0.0046 | −0.0597 | 0.0597 | 0.0878 | 0.0928 | 0.0340 |
| Iodobacter | −0.2031 | −0.1007 | −0.2025 | 0.1766 | −0.2113 | −0.0838 | −0.1790 | −0.1601 | −0.0930 | −0.0816 |
| Jiangella | −0.1124 | 0.1317 | −0.1533 | 0.1763 | −0.1969 | −0.2574 | 0.0977 | 0.1393 | −0.0065 | 0.1292 |
| Kangiella | 0.0854 | −0.0065 | 0.0770 | −0.0345 | 0.0517 | −0.0236 | 0.0680 | 0.0407 | 0.0501 | −0.0284 |
| Kineobactrum | 0.0019 | 0.0038 | −0.0054 | 0.0059 | −0.0115 | −0.0229 | 0.0051 | 0.0236 | −0.0200 | −0.0019 |
| Kluyveromyces | −0.0211 | 0.0043 | −0.0469 | 0.0490 | −0.0887 | −0.0408 | −0.1416 | −0.0124 | −0.1000 | −0.0145 |
| Komagataella | −0.0115 | −0.0103 | 0.0018 | 0.0065 | −0.0187 | −0.0163 | −0.0406 | −0.0120 | −0.0297 | −0.0093 |
| Labilibaculum | −0.0182 | −0.0401 | 0.0001 | 0.0250 | 0.0647 | 0.0355 | 0.0651 | 0.0276 | 0.0930 | 0.0011 |
| Lachancea | −0.0709 | −0.0338 | −0.0820 | −0.0499 | −0.1721 | −0.1030 | −0.2772 | −0.1085 | −0.1814 | −0.1039 |
| Methylobacterium | −0.0020 | −0.0161 | 0.0011 | 0.0119 | 0.0099 | −0.0257 | 0.0092 | 0.0035 | −0.0039 | −0.0066 |
| Neisseria | 0.0298 | −0.0761 | 0.0404 | 0.0227 | 0.1335 | 0.0594 | 0.0086 | −0.0301 | 0.0078 | −0.0198 |
| Nocardiopsis | −0.1793 | −0.0020 | −0.1817 | 0.1459 | 0.0776 | −0.0715 | −0.0382 | −0.0206 | 0.0238 | 0.0337 |
| Pochonia | −0.0156 | −0.1210 | −0.0100 | 0.0090 | −0.0179 | −0.0970 | −0.0424 | 0.0063 | −0.0696 | −0.0741 |
| Pseudolysobacter | 0.0027 | 0.0063 | −0.0212 | 0.0339 | 0.0155 | −0.0072 | 0.0297 | 0.0562 | 0.0094 | 0.0288 |
| Pseudomonas | −0.0309 | 0.0090 | −0.0216 | −0.0098 | 0.0604 | 0.0204 | 0.0437 | 0.0199 | 0.0357 | 0.0446 |
| Ralstonia | −0.0054 | 0.0155 | −0.0088 | −0.0066 | 0.0085 | 0.0018 | −0.0049 | 0.0045 | 0.0060 | 0.0134 |
| Rhodococcus | 0.0039 | 0.0172 | 0.0057 | −0.0098 | 0.0327 | 0.0359 | −0.0249 | 0.0051 | 0.0196 | 0.0171 |
| Solitalea | 0.1206 | 0.0399 | 0.1188 | −0.1477 | 0.1274 | 0.1377 | 0.0160 | 0.0819 | 0.0534 | −0.0033 |
| Sphingobacterium | 0.3549 | −0.0286 | 0.1362 | −0.0448 | 0.1265 | 0.1957 | −0.0603 | 0.1566 | −0.0585 | −0.0394 |
| Sporisorium | 0.0319 | −0.0015 | 0.0245 | −0.0514 | −0.0660 | −0.0138 | −0.1113 | 0.0138 | −0.0836 | −0.0205 |
| Thermomonospora | −0.0279 | 0.0535 | −0.0278 | 0.0265 | −0.0240 | −0.0187 | −0.0166 | 0.0344 | 0.0101 | 0.0321 |
| Thioalkalivibrio | 0.0531 | 0.0276 | −0.0413 | 0.0622 | 0.0310 | 0.1029 | 0.0647 | 0.0814 | 0.0781 | 0.0015 |
| Virgibacillus | −0.0031 | 0.0060 | −0.0043 | 0.0070 | −0.0011 | 0.0005 | 0.0008 | 0.0043 | 0.0048 | 0.0082 |
| Xanthomonas | −0.0258 | 0.0248 | −0.0266 | 0.0306 | −0.0099 | 0.0137 | −0.0666 | 0.0560 | 0.0250 | 0.0332 |
| Yarrowia | −0.0003 | −0.0015 | 0.0001 | 0.0004 | −0.0004 | −0.0016 | −0.0006 | 0.0001 | −0.0009 | −0.0005 |

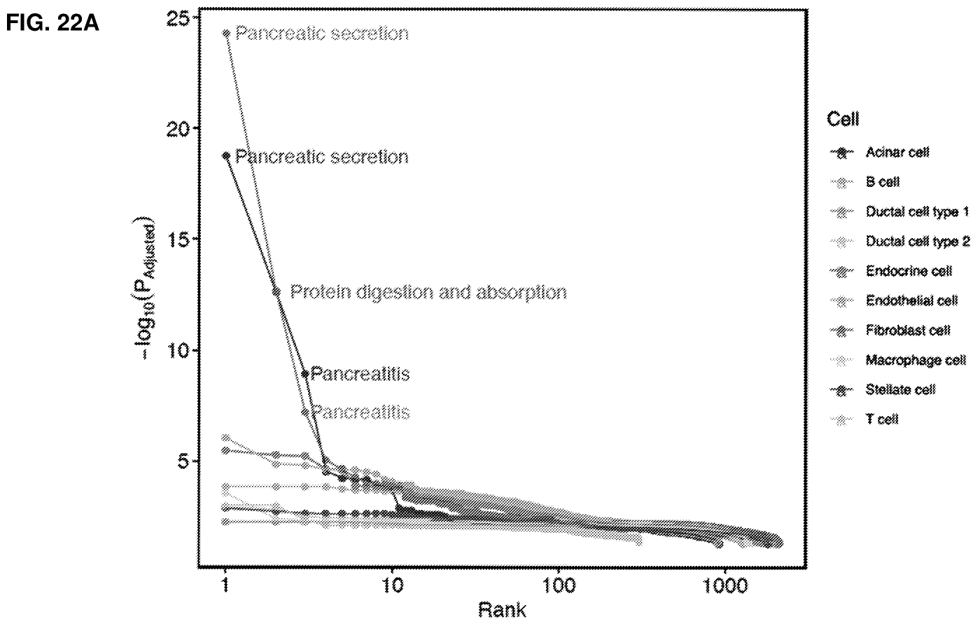

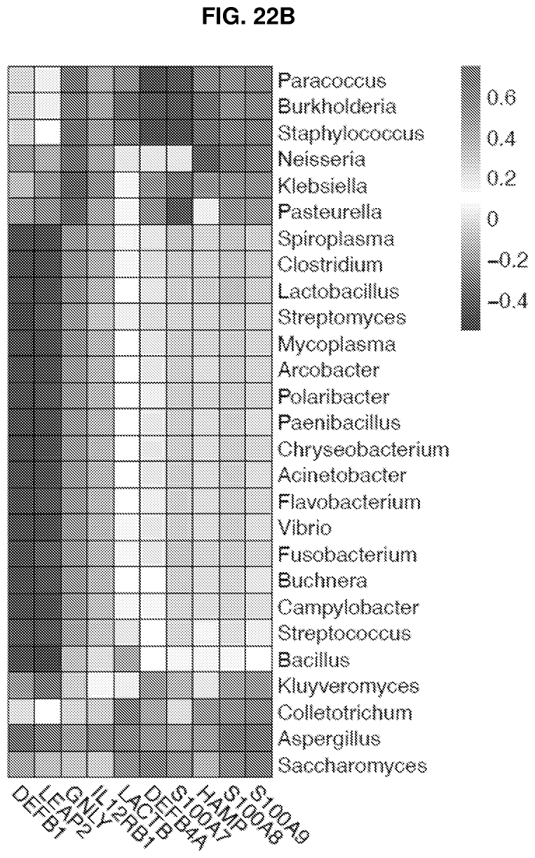

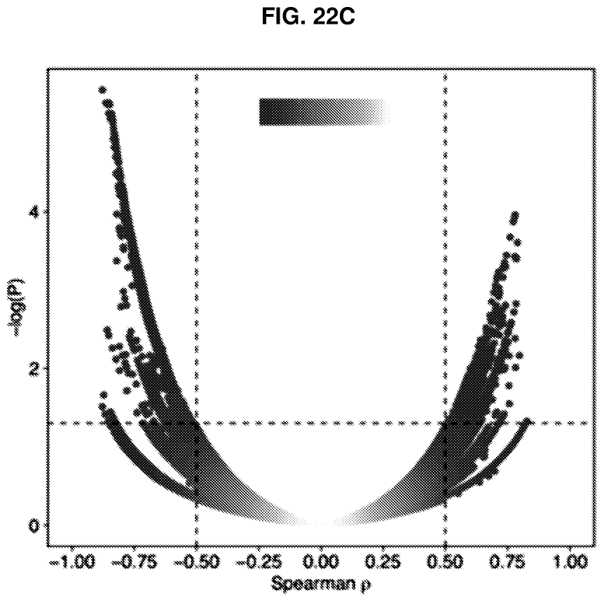

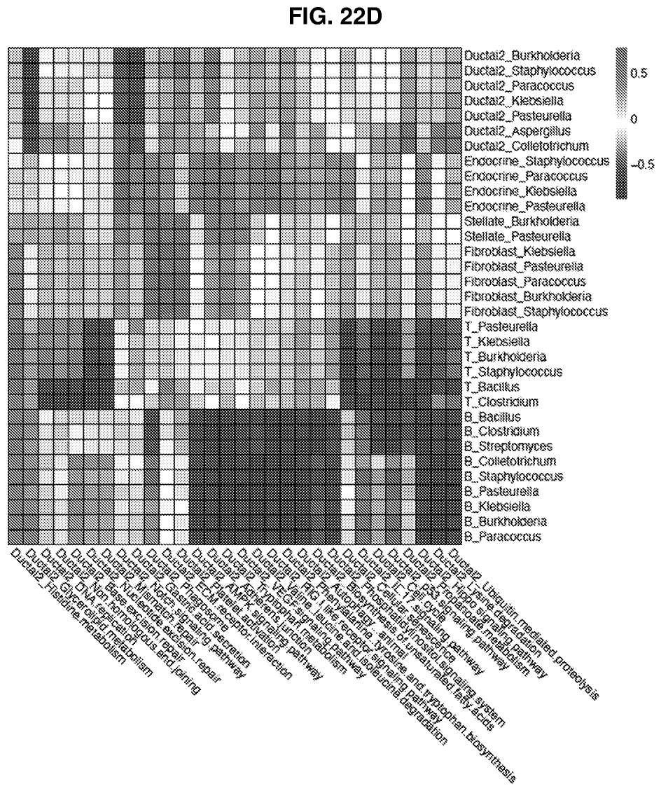

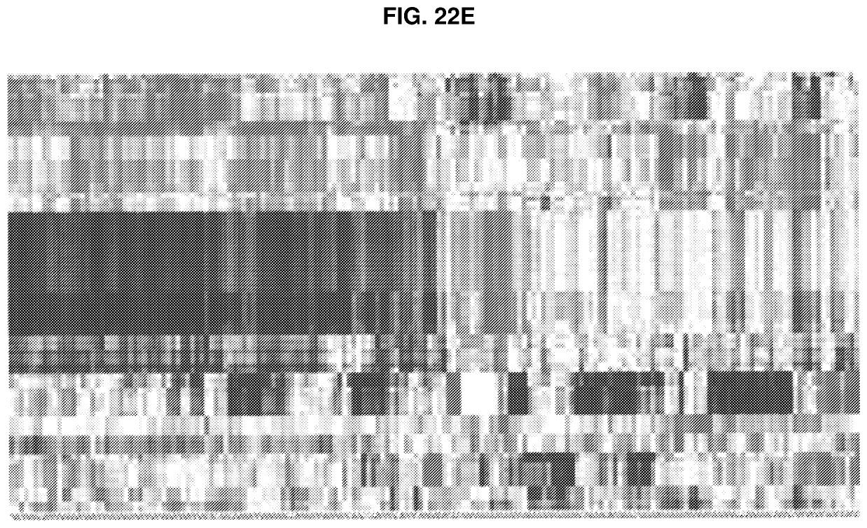

First, interactions between microbiota and receptor gene-expression in their associated host-cell types were examined (FIG. 17A). Expression of particular cell-type specific receptors was strongly associated with the presence of particular microbes in PDA and non-malignant tissues, in largely non-overlapping patterns. In particular, tumor-associated fungi were associated with large groups of receptor expression in T-cells and stellate cells, and these receptors were significantly enriched in pathways for hematopoietic lineage, proteoglycan interactions, the complement cascade, PI3K-AKT signaling, Rapt signaling, and cell adhesion. Aykut et al. (Aykut et al. Nature, 574: 264-267, 2019) recently showed that pathogenic fungi promote PDA via lectin-induced activation of the complement cascade. The putative commensal bacteria were associated with receptors mostly in acinar and stellate cells that were involved in normal pancreatic functions. Tumor-associated bacteria were strongly associated with receptors involved in PI3K-AKT signaling, adhesion pathways, and cytotoxicity in acinar, endothelial, and T-cells (FIG. 17A). Tumor-associated bacteria also were negatively associated with MET expression in malignant ductal 2 cells and were positively associated with LIFR expression in several cell types, as was recently implicated in PDA pathogenesis (Shi et al. Nature, 569: 131-135, 2019). At the individual cell-level, the microbe-gene expression associations revealed decreases in normal pancreatic secretory activities and increased inflammatory pathways, most strongly in acinar cells and fibroblasts that were rich in profiled microbiome (FIG. 22A).

Second, analysis of microbiome associations with downstream cell-type specific cancer-related pathway activities revealed several known and novel major patterns of interactions (FIGS. 18A-18C). Nearly all tumor-associated bacteria were strongly negatively associated with DNA replication and repair pathways in malignant ductal 2 cells. Infection by Escherichia coli and other microbes can deplete host DNA repair proteins (Sahan et al. Front. Microbiol. 9: 663, 2018; Maddocks et al. MBio. 4: e00152, 2013). Tumor-associated fungi positively correlated with cell cycle, apoptosis, and catabolic pathways in stellate cells, as shown in hepatic stellate cells via Aspergillus-derived gliotoxin (Kweon et al. J. Hepatol. 39: 38-46, 2003). Abundances of a subset of bacteria positively correlated with the PD-1/PD-L1 checkpoint pathway and immune transmigration and with sphingolipid signaling in both immune and endothelial cells, which was consistent with intestinal microbiome influence on anti-PD-1 immunotherapy responses in multiple cancer types (Pushalkar et al. Cancer Discov. 8: 403-416, 2018; Gopalakrishnan et al. Science, 359(6371):97-103, 2018; Xu et al. Front. Microbiol. 11: 814, 2020). Sphingolipids have been identified as mediators of intestinal-microbiota crosstalk (Bryan et al. Mediators Inflamm. 2016:9890141, 2016). Microbes also selectively associated with metabolic activities in host cells, including galactose, pentose phosphate, and propanoate metabolism in acinar and T-cells (FIG. 18B). Nearly all bacteria and fungi were associated with increased Hippo signaling in acinar and T-cells, which activates fibroinflammatory programs leading to stromal activation that promotes tumor growth (Liu et al. PLOS Biol. 17: e3000418, 2019; Ansari et al. Anticancer Res. 39: 3317-3321, 2019). At the microenvironment level, particular microbes correlated with inflammatory and antimicrobial gene expression (FIG. 17C, FIG. 22B). Numerous cell-type specific pathway activities correlated with abundances of microbes localized with other cell-types (FIGS. 22C-22D).

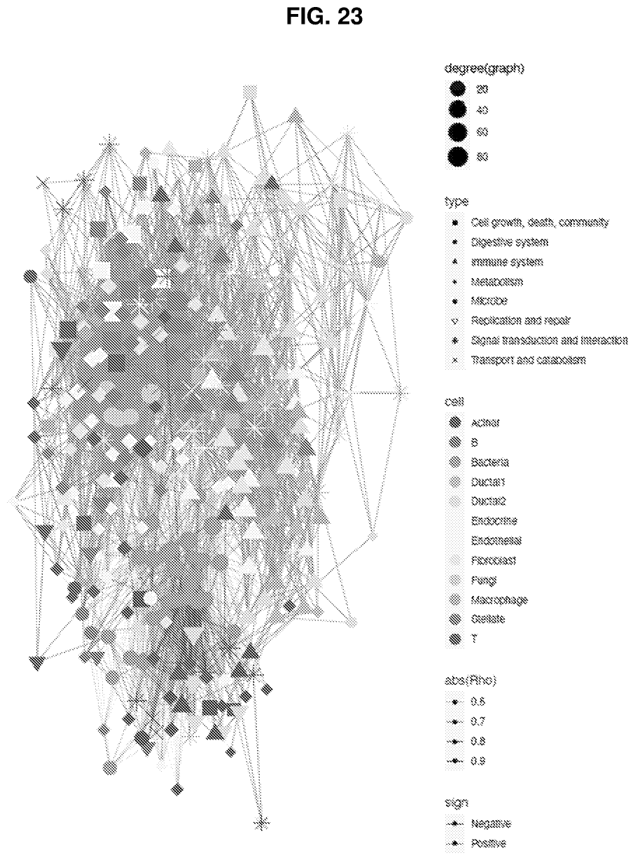

Next, microbe-pathway and cell-specific pathway-pathway interactions were visualized in a network graph, in which the nodes where either microbes or cellular pathways (e.g. T-cell Hippo signaling), and the edges represented significant positive or negative correlations (FIG. 17D, full-size image in FIG. 23). Analysis revealed four major hubs of interactions. Tumor-associated bacteria were closely associated with malignant ductal 2 DNA repair pathways and with acinar and T-cell signaling and metabolism. The other major clusters consisted of tumor microenvironment (TME) growth and metabolic activities, TME immune-related pathways, and ductal 2 specific signaling. Microbes were highly inter-connected in this network and were significantly over-represented in interactions with high edge centrality (FIG. 17E), suggesting that their interactions are common links between multiple TME aspects.

To benchmark these observations, the patterns of microbe-gene/pathway associations detected in our analysis were compared with those inferred from bulk sequencing data in the TCGA pancreatic cancer cohort, and consistent associations were found (FIGS. 17F-17G). For example, strong associations between LYZ expression and Bacteroidetes spp. and between Hippo signaling and Campylobacter spp. were detected in both cohorts. The number of statistically significant microbe-gene/pathway associations that were shared between the two datasets were then compared for both subsampled and label-shuffled data. Analysis indicated significantly more frequent shared associations compared to chance (p<2e-16, FIG. 17H). These observations suggested that microbes are not passive bystanders of tumor progression but may influence key cancer-related cellular processes in individual cell-types in the tumor-microenvironment.

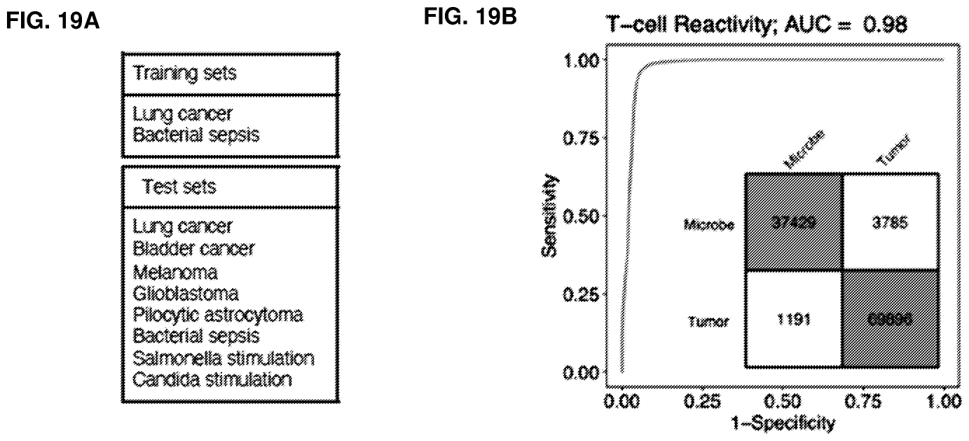

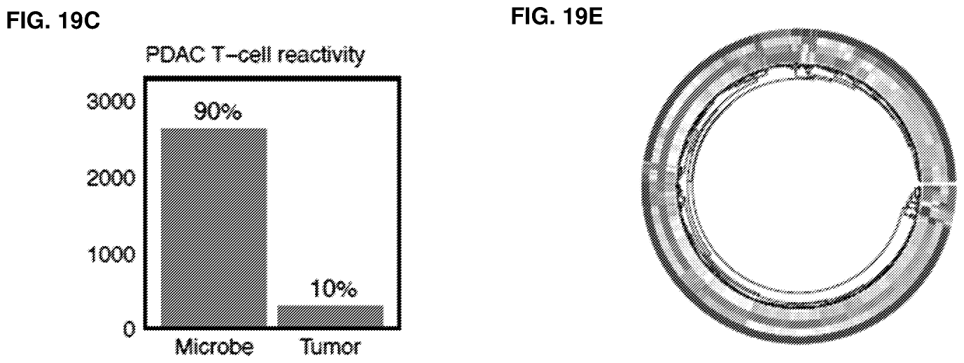

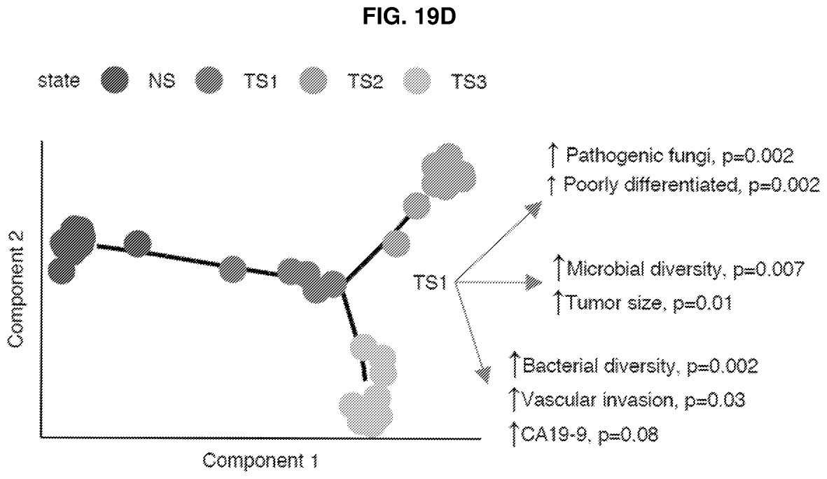

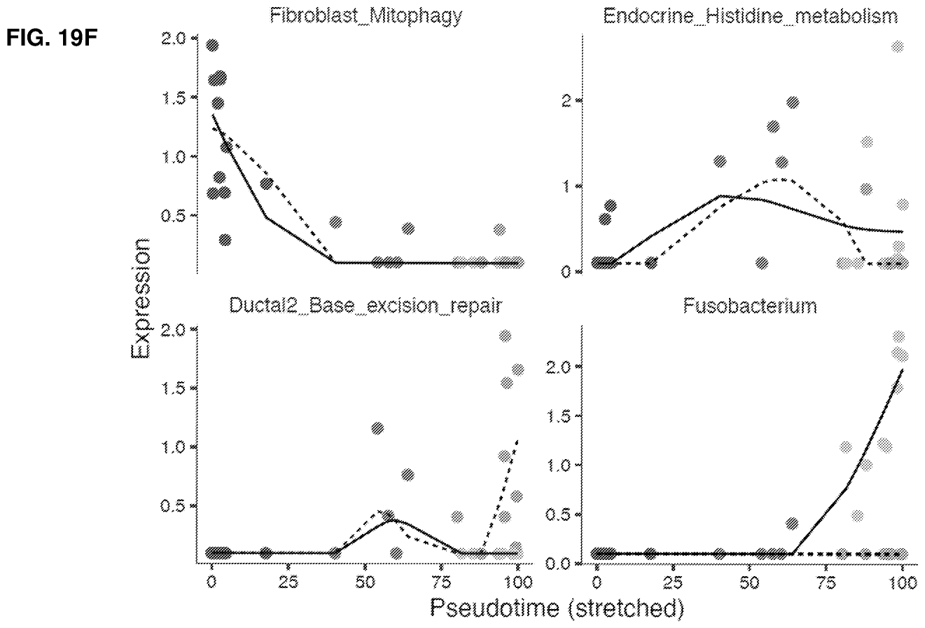

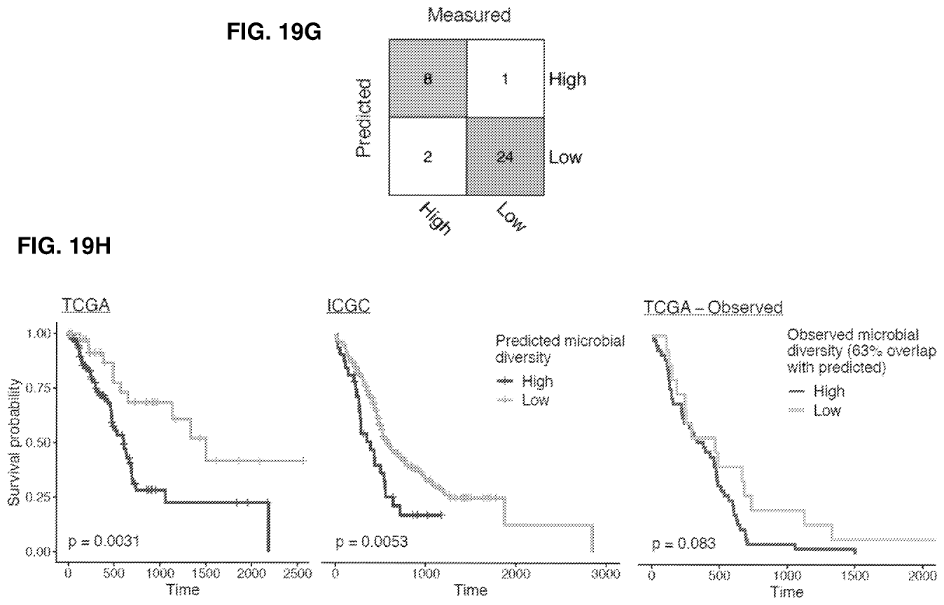

A majority of PDA T-cells were microbe-responsive: In light of the observations that the TME contains Thl7 cells commonly involved in antimicrobial responses (Knochelmann et al. Cell. Mol. Immunol. 15: 458-469, 2018) (FIG. 16F), that microbial diversity correlates with immune cell infiltration and diversity (FIG. 16G), and that particular microbial populations correlate with inflammatory and immune processes (FIGS. 17-18), it was postulated that a fraction of the immune response in the TME is directed against the microbiome and not the malignant T-cells. To test this hypothesis, a random forest model was constructed to distinguish between microbe-reactive and tumor-reactive T-cells based on their gene expression (Methods, FIGS. 19A-19C). First, a model was trained to classify T-cells as either microbe-responding or tumor-responding using T-cells sampled from patients with sepsis and tumors known to have a low microbiome burden (Poore et al. Nature 579: 567-574, 2020; Nejman et al. Science, 368(6494):973-980, 2020). The model was then tested on>100,000 cells taken from each of five cancer types with similarly known low microbiome burden and from three datasets representing either bacterial or fungal infection or stimulation (FIGS. 19A-19B). The model performed exceptionally well in classifying T-cell reactivity, with an AUC of 0.98 (FIG. 19B). Next, this model was used to predict T-cell reactivity in the pancreatic TME. Surprisingly, 90% of the T-cells sequenced in the Peng et al (Peng et al. Cell Res. 29(9):725-738, 2019) cohort were classified as microbe-responding.

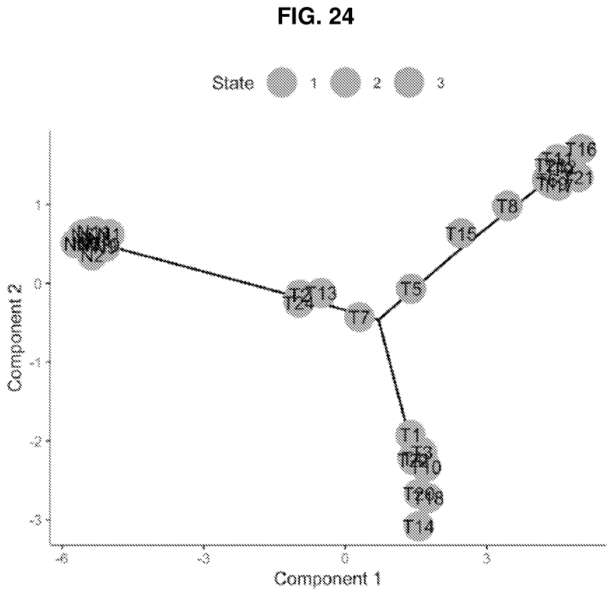

Pseudotime analysis identified tumor-microbiome coevolution and distinct tumor states: To examine how the microbiome might be associated with evolution of the PDA TME, a pseudotime analysis was conducted using Monocle (Trapnell et al. Nat. Biotechnol. 32: 381-386, 2014), which was originally developed for temporal ordering during normal development. TMEs were ordered along a progressive process in a data-driven manner based on their microbiome and cellular activities (FIG. 19D). The results revealed a branching evolutionary process in which pancreatic tissue progressed from a normal state to tumor state 1 (TS1), and then either towards tumor state 2 (TS2), characterized by increased levels of pathogenic fungi (t-test, p=0.00) and poorly differentiated histopathology (Fisher's exact test, p=0.00), or tumor state 3 (TS3), characterized by increased bacterial diversity (t-test, p=0.00), vascular invasion (Fisher's test, p=0.0), and CA19-9 antigen (t-test, p=0.08). Tumor states 2 and 3 were also characterized by a general increase in microbial diversity (t-test, p=0.007) and increased tumor size (t-test, p=0.0). The normal and tumor states had hundreds of significant T-cell-type specific pathway level differences, with the three tumor states clearly distinct from the normal state but retaining state-specific pathway and microbiome signatures (FIGS. 19E-19F, Table 5). For example, TS1 had increased normal ductal 1 arginine biosynthesis, TS2 increased ductal 1 Hippo signaling, and TS3 had decreased DNA repair. These normal and tumor states were observable even when pseudotime analysis was conducted using pathway scores alone, providing further validation of both the microbiome profiles generated herein and their marked relationship to tumor subtype (FIG. 24). Taken together, these results suggest that intra-tumoral microbial dysbiosis is linked with tumor histopathological and clinical attributes and the overall trajectory of tumor evolution.

TABLE 5
Exemplary significant microbe-cell-type specific gene correlations.

| Genus | Gene | Cell | Rho | Padj |
|---|---|---|---|---|
| Acinetobacter | UBD | Acinar | 0.794 | 2.92E−05 |
| Acinetobacter | PODXL | Acinar | 0.788 | 6.23E−05 |
| Acinetobacter | RAB11FIP1 | Acinar | 0.798 | 2.44E−05 |
| Acinetobacter | NNMT | Acinar | 0.770 | 7.18E−05 |
| Acinetobacter | C15orf48 | Acinar | 0.850 | 2.13E−06 |
| Acinetobacter | IL32 | Acinar | 0.812 | 1.38E−05 |
| Acinetobacter | GP2 | Acinar | −0.770 | 7.18E−05 |
| Acinetobacter | CLPS | Acinar | −0.770 | 7.18E−05 |
| Arcobacter | CTSS | Acinar | 0.766 | 3.19E−05 |
| Arcobacter | UBD | Acinar | 0.813 | 4.35E−06 |
| Arcobacter | CFB | Acinar | 0.808 | 5.41E−06 |
| Arcobacter | PODXL | Acinar | 0.823 | 4.54E−06 |
| Arcobacter | RAB11FIP1 | Acinar | 0.825 | 2.32E−06 |
| Arcobacter | RHOD | Acinar | 0.765 | 5.37E−05 |
| Arcobacter | UCP2 | Acinar | 0.817 | 1.96E−05 |
| Arcobacter | NNMT | Acinar | 0.790 | 1.23E−05 |
| Arcobacter | CHPT1 | Acinar | 0.760 | 6.46E−05 |
| Arcobacter | RNASE1 | Acinar | −0.757 | 4.51E−05 |
| Arcobacter | C15orf48 | Acinar | 0.864 | 2.13E−07 |
| Arcobacter | IL32 | Acinar | 0.793 | 1.06E−05 |
| Arcobacter | GP2 | Acinar | −0.775 | 2.26E−05 |
| Arcobacter | INSR | Acinar | 0.783 | 2.70E−05 |
| Arcobacter | NKG7 | Acinar | 0.744 | 7.08E−05 |
| Arcobacter | CLPS | Acinar | −0.782 | 1.71E−05 |
| Arcobacter | CTRL | Acinar | −0.763 | 3.65E−05 |
| Bacillus | UBD | Acinar | 0.795 | 2.75E−05 |
| Bacillus | CFB | Acinar | 0.785 | 4.15E−05 |
| Bacillus | RAB11FIP1 | Acinar | 0.798 | 2.44E−05 |
| Bacillus | FTH1 | Acinar | 0.782 | 4.65E−05 |
| Bacillus | C15orf48 | Acinar | 0.798 | 2.44E−05 |
| Bacillus | GP2 | Acinar | −0.800 | 2.29E−05 |
| Bacteroides | ALCAM | Acinar | 0.793 | 5.13E−05 |
| Bacteroides | SLC12A2 | Acinar | 0.826 | 4.39E−05 |
| Bacteroides | KPNA2 | Acinar | 0.841 | 4.41E−05 |
| Buchnera | TUBB2A | Acinar | 0.831 | 3.61E−05 |
| Buchnera | UBD | Acinar | 0.815 | 1.20E−05 |
| Buchnera | CFB | Acinar | 0.770 | 7.18E−05 |
| Buchnera | PODXL | Acinar | 0.839 | 7.29E−06 |
| Buchnera | RAB11FIP1 | Acinar | 0.880 | 3.21E−07 |
| Buchnera | RARRES3 | Acinar | 0.783 | 4.39E−05 |
| Buchnera | RHOD | Acinar | 0.805 | 3.19E−05 |
| Buchnera | UCP2 | Acinar | 0.867 | 6.67E−06 |
| Buchnera | NNMT | Acinar | 0.824 | 7.95E−06 |
| Buchnera | C15orf48 | Acinar | 0.887 | 1.85E−07 |
| Buchnera | IL32 | Acinar | 0.875 | 4.40E−07 |
| Buchnera | GP2 | Acinar | −0.785 | 4.15E−05 |
| Buchnera | SRCAP | Acinar | 0.782 | 7.52E−05 |
| Buchnera | HN1 | Acinar | 0.805 | 1.90E−05 |
| Buchnera | CLPS | Acinar | −0.824 | 7.95E−06 |
| Buchnera | CTRL | Acinar | −0.803 | 2.02E−05 |
| Campylobacter | F3 | Acinar | 0.794 | 1.01E−05 |
| Campylobacter | CTSS | Acinar | 0.751 | 5.71E−05 |
| Campylobacter | TUBB2A | Acinar | 0.816 | 2.07E−05 |
| Campylobacter | UBD | Acinar | 0.833 | 1.51E−06 |
| Campylobacter | CFB | Acinar | 0.817 | 3.48E−06 |
| Campylobacter | PODXL | Acinar | 0.840 | 1.87E−06 |
| Campylobacter | RAB11FIP1 | Acinar | 0.871 | 1.31E−07 |
| Campylobacter | FTH1 | Acinar | 0.763 | 3.65E−05 |
| Campylobacter | RHOD | Acinar | 0.814 | 7.04E−06 |
| Campylobacter | UCP2 | Acinar | 0.819 | 1.82E−05 |
| Campylobacter | NNMT | Acinar | 0.814 | 4.12E−06 |
| Campylobacter | CHPT1 | Acinar | 0.799 | 1.42E−05 |
| Campylobacter | RNASE1 | Acinar | −0.770 | 2.82E−05 |
| Campylobacter | MEG3 | Acinar | 0.747 | 6.48E−05 |
| Campylobacter | C15orf48 | Acinar | 0.890 | 2.84E−08 |
| Campylobacter | IL32 | Acinar | 0.829 | 1.82E−06 |
| Campylobacter | GP2 | Acinar | −0.816 | 3.68E−06 |
| Campylobacter | SRCAP | Acinar | 0.803 | 1.20E−05 |
| Campylobacter | CLDN7 | Acinar | 0.768 | 4.88E−05 |
| Campylobacter | HN1 | Acinar | 0.748 | 6.23E−05 |
| Campylobacter | INSR | Acinar | 0.799 | 1.42E−05 |
| Campylobacter | CELA3B | Acinar | −0.782 | 1.71E−05 |
| Campylobacter | CLPS | Acinar | −0.774 | 2.36E−05 |
| Campylobacter | CTRL | Acinar | −0.799 | 8.23E−06 |
| Chryseobacterium | CLDN7 | Acinar | 0.800 | 6.78E−05 |
| Clostridium | F3 | Acinar | 0.805 | 3.19E−05 |
| Clostridium | TUBB2A | Acinar | 0.856 | 2.34E−05 |
| Clostridium | UBD | Acinar | 0.802 | 3.66E−05 |
| Clostridium | CFB | Acinar | 0.825 | 1.41E−05 |
| Clostridium | HLA.DRB1 | Acinar | 0.826 | 1.30E−05 |
| Clostridium | SOD2 | Acinar | 0.784 | 7.06E−05 |
| Clostridium | RAB11FIP1 | Acinar | 0.854 | 3.22E−06 |
| Clostridium | FTH1 | Acinar | 0.814 | 2.23E−05 |
| Clostridium | RHOD | Acinar | 0.833 | 1.79E−05 |
| Clostridium | NNMT | Acinar | 0.793 | 5.13E−05 |
| Clostridium | KRT7 | Acinar | 0.793 | 5.13E−05 |
| Clostridium | OLFM4 | Acinar | 0.775 | 9.60E−05 |
| Clostridium | C15orf48 | Acinar | 0.868 | 1.43E−06 |
| Clostridium | IL32 | Acinar | 0.839 | 7.29E−06 |
| Clostridium | FXYD5 | Acinar | 0.777 | 9.04E−05 |
| Clostridium | CELA2B | Acinar | −0.825 | 1.41E−05 |
| Clostridium | AMY2A | Acinar | −0.809 | 2.77E−05 |
| Clostridium | REG3G | Acinar | 0.788 | 6.23E−05 |
| Clostridium | PNLIP | Acinar | −0.791 | 5.47E−05 |
| Clostridium | SYCN | Acinar | −0.825 | 1.41E−05 |
| Flavobacterium | TUBB2A | Acinar | 0.809 | 8.46E−05 |
| Flavobacterium | RAB11FIP1 | Acinar | 0.845 | 2.74E−06 |
| Flavobacterium | RHOD | Acinar | 0.814 | 2.23E−05 |
| Flavobacterium | C15orf48 | Acinar | 0.860 | 1.16E−06 |
| Flavobacterium | IL32 | Acinar | 0.835 | 4.75E−06 |
| Flavobacterium | GP2 | Acinar | −0.765 | 8.40E−05 |
| Flavobacterium | SRCAP | Acinar | 0.802 | 3.66E−05 |
| Flavobacterium | CLDN7 | Acinar | 0.784 | 7.06E−05 |
| Flavobacterium | HN1 | Acinar | 0.771 | 6.81E−05 |
| Flavobacterium | CTRL | Acinar | −0.764 | 8.85E−05 |
| Fusobacterium | F3 | Acinar | 0.765 | 5.37E−05 |
| Fusobacterium | CTSS | Acinar | 0.807 | 9.96E−06 |
| Fusobacterium | DUSP23 | Acinar | 0.770 | 7.18E−05 |
| Fusobacterium | CTSE | Acinar | 0.788 | 3.66E−05 |
| Fusobacterium | TUBB2A | Acinar | 0.853 | 6.68E−06 |
| Fusobacterium | UBD | Acinar | 0.839 | 2.01E−06 |
| Fusobacterium | CFB | Acinar | 0.818 | 5.85E−06 |
| Fusobacterium | PODXL | Acinar | 0.776 | 5.80E−05 |
| Fusobacterium | RAB11FIP1 | Acinar | 0.819 | 5.50E−06 |
| Fusobacterium | PLA2G16 | Acinar | 0.773 | 6.46E−05 |
| Fusobacterium | RHOD | Acinar | 0.798 | 2.44E−05 |
| Fusobacterium | UCP2 | Acinar | 0.840 | 1.26E−05 |
| Fusobacterium | NNMT | Acinar | 0.783 | 2.70E−05 |
| Fusobacterium | CHPT1 | Acinar | 0.780 | 4.91E−05 |
| Fusobacterium | MEG3 | Acinar | 0.804 | 1.13E−05 |
| Fusobacterium | C15orf48 | Acinar | 0.877 | 1.87E−07 |
| Fusobacterium | IL32 | Acinar | 0.800 | 1.34E−05 |
| Fusobacterium | GP2 | Acinar | −0.799 | 1.42E−05 |
| Fusobacterium | CORO1A | Acinar | 0.792 | 9.10E−05 |
| Fusobacterium | NKG7 | Acinar | 0.770 | 4.49E−05 |
| Klebsiella | FTH1 | Acinar | 0.779 | 5.19E−05 |
| Klebsiella | TUBA1B | Acinar | 0.804 | 3.42E−05 |
| Megamonas | TXNRD1 | Acinar | 0.866 | 1.42E−05 |
| Mycoplasma | FBXO2 | Acinar | 0.764 | 8.90E−05 |
| Mycoplasma | RNF186 | Acinar | 0.810 | 8.49E−06 |
| Mycoplasma | CTSS | Acinar | 0.869 | 3.26E−07 |
| Mycoplasma | DUSP23 | Acinar | 0.809 | 1.57E−05 |
| Mycoplasma | CTSE | Acinar | 0.761 | 9.86E−05 |
| Mycoplasma | GNLY | Acinar | 0.795 | 4.74E−05 |
| Mycoplasma | MECOM | Acinar | 0.761 | 9.86E−05 |
| Mycoplasma | TUBB2A | Acinar | 0.802 | 6.28E−05 |
| Mycoplasma | UBD | Acinar | 0.783 | 2.67E−05 |
| Mycoplasma | MEST | Acinar | 0.812 | 8.00E−06 |
| Mycoplasma | DNAJC12 | Acinar | 0.754 | 7.76E−05 |
| Mycoplasma | RHOD | Acinar | 0.783 | 4.39E−05 |
| Mycoplasma | UCP2 | Acinar | 0.850 | 8.05E−06 |
| Mycoplasma | CHPT1 | Acinar | 0.780 | 4.91E−05 |
| Mycoplasma | C15orf48 | Acinar | 0.769 | 4.61E−05 |
| Mycoplasma | HCST | Acinar | 0.764 | 8.87E−05 |
| Mycoplasma | NKG7 | Acinar | 0.827 | 3.73E−06 |
| Paenibacillus | CTSS | Acinar | 0.781 | 8.05E−05 |
| Paenibacillus | SLC12A2 | Acinar | 0.809 | 8.53E−05 |
| Paenibacillus | GP2 | Acinar | −0.782 | 7.66E−05 |
| Pasteurella | TFF1 | Acinar | −0.846 | 1.88E−05 |
| Polaribacter | ITGA2 | Acinar | 0.843 | 1.11E−05 |
| Polaribacter | UCP2 | Acinar | 0.882 | 6.39E−06 |
| Polaribacter | NNMT | Acinar | 0.788 | 6.23E−05 |
| Polaribacter | C15orf48 | Acinar | 0.779 | 8.50E−05 |
| Prevotella | MEST | Acinar | 0.822 | 1.61E−05 |
| Ralstonia | RP11.14N7.2 | Acinar | 0.762 | 5.89E−05 |
| Ralstonia | SOD2 | Acinar | 0.749 | 9.24E−05 |
| Ralstonia | RNASE1 | Acinar | −0.777 | 3.47E−05 |
| Spiroplasma | CTSS | Acinar | 0.851 | 1.04E−06 |
| Spiroplasma | DUSP23 | Acinar | 0.815 | 1.20E−05 |
| Spiroplasma | ALCAM | Acinar | 0.771 | 4.25E−05 |
| Spiroplasma | SLC12A2 | Acinar | 0.835 | 8.71E−06 |
| Spiroplasma | UBD | Acinar | 0.782 | 2.81E−05 |
| Spiroplasma | MAL2 | Acinar | 0.791 | 1.98E−05 |
| Spiroplasma | UCP2 | Acinar | 0.794 | 8.34E−05 |
| Spiroplasma | CHPT1 | Acinar | 0.762 | 9.31E−05 |
| Spiroplasma | C15orf48 | Acinar | 0.770 | 4.40E−05 |
| Spiroplasma | GP2 | Acinar | −0.765 | 5.45E−05 |
| Spiroplasma | SRCAP | Acinar | 0.792 | 3.10E−05 |
| Spiroplasma | INSR | Acinar | 0.764 | 8.85E−05 |
| Spiroplasma | NKG7 | Acinar | 0.757 | 7.09E−05 |
| Staphylococcus | UBD | Acinar | 0.771 | 6.81E−05 |
| Staphylococcus | GSTA1 | Acinar | 0.771 | 6.81E−05 |
| Staphylococcus | FTH1 | Acinar | 0.812 | 1.38E−05 |
| Staphylococcus | RHOD | Acinar | 0.795 | 4.80E−05 |
| Staphylococcus | TUBA1B | Acinar | 0.779 | 8.50E−05 |
| Staphylococcus | CELA2B | Acinar | −0.765 | 8.40E−05 |
| Staphylococcus | AMY2A | Acinar | −0.800 | 2.29E−05 |
| Staphylococcus | PNLIP | Acinar | −0.761 | 9.80E−05 |
| Staphylococcus | CTRL | Acinar | −0.800 | 2.29E−05 |
| Streptococcus | TUBB2A | Acinar | 0.811 | 7.74E−05 |
| Streptococcus | UBD | Acinar | 0.795 | 2.75E−05 |
| Streptococcus | CFB | Acinar | 0.795 | 2.75E−05 |
| Streptococcus | PODXL | Acinar | 0.811 | 2.58E−05 |
| Streptococcus | RAB11FIP1 | Acinar | 0.823 | 8.53E−06 |
| Streptococcus | RHOD | Acinar | 0.777 | 9.04E−05 |
| Streptococcus | NNMT | Acinar | 0.802 | 2.15E−05 |
| Streptococcus | RNASE1 | Acinar | −0.776 | 5.80E−05 |
| Streptococcus | C15orf48 | Acinar | 0.863 | 9.62E−07 |
| Streptococcus | IL32 | Acinar | 0.818 | 1.05E−05 |
| Streptococcus | GP2 | Acinar | −0.789 | 3.49E−05 |
| Streptomyces | CTSS | Acinar | 0.786 | 2.43E−05 |
| Streptomyces | DUSP23 | Acinar | 0.795 | 1.67E−05 |
| Streptomyces | CPB1 | Acinar | −0.755 | 7.74E−05 |
| Streptomyces | UBD | Acinar | 0.855 | 8.16E−07 |
| Streptomyces | CFB | Acinar | 0.827 | 3.73E−06 |
| Streptomyces | GSTA1 | Acinar | 0.813 | 7.49E−06 |
| Streptomyces | SOD2 | Acinar | 0.788 | 2.19E−05 |
| Streptomyces | PODXL | Acinar | 0.791 | 3.29E−05 |
| Streptomyces | RAB11FIP1 | Acinar | 0.822 | 4.84E−06 |
| Streptomyces | EIF4EBP1 | Acinar | 0.749 | 9.24E−05 |
| Streptomyces | FTH1 | Acinar | 0.826 | 3.99E−06 |
| Streptomyces | PLA2G16 | Acinar | 0.798 | 2.44E−05 |
| Streptomyces | UCP2 | Acinar | 0.806 | 5.38E−05 |
| Streptomyces | NNMT | Acinar | 0.801 | 1.27E−05 |
| Streptomyces | KRT7 | Acinar | 0.799 | 1.42E−05 |
| Streptomyces | CHPT1 | Acinar | 0.815 | 1.20E−05 |
| Streptomyces | OLFM4 | Acinar | 0.825 | 4.26E−06 |
| Streptomyces | MEG3 | Acinar | 0.773 | 4.03E−05 |
| Streptomyces | C15orf48 | Acinar | 0.879 | 1.54E−07 |
| Streptomyces | IL32 | Acinar | 0.779 | 3.14E−05 |
| Streptomyces | GP2 | Acinar | −0.805 | 1.07E−05 |
| Streptomyces | SRCAP | Acinar | 0.785 | 4.15E−05 |
| Streptomyces | SDC4 | Acinar | 0.773 | 4.03E−05 |
| Streptomyces | WFDC2 | Acinar | 0.770 | 7.18E−05 |
| Streptomyces | INSR | Acinar | 0.829 | 6.40E−06 |
| Streptomyces | C19orf33 | Acinar | 0.753 | 8.10E−05 |
| Streptomyces | RPS16 | Acinar | 0.764 | 5.62E−05 |
| Streptomyces | CELA3B | Acinar | −0.781 | 2.99E−05 |
| Streptomyces | CELA3A | Acinar | −0.789 | 3.49E−05 |
| Streptomyces | AMY2A | Acinar | −0.758 | 6.76E−05 |
| Streptomyces | CLPS | Acinar | −0.771 | 4.23E−05 |
| Streptomyces | CTRL | Acinar | −0.757 | 7.08E−05 |
| Streptomyces | CTRB1 | Acinar | −0.762 | 5.89E−05 |
| Streptomyces | SYCN | Acinar | −0.749 | 9.24E−05 |
| Vibrio | FBXO2 | Acinar | 0.812 | 2.41E−05 |
| Vibrio | CTSS | Acinar | 0.828 | 6.44E−06 |
| Vibrio | DUSP23 | Acinar | 0.777 | 9.04E−05 |
| Vibrio | MECOM | Acinar | 0.781 | 8.05E−05 |
| Vibrio | UBD | Acinar | 0.763 | 9.22E−05 |
| Vibrio | RHOD | Acinar | 0.795 | 4.80E−05 |
| Vibrio | UCP2 | Acinar | 0.809 | 8.31E−05 |
| Vibrio | PMAIP1 | Acinar | 0.784 | 7.24E−05 |
| Megamonas | PLK1 | B_cell | −0.939 | 5.62E−05 |
| Sphingobacterium | KIF2C | B_cell | −0.918 | 6.80E−05 |
| Sphingobacterium | CENPE | B_cell | −0.918 | 6.80E−05 |
| Sphingobacterium | KIFC1 | B_cell | −0.922 | 5.29E−05 |
| Sphingobacterium | SCG5 | B_cell | −0.924 | 4.78E−05 |
| Sphingobacterium | UBE2C | B_cell | −0.925 | 4.59E−05 |
| Aspergillus | SCTR | B_cell | −0.942 | 4.54E−05 |
| Colletotrichum | SCTR | B_cell | −0.930 | 9.60E−05 |
| Acinetobacter | CYR61 | Ductal1 | −0.675 | 2.24E−05 |
| Acinetobacter | S100A6 | Ductal1 | 0.627 | 9.55E−05 |
| Acinetobacter | TAGLN3 | Ductal1 | −0.700 | 3.43E−05 |
| Acinetobacter | MMP7 | Ductal1 | 0.632 | 7.88E−05 |
| Acinetobacter | ADCYAP1 | Ductal1 | −0.697 | 2.70E−05 |
| Acinetobacter | FOSB | Ductal1 | −0.653 | 3.73E−05 |
| Acinetobacter | CTRL | Ductal1 | −0.651 | 7.20E−05 |
| Campylobacter | CUZD1 | Ductal1 | −0.673 | 4.57E−05 |
| Campylobacter | MDK | Ductal1 | 0.678 | 3.80E−05 |
| Campylobacter | PCDH17 | Ductal1 | −0.702 | 1.53E−05 |
| Campylobacter | CTRB1 | Ductal1 | −0.680 | 3.58E−05 |
| Chryseobacterium | TAGLN3 | Ductal1 | −0.725 | 4.18E−05 |
| Chryseobacterium | MDK | Ductal1 | 0.683 | 3.17E−05 |
| Chryseobacterium | LINC00261 | Ductal1 | −0.664 | 8.48E−05 |
| Clostridium | MDK | Ductal1 | 0.674 | 4.46E−05 |
| Fusobacterium | TAGLN3 | Ductal1 | −0.724 | 4.34E−05 |
| Megamonas | CD2 | Ductal1 | 0.854 | 1.45E−08 |
| Megamonas | CAPN8 | Ductal 1 | 0.701 | 6.66E−05 |
| Megamonas | IL7R | Ductal1 | 0.754 | 8.50E−06 |
| Megamonas | LST1 | Ductal 1 | 0.707 | 3.79E−05 |
| Megamonas | FAM26F | Ductal1 | 0.758 | 2.93E−06 |
| Megamonas | AZGP1 | Ductal 1 | 0.716 | 5.61E−05 |
| Megamonas | FAM214B | Ductal1 | 0.745 | 8.42E−06 |
| Megamonas | CHRDL2 | Ductal1 | 0.719 | 3.46E−05 |
| Megamonas | VSIG2 | Ductal1 | 0.726 | 3.96E−05 |
| Megamonas | MSLN | Ductal1 | 0.723 | 6.68E−05 |
| Megamonas | MAFB | Ductal1 | 0.753 | 5.78E−06 |
| Megamonas | C19orf77 | Ductal1 | 0.801 | 8.98E−07 |
| Megamonas | CEACAM6 | Ductal1 | 0.733 | 1.38E−05 |
| Megamonas | TFF3 | Ductal1 | 0.759 | 6.97E−06 |
| Paenibacillus | GRB7 | Ductal1 | −0.703 | 8.94E−05 |
| Polaribacter | LINC00261 | Ductal1 | −0.663 | 8.91E−05 |
| Prevotella | RP11.528G1.2 | Ductal1 | −0.689 | 1.82E−05 |
| Prevotella | HLA.DRB1 | Ductal1 | 0.691 | 1.67E−05 |
| Prevotella | HLA.DPA1 | Ductal1 | 0.656 | 6.15E−05 |
| Prevotella | MDK | Ductal1 | 0.671 | 3.66E−05 |
| Prevotella | MMP7 | Ductal 1 | 0.662 | 4.97E−05 |
| Prevotella | LYZ | Ductal1 | 0.686 | 2.02E−05 |
| Prevotella | PCDH17 | Ductal1 | −0.700 | 1.16E−05 |
| Prevotella | HSD17B2 | Ductal1 | 0.769 | 4.44E−07 |
| Prevotella | KRT19 | Ductal1 | 0.686 | 2.06E−05 |
| Prevotella | CLPS | Ductal1 | −0.643 | 9.63E−05 |
| Prevotella | CTRB1 | Ductal1 | −0.689 | 1.85E−05 |
| Prevotella | SNORD3D | Ductal1 | −0.653 | 9.22E−05 |
| Spiroplasma | ERO1LB | Ductal1 | −0.723 | 4.32E−06 |
| Aspergillus | HSPD1 | Ductal2 | 0.729 | 7.89E−05 |
| Aspergillus | ZFAND2A | Ductal2 | 0.748 | 4.06E−05 |
| Aspergillus | LDHA | Ductal2 | 0.725 | 9.01E−05 |
| Colletotrichum | HSPD1 | Ductal2 | 0.765 | 2.14E−05 |
| Colletotrichum | ZFAND2A | Ductal2 | 0.746 | 4.37E−05 |
| Colletotrichum | LDHA | Ductal2 | 0.786 | 8.94E−06 |
| Colletotrichum | RHOD | Ductal2 | 0.732 | 7.13E−05 |
| Saccharomyces | ZFAND2A | Ductal2 | 0.799 | 4.74E−06 |
| Saccharomyces | LDHA | Ductal2 | 0.792 | 6.85E−06 |
| Saccharomyces | RHOD | Ductal2 | 0.749 | 3.92E−05 |
| Thermothielavioides | HSPD1 | Ductal2 | 0.737 | 6.01E−05 |
| Thermothielavioides | ZFAND2A | Ductal2 | 0.779 | 1.21E−05 |
| Thermothielavioides | LDHA | Ductal2 | 0.781 | 1.11E−05 |
| Thermothielavioides | RHOD | Ductal2 | 0.753 | 3.38E−05 |
| Campylobacter | PDPN | Endocrine | −0.754 | 5.13E−05 |
| Megamonas | AMN | Endocrine | 0.704 | 8.54E−05 |
| Megamonas | BIK | Endocrine | 0.727 | 1.78E−05 |
| Pasteurella | TMEM97 | Endocrine | 0.760 | 4.12E−05 |
| Spiroplasma | TCN1 | Endocrine | 0.684 | 8.30E−05 |
| Staphylococcus | C10orf10 | Endocrine | 0.760 | 6.46E−05 |
| Aspergillus | LINC01133 | Endocrine | 0.725 | 9.14E−05 |
| Aspergillus | FMO3 | Endocrine | 0.741 | 7.91E−05 |
| Aspergillus | CD8A | Endocrine | 0.691 | 9.21E−05 |
| Aspergillus | TNNC1 | Endocrine | 0.758 | 7.28E−06 |
| Aspergillus | CITED1 | Endocrine | 0.761 | 3.96E−05 |
| Aspergillus | LCN6.1 | Endocrine | 0.769 | 1.13E−05 |
| Aspergillus | NKX2.3 | Endocrine | 0.717 | 5.51E−05 |
| Aspergillus | CLEC14A | Endocrine | 0.710 | 4.78E−05 |
| Aspergillus | WFDC1 | Endocrine | 0.818 | 3.25E−06 |
| Aspergillus | ADAMTS5 | Endocrine | 0.731 | 7.34E−05 |
| Colletotrichum | CD8A | Endocrine | 0.744 | 2.03E−05 |
| Colletotrichum | ACKR3 | Endocrine | 0.750 | 9.04E−05 |
| Colletotrichum | TNNC1 | Endocrine | 0.718 | 5.40E−05 |
| Colletotrichum | AK8 | Endocrine | 0.769 | 2.84E−05 |
| Colletotrichum | LCN6.1 | Endocrine | 0.772 | 1.61E−05 |
| Colletotrichum | WFDC1 | Endocrine | 0.855 | 8.06E−07 |
| Colletotrichum | ADAMTS5 | Endocrine | 0.738 | 8.84E−05 |
| Kluyveromyces | ALPL | Endocrine | 0.828 | 1.20E−05 |
| Kluyveromyces | FMO3 | Endocrine | 0.735 | 9.84E−05 |
| Kluyveromyces | TNNC1 | Endocrine | 0.804 | 2.24E−06 |
| Kluyveromyces | MYCT1 | Endocrine | 0.828 | 1.20E−05 |
| Kluyveromyces | IL3RA | Endocrine | 0.794 | 1.04E−05 |
| Kluyveromyces | CITED1 | Endocrine | 0.784 | 2.64E−05 |
| Kluyveromyces | GPIHBP1 | Endocrine | 0.980 | 1.01E−12 |
| Kluyveromyces | IL33 | Endocrine | 0.892 | 1.30E−07 |
| Kluyveromyces | LCN6.1 | Endocrine | 0.735 | 9.65E−05 |
| Kluyveromyces | MRC1 | Endocrine | 0.810 | 1.51E−05 |
| Kluyveromyces | KLRC2 | Endocrine | 0.775 | 9.76E−05 |
| Kluyveromyces | KRT86 | Endocrine | 0.804 | 1.92E−05 |
| Kluyveromyces | RP11.841O20.2 | Endocrine | 0.790 | 3.47E−05 |
| Kluyveromyces | WFDC1 | Endocrine | 0.756 | 7.27E−05 |
| Saccharomyces | LINC01133 | Endocrine | 0.749 | 3.89E−05 |
| Saccharomyces | CD8A | Endocrine | 0.697 | 7.64E−05 |
| Saccharomyces | ACKR3 | Endocrine | 0.738 | 8.70E−05 |
| Saccharomyces | TNNC1 | Endocrine | 0.761 | 6.41E−06 |
| Saccharomyces | CITED1 | Endocrine | 0.793 | 1.08E−05 |
| Saccharomyces | LCN6.1 | Endocrine | 0.755 | 2.00E−05 |
| Saccharomyces | NKX2.3 | Endocrine | 0.733 | 3.12E−05 |
| Saccharomyces | CLEC14A | Endocrine | 0.710 | 4.91E−05 |
| Saccharomyces | WFDC1 | Endocrine | 0.817 | 3.48E−06 |
| Saccharomyces | ADAMTS5 | Endocrine | 0.754 | 3.26E−05 |
| Thermothielavioides | LINC01133 | Endocrine | 0.757 | 2.91E−05 |
| Thermothielavioides | CD8A | Endocrine | 0.693 | 8.73E−05 |
| Thermothielavioides | TNNC1 | Endocrine | 0.742 | 1.44E−05 |
| Thermothielavioides | CITED1 | Endocrine | 0.747 | 6.50E−05 |
| Thermothielavioides | LCN6.1 | Endocrine | 0.764 | 1.40E−05 |
| Thermothielavioides | NKX2.3 | Endocrine | 0.711 | 6.68E−05 |
| Thermothielavioides | CLEC14A | Endocrine | 0.720 | 3.40E−05 |
| Thermothielavioides | WFDC1 | Endocrine | 0.820 | 3.03E−06 |
| Thermothielavioides | ADAMTS5 | Endocrine | 0.731 | 7.34E−05 |
| Arcobacter | CD2 | Endothelial | 0.656 | 6.22E−05 |
| Arcobacter | DNAJC12 | Endothelial | 0.669 | 5.38E−05 |
| Arcobacter | KCNN4 | Endothelial | 0.702 | 1.10E−05 |
| Bacteroides | CD53 | Endothelial | 0.667 | 7.90E−05 |
| Bacteroides | HIST2H2AA3 | Endothelial | 0.689 | 7.00E−05 |
| Bacteroides | MNDA | Endothelial | 0.700 | 4.85E−05 |
| Bacteroides | FCGR2B | Endothelial | 0.682 | 4.54E−05 |
| Bacteroides | SLC11A1 | Endothelial | 0.716 | 1.85E−05 |
| Bacteroides | CXCL5 | Endothelial | 0.705 | 8.28E−05 |
| Bacteroides | CSF2RA | Endothelial | 0.701 | 6.71E−05 |
| Bacteroides | SPI1 | Endothelial | 0.674 | 8.42E−05 |
| Bacteroides | TCN1 | Endothelial | 0.689 | 5.00E−05 |
| Bacteroides | PTPRCAP | Endothelial | 0.692 | 3.25E−05 |
| Bacteroides | AMICA1 | Endothelial | 0.722 | 9.82E−06 |
| Bacteroides | CD3D | Endothelial | 0.725 | 8.64E−06 |
| Bacteroides | RNASE6 | Endothelial | 0.687 | 7.66E−05 |
| Bacteroides | BATF | Endothelial | 0.749 | 3.01E−06 |
| Bacteroides | LIMD2 | Endothelial | 0.696 | 3.88E−05 |
| Bacteroides | CD7 | Endothelial | 0.720 | 1.08E−05 |
| Bacteroides | CST7 | Endothelial | 0.660 | 9.67E−05 |
| Bacteroides | HCST | Endothelial | 0.731 | 6.64E−06 |
| Bacteroides | KCNN4 | Endothelial | 0.707 | 1.82E−05 |
| Bacteroides | RAC2 | Endothelial | 0.688 | 3.78E−05 |
| Bacteroides | LGALS1 | Endothelial | 0.695 | 8.13E−05 |
| Bacteroides | ITGB2 | Endothelial | 0.689 | 3.58E−05 |
| Burkholderia | NOX5 | Endothelial | −0.676 | 5.66E−05 |
| Chryseobacterium | CCND1 | Endothelial | −0.666 | 1.27E−05 |
| Chryseobacterium | PLXDC1 | Endothelial | 0.630 | 4.93E−05 |
| Clostridium | CXCL5 | Endothelial | 0.706 | 3.92E−05 |
| Clostridium | KCNN4 | Endothelial | 0.651 | 9.65E−05 |
| Flavobacterium | GPAT2 | Endothelial | 0.660 | 7.23E−05 |
| Flavobacterium | CCND1 | Endothelial | −0.689 | 2.55E−05 |
| Fusobacterium | CENPW | Endothelial | 0.656 | 6.24E−05 |
| Fusobacterium | CCND1 | Endothelial | −0.652 | 2.19E−05 |
| Fusobacterium | PLXDC1 | Endothelial | 0.633 | 4.54E−05 |
| Fusobacterium | KCNN4 | Endothelial | 0.665 | 1.75E−05 |
| Megamonas | CD8A | Endothelial | 0.737 | 7.69E−06 |
| Megamonas | COL7A1 | Endothelial | 0.693 | 4.29E−05 |
| Megamonas | EREG | Endothelial | 0.720 | 3.32E−05 |
| Megamonas | CYBB | Endothelial | 0.727 | 2.57E−05 |
| Megamonas | BATF | Endothelial | 0.670 | 7.00E−05 |
| Mycoplasma | CXCL5 | Endothelial | 0.675 | 8.07E−05 |
| Mycoplasma | DNAJC12 | Endothelial | 0.733 | 4.14E−06 |
| Mycoplasma | KCNN4 | Endothelial | 0.695 | 1.45E−05 |
| Paenibacillus | CD3D | Endothelial | 0.658 | 7.84E−05 |
| Paracoccus | NOX5 | Endothelial | −0.726 | 8.36E−06 |
| Spiroplasma | CADM3 | Endothelial | 0.657 | 5.98E−05 |
| Spiroplasma | CXCL5 | Endothelial | 0.733 | 9.17E−06 |
| Spiroplasma | GPR110 | Endothelial | 0.654 | 8.89E−05 |
| Spiroplasma | LINC00035 | Endothelial | 0.662 | 9.06E−05 |
| Spiroplasma | DNAJC12 | Endothelial | 0.719 | 7.45E−06 |
| Spiroplasma | CCND1 | Endothelial | −0.654 | 4.95E−05 |
| Spiroplasma | KCNN4 | Endothelial | 0.648 | 8.19E−05 |
| Staphylococcus | NOX5 | Endothelial | −0.652 | 9.47E−05 |
| Streptococcus | CD8A | Endothelial | 0.669 | 1.52E−05 |
| Streptococcus | CCND1 | Endothelial | −0.654 | 2.08E−05 |
| Streptococcus | KLRD1 | Endothelial | 0.669 | 1.51E−05 |
| Streptococcus | PLXDC1 | Endothelial | 0.653 | 2.11E−05 |
| Streptomyces | CADM3 | Endothelial | 0.625 | 9.97E−05 |
| Streptomyces | SPTSSB | Endothelial | 0.646 | 8.69E−05 |
| Streptomyces | HOPX | Endothelial | 0.626 | 9.70E−05 |
| Streptomyces | HPGD | Endothelial | 0.717 | 5.63E−06 |
| Streptomyces | PITX1 | Endothelial | 0.707 | 2.63E−05 |
| Streptomyces | GPR110 | Endothelial | 0.659 | 4.11E−05 |
| Streptomyces | PKIB | Endothelial | 0.662 | 2.77E−05 |
| Streptomyces | ANKRD22 | Endothelial | 0.645 | 6.63E−05 |
| Streptomyces | MUC5B | Endothelial | 0.650 | 7.46E−05 |
| Streptomyces | CCND1 | Endothelial | −0.715 | 2.06E−06 |
| Streptomyces | KLRD1 | Endothelial | 0.640 | 6.05E−05 |
| Streptomyces | PHGR1 | Endothelial | 0.714 | 1.36E−05 |
| Streptomyces | ONECUT3 | Endothelial | 0.656 | 4.50E−05 |
| Streptomyces | CEACAM6 | Endothelial | 0.661 | 2.11E−05 |
| Streptomyces | KCNN4 | Endothelial | 0.642 | 5.59E−05 |
| Vibrio | CD2 | Endothelial | 0.695 | 2.02E−05 |
| Vibrio | GZMA | Endothelial | 0.716 | 5.82E−06 |
| Vibrio | IFITM1 | Endothelial | 0.673 | 3.40E−05 |
| Vibrio | PTPRCAP | Endothelial | 0.664 | 4.63E−05 |
| Vibrio | AMICA1 | Endothelial | 0.744 | 1.59E−06 |
| Vibrio | CD3D | Endothelial | 0.708 | 8.39E−06 |
| Vibrio | LAG3 | Endothelial | 0.694 | 2.94E−05 |
| Vibrio | CD163 | Endothelial | 0.666 | 5.80E−05 |
| Vibrio | KLRB1 | Endothelial | 0.682 | 2.35E−05 |
| Vibrio | CD7 | Endothelial | 0.676 | 2.99E−05 |
| Vibrio | NKG7 | Endothelial | 0.702 | 1.07E−05 |
| Aspergillus | ALCAM | Endothelial | 0.665 | 4.51E−05 |
| Aspergillus | KCNN4 | Endothelial | 0.666 | 5.80E−05 |
| Colletotrichum | ALCAM | Endothelial | 0.695 | 1.03E−05 |
| Colletotrichum | RP11.290F20.3 | Endothelial | 0.665 | 8.27E−05 |
| Saccharomyces | ALCAM | Endothelial | 0.696 | 9.69E−06 |
| Saccharomyces | RP11.290F20.3 | Endothelial | 0.664 | 8.62E−05 |
| Saccharomyces | KCNN4 | Endothelial | 0.649 | 7.86E−05 |
| Thermothielavioides | ALCAM | Endothelial | 0.692 | 1.13E−05 |
| Acinetobacter | CEACAM7 | Fibroblast | −0.801 | 3.82E−05 |
| Bacillus | ASPM | Fibroblast | −0.727 | 8.65E−05 |
| Bacteroides | CD53 | Fibroblast | 0.661 | 9.35E−05 |
| Bacteroides | CTSS | Fibroblast | 0.672 | 8.99E−05 |
| Bacteroides | SELL | Fibroblast | 0.743 | 9.10E−06 |
| Bacteroides | HTRA3 | Fibroblast | 0.733 | 6.06E−06 |
| Bacteroides | UBD | Fibroblast | 0.714 | 2.02E−05 |
| Bacteroides | UCP2 | Fibroblast | 0.728 | 1.69E−05 |
| Bacteroides | GPR183 | Fibroblast | 0.686 | 5.62E−05 |
| Bacteroides | ITGA3 | Fibroblast | 0.689 | 9.86E−05 |
| Burkholderia | RGS4 | Fibroblast | −0.743 | 3.17E−05 |
| Burkholderia | G0S2 | Fibroblast | 0.719 | 7.50E−05 |
| Klebsiella | RGS4 | Fibroblast | −0.724 | 6.33E−05 |
| Klebsiella | AKR1C2 | Fibroblast | 0.719 | 3.44E−05 |
| Megamonas | UCP2 | Fibroblast | 0.725 | 2.80E−05 |
| Megamonas | KLK11 | Fibroblast | 0.785 | 2.09E−06 |
| Megamonas | KCNJ6 | Fibroblast | 0.799 | 2.80E−06 |
| Paracoccus | RGS4 | Fibroblast | −0.722 | 6.72E−05 |
| Pasteurella | AKR1C2 | Fibroblast | 0.761 | 6.51E−06 |
| Prevotella | UCP2 | Fibroblast | 0.781 | 2.53E−06 |
| Prevotella | CD27 | Fibroblast | 0.692 | 6.40E−05 |
| Prevotella | CST4 | Fibroblast | 0.692 | 8.96E−05 |
| Prevotella | KLK11 | Fibroblast | 0.712 | 4.59E−05 |
| Prevotella | KCNJ6 | Fibroblast | 0.721 | 6.95E−05 |
| Sphingobacterium | MACC1 | Fibroblast | 0.689 | 7.13E−05 |
| Staphylococcus | RGS4 | Fibroblast | −0.731 | 4.95E−05 |
| Streptomyces | GJA5 | Fibroblast | −0.683 | 6.10E−05 |
| Streptomyces | CYTL1 | Fibroblast | −0.702 | 9.17E−05 |
| Kluyveromyces | TSPAN1 | Fibroblast | 0.709 | 5.01E−05 |
| Kluyveromyces | HIST2H2AA3 | Fibroblast | 0.761 | 9.84E−06 |
| Kluyveromyces | IL1RN | Fibroblast | 0.692 | 6.36E−05 |
| Kluyveromyces | TIGIT | Fibroblast | 0.714 | 4.19E−05 |
| Kluyveromyces | AREG | Fibroblast | 0.709 | 7.29E−05 |
| Kluyveromyces | PITX1 | Fibroblast | 0.729 | 2.43E−05 |
| Kluyveromyces | LINC00035 | Fibroblast | 0.728 | 5.52E−05 |
| Kluyveromyces | CYBB | Fibroblast | 0.683 | 6.31E−05 |
| Kluyveromyces | PHLDA2 | Fibroblast | 0.688 | 5.21E−05 |
| Kluyveromyces | CTSW | Fibroblast | 0.685 | 8.01E−05 |
| Kluyveromyces | TAGLN | Fibroblast | 0.716 | 1.81E−05 |
| Kluyveromyces | ITGA5 | Fibroblast | 0.722 | 2.16E−05 |
| Kluyveromyces | OASL | Fibroblast | 0.690 | 4.94E−05 |
| Kluyveromyces | GREM1 | Fibroblast | 0.690 | 4.86E−05 |
| Kluyveromyces | C15orf48 | Fibroblast | 0.757 | 1.18E−05 |
| Kluyveromyces | SLC16A3 | Fibroblast | 0.726 | 1.79E−05 |
| Thermothielavioides | CDC20 | Fibroblast | 0.712 | 4.49E−05 |
| Bacteroides | CAPN8 | Macrophage | 0.715 | 5.85E−05 |
| Bacteroides | ANXA10 | Macrophage | 0.737 | 4.03E−05 |
| Klebsiella | KLRC1 | Macrophage | −0.703 | 8.82E−05 |
| Mycoplasma | KLRC1 | Macrophage | 0.673 | 8.69E−05 |
| Pasteurella | KLRC1 | Macrophage | −0.712 | 6.62E−05 |
| Ralstonia | KLRC1 | Macrophage | −0.739 | 5.70E−05 |
| Ralstonia | CD7 | Macrophage | −0.754 | 3.26E−05 |
| Bacteroides | AQP3 | Stellate | 0.710 | 6.94E−05 |
| Burkholderia | F3 | Stellate | −0.667 | 7.81E−05 |
| Burkholderia | FAM150B | Stellate | 0.709 | 3.46E−05 |
| Burkholderia | PDLIM3 | Stellate | −0.687 | 5.41E−05 |
| Burkholderia | CFTR | Stellate | 0.751 | 4.13E−06 |
| Burkholderia | GIMAP5 | Stellate | 0.673 | 8.69E−05 |
| Burkholderia | CERCAM | Stellate | −0.683 | 8.78E−05 |
| Burkholderia | FXYD2 | Stellate | 0.720 | 1.09E−05 |
| Burkholderia | MMP19 | Stellate | −0.678 | 7.36E−05 |
| Burkholderia | CCT2 | Stellate | −0.727 | 7.91E−06 |
| Burkholderia | EGLN3 | Stellate | −0.776 | 8.49E−06 |
| Burkholderia | FAM83D | Stellate | −0.692 | 8.91E−05 |
| Burkholderia | KLK10 | Stellate | −0.672 | 8.92E−05 |
| Burkholderia | TFF2 | Stellate | −0.709 | 5.01E−05 |
| Burkholderia | PNLIPRP1 | Stellate | 0.711 | 9.87E−05 |
| Burkholderia | CTRB2 | Stellate | 0.665 | 8.28E−05 |
| Chryseobacterium | PDIA2 | Stellate | 0.757 | 1.19E−05 |
| Flavobacterium | UGT2A3 | Stellate | 0.725 | 6.15E−05 |
| Flavobacterium | PDIA2 | Stellate | 0.761 | 1.01E−05 |
| Klebsiella | FAM150B | Stellate | 0.720 | 2.28E−05 |
| Klebsiella | GALNT5 | Stellate | −0.697 | 5.31E−05 |
| Klebsiella | PDLIM3 | Stellate | −0.707 | 2.64E−05 |
| Klebsiella | ACHE | Stellate | −0.671 | 9.23E−05 |
| Klebsiella | CFTR | Stellate | 0.719 | 1.64E−05 |
| Klebsiella | CERCAM | Stellate | −0.688 | 7.30E−05 |
| Klebsiella | FXYD2 | Stellate | 0.715 | 1.30E−05 |
| Klebsiella | MMP19 | Stellate | −0.706 | 2.71E−05 |
| Klebsiella | EGLN3 | Stellate | −0.807 | 1.87E−06 |
| Klebsiella | KLK10 | Stellate | −0.680 | 6.81E−05 |
| Klebsiella | PNLIPRP1 | Stellate | 0.719 | 7.56E−05 |
| Klebsiella | CTRB2 | Stellate | 0.661 | 9.44E−05 |
| Megamonas | MOXD1 | Stellate | 0.704 | 4.13E−05 |
| Megamonas | FGF7 | Stellate | 0.742 | 9.47E−06 |
| Megamonas | APOE | Stellate | 0.694 | 5.98E−05 |
| Mycoplasma | PDIA2 | Stellate | 0.724 | 6.27E−05 |
| Paracoccus | TNC | Stellate | −0.710 | 3.37E−05 |
| Paracoccus | PNLIPRP1 | Stellate | 0.712 | 9.59E−05 |
| Pasteurella | F3 | Stellate | −0.664 | 8.62E−05 |
| Pasteurella | HSD11B1 | Stellate | −0.725 | 1.92E−05 |
| Pasteurella | FAM150B | Stellate | 0.723 | 2.08E−05 |
| Pasteurella | GALNT5 | Stellate | −0.685 | 7.96E−05 |
| Pasteurella | PDLIM3 | Stellate | −0.715 | 1.88E−05 |
| Pasteurella | ACHE | Stellate | −0.675 | 8.27E−05 |
| Pasteurella | CFTR | Stellate | 0.734 | 8.74E−06 |
| Pasteurella | GIMAP5 | Stellate | 0.670 | 9.68E−05 |
| Pasteurella | PLAT | Stellate | −0.694 | 4.20E−05 |
| Pasteurella | DKK3 | Stellate | −0.671 | 6.79E−05 |
| Pasteurella | ANO1 | Stellate | −0.683 | 4.41E−05 |
| Pasteurella | FXYD2 | Stellate | 0.740 | 4.42E−06 |
| Pasteurella | MMP19 | Stellate | −0.707 | 2.60E−05 |
| Pasteurella | CCT2 | Stellate | −0.691 | 3.31E−05 |
| Pasteurella | EGLN3 | Stellate | −0.815 | 1.22E−06 |
| Pasteurella | SERPINA5 | Stellate | 0.710 | 2.29E−05 |
| Pasteurella | KLK10 | Stellate | −0.682 | 6.37E−05 |
| Pasteurella | TFF2 | Stellate | −0.727 | 2.60E−05 |
| Pasteurella | CTRB2 | Stellate | 0.667 | 7.74E−05 |
| Prevotella | KLRC1 | Stellate | 0.838 | 2.08E−06 |
| Ralstonia | HSD11B1 | Stellate | −0.702 | 4.46E−05 |
| Ralstonia | CTRB2 | Stellate | 0.672 | 6.60E−05 |
| Spiroplasma | TUBA1A | Stellate | −0.715 | 4.10E−05 |
| Staphylococcus | CFTR | Stellate | 0.680 | 6.96E−05 |
| Staphylococcus | FXYD2 | Stellate | 0.674 | 6.06E−05 |
| Staphylococcus | CCT2 | Stellate | −0.660 | 9.93E−05 |
| Staphylococcus | EGLN3 | Stellate | −0.754 | 2.13E−05 |
| Staphylococcus | FAM83D | Stellate | −0.689 | 9.99E−05 |
| Staphylococcus | CTRB2 | Stellate | 0.666 | 8.01E−05 |
| Streptomyces | PDIA2 | Stellate | 0.745 | 1.93E−05 |
| Aspergillus | ISG15 | Stellate | 0.660 | 9.94E−05 |
| Aspergillus | CDCA8 | Stellate | 0.709 | 2.41E−05 |
| Aspergillus | F3 | Stellate | 0.707 | 1.79E−05 |
| Aspergillus | ECM1 | Stellate | 0.672 | 9.09E−05 |
| Aspergillus | NUF2 | Stellate | 0.775 | 2.09E−06 |
| Aspergillus | UBE2T | Stellate | 0.721 | 1.00E−05 |
| Aspergillus | CD55 | Stellate | 0.692 | 3.21E−05 |
| Aspergillus | FAM150B | Stellate | −0.815 | 2.20E−07 |
| Aspergillus | REG1A | Stellate | −0.676 | 5.60E−05 |
| Aspergillus | SCTR | Stellate | −0.753 | 3.32E−05 |
| Aspergillus | COL5A2 | Stellate | 0.692 | 3.24E−05 |
| Aspergillus | FN1 | Stellate | 0.688 | 3.72E−05 |
| Aspergillus | FBLN2 | Stellate | 0.687 | 3.88E−05 |
| Aspergillus | FAM107A | Stellate | −0.693 | 8.68E−05 |
| Aspergillus | CXCL5 | Stellate | 0.710 | 7.09E−05 |
| Aspergillus | EREG | Stellate | 0.713 | 2.96E−05 |
| Aspergillus | PDLIM3 | Stellate | 0.810 | 1.78E−07 |
| Aspergillus | SPARC | Stellate | 0.718 | 1.17E−05 |
| Aspergillus | AQP1 | Stellate | −0.679 | 7.11E−05 |
| Aspergillus | AEBP1 | Stellate | 0.696 | 2.80E−05 |
| Aspergillus | CFTR | Stellate | −0.778 | 1.11E−06 |
| Aspergillus | CALD1 | Stellate | 0.702 | 6.46E−05 |
| Aspergillus | GIMAP5 | Stellate | −0.764 | 2.19E−06 |
| Aspergillus | EGFL6 | Stellate | 0.741 | 4.27E−06 |
| Aspergillus | LOXL2 | Stellate | 0.750 | 2.84E−06 |
| Aspergillus | SULF1 | Stellate | 0.722 | 1.46E−05 |
| Aspergillus | FABP4 | Stellate | −0.671 | 6.77E−05 |
| Aspergillus | SDC2 | Stellate | 0.703 | 2.08E−05 |
| Aspergillus | CERCAM | Stellate | 0.702 | 4.49E−05 |
| Aspergillus | AKR1C3 | Stellate | 0.671 | 6.75E−05 |
| Aspergillus | CUZD1 | Stellate | −0.704 | 8.61E−05 |
| Aspergillus | SERPINH1 | Stellate | 0.667 | 7.69E−05 |
| Aspergillus | FXYD2 | Stellate | −0.771 | 9.76E−07 |
| Aspergillus | TUBA1C | Stellate | 0.679 | 5.18E−05 |
| Aspergillus | CCT2 | Stellate | 0.744 | 3.78E−06 |
| Aspergillus | COL4A1 | Stellate | 0.707 | 1.78E−05 |
| Aspergillus | COL4A2 | Stellate | 0.712 | 1.50E−05 |
| Aspergillus | EGLN3 | Stellate | 0.721 | 7.02E−05 |
| Aspergillus | LGALS3 | Stellate | 0.672 | 6.60E−05 |
| Aspergillus | LGMN | Stellate | 0.723 | 2.04E−05 |
| Aspergillus | SERPINA5 | Stellate | −0.748 | 4.67E−06 |
| Aspergillus | CDH11 | Stellate | 0.679 | 5.12E−05 |
| Aspergillus | HSD11B2 | Stellate | 0.671 | 9.37E−05 |
| Aspergillus | KPNA2 | Stellate | 0.671 | 6.89E−05 |
| Aspergillus | TK1 | Stellate | 0.672 | 6.47E−05 |
| Aspergillus | TPX2 | Stellate | 0.722 | 9.90E−06 |
| Aspergillus | FAM83D | Stellate | 0.841 | 7.61E−08 |
| Aspergillus | RCN3 | Stellate | 0.694 | 8.47E−05 |
| Aspergillus | KLK10 | Stellate | 0.712 | 2.14E−05 |
| Aspergillus | CTRB2 | Stellate | −0.735 | 5.57E−06 |
| Colletotrichum | ISG15 | Stellate | 0.676 | 5.60E−05 |
| Colletotrichum | CDCA8 | Stellate | 0.721 | 1.52E−05 |
| Colletotrichum | F3 | Stellate | 0.706 | 1.86E−05 |
| Colletotrichum | RP11.14N7.2 | Stellate | 0.673 | 8.84E−05 |
| Colletotrichum | ECM1 | Stellate | 0.672 | 9.09E−05 |
| Colletotrichum | S100A4 | Stellate | 0.662 | 9.16E−05 |
| Colletotrichum | NUF2 | Stellate | 0.773 | 2.30E−06 |
| Colletotrichum | UBE2T | Stellate | 0.723 | 9.24E−06 |
| Colletotrichum | CD55 | Stellate | 0.698 | 2.57E−05 |
| Colletotrichum | FAM150B | Stellate | −0.825 | 1.17E−07 |
| Colletotrichum | REG1A | Stellate | −0.675 | 5.90E−05 |
| Colletotrichum | SCTR | Stellate | −0.767 | 1.93E−05 |
| Colletotrichum | COL5A2 | Stellate | 0.702 | 2.23E−05 |
| Colletotrichum | FN1 | Stellate | 0.698 | 2.52E−05 |
| Colletotrichum | FBLN2 | Stellate | 0.692 | 3.17E−05 |
| Colletotrichum | FAM107A | Stellate | −0.704 | 5.96E−05 |
| Colletotrichum | SMC4 | Stellate | 0.682 | 6.49E−05 |
| Colletotrichum | CXCL5 | Stellate | 0.718 | 5.24E−05 |
| Colletotrichum | EREG | Stellate | 0.708 | 3.61E−05 |
| Colletotrichum | PDLIM3 | Stellate | 0.811 | 1.67E−07 |
| Colletotrichum | VCAN | Stellate | 0.665 | 8.41E−05 |
| Colletotrichum | SPARC | Stellate | 0.727 | 8.02E−06 |
| Colletotrichum | AQP1 | Stellate | −0.677 | 7.66E−05 |
| Colletotrichum | AEBP1 | Stellate | 0.709 | 1.70E−05 |
| Colletotrichum | COL1A2 | Stellate | 0.665 | 8.22E−05 |
| Colletotrichum | CFTR | Stellate | −0.781 | 9.17E−07 |
| Colletotrichum | CALD1 | Stellate | 0.692 | 8.96E−05 |
| Colletotrichum | GIMAP5 | Stellate | −0.762 | 2.51E−06 |
| Colletotrichum | EGFL6 | Stellate | 0.747 | 3.31E−06 |
| Colletotrichum | LOXL2 | Stellate | 0.759 | 1.84E−06 |
| Colletotrichum | SULF1 | Stellate | 0.728 | 1.14E−05 |
| Colletotrichum | FABP4 | Stellate | −0.668 | 7.63E−05 |
| Colletotrichum | SDC2 | Stellate | 0.712 | 1.49E−05 |
| Colletotrichum | CERCAM | Stellate | 0.708 | 3.59E−05 |
| Colletotrichum | AKR1C3 | Stellate | 0.685 | 4.21E−05 |
| Colletotrichum | CUZD1 | Stellate | −0.707 | 7.65E−05 |
| Colletotrichum | SERPINH1 | Stellate | 0.675 | 5.85E−05 |
| Colletotrichum | FXYD2 | Stellate | −0.773 | 9.02E−07 |
| Colletotrichum | TUBA1C | Stellate | 0.688 | 3.76E−05 |
| Colletotrichum | CCT2 | Stellate | 0.753 | 2.48E−06 |
| Colletotrichum | COL4A1 | Stellate | 0.713 | 1.43E−05 |
| Colletotrichum | COL4A2 | Stellate | 0.711 | 1.54E−05 |
| Colletotrichum | EGLN3 | Stellate | 0.717 | 8.17E−05 |
| Colletotrichum | LGALS3 | Stellate | 0.671 | 6.72E−05 |
| Colletotrichum | LGMN | Stellate | 0.738 | 1.12E−05 |
| Colletotrichum | SERPINA5 | Stellate | −0.752 | 3.91E−06 |
| Colletotrichum | CDH11 | Stellate | 0.689 | 3.58E−05 |
| Colletotrichum | KPNA2 | Stellate | 0.675 | 5.80E−05 |
| Colletotrichum | TK1 | Stellate | 0.691 | 3.29E−05 |
| Colletotrichum | TPX2 | Stellate | 0.730 | 7.04E−06 |
| Colletotrichum | FAM83D | Stellate | 0.853 | 3.18E−08 |
| Colletotrichum | PLAUR | Stellate | 0.676 | 7.91E−05 |
| Colletotrichum | RCN3 | Stellate | 0.706 | 5.66E−05 |
| Colletotrichum | KLK10 | Stellate | 0.717 | 1.80E−05 |
| Colletotrichum | CTRB2 | Stellate | −0.730 | 7.08E−06 |
| Kluyveromyces | ISG15 | Stellate | 0.715 | 8.50E−05 |
| Kluyveromyces | CTSS | Stellate | 0.722 | 9.94E−05 |
| Kluyveromyces | S100A4 | Stellate | 0.714 | 9.02E−05 |
| Kluyveromyces | NUF2 | Stellate | 0.816 | 1.16E−06 |
| Kluyveromyces | UBE2T | Stellate | 0.767 | 1.22E−05 |
| Kluyveromyces | FAM150B | Stellate | −0.808 | 5.38E−06 |
| Kluyveromyces | CYS1 | Stellate | −0.748 | 6.17E−05 |
| Kluyveromyces | HK2 | Stellate | 0.742 | 5.06E−05 |
| Kluyveromyces | IL1RN | Stellate | 0.775 | 1.39E−05 |
| Kluyveromyces | FN1 | Stellate | 0.769 | 1.13E−05 |
| Kluyveromyces | CCNA2 | Stellate | 0.784 | 9.50E−06 |
| Kluyveromyces | SLC7A11 | Stellate | 0.755 | 3.09E−05 |
| Kluyveromyces | VCAN | Stellate | 0.729 | 5.40E−05 |
| Kluyveromyces | DLX5 | Stellate | 0.773 | 1.54E−05 |
| Kluyveromyces | CFTR | Stellate | −0.791 | 6.86E−06 |
| Kluyveromyces | GIMAP5 | Stellate | −0.809 | 2.97E−06 |
| Kluyveromyces | EGFL6 | Stellate | 0.785 | 5.57E−06 |
| Kluyveromyces | LOXL2 | Stellate | 0.749 | 2.53E−05 |
| Kluyveromyces | SULF1 | Stellate | 0.724 | 6.33E−05 |
| Kluyveromyces | SDC2 | Stellate | 0.729 | 5.36E−05 |
| Kluyveromyces | TSTA3 | Stellate | 0.748 | 6.26E−05 |
| Kluyveromyces | AKR1C3 | Stellate | 0.798 | 3.02E−06 |
| Kluyveromyces | SFTA1P | Stellate | 0.770 | 1.76E−05 |
| Kluyveromyces | COL17A1 | Stellate | 0.796 | 3.25E−06 |
| Kluyveromyces | FXYD2 | Stellate | −0.791 | 4.27E−06 |
| Kluyveromyces | CDCA3 | Stellate | 0.753 | 2.16E−05 |
| Kluyveromyces | MGST1 | Stellate | 0.717 | 8.08E−05 |
| Kluyveromyces | OASL | Stellate | 0.768 | 1.91E−05 |
| Kluyveromyces | COL4A1 | Stellate | 0.765 | 1.36E−05 |
| Kluyveromyces | COL4A2 | Stellate | 0.782 | 6.52E−06 |
| Kluyveromyces | SERPINA5 | Stellate | −0.809 | 2.94E−06 |
| Kluyveromyces | DUOX2 | Stellate | 0.759 | 2.72E−05 |
| Kluyveromyces | DUOXA2 | Stellate | 0.811 | 8.02E−06 |
| Kluyveromyces | C15orf48 | Stellate | 0.818 | 5.92E−06 |
| Kluyveromyces | CDH11 | Stellate | 0.747 | 2.70E−05 |
| Kluyveromyces | COTL1 | Stellate | 0.762 | 1.52E−05 |
| Kluyveromyces | IRF8 | Stellate | 0.769 | 1.78E−05 |
| Kluyveromyces | CDT1 | Stellate | 0.772 | 1.62E−05 |
| Kluyveromyces | CCL18 | Stellate | 0.734 | 6.79E−05 |
| Kluyveromyces | LINC00671 | Stellate | −0.779 | 1.95E−05 |
| Kluyveromyces | HN1 | Stellate | 0.726 | 5.89E−05 |
| Kluyveromyces | TK1 | Stellate | 0.739 | 3.67E−05 |
| Kluyveromyces | TYMS | Stellate | 0.732 | 4.76E−05 |
| Kluyveromyces | PMAIP1 | Stellate | 0.842 | 9.16E−07 |
| Kluyveromyces | TPX2 | Stellate | 0.787 | 5.15E−06 |
| Kluyveromyces | FAM83D | Stellate | 0.814 | 2.26E−06 |
| Kluyveromyces | RP11.290F20.3 | Stellate | 0.809 | 5.24E−06 |
| Saccharomyces | F3 | Stellate | 0.685 | 5.71E−05 |
| Saccharomyces | S100A4 | Stellate | 0.671 | 9.43E−05 |
| Saccharomyces | NUF2 | Stellate | 0.773 | 3.67E−06 |
| Saccharomyces | UBE2T | Stellate | 0.683 | 6.23E−05 |
| Saccharomyces | CD55 | Stellate | 0.770 | 1.66E−06 |
| Saccharomyces | FAM150B | Stellate | −0.805 | 7.16E−07 |
| Saccharomyces | MXD1 | Stellate | 0.696 | 7.78E−05 |
| Saccharomyces | REG1A | Stellate | −0.676 | 7.99E−05 |
| Saccharomyces | SCTR | Stellate | −0.754 | 5.01E−05 |
| Saccharomyces | COL5A2 | Stellate | 0.678 | 7.34E−05 |
| Saccharomyces | FN1 | Stellate | 0.683 | 6.25E−05 |
| Saccharomyces | FBLN2 | Stellate | 0.700 | 3.34E−05 |
| Saccharomyces | SMC4 | Stellate | 0.693 | 6.15E−05 |
| Saccharomyces | PDLIM3 | Stellate | 0.780 | 1.64E−06 |
| Saccharomyces | VCAN | Stellate | 0.671 | 9.23E−05 |
| Saccharomyces | SPARC | Stellate | 0.700 | 3.32E−05 |
| Saccharomyces | DCDC2 | Stellate | −0.693 | 8.59E−05 |
| Saccharomyces | AEBP1 | Stellate | 0.676 | 7.91E−05 |
| Saccharomyces | CFTR | Stellate | −0.807 | 3.75E−07 |
| Saccharomyces | GIMAP5 | Stellate | −0.705 | 3.95E−05 |
| Saccharomyces | EGFL6 | Stellate | 0.727 | 1.16E−05 |
| Saccharomyces | LOXL2 | Stellate | 0.736 | 8.23E−06 |
| Saccharomyces | SULF1 | Stellate | 0.719 | 1.64E−05 |
| Saccharomyces | SDC2 | Stellate | 0.685 | 5.79E−05 |
| Saccharomyces | FXYD2 | Stellate | −0.729 | 1.06E−05 |
| Saccharomyces | CCT2 | Stellate | 0.720 | 1.54E−05 |
| Saccharomyces | COL4A1 | Stellate | 0.675 | 8.14E−05 |
| Saccharomyces | COL4A2 | Stellate | 0.673 | 8.84E−05 |
| Saccharomyces | LGALS3 | Stellate | 0.669 | 9.78E−05 |
| Saccharomyces | LGMN | Stellate | 0.696 | 7.99E−05 |
| Saccharomyces | SERPINA5 | Stellate | 0.714 | 2.91E−05 |
| Saccharomyces | CDH11 | Stellate | 0.676 | 7.81E−05 |
| Saccharomyces | TPX2 | Stellate | 0.680 | 6.85E−05 |
| Saccharomyces | FAM83D | Stellate | 0.840 | 7.99E−08 |
| Saccharomyces | PLAUR | Stellate | 0.699 | 4.96E−05 |
| Saccharomyces | KLK10 | Stellate | 0.702 | 4.52E−05 |
| Saccharomyces | CTRB2 | Stellate | −0.720 | 1.58E−05 |
| Thermothielavioides | CDCA8 | Stellate | 0.727 | 1.15E−05 |
| Thermothielavioides | F3 | Stellate | 0.691 | 3.36E−05 |
| Thermothielavioides | NUF2 | Stellate | 0.763 | 3.75E−06 |
| Thermothielavioides | UBE2T | Stellate | 0.694 | 2.97E−05 |
| Thermothielavioides | CD55 | Stellate | 0.668 | 7.50E−05 |
| Thermothielavioides | FAM150B | Stellate | −0.807 | 3.71E−07 |
| Thermothielavioides | REG1A | Stellate | −0.676 | 5.70E−05 |
| Thermothielavioides | SCTR | Stellate | −0.760 | 2.54E−05 |
| Thermothielavioides | COL5A2 | Stellate | 0.684 | 4.34E−05 |
| Thermothielavioides | FN1 | Stellate | 0.677 | 5.51E−05 |
| Thermothielavioides | FBLN2 | Stellate | 0.685 | 4.10E−05 |
| Thermothielavioides | FAM107A | Stellate | −0.698 | 7.46E−05 |
| Thermothielavioides | CXCL5 | Stellate | 0.732 | 3.18E−05 |
| Thermothielavioides | EREG | Stellate | 0.699 | 5.05E−05 |
| Thermothielavioides | PDLIM3 | Stellate | 0.797 | 3.96E−07 |
| Thermothielavioides | SPARC | Stellate | 0.692 | 3.16E−05 |
| Thermothielavioides | AEBP1 | Stellate | 0.719 | 1.11E−05 |
| Thermothielavioides | CFTR | Stellate | −0.773 | 1.39E−06 |
| Thermothielavioides | GIMAP5 | Stellate | −0.743 | 5.83E−06 |
| Thermothielavioides | EGFL6 | Stellate | 0.740 | 4.57E−06 |
| Thermothielavioides | LOXL2 | Stellate | 0.731 | 6.72E−06 |
| Thermothielavioides | SULF1 | Stellate | 0.704 | 2.92E−05 |
| Thermothielavioides | SDC2 | Stellate | 0.677 | 5.41E−05 |
| Thermothielavioides | CERCAM | Stellate | 0.689 | 7.06E−05 |
| Thermothielavioides | AKR1C3 | Stellate | 0.681 | 4.86E−05 |
| Thermothielavioides | CUZD1 | Stellate | −0.716 | 5.69E−05 |
| Thermothielavioides | FXYD2 | Stellate | −0.761 | 1.67E−06 |
| Thermothielavioides | CCT2 | Stellate | 0.730 | 6.90E−06 |
| Thermothielavioides | COL4A1 | Stellate | 0.682 | 4.62E−05 |
| Thermothielavioides | COL4A2 | Stellate | 0.684 | 4.34E−05 |
| Thermothielavioides | EGLN3 | Stellate | 0.730 | 5.13E−05 |
| Thermothielavioides | LGMN | Stellate | 0.707 | 3.75E−05 |
| Thermothielavioides | SERPINA5 | Stellate | −0.756 | 3.35E−06 |
| Thermothielavioides | CDH11 | Stellate | 0.667 | 7.74E−05 |
| Thermothielavioides | TK1 | Stellate | 0.668 | 7.42E−05 |
| Thermothielavioides | TPX2 | Stellate | 0.683 | 4.44E−05 |
| Thermothielavioides | FAM83D | Stellate | 0.825 | 2.23E−07 |
| Thermothielavioides | KLK10 | Stellate | 0.692 | 4.49E−05 |
| Thermothielavioides | CTRB2 | Stellate | −0.722 | 9.64E−06 |
| Chryseobacterium | HIST1H4C | T_cell | −0.804 | 9.90E−05 |
| Aspergillus | THBS4 | T cell | 0.890 | 2.05E−05 |
| Aspergillus | LPL | T_cell | 0.881 | 1.44E−05 |
| Colletotrichum | LPL | T_cell | 0.870 | 5.31E−05 |
| Kluyveromyces | PLA2G2A | T_cell | 0.863 | 3.41E−05 |
| Kluyveromyces | CD34 | T_cell | 0.887 | 2.36E−05 |
| Kluyveromyces | UCHL1 | T_cell | 0.846 | 7.12E−05 |
| Saccharomyces | LPL | T_cell | 0.870 | 5.31E−05 |
| Thermothielavioides | LPL | T_cell | 0.870 | 5.31E−05 |

Microbiome predicted patient survival: Whether intra-tumoral microbial diversity and associated gene expression signatures could predict patients at risk of poor survival was determined. First, pseudo-bulk gene expression profiles were created from the Peng et al. (Peng et al. Cell Res. 29(9):725-738, 2019) cohort by summing the gene counts across all cells in a given sample. Regularized logistic regression was then used to identify a six-gene signature that accurately classified the samples as having low or high microbial diversity, defined as having a Shannon index below or above the median for the cohort (Example 1, FIG. 19G, Appendix II). Next, the model was used to predict whether individual pancreatic tumors profiled with bulk-RNA sequencing from TCGA (Raphael et al. Cancer Cell 32: 185-203.e13, 2017) and the International Cancer Genomics Consortium (ICGC) (Hudson et al. Nature, 464: 993-998, 2010) had high or low intra-tumoral microbial diversity. Patients were then stratified by the predicted microbial diversity of their tumor and the relationship with survival was tested using a univariate Cox proportional hazards model (FIGS. 19G-19H). In both datasets, high microbial diversity was associated with significantly decreased overall survival (TCGA: Hazard Ratio [HR]=2.6, 95% Confidence Interval [CI]: 1.4-5.3, p=0.0031; ICGC: HR=1.9, 95% CI: 1.2-2.9, p=0.0053; FIG. 19H). A similar trend was observed when stratifying TCGA patients by microbiome diversity calculated from microbial profiles directly measured from the same samples and reported by Poore et al. (Poore et al. Nature 579: 567-574, 2020)., albeit with a smaller effect size (p=0.083, FIG. 19H), highlighting the increased resolution possible when single-cell data are used. Of note, there was a 63% overlap between predicted and observed TCGA diversity. These results indicated that microbial composition and associated gene expression signatures in host cells can identify PDA patients at risk of poor outcomes, and that the model derived from single cell genomic data outperforms that derived from genomic data from bulk tumor tissues, due to its greater resolving power.

### Example 26—Example Quality Control Analysis

False-positive identifications are a significant problem in metagenomics classification systems. This example describes a particular embodiment of the SAHMI (Single-cell Analysis of Host-Microbiome Interactions) method to identify microbes and viruses in subjects at single cell resolution using genomic approaches, including criteria for improved identification of true species versus contaminants and false positives. These criteria can be used to reduce the occurrence of false positives and contaminants in any of the methods disclosed herein.

As described in Examples 1 and 2, metagenomic classification of paired-end reads from scRNAseq fastq files was done using Kraken 2 (Wood et al. Genome Biol. 20: 257, 2019). The present example also employed KrakenUniq (Breitwieser et al. Genome Biology. 19:198, 2018), which combines very fast k-mer-based classification with a fast k-mer cardinality estimation. KrakenUniq adds a method for counting the number of unique k-mers identified for each taxon using the cardinality estimation algorithm HyperLogLog. By counting how many of each genome's unique k-mers are covered by reads, KrakenUniq can more effectively discern false-positive from true-positive matches.

To mitigate the influence of classification errors, contamination, and noise, results from Kraken 2 and KrakenUniq analyses were assessed against four criteria for selecting true species in a set of samples and reducing or eliminating false positives and contaminants. Common contaminants and false positive signatures were identified using a wide variety of cell lines. The four criteria were as follows: (1) a true species had a positive relationship between the number of reads assigned and number of minimizers assigned; (2) a true species has a positive relationship between number of reads assigned and number of unique minimizers assigned; (3) a true species has a positive relationship between number of minimizers assigned and number of unique minimizers assigned; and (4) a true species has a fractional composition of the detected microbiomes that is greater than that found in negative controls samples. In the absence of paired negative controls, cell line experiments can be used (wherein only false positives and contaminants would be expected to be found). Microbes and viruses identified using Kraken 2 and KrakenUniq that fit the criteria (i.e., species that were present in samples in greater numbers than in negative controls) were maintained for further processing and analysis. Reads were then deduplicated and demultiplexed based on their cell barcode and unique molecular identifiers, sparse barcodes were filtered out, and barcode taxa reassignment was performed.

Mapped metagenomic reads first underwent a series of filters. ShortRead (Morgan et al. Bioinformatics 25: 2607-2608, 2009) was used to remove low complexity reads (<20 non-sequentially repeated nucleotides), low quality reads (PHRED score<20), and PCR duplicates tagged with the same unique molecular identifier and cellular barcode. Non-sparse cellular barcodes were then selected by using an elbow-plot of barcode rank vs. total reads, smoothed with a moving average of 5, and with a cutoff at a change in slope<10′, in a manner analogous to how cellular barcodes are typically selected in single-cell sequencing data (CellRanger (10× Genomics), Drop-seq Core Computational Protocol v2.0.0 (McCarroll laboratory)). Lastly, taxizedb (Chamberlain et al. Tools for Working with ‘Taxonomic’ Databases, 2020) was used to obtain full taxonomic classifications for all resulting reads, and the number of reads assigned to each clade was counted.

Next, sample-level normalized metagenomic levels were calculated as log 2 (counts/total_counts*10,000+1). For analyses that compared cell-level metagenome and somatic gene expression, the default Seurat normalization was used. To identify bacteria, fungi, and viruses that were differentially present in case samples compared to controls, or that were present in both case samples and in positive controls, a linear model was constructed to predict sample-level normalized microbe or virus levels as a function of tissue status, somatic cellular composition (to account for potential tropisms), and total metagenomic reads. Cellular counts and total metagenomic counts were log-normalized prior to model fitting.

### Example 27—Detecting an Infection

This example describes a particular embodiment of the SAHMI (Single-cell Analysis of Host-Microbiome Interactions) method to identify microbes and viruses in subjects (such as in a sample from a subject) at single cell resolution using genomic approaches.

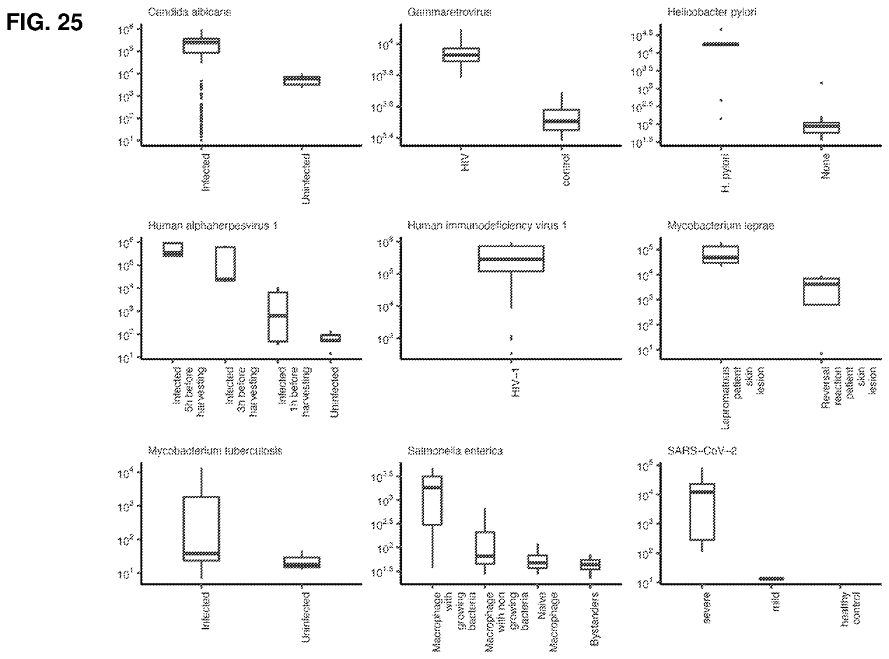

SAHMI was used herein to identify infectious disease agents (e.g., microbes and viruses) using scRNAseq data from various types of human tissues, including blood, skin, stomach, and lung samples. SAHMI identified relevant infectious disease agents in samples as compared to controls for each agent tested (Candida albicans, HIV (with and without controls), Helicobacter pylori, alphaherpesvirus 1, Mycobacterium leprae, Mycobacterium tuberculosis, Salmonella enterica, and SARS-CoV-2) (FIG. 25).

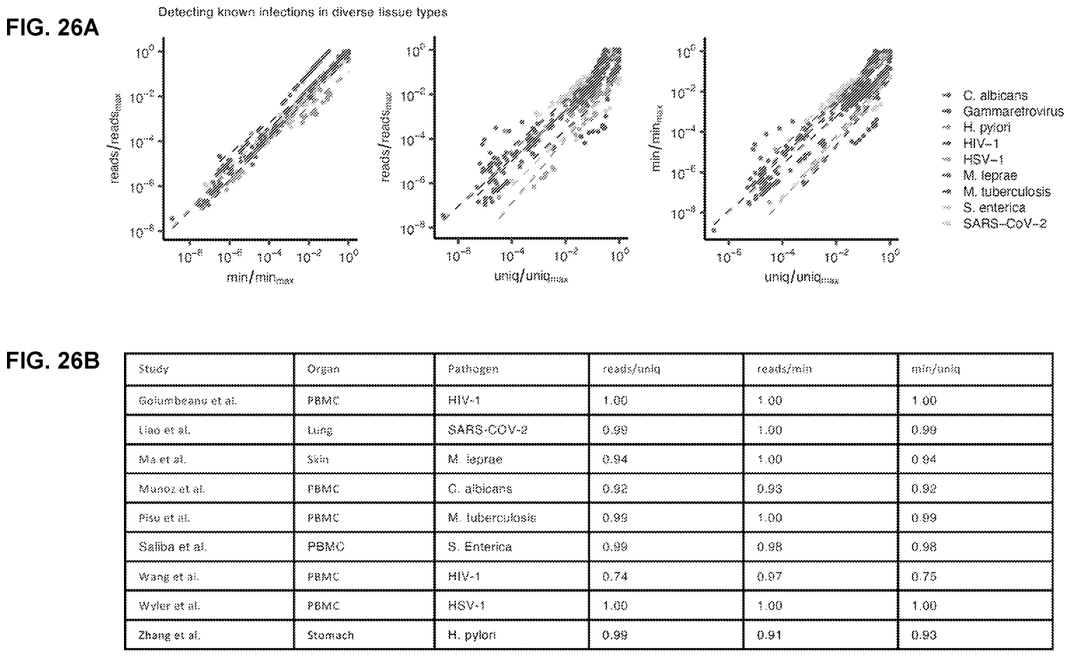

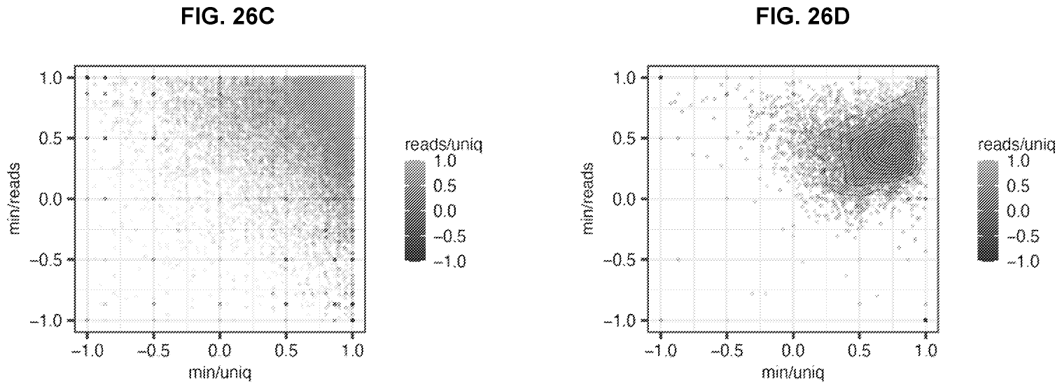

The criteria described in Example 3 were applied for detecting and de-noising the microbiome signals. Sequencing reads from true species had positive relationships between (1) the number of reads assigned and number of minimizers assigned, (2) number of minimizers assigned and number of unique minimizers assigned, and (3) number of reads assigned and number of unique minimizers assigned (FIGS. 26A-26B). Low correlation values for the three criteria indicated the presence of false positive results, whereas high values suggested the presence of other species, including contaminants (FIGS. 26C-26D). In test samples, species not detected above the thresholds found in negative controls (FIG. 26D) were assumed to be false positive or contaminant species.

These data indicate that SAMHI can identify infectious agents, including bacteria, fungi, and viruses, using scRNAseq data from various tissue types collected from subjects that have, or are suspected of having, an infection.

### Example 28—Example Computing System

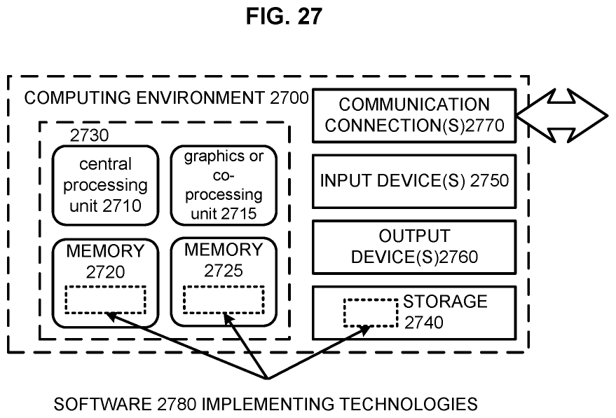

FIG. 27 illustrates a generalized example of a suitable computing system 2700 in which any of the described technologies may be implemented. The computing system 2700 is not intended to suggest any limitation as to scope of use or functionality, as the innovations may be implemented in diverse computing systems, including special-purpose computing systems. In practice, a computing system can comprise multiple networked instances of the illustrated computing system.

With reference to FIG. 27, the computing system 2700 includes one or more processing units 2710, 2715 and memory 2720, 2725. In FIG. 27, this basic configuration 2730 is included within a dashed line. The processing units 2710, 2715 execute computer-executable instructions. A processing unit can be a central processing unit (CPU), processor in an application-specific integrated circuit (ASIC), or any other type of processor. In a multi-processing system, multiple processing units execute computer-executable instructions to increase processing power. For example, FIG. 27 shows a central processing unit 2710 as well as a graphics processing unit or co-processing unit 2715. The tangible memory 2720, 2725 may be volatile memory (e.g., registers, cache, RAM), non-volatile memory (e.g., ROM, EEPROM, flash memory, etc.), or some combination of the two, accessible by the processing unit(s). The memory 2720, 2725 stores software 2780 implementing one or more innovations described herein, in the form of computer-executable instructions suitable for execution by the processing unit(s).

A computing system may have additional features. For example, the computing system 2700 includes storage 2740, one or more input devices 2750, one or more output devices 2760, and one or more communication connections 2770. An interconnection mechanism (not shown) such as a bus, controller, or network interconnects the components of the computing system 2700. Typically, operating system software (not shown) provides an operating environment for other software executing in the computing system 2700, and coordinates activities of the components of the computing system 2700.

The tangible storage 2740 may be removable or non-removable, and includes magnetic disks, magnetic tapes or cassettes, CD-ROMs, DVDs, or any other medium which can be used to store information in a non-transitory way and which can be accessed within a computing system. The storage 2740 stores instructions for the software 2780 implementing one or more innovations described herein.

The input device(s) 2750 may be a touch input device such as a keyboard, mouse, pen, or trackball, a voice input device, a scanning device, or another device that provides input to the computing system 2700. For video encoding, the input device(s) 2750 may be a camera, video card, TV tuner card, or similar device that accepts video input in analog or digital form, or a CD-ROM or CD-RW that reads video samples into the computing system 2700. The output device(s) 160 may be a display, printer, speaker, CD-writer, or another device that provides output from the computing system 2700.

The communication connection(s) 2770 enable communication over a communication medium to another computing entity. The communication medium conveys information such as computer-executable instructions, audio or video input or output, or other data in a modulated data signal. A modulated data signal is a signal that has one or more of its characteristics set or changed in such a manner as to encode information in the signal. By way of example, and not limitation, communication media can use an electrical, optical, RF, or other carrier.

The innovations can be described in the general context of computer-executable instructions, such as those included in program modules, being executed in a computing system on a target real or virtual processor (e.g., that is ultimately implemented on a hardware processor). Generally, program modules include routines, programs, libraries, objects, classes, components, data structures, etc. that perform particular tasks or implement particular abstract data types. The functionality of the program modules may be combined or split between program modules as desired in various embodiments. Computer-executable instructions for program modules may be executed within a local or distributed computing system.

For the sake of presentation, the detailed description uses terms like “determine” and “use” to describe computer operations in a computing system. These terms are high-level abstractions for operations performed by a computer and should not be confused with acts performed by a human being. The actual computer operations corresponding to these terms vary depending on implementation.

### Example 29—Example Cloud Computing Environment

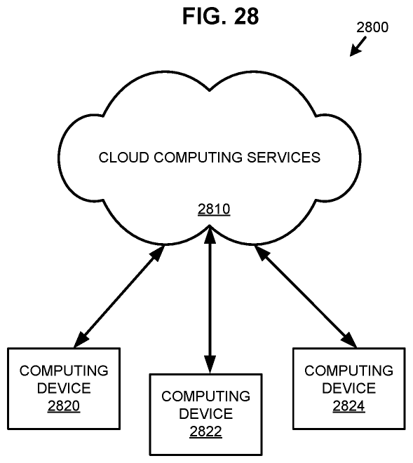

FIG. 28 depicts an example cloud computing environment 2800 in which the described technologies can be implemented, including, e.g., the systems of the drawings described herein. The cloud computing environment 2800 comprises cloud computing services 2810. The cloud computing services 2810 can comprise various types of cloud computing resources, such as computer servers, data storage repositories, networking resources, etc. The cloud computing services 2810 can be centrally located (e.g., provided by a data center of a business or organization) or distributed (e.g., provided by various computing resources located at different locations, such as different data centers and/or located in different cities or countries).

The cloud computing services 2810 are utilized by various types of computing devices (e.g., client computing devices), such as computing devices 2820, 2822, and 2824. For example, the computing devices (e.g., 2820, 2822, and 2824) can be computers (e.g., desktop or laptop computers), mobile devices (e.g., tablet computers or smart phones), or other types of computing devices. For example, the computing devices (e.g., 2820, 2822, and 2824) can utilize the cloud computing services 2810 to perform computing operations (e.g., data processing, data storage, and the like).

In practice, cloud-based, on-premises-based, or hybrid scenarios can be supported.

### Example 30—Example Computer-Readable Media

Any of the computer-readable media herein can be non-transitory (e.g., volatile memory such as DRAM or SRAM, nonvolatile memory such as magnetic storage, optical storage, or the like) and/or tangible. Any of the storing actions described herein can be implemented by storing in one or more computer-readable media (e.g., computer-readable storage media or other tangible media). Any of the things (e.g., data created and used during implementation) described as stored can be stored in one or more computer-readable media (e.g., computer-readable storage media or other tangible media). Computer-readable media can be limited to implementations not consisting of a signal.

### Example 31—Example Implementations

Although the operations of some of the disclosed methods are described in a particular, sequential order for convenient presentation, such manner of description encompasses rearrangement, unless a particular ordering is required by specific language set forth herein. For example, operations described sequentially can in some cases be rearranged or performed concurrently.

### Example 32—Example Computer-Executable Implementation

Any of the methods described herein can be performed by computer-executable instructions (e.g., causing a computing system to perform the method, when executed) stored in one or more computer-readable media (e.g., storage or other tangible media) or stored in one or more computer-readable storage devices. Such methods can be performed in software, firmware, hardware, or combinations thereof. Such methods can be performed at least in part by a computing system (e.g., one or more computing devices).

Such acts of the methods described herein can be implemented by computer-executable instructions in (e.g., stored on, encoded on, or the like) one or more computer-readable media (e.g., computer-readable storage media or other tangible media) or one or more computer-readable storage devices (e.g., memory, magnetic storage, optical storage, or the like). Such instructions can cause a computing device to perform the method. The technologies described herein can be implemented in a variety of programming languages.

In any of the technologies described herein, the illustrated actions can be described from alternative perspectives while still implementing the technologies. For example, “receiving” can also be described as “sending” for a different perspective.

### Example 33—Further Embodiments

Any of the following can be implemented.
Clause 1. A method of identifying a microbe or a virus in a sample, comprising:
   (i) receiving a single cell RNA sequencing dataset for the sample;
   (ii) detecting microbial or viral nucleic acids in the dataset; and
   (iii) identifying the microbe or the virus in the sample when a microbial or viral nucleic acid indicative of the presence of the microbe or the virus is detected in the dataset.
Clause 2. A method of diagnosing a subject with an infectious disease caused by a microbe or a virus, comprising:
   (i) receiving a single cell RNA sequencing dataset for a sample from the subject;
   (ii) detecting microbial or viral nucleic acids in the dataset;
   (iii) identifying the microbe or the virus in the sample when a microbial or viral nucleic acid indicative of the presence of the microbe or the virus is detected in the dataset; thereby diagnosing the subject with the infectious disease.
Clause 3. The method of clause 1, wherein the sample is a sample from a subject.
Clause 4. The method of clause 2 or clause 3, wherein the subject is a subject suspected of having an infectious disease caused by the microbe or the virus.
Clause 5. The method of any one of clauses 1-4, wherein the microbe is a bacterium or a fungus.
Clause 6. A method of identifying biomarkers for diagnosing a cancer in a subject, comprising:
   (i) receiving single cell RNA sequencing datasets for at least two cohorts, wherein at least one cohort comprises one or more cancer subjects and at least one cohort comprises one or more non-cancer subjects;
   (ii) identifying microbial genera using the datasets, wherein the identifying generates at least one microbial genera signature for the one or more cancer subjects and at least one microbial genera signature for the one or more non-cancer subjects; and
   (iii) selecting microbial genera differentially present in the at least one microbial genera signature for the one or more cancer subjects compared to the at least one microbial genera signature for the one or more non-cancer subjects, wherein the selecting generates a differentiating microbial genera signature that distinguishes a cancer subject from a non-cancer subject.
Clause 7. The method of clause 6, further comprising:
   receiving a single cell RNA sequencing dataset for a subject at risk of having the cancer;
   identifying a set of microbial genera in the dataset for the subject at risk of having the cancer; and
   comparing the differentiating microbial genera signature to the set of microbial genera identified in the dataset from the subject at risk of having the cancer;
   thereby determining whether the subject at risk of having the cancer has the cancer.
Clause 8. A method of determining whether a subject at risk of having a cancer has the cancer, comprising:
   receiving a single cell RNA sequencing dataset for a subject at risk of having the cancer;
   identifying a set of microbial genera in the dataset for the subject at risk of having the cancer; and
   comparing a differentiating microbial genera signature to the set of microbial genera identified in the dataset from the subject at risk of having the cancer;
   thereby determining whether the subject at risk of having the cancer has the cancer;
   wherein the differentiating microbial genera signature is generated by:
      (i) receiving single cell RNA sequencing datasets for at least two cohorts, wherein at least one cohort comprises one or more cancer subjects and at least one cohort comprises one or more non-cancer subjects;
      (ii) identifying microbial genera in the datasets, wherein the identifying generates at least one microbial genera signature for the one or more cancer subjects and at least one microbial genera signature for the one or more non-cancer subjects; and
      (iii) selecting microbial genera differentially present in the at least one microbial genera signature for the one or more cancer subjects compared to the at least one microbial genera signature for the one or more non-cancer subjects, wherein the selecting generates a differentiating microbial genera signature that distinguishes a cancer subject from a non-cancer subject.
Clause 9. The method of any one of clauses 6-8, wherein:
   the at least one microbial genera signature for the one or more cancer subjects comprises a signed microbial genera signature and/or an absolute valued microbial genera signature; and
   the at least one microbial genera signature for the one or more cancer subjects comprises a signed microbial genera signature and/or an absolute valued microbial genera signature.
Clause 10. A method of identifying biomarkers for predicting a survival outcome in a cancer subject, comprising:
   (i) receiving single cell RNA sequencing datasets for at least two cohorts, wherein at least one cohort comprises one or more poor survival outcome cancer subjects and at least one cohort comprises one or more good survival outcome cancer subjects;
   (ii) identifying microbial genera in the datasets, wherein the identifying generates at least one microbial genera signature for the one or more good survival outcome cancer subjects and at least one microbial genera signature for the one or more poor survival outcome cancer subjects; and
   (iii) selecting microbial genera differentially present in the at least one microbial genera signature for the one or more good survival outcome cancer subjects compared to the at least one microbial genera signature for the one or more poor survival outcome cancer subjects, wherein the selecting generates a differentiating microbial genera signature that distinguishes a good survival outcome subject from a poor survival outcome subject.
Clause 11. The method of clause 10, further comprising:
   receiving a single cell RNA sequencing dataset for the cancer subject;
   identifying a set of microbial genera in the dataset for the cancer subject; and
   comparing the differentiating microbial genera signature to the set of microbial genera identified in the dataset from the cancer subject;
   thereby predicting whether the cancer subject will have a good survival outcome or a poor survival outcome.
Clause 12. A method of predicting whether a cancer subject will have a good survival outcome or a poor survival outcome, comprising:
   receiving a single cell RNA sequencing dataset for the cancer subject;
   identifying a set of microbial genera in the dataset for the cancer subject; and
   comparing the differentiating microbial genera signature to the set of microbial genera identified in the dataset from the cancer subject;
   thereby predicting whether the cancer subject will have a good survival outcome or a poor survival outcome;
   wherein the differentiating microbial genera signature is generated by:
      (i) receiving single cell RNA sequencing datasets for at least two cohorts, wherein at least one cohort comprises one or more poor survival outcome cancer subjects and at least one cohort comprises one or more good survival outcome cancer subjects;
      (ii) identifying microbial genera in the datasets, wherein the identifying generates at least one microbial genera signature for the one or more good survival outcome cancer subjects and at least one microbial genera signature for the one or more poor survival outcome cancer subjects; and
      (iii) selecting microbial genera differentially present in the at least one microbial genera signature for the one or more good survival outcome cancer subjects compared to the at least one microbial genera signature for the one or more poor survival outcome cancer subjects, wherein the selecting generates a differentiating microbial genera signature that distinguishes a good survival outcome subject from a poor survival outcome subject.
Clause 13. The method of any one of clauses 10-12, wherein:
   the at least one microbial genera signature for the one or more good survival outcome cancer subjects comprises a signed microbial genera signature and/or an absolute valued microbial genera signature; and
   the at least one microbial genera signature for the one or more poor survival outcome cancer subjects comprises a signed microbial genera signature and/or an absolute valued microbial genera signature.
Clause 14. A method of determining T-cell microenvironment reaction in a cancer subject, comprising:
   (i) receiving a single cell RNA sequencing dataset for T-cells from the subject;
   (ii) determining the expression level of one or more of the genes of Table 2 in the T-cells; and
   (iii) comparing the expression level of the one or more genes of Table 2 in the T-cells to a control using a random forest model,
   thereby classifying the individual T-cells as infection microenvironment reactive or tumor microenvironment reactive.
Clause 15. The method of any one of clauses 6-14, wherein selecting microbial genera comprises removing microbial genera from the differentiating microbial genera signature that are not present with a p value of less than 0.05.
Clause 16. The method of any one of clauses 6-15, wherein the at least one microbial genera signature comprises gene expression datapoints.
Clause 17. The method of any one of clauses 6-16, wherein the at least one microbial genera signature comprises genes ranked based on level of differentiation.
Clause 18. The method of any one of clauses 6-17, wherein the datapoints are normalized before identifying differential microbial genera in the datasets.
Clause 19. The method of any one of clauses 6-18, further comprising validating the clinical significance, non-randomness, and/or accuracy of the differentiating microbial genera signature.
Clause 20. The method of clause 19, wherein validating the clinical significance comprises:
   receiving single cell RNA sequencing datasets for a group of validation subjects, wherein whether the subject has the cancer and/or whether the subject has a good or poor survival outcome is known;
   identifying differentially present microbial genera in the datasets, wherein the identifying generates at least one single-sample signature for each validation subject in the group;
   determining the presence of microbial genera from the differentiating microbial genera signature in the at least one single-sample signature for each validation subject in the group, wherein the determining generates a microbial genera signature for each validation subject;
   clustering the validation subjects in the group into cancer status clusters and/or survival outcome clusters based on the microbial genera signature for each validation subject; and
   comparing the cancer status clusters with the known cancer status for the validation subjects in the group; and/or
   comparing the survival outcome clusters with the known survival outcome for the validation subjects in the group.
Clause 21. The method of clause 20, wherein comparing the cancer status clusters with the known cancer statuses comprises statistically analyzing the two clusters for a difference in the known cancer status.
Clause 22. The method of clause 20, wherein comparing the survival outcome clusters with the known survival outcome comprises statistically analyzing the two clusters for a difference in the known survival outcome.
Clause 23. The method of clause 21 wherein the two clusters show a difference in the known cancer status with a p value of less than 0.05.
Clause 24. The method of clause 22, wherein the two clusters show a difference in the known survival outcome with a p value of less than 0.05.
Clause 25. The method of any one of clauses 20-24, wherein generating at least one single-sample signature for each validation subject in the group comprises generating a signed single-sample signature and/or an absolute valued single-sample signature.
Clause 26. A method of identifying biomarkers for diagnosing cancer in a subject, comprising:
   (i) receiving single cell RNA sequencing datasets for at least two cohorts, wherein at least one cohort comprises one or more cancer subjects and at least one cohort comprises one or more non-cancer subjects;
   (ii) identifying microbial genera in the datasets, wherein the identifying generates at least one microbial genera signature for the one or more cancer subjects and at least one microbial genera signature for the one or more non-cancer subjects;
   (iii) selecting microbial genera differentially present in the at least one microbial genera signature for the one or more cancer subjects compared to the at least one microbial genera signature for the one or more non-cancer subjects, wherein the selecting generates a differentiating microbial genera signature that distinguishes a cancer subject from a non-cancer subject;
   (iv) receiving a single cell RNA sequencing dataset for a subject at risk of having a cancer;
   (v) identifying a set of microbial genera in the dataset for the subject at risk of having the cancer; and
   (vi) comparing the differentiating microbial genera signature to the set of microbial genera identified in the dataset from the subject at risk of having the cancer;
   thereby determining whether the subject at risk of having the cancer has the cancer.
Clause 27. A method of identifying biomarkers for predicting a survival outcome in a cancer subject, comprising:
   (i) receiving single cell RNA sequencing datasets for at least two cohorts, wherein at least one cohort comprises one or more poor survival outcome cancer subjects and at least one cohort comprises one or more good survival outcome cancer subjects;
   (ii) identifying microbial genera in the datasets, wherein the identifying generates at least one microbial genera signature for the one or more good survival outcome cancer subjects and at least one microbial genera signature for the one or more poor survival outcome cancer subjects;
   (iii) selecting microbial genera differentially present in the at least one microbial genera signature for the one or more good survival outcome cancer subjects compared to the at least one microbial genera signature for the one or more poor survival outcome cancer subjects, wherein the selecting generates a differentiating microbial genera signature that distinguishes a good survival outcome subject from a poor survival outcome subject;
   (iv) receiving a single cell RNA sequencing dataset for the cancer subject;
   (v) identifying a set of microbial genera in the dataset for the cancer subject; and
   (vi) comparing the differentiating microbial genera signature to the set of microbial genera identified in the dataset from the cancer subject;
   thereby predicting whether the cancer subject will have a good survival outcome or a poor survival outcome.
Clause 28. The method of any one of clauses 6-27, wherein the cancer is a pancreatic cancer.
Clause 29. The method of any one of clauses 1-28, wherein the identifying microbial genera in the datasets or the detecting microbial or viral nucleic acids in the dataset further comprises:
   (i) mapping reads from the single cell RNA sequencing dataset to microbial and/or viral genomes using a metagenomics classifier, thereby assigning a genus and/or species identity to each read in the dataset;
   (ii) for each genus and/or species identified in (i):
      (a) comparing the number of reads assigned and the number of minimizers assigned;
      (b) comparing the number of minimizers assigned and the number of unique minimizers assigned; and
      (c) comparing the number of reads assigned and the number of unique minimizers assigned; and
   (iii) classifying the genus and/or species as a true positive result when a correlation value for each comparison in (ii)(a)-(ii)(c) is positive, and when a number of reads detected for the species is greater in the single cell RNA sequencing dataset as compared to a control.
Clause 30. The method of clause 29, wherein the correlation value for each comparison is greater than 0.5.
Clause 31. The method of clause 29, wherein the correlation value for each comparison is greater than 0.7.
Clause 32. The method of clause 29, wherein the correlation value for each comparison is greater than 0.9.
Clause 33. The method of clause 29, wherein the correlation value for each comparison is greater than 0.95.
Clause 34. The method of clause 29, wherein the correlation value is determined using a Spearman correlation.
Clause 35. The method of any one of clauses 1-34, wherein the control is a sample from a subject or a group of subjects that does not have the cancer or the infection, or a sample from at least one cell line that does not have the cancer or the infection.
Clause 36. A microbe or a virus identification system, comprising:
   one or more processors; and
   memory coupled to the one or more processors, wherein the memory comprises computer-executable instructions causing the one or more processors to perform a process comprising:
   (i) receiving a single cell RNA sequencing dataset for the sample;
   (ii) detecting microbial or viral nucleic acids in the dataset; and
   (iii) identifying the microbe or the virus in the sample when a microbial or viral nucleic acid indicative of the presence of the microbe or virus is detected in the dataset.
Clause 37. One or more computer-readable media having encoded thereon computer-executable instructions that, when executed, cause a computing system to perform a microbe or a virus identification method comprising:
   (i) receiving a single cell RNA sequencing dataset for the sample;
   (ii) detecting microbial or viral nucleic acids in the dataset; and
   (iii) identifying the microbe or the virus in the sample when a microbial or viral nucleic acid indicative of the presence of the microbe or the virus is detected in the dataset.
Clause 38. An infectious disease diagnosis system, comprising:
   one or more processors; and
   memory coupled to the one or more processors, wherein the memory comprises computer-executable instructions causing the one or more processors to perform a process comprising:
      (i) receiving a single cell RNA sequencing dataset for the subject;
      (ii) detecting microbial or viral nucleic acids in the dataset;
      (iii) identifying a microbe or a virus in the sample when a microbial or viral nucleic acid indicative of the presence of the microbe or the virus is detected in the dataset, wherein the microbe or the virus is a causative agent of the infectious disease;
   thereby diagnosing the subject with the infectious disease.
Clause 39. One or more computer-readable media having encoded thereon computer-executable instructions that, when executed, cause a computing system to perform an infectious disease diagnosis method comprising:
   (i) receiving a single cell RNA sequencing dataset for the subject;
   (ii) detecting microbial or viral nucleic acids in the dataset;
   (iii) identifying a microbe or a virus in the sample when a microbial or viral nucleic acid indicative of the presence of the microbe or the virus is detected in the dataset, wherein the microbe or the virus is a causative agent of the infectious disease;
   thereby diagnosing the subject with the infectious disease.
Clause 40. The system of clause 36 or clause 38, or the computer readable media of clause 37 or clause 39, wherein the detecting microbial or viral nucleic acids in the dataset further comprises:
   (i) mapping reads from the single cell RNA sequencing dataset to microbial and/or viral genomes using a metagenomics classifier, thereby assigning a genus and/or species identity to each read in the dataset;
   (ii) for each genus and/or species identified in (i):
      (a) comparing the number of reads assigned and the number of minimizers assigned; (b) comparing the number of minimizers assigned and the number of unique minimizers assigned; and
      (c) comparing the number of reads assigned and the number of unique minimizers assigned; and
   (iii) classifying the genus and/or species as a true positive result when a correlation value for each comparison in (ii)(a)-(ii)(c) is positive, and when a number of reads detected for the species is greater in the single cell RNA sequencing dataset as compared to a control.
Clause 41. A cancer diagnosing biomarker identification system, comprising:
   one or more processors; and
   memory coupled to the one or more processors, wherein the memory comprises computer-executable instructions causing the one or more processors to perform a process comprising:
      (i) receiving single cell RNA sequencing datasets for at least two cohorts, wherein at least one cohort comprises one or more cancer subjects and at least one cohort comprises one or more non-cancer subjects;
      (ii) identifying microbial genera in the datasets, wherein the identifying generates at least one microbial genera signature for the one or more cancer subjects and at least one microbial genera signature for the one or more non-cancer subjects;
      (iii) selecting microbial genera differentially present in the at least one microbial genera signature for the one or more cancer subjects compared to the at least one microbial genera signature for the one or more non-cancer subjects, wherein the selecting generates a differentiating microbial genera signature that distinguishes a cancer subject from a non-pancreatic cancer subject;
      (iv) receiving a single cell RNA sequencing dataset for a subject at risk of having a cancer.
Clause 42. One or more computer-readable media having encoded thereon computer-executable instructions that, when executed, cause a computing system to perform a cancer diagnosing biomarker identification method comprising:
   (i) receiving single cell RNA sequencing datasets for at least two cohorts, wherein at least one cohort comprises one or more cancer subjects and at least one cohort comprises one or more non-cancer subjects;
   (ii) identifying microbial genera in the datasets, wherein the identifying generates at least one microbial genera signature for the one or more cancer subjects and at least one microbial genera signature for the one or more cancer subjects;
   (iii) selecting microbial genera differentially present in the at least one microbial genera signature for the one or more cancer subjects compared to the at least one microbial genera signature for the one or more non-cancer subjects, wherein the selecting generates a differentiating microbial genera signature that distinguishes a cancer subject from a non-cancer subject.
Clause 43. A whether a subject at risk of having a cancer has the cancer determination system, comprising:
   one or more processors; and
   memory coupled to the one or more processors, wherein the memory comprises computer-executable instructions causing the one or more processors to perform a process comprising:
   receiving a single cell RNA sequencing dataset for a subject at risk of having the cancer;
   identifying a set of microbial genera in the dataset for the subject at risk of having the cancer; and
   comparing a differentiating microbial genera signature to the set of microbial genera identified in the dataset from the subject at risk of having the cancer;
   thereby determining whether the subject at risk of having the cancer has the cancer;
   wherein the differentiating microbial genera signature is generated by:
      (i) receiving single cell RNA sequencing datasets for at least two cohorts, wherein at least one cohort comprises one or more cancer subjects and at least one cohort comprises one or more non-cancer subjects;
      (ii) identifying microbial genera in the datasets, wherein the identifying generates at least one microbial genera signature for the one or more cancer subjects and at least one microbial genera signature for the one or more non-cancer subjects; and
      (iii) selecting microbial genera differentially present in the at least one microbial genera signature for the one or more cancer subjects compared to the at least one microbial genera signature for the one or more non-cancer subjects, wherein the selecting generates a differentiating microbial genera signature that distinguishes a cancer subject from a non-cancer subject.
Clause 44. One or more computer-readable media having encoded thereon computer-executable instructions that, when executed, cause a computing system to perform a whether a subject at risk of having a cancer has the cancer determination method comprising:
   receiving a single cell RNA sequencing dataset for a subject at risk of having the cancer;
   identifying a set of microbial genera in the dataset for the subject at risk of having the cancer; and
   comparing a differentiating microbial genera signature to the set of microbial genera identified in the dataset from the subject at risk of having the cancer;
   thereby determining whether the subject at risk of having the cancer has the cancer;
   wherein the differentiating microbial genera signature is generated by:
      (i) receiving single cell RNA sequencing datasets for at least two cohorts, wherein at least one cohort comprises one or more cancer subjects and at least one cohort comprises one or more non-cancer subjects;
      (ii) identifying microbial genera in the datasets, wherein the identifying generates at least one microbial genera signature for the one or more cancer subjects and at least one microbial genera signature for the one or more non-cancer subjects; and
      (iii) selecting microbial genera differentially present in the at least one microbial genera signature for the one or more cancer subjects compared to the at least one microbial genera signature for the one or more non-cancer subjects, wherein the selecting generates a differentiating microbial genera signature that distinguishes a cancer subject from a non-cancer subject.
Clause 45. A cancer survival outcome biomarker identification system, comprising:
   one or more processors; and
   memory coupled to the one or more processors, wherein the memory comprises computer-executable instructions causing the one or more processors to perform a process comprising:
      (i) receiving single cell RNA sequencing datasets for at least two cohorts, wherein at least one cohort comprises one or more poor survival outcome cancer subjects and at least one cohort comprises one or more good survival outcome cancer subjects;
      (ii) identifying microbial genera in the datasets, wherein the identifying generates at least one microbial genera signature for the one or more good survival outcome cancer subjects and at least one microbial genera signature for the one or more poor survival outcome cancer subjects; and
      (iii) selecting microbial genera differentially present in the at least one microbial genera signature for the one or more good survival outcome cancer subjects compared to the at least one microbial genera signature for the one or more poor survival outcome cancer subjects, wherein the selecting generates a differentiating microbial genera signature that distinguishes a good survival outcome subject from a poor survival outcome subject.
Clause 46. One or more computer-readable media having encoded thereon computer-executable instructions that, when executed, cause a computing system to perform a cancer survival outcome biomarker identification method comprising:
   (i) receiving single cell RNA sequencing datasets for at least two cohorts, wherein at least one cohort comprises one or more poor survival outcome cancer subjects and at least one cohort comprises one or more good survival outcome cancer subjects;
   (ii) identifying microbial genera in the datasets, wherein the identifying generates at least one microbial genera signature for the one or more good survival outcome cancer subjects and at least one microbial genera signature for the one or more poor survival outcome cancer subjects;
   (iii) selecting microbial genera differentially present in the at least one microbial genera signature for the one or more good survival outcome cancer subjects compared to the at least one microbial genera signature for the one or more poor survival outcome cancer subjects, wherein the selecting generates a differentiating microbial genera signature that distinguishes a good survival outcome subject from a poor survival outcome subject.
Clause 47. A whether a cancer subject will have a good survival outcome or a poor survival outcome determination system, comprising:
   one or more processors; and
   memory coupled to the one or more processors, wherein the memory comprises computer-executable instructions causing the one or more processors to perform a process comprising:
   receiving a single cell RNA sequencing dataset for the cancer subject;
   identifying a set of microbial genera in the dataset for the cancer subject; and
   comparing the differentiating microbial genera signature to the set of microbial genera identified in the dataset from the cancer subject;
   thereby predicting whether the cancer subject will have a good survival outcome or a poor survival outcome;
   wherein the differentiating microbial genera signature is generated by:
      (i) receiving single cell RNA sequencing datasets for at least two cohorts, wherein at least one cohort comprises one or more poor survival outcome cancer subjects and at least one cohort comprises one or more good survival outcome cancer subjects;
      (ii) identifying microbial genera in the datasets, wherein the identifying generates at least one microbial genera signature for the one or more good survival outcome cancer subjects and at least one microbial genera signature for the one or more poor survival outcome cancer subjects; and
      (iii) selecting microbial genera differentially present in the at least one microbial genera signature for the one or more good survival outcome cancer subjects compared to the at least one microbial genera signature for the one or more poor survival outcome cancer subjects, wherein the selecting generates a differentiating microbial genera signature that distinguishes a good survival outcome subject from a poor survival outcome subject.
Clause 48. One or more computer-readable media having encoded thereon computer-executable instructions that, when executed, cause a computing system to perform a whether a cancer subject will have a good survival outcome or a poor survival outcome determination method comprising:
   receiving a single cell RNA sequencing dataset for the cancer subject;
   identifying a set of microbial genera in the dataset for the cancer subject; and
   comparing the differentiating microbial genera signature to the set of microbial genera identified in the dataset from the cancer subject;
   thereby predicting whether the cancer subject will have a good survival outcome or a poor survival outcome;
   wherein the differentiating microbial genera signature is generated by:
      (i) receiving single cell RNA sequencing datasets for at least two cohorts, wherein at least one cohort comprises one or more poor survival outcome cancer subjects and at least one cohort comprises one or more good survival outcome cancer subjects;
      (ii) identifying microbial genera in the datasets, wherein the identifying generates at least one microbial genera signature for the one or more good survival outcome cancer subjects and at least one microbial genera signature for the one or more poor survival outcome cancer subjects; and
      (iii) selecting microbial genera differentially present in the at least one microbial genera signature for the one or more good survival outcome cancer subjects compared to the at least one microbial genera signature for the one or more poor survival outcome cancer subjects, wherein the selecting generates a differentiating microbial genera signature that distinguishes a good survival outcome subject from a poor survival outcome subject.
Clause 49. The system of any one of clauses 41, 43, 45, or 47, or the computer readable media of any one of clauses 42, 44, 46, or 48, wherein the identifying microbial genera in the datasets further comprises:
   (i) mapping reads from the single cell RNA sequencing dataset to microbial and/or viral genomes using a metagenomics classifier, thereby assigning a genus and/or species identity to each read in the dataset;
   (ii) for each genus and/or species identified in (i):
      (a) comparing the number of reads assigned and the number of minimizers assigned; (b) comparing the number of minimizers assigned and the number of unique minimizers assigned; and
      (c) comparing the number of reads assigned and the number of unique minimizers assigned; and
   (iii) classifying the genus and/or species as a true positive result when a correlation value for each comparison in (ii)(a)-(ii)(c) is positive, and when a number of reads detected for the species is greater in the single cell RNA sequencing dataset as compared to a control.
Clause 50. A T-cell microenvironment reaction determination system, comprising:
   (i) receiving a single cell RNA sequencing dataset for T-cells from the subject;
   (ii) determining the expression level of one or more of the genes of Table 2 in the T-cells; and
   (iii) comparing the expression level of the one or more genes of Table 2 in the T-cells to a control using a random forest model,
   thereby classifying the individual T-cells as infection microenvironment reactive or tumor microenvironment reactive.
Clause 51. One or more computer-readable media having encoded thereon computer-executable instructions that, when executed, cause a computing system to perform a T-cell microenvironment reaction determination method comprising:
   (i) receiving a single cell RNA sequencing dataset for T-cells from the subject;
   (ii) determining the expression level of one or more of the genes of Table 2 in the T-cells; and
   (iii) comparing the expression level of the one or more genes of Table 2 in the T-cells to a control using a random forest model,
thereby classifying the individual T-cells as infection microenvironment reactive or tumor microenvironment reactive.
Clause 52. A system comprising:
   one or more processors; and
   memory coupled to the one or more processors;
   wherein the memory comprises computer-executable instructions causing the one or more processors to perform the method of any of clauses 1-35 Clause 53. One or more computer-readable media having encoded thereon computer-executable instructions that when executed cause a computing system to perform the method of any of clauses 1-35.

### Example 34—Example Alternatives

The technologies from any example can be combined with the technologies described in any one or more of the other examples. In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

APPENDIX I

```
#### Example script for processing the output of Kraken on single-cell RNA seq fastq files to produce a
      table of barcode, UMI, and counts
library(optparse)
library(stringr)
library(ShortRead)
library(dplyr)
library(Matrix)
library(taxizedb)
library(data.table)
#source(‘/home/bcg68/taxonomy_functions.r’)
option_list = list(
  make_option(c(“--dataPath”), action=“store”, help = “directory must end in a backslash”),
  make_option(c(“--sampleName”), action=“store”, help = “sample name”),
  make_option(c(“--bcStart”), action=“store”, default = 1, help = “starting index of cell barcode”),
  make_option(c(“--bcEnd”), action=“store”, default = 16, help = “ending inedex of cell barcode”),
  make_option(c(“--umiStart”), action=“store”, default = 17, help = “starting index of UMI barcode”),
  make_option(c(“--umiEnd”), action=“store”, default = 26, help = “ending index of UMI barcode”),
  make_option(c(“--movingAverage”), action=“store”, default = 5, help = “window for sliding avgerage”),
  make_option(c(“--outputPath”), action=“store”, default = NA, help = “must end in backslash”),
  make_option(c(“--nFilter”), action=“store”, default = 130, help = “filter reads with >n of one nucleotide”)
)
opt = parse_args(OptionParser(option_list = option_list))
if(is.na(opt$outputPath)){ opt$outputPath = opt$dataPath}
# get barcodes, umis, and tax-ids
print(paste(‘Started extracting barcode data from fastq files for’, opt$sampleName))
# bc = list( )
# for(i in 1:2){
  # reads = readFastq(paste0(opt$dataPath, opt$sampleName, ‘_’, i, ‘.fq’))
  reads = readFastq(paste0(opt$dataPath, opt$sampleName, ‘_1.fq’))
  # Removes reads with >=20 of one nucleotide
  filter <- polynFilter(threshold=opt$nFilter, nuc=c(“A”,“T”,“G”,“C”) %>% compose( )
  reads = reads [filter(reads)]
  sequences = sread(reads)
  headers = ShortRead::id(reads)
  barcode = substr(sequences, opt$bcStart, opt$bcEnd)
  umi = substr(sequences, opt$umiStart, opt$umiEnd)
  taxid = gsub(‘.* taxid\\l’, ″, headers)
  # bc[[i] = cbind(barcode, umi, taxid)
  bc = cbind(barcode, umi, taxid)
# }
# s.bc = rbind(bc[1]], bc[2]]) %>% unique( ) %>% data.frame( )
s.bc = bc %>% unique( ) %>% data.frame( )
s.bc$umi = 1
s.bc = s.bc %>% group_by(barcode, taxid) %>% summarize(umi = sum(umi)) %>% arrange(desc(umi))
rm(bc)
write.table(s.bc, file = paste0(opt$outputPath, opt$sampleName, ‘.all.barcodes.txt’),
      quote = F, sep=‘\t’, row.names = F, col.names = T)
print(paste(‘Finished extracting barcode data from fastq files for’, opt$sampleName))
# create full sparse matrix
s.mat = sparseMatrix(as.integer(s.bc$barcode), as.integer(s.bc$taxid), x=s.bc$umi)
colnames(s.mat) = levels(s.bc$taxid)
rownames(s.mat) = levels(s.bc$barcode)
s.mat = t(s.mat)
# remove empty barcodes
moving.average <- function(x, n = opt$movingAverage){stats::filter(x, rep(1 / n, n), sides = 2)}
bc.depth = colSums(s.mat) %>% sort(decreasing = T)
slope = bc.depth %>% moving.average(n = opt$movingAverage) %>% diff(na.rm = T)
n_bc = which(abs(slope) < 10\hat{ }-3)[1]
s.mat = s.mat[, names(bc.depth)[1:n_bc]]
ind = which(rowSums(s.mat) == 0)
if(length(ind)>0){s.mat = s.mat[-ind, ]}
print(paste(‘Started classifying reads for’, opt$sampleName))
# count parent classifications for each read
df = list( )
ncbi_db = src_ncbi( )
counter = 0
for(i in 1:nrow(s.mat)){
  tax = tryCatch(
    {ncbi_classification(ncbi_db, rownames(s.mat)[i])[1]]},
    error = function(e){
    closeAllConnections( )
    ncbi_classification(ncbi_db, rownames(s.mat)[i])[1]]
  }
)
tax = ncbi_classification(ncbi_db, rownames(s.mat)[i])[[1]]
if(is.na(tax)){next}
tax = tax[str_which(tax$rank, ‘superkingdom|\hat{ }phylum|\hat{ }class|\hat{ }order|\hat{ }family|\hat{ }genus|\hat{ }species’),]
row = s.mat[i,]
row = row[row>0]
for(j in 1:nrow(tax)){
  counter = counter + 1
  df[counter] = tibble(barcode = names(row), counts = row,
                        taxid = tax$id[j], rank = tax$rank[j], name = tax$name[j])
  }
}
df = rbindlist(df, use.names = T)
df$name = str_replace_all(df$name,“\\s+”, “-”)
df = df %>% group_by(barcode, taxid, rank, name) %>% summarize(counts = sum(counts) %>%
            arrange(desc(counts))
# kingdom = df[frank = ‘superkingdom’,] %>% arrange(desc(counts))
# phylum = df[df$rank = ‘phylum’,] %>% arrange(desc(counts))
# class = df[df$rank == ‘class’,] %>% arrange(desc(counts))
# order = df[dfrank = ‘order’,] %>% arrange(desc(counts))
# family = df[df$rank = ‘family’,] %>% arrange(desc(counts))
# genus = df[df$rank == ‘genus’,] %>% arrange(desc(counts))
# species = df[df$rank = ‘species’,] %>% arrange(desc(counts))
# save
write.table(df, file = paste0(opt$outputPath, opt$sampleName, ‘.counts.txt’),
            quote = F, sep=‘\t’, row.names = F, col.names = T)
print(‘Finished’)
```

APPENDIX II

```
### Example script identifying a six-gene microbial diversity and survival signature
# load data
### BACTERIAL BARCODES MERGED
f = list.files(‘/scratch/bcg68/DTC_datasets/PDAC/kraken/’, full.names = T)
f = f[str_which(f,‘.counts.txt’)]
# f = f[str_which(f, ‘all’, negate = T)]
sample.name = c(paste0(‘T’, 1:24), paste0(‘N’, 1:11))
b.list = list( )
for(i in 1:length(f)){
  print(i)
  mat = read.delim(f[i])
  mat = mat[mat$rank==‘genus’,]; mat = droplevels(mat)
  y = sparseMatrix(as.integer(as.factor(mat$barcode)), as.integer(as.factor(mat$name)), x = mat$counts)
  colnames(y) = levels(as.factor(mat$name))
  rownames(y) = paste0(sample.name[i], ‘_’, levels(as.factor(mat$barcode)))
  b.list[i] = CreateSeuratObject(t(y))
}
# Add metadata
type = c(rep(‘T’,24), rep(‘N’, 11))
sample.name = c(paste0(‘T’, 1:24), paste0(‘N’, 1:11))
for(i in 1:length(b.list)){
  b.list[i] = AddMetaData(b.list[i], type[i], col.name = ‘Type’)
  b.list[i]] = AddMetaData(b.list[i], sample.name[i], col.name = ‘Sample’)
}
# merge and cluster
bacteria.seurat = merge(x = b.list[[1]], y = b.list[2:length(b.list)])
### FUNGAL BARCODES MERGED
f = list.files(‘/scratch/bcg68/DTC_datasets/PDAC/kraken/fungi’, full.names = T)
f = f[str_which(f,‘.counts.txt’)]
sample.name = c(paste0(‘T’, 1:24), paste0(‘N’, 1:11))
f.list = list( )
for(i in 1:length(f)){
  print(i)
  mat = read.delim(f[i])
  mat = mat[mat$rank==‘genus’,]; mat = droplevels(mat)
  y = sparseMatrix(as.integer(as.factor(mat$barcode)), as.integer(as.factor(mat$name)), x = mat$counts)
  colnames(y) = levels(as.factor(mat$name))
  rownames(y) = levels(as.factor(mat$barcode))
  rownames(y) = paste0(sample.name[i], ‘_’, levels(as.factor(mat$barcode)))
  f.list[i] = CreateSeuratObject(t(y))
}
# Add metadata
type = c(rep(‘T’,24), rep(‘N’, 11))
sample.name = c(paste0(‘T’, 1:24), paste0(‘N’, 1:11))
for(i in 1:length(f.list)){
  f.list[i] = AddMetaData(f.list[i]], type[i], col.name = ‘Type’)
  f.list[i] = AddMetaData(f.list[i], sample.name[i], col.name = ‘Sample’)
}
# merge and cluster
fungi.seurat = merge(x = f.list[1], y = f.list[2:length(f.list)])
# load peng
peng = lapply(b.list, function(x) tibble(sample = unique(x$Sample),
                    genus = rownames(x),
                    counts = rowSums(x@assays$RNA@counts))) %>%
  rbindlist( ) %>%
  pivot_wider(id_cols = sample, names_from = genus, values_from = counts, values_fill = list(counts=0))
        %>%
  column_to_rownames(‘sample’)
peng = colSums(peng)/sum(peng)
peng = peng[-which(peng<10\hat{ }-4)]
# load muraro
f = list.files(‘/scratch/bcg68/datasets/pancreas-murano/kraken/’, full.names = T)
f = f[str_which(f, ‘counts.txt’)]
muraro.list = list( )
for(i in 1: length(f)){
  mat = read.delim(f[i])
  mat = mat[mat$rank==‘genus’,]; mat = droplevels(mat)
  y = sparseMatrix(as.integer(as.factor(mat$barcode)), as.integer(as.factor(mat$name)), x = mat$counts)
  colnames(y) = levels(as.factor(mat$name))
  rownames(y) = levels(as.factor(mat$barcode))
  muraro.list[[i]] = CreateSeuratObject(t(y))
  muraro.list[i]$Sample = paste0(‘muraro’,i)
}
muraro = lapply(muraro.list, function(x) tibble(sample = unique(x$Sample),
                        genus = rownames(x),
                        counts = rowSums(x@assays$RNA@counts) %>%
  rbindlist( ) %>%
  pivot_wider(id_cols = sample, names_from = genus, values_from = counts, values_fill = list(counts=0))
      %>%
  column_to_rownames(‘sample’)
muraro = colSums(muraro)/sum(muraro)
muraro = muraro[-which(muraro<10\hat{ }-4)]
# load baron
f = list.files(‘/scratch/bcg68/datasets/pancreas-baron/kraken/’, full.names = T)
f = f[str_which(f, ‘counts.txt’)]
baron.list = list( )
for(i in 1:length(f)){
  mat = read.delim(f[i])
  mat = mat[mat$rank==‘genus’,]; mat = droplevels(mat)
  y = sparseMatrix(as.integer(as.factor(mat$barcode)), as.integer(as.factor(mat$name)), x = mat$counts)
  colnames(y) = levels(as.factor(mat$name))
  rownames(y) = levels(as.factor(mat$barcode))
  baron.list[i] = CreateSeuratObject(t(y))
  baron.list[i]$Sample = paste0(‘Baron’,i)
}
baron = lapply(baron.list, function(x) tibble(sample = unique(x$Sample),
                        genus = rownames(x),
                        counts = rowSums(x@assays$RNA@counts) %>%
  rbindlist( ) %>%
  pivot_wider(id_cols = sample, names_from = genus, values_from = counts, values_fill = list(counts=0))
      %>%
  column_to_rownames(‘sample’)
baron = colSums(baron)/sum(baron)
baron = baron [-which(baron<10\hat{ }-4)]
# load decontaminated TCGA data
meta = read.csv(‘/scratch/bcg68/DTC_datasets/PDAC/other/Metadata-TCGA-All-18116-Samples.csv’,
      row.names = 1)
meta = meta[meta$disease_type == ‘Pancreatic Adenocarcinoma’,]
tcga = read.csv(‘/scratch/bcg68/DTC_datasets/PDAC/other/Kraken-TCGA-Voom-SNM-All-Putative-
      Contaminants-Removed-Data.csv’, row.names = 1)
tcga = tcga[rownames(meta), ]
tcga = tcga[, str_which(colnames(tcga), ‘k_Bacteria’)]
colnames(tcga) = sub(“.*_”, “”, colnames(tcga))
tcga.freq = colSums(tcga)/sum(tcga)
tcga.freq = tcga.freq[-which(tcga.freq<10\hat{ }-4)]
# load decontaminated Nejman data; get genera that passed all filters exept multi-study
science.decont = read_xlsx(‘/scratch/bcg68/DTC_datasets/PDAC/other/aay9189_TableS4.xlsx’, sheet =
      ‘All_filters’)
x = science.decont[, c(7, 42)]
x = x[which(x[,2] == 1),1] %>% unique( )
decont.genus = x$\grave{ }...7\grave{ }; decont.genus = decont.genus[-str_which(decont.genus, ‘Unknown’)]
decont.genus = decont.genus[-which(is.na(decont.genus))]; decont.genus = sort(decont.genus)
# by genus
science = read_xlsx(‘/scratch/bcg68/DTC_datasets/PDAC/other/aay9189_TableS2.xlsx’)
x=science
x1=x[29:nrow(x), 4:9]
x2=x[29:nrow(x), str_which(x[2,], ‘Pancreas’)]
x3=cbind(x1,x2)
colnames(x3) = x3[1,]; x3 = x3[−1,]
x3 = na.omit(x3)
x=apply(x3[, 7:ncol(x3)], 2, as.numeric) %>% rowMeans( )
x3 = data.frame(x3[,1:6], counts = x)
nejman = tapply(x3$counts, x3$genus, FUN=sum)
nejman = nejman[decont.genus]
nejman = nejman/sum(nejman)
# combine and remove genera present in <2 studies
combined.mat = bind_rows(peng, baron, muraro, toga.freq, nejman) %>% data.frame( )
rownames(combined.mat) = c(‘Peng’, ‘Baron’, ‘Muraro’, ‘Poore’, ‘Nejman’)
mat = combined.mat; mat[is.na(mat)] = 0;
genus.keep = apply(mat, 2, nnzero)
combined.mat = combined.mat[, genus.keep > 1]
# combine bacteria and fungi into one object and get associated cell types
b.mat = bacteria.seurat@assays$RNA@counts %>% data.frame( )
f.mat = fungi.seurat@assays$RNA@counts %>% data.frame( )
combined.seurat = bind_rows(b.mat, f.mat); combined.seurat[is.na(combined.seurat)] = 0
b.keep = colnames(combined.mat)[which(combined.mat[1] > 0)] %>% str_replace(‘[.]’, ‘-’)
f.keep = rownames(fungi.seurat)
combined.seurat = combined.seurat[c(b.keep, f.keep), ]
combined.seurat = CreateSeuratObject(combined.seurat)
type = colnames(combined.seurat); type = substr(type,1,1)
combined.seurat$Type = type
combined.seurat$Sample = gsub(‘_.*’,″, colnames(combined.seurat))
# SHANNON DIVERSITY of microbiome in Peng samples
m.abun = combined.seurat@assays$RNA@counts %>% t( ) %>% data.frame( )
m.abun$Sample = combined.seurat$Sample
m.abun = m.abun %>%
  pivot_longer(-c(Sample), names_to =‘Genus’, values_to =‘Counts’) %>%
  group_by(Sample, Genus) %>%
  summarize(Counts = sum(Counts) %>%
  pivot_wider(id_cols = Sample, values_from = Counts, names_from = Genus, values_fill = list(Counts=0))
      %>%
  column_to_rownames(‘Sample’)
shannon = vegan::diversity(m.abun, index=‘shannon’)
# load TCGA and ICGC PDAC profiles
tcga.rna = read.table(‘/Users/bassel/Documents/CINJ/Metagenomics/TCGA_PAAD_RNA.txt’)
icgc = read.table(‘/Users/bassel/Documents/CINJ/Metagenomics/icgc.paad.txt’, header = T, row.names = 1)
## DEG between samples with low vs. high shannon diveristy
ref = read.table(‘ref2.txt’) # somatic scRNAseq for Peng samples
bc.samples = gsub(‘_.*’, ″, colnames(ref))
samples = bc.samples %>% unique( )
ref.bulk = c( )
for(i in 1:length(samples)){
  ref.bulk = rbind(ref.bulk, ref[, bc.samples %in% samples[i] %>% rowSums( ))
}
rownames(ref.bulk) = samples
ref.bulk2 = ref.bulk[, intersect(colnames(ref.bulk), intersect(rownames(tcga.rna), rownames(icgc)))]
ref.bulk2 = apply(ref.bulk2, 1, rank) %>% t( )
shannon = shannon [rownames(ref.bulk)]
ind = which(shannon > mean(shannon))
p = apply(ref.bulk2, 2, function(x) wilcox.test(x[ind], x[−ind])$p.value)
ref.bulk = ref.bulk[, which(p < 0.01) %>% names( )]
ref.bulk = apply(ref.bulk, 1, rank) %>% t( ) %>% data.frame( )
ref.bulk$type = ifelse(shannon > mean(shannon), ‘High’, ‘Low’); ref.bulk$type = factor(ref.bulk$type)
# model diversity in peng samples
set.seed(1)
fit = cv.glmnet(as.matrix(ref.bulk[1:(ncol(ref.bulk)-1)]), ref.bulk$type, alpha = 1,
        lambda = 10\hat{ }seq(−0.5, −3, by = −.1), family =‘binomial’)
pred = predict(fit, as.matrix(ref.bulk[1:(ncol(ref.bulk)-1)]), type = ‘class’, s = ‘lambda.min’)
mean(pred == ref.bulk$type)
table(pred, ref.bulk$type)
fit
coef(fit, s = ‘lambda.min’)
```

1. A method of identifying a microbe or a virus in a sample, comprising:
(i) receiving a single cell RNA sequencing dataset for the sample;
(ii) detecting microbial or viral nucleic acids in the dataset; and
(iii) identifying the microbe or the virus in the sample when a microbial or viral nucleic acid indicative of the presence of the microbe or the virus is detected in the dataset.

2-5. (canceled)

6. A method of identifying biomarkers for diagnosing a cancer in a subject, or predicting a survival outcome in a cancer subject, comprising:
(i) receiving single cell RNA sequencing datasets for at least two cohorts, wherein at least one cohort comprises one or more first subjects and at least one cohort comprises one or more second subjects;
(ii) identifying microbial genera using the datasets, wherein the identifying generates at least one microbial genera signature for the first subjects and at least one microbial genera signature for the second subjects; and
(iii) selecting microbial genera differentially present in the at least one microbial genera signature for the one or more first subjects compared to the at least one microbial genera signature for the one or more second subjects, wherein the selecting generates a differentiating microbial genera signature that distinguishes a first subject from a second subject: wherein
the first subject is a cancer subject, and the second subject is a non-cancer subject; or
the first subject is a good survival outcome cancer subject, and the second subject is a poor survival outcome cancer subject.

7-8. (canceled)

9. The method of claim 6, wherein:
the at least one microbial genera signature for the one or more first subjects comprises a signed microbial genera signature and/or an absolute valued microbial genera signature.

10-13. (canceled)

14. A method of determining T-cell microenvironment reaction in a cancer subject, comprising:
(i) receiving a single cell RNA sequencing dataset for T-cells from the subject;
(ii) determining the expression level of one or more of the genes of Table 2 in the T-cells; and
(iii) comparing the expression level of the one or more genes of Table 2 in the T-cells to a control using a random forest model,
thereby classifying the individual T-cells as infection microenvironment reactive or tumor microenvironment reactive.

15. The method of claim 6, wherein selecting microbial genera comprises removing microbial genera from the differentiating microbial genera signature that are not present with a p value of less than 0.05.

16. The method of claim 6, wherein the at least one microbial genera signature comprises gene expression datapoints.

17. The method of claim 6, wherein the at least one microbial genera signature comprises genes ranked based on level of differentiation.

18. The method of claim 6, wherein the datapoints are normalized before identifying differential microbial genera in the datasets.

19. The method of claim 6, further comprising validating the clinical significance, non-randomness, and/or accuracy of the differentiating microbial genera signature.

20. The method of claim 19, wherein validating the clinical significance comprises:
receiving single cell RNA sequencing datasets for a group of validation subjects, wherein whether the subject has the cancer and/or whether the subject has a good or poor survival outcome is known;
identifying differentially present microbial genera in the datasets, wherein the identifying generates at least one single-sample signature for each validation subject in the group;
determining the presence of microbial genera from the differentiating microbial genera signature in the at least one single-sample signature for each validation subject in the group, wherein the determining generates a microbial genera signature for each validation subject;
clustering the validation subjects in the group into cancer status clusters and/or survival outcome clusters based on the microbial genera signature for each validation subject; and
comparing the cancer status clusters with the known cancer status for the validation subjects in the group; and/or
comparing the survival outcome clusters with the known survival outcome for the validation subjects in the group.

21-24. (canceled)

25. The method of claim 20, wherein generating at least one single-sample signature for each validation subject in the group comprises generating a signed single-sample signature and/or an absolute valued single-sample signature.

26. The method of claim 6, further comprising:
(iv) receiving a single cell RNA sequencing dataset for a subject at risk of having a cancer;
(v) identifying a set of microbial genera in the dataset for the subject at risk of having the cancer; and
(vi) comparing the differentiating microbial genera signature to the set of microbial genera identified in the dataset from the subject at risk of having the cancer;
thereby determining whether the subject at risk of having the cancer has the cancer
wherein the first subject is a cancer subject, and the second subject is a non-cancer subject.

27. The method of claim 6, further comprising:
(iv) receiving a single cell RNA sequencing dataset for a cancer subject;
(v) identifying a set of microbial genera in the dataset for the cancer subject; and
(vi) comparing the differentiating microbial genera signature to the set of microbial genera identified in the dataset from the cancer subject;
thereby predicting whether the cancer subject will have a good survival outcome or a poor survival outcome;
wherein the first subject is a good survival outcome cancer subject, and the second subject is a poor survival outcome cancer subject.

28. (canceled)

29. The method of claim 1, wherein the detecting microbial or viral nucleic acids in the dataset comprises:
(i) mapping reads from the single cell RNA sequencing dataset to microbial and/or viral genomes using a metagenomics classifier, thereby assigning a genus and/or species identity to each read in the dataset;
(ii) for each genus and/or species identified in (i):
(a) comparing the number of reads assigned and the number of minimizers assigned;
(b) comparing the number of minimizers assigned and the number of unique minimizers assigned; and
(c) comparing the number of reads assigned and the number of unique minimizers assigned; and
(iii) classifying the genus and/or species as a true positive result when a correlation value for each comparison in (ii)(a)-(ii)(c) is positive, and when a number of reads detected for the species is greater in the single cell RNA sequencing dataset as compared to a control.

30. The method of claim 29, wherein the correlation value for each comparison is greater than 0.5, 0.7, 0.9, or 0.95.

31-35. (canceled)

36. A microbe or a virus identification system, comprising:
one or more processors; and
memory coupled to the one or more processors, wherein the memory comprises computer-executable instructions causing the one or more processors to perform a process comprising:
(i) receiving a single cell RNA sequencing dataset for the sample;
(ii) detecting microbial or viral nucleic acids in the dataset; and
(iii) identifying the microbe or the virus in the sample when a microbial or viral nucleic acid indicative of the presence of the microbe or virus is detected in the dataset.

37. One or more computer-readable media having encoded thereon computer-executable instructions that, when executed, cause a computing system to perform a microbe or a virus identification method comprising:
(i) receiving a single cell RNA sequencing dataset for the sample;
(ii) detecting microbial or viral nucleic acids in the dataset; and
(iii) identifying the microbe or the virus in the sample when a microbial or viral nucleic acid indicative of the presence of the microbe or the virus is detected in the dataset.

38. The system of claim 36,
wherein the microbe or the virus is a causative agent of an infectious disease.

39. The Oone or more computer-readable media of claim 37,
wherein the microbe or the virus is a causative agent of an infectious disease.

40. The system of claim 36, wherein the detecting microbial or viral nucleic acids in the dataset further comprises:
(i) mapping reads from the single cell RNA sequencing dataset to microbial and/or viral genomes using a metagenomics classifier, thereby assigning a genus and/or species identity to each read in the dataset;
(ii) for each genus and/or species identified in (i):
(a) comparing the number of reads assigned and the number of minimizers assigned; (b) comparing the number of minimizers assigned and the number of unique minimizers assigned; and
(c) comparing the number of reads assigned and the number of unique minimizers assigned; and
(iii) classifying the genus and/or species as a true positive result when a correlation value for each comparison in (ii)(a)-(ii)(c) is positive, and when a number of reads detected for the species is greater in the single cell RNA sequencing dataset as compared to a control.

41-53. (canceled)
